(19) United States
(12) Patent Application Publication
(10) Pub. No.: US 2003/0059768 A1
(43) Pub. Date: Mar. 27, 2003

(54) Novel polypeptides and nucleic acids encoding same

(76) Inventors: Corine Vernet, North Branford, CT (US); Elma Fernandes, Branford, CT (US); Richard A. Shimkets, West Haven, CT (US); John MacDougall, Hamden, CT (US); Steven K. Spaderna, Berlin, CT (US)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 09/789,390

(22) Filed: Feb. 23, 2001

Related U.S. Application Data
(60) Provisional application No. 60/185,548, filed on Feb. 28, 2000.
(60) Provisional application No. 60/199,957, filed on Apr. 27, 2000.
(60) Provisional application No. 60/184,951, filed on Feb. 25, 2000.
(60) Provisional application No. 60/185,967, filed on Mar. 1, 2000.
(60) Provisional application No. 60/197,723, filed on Apr. 18, 2000.

(51) Int. Cl. C12Q 1/68; C07H 21/04; C12N 9/00; C12P 21/02; C12N 5/06

(52) U.S. Cl. 435/6; 435/69.1; 435/183; 435/325; 435/320.1; 536/23.2

(57) ABSTRACT

The present invention provides novel isolated NOVX polynucleotides and polypeptides encoded by the NOVX polynucleotides. Also provided are the antibodies that immunospecifically bind to a NOVX polypeptide or any derivative, variant, mutant or fragment of the NOVX polypeptide, polynucleotide or antibody. The invention additionally provides methods in which the NOVX polypeptide, polynucleotide and antibody are utilized in the detection and treatment of a broad range of pathological states, as well as to other uses.

### RELATED APPLICATIONS

This application claims priority to U.S. Ser. No. 60/185,548, filed Feb. 28, 2000; U.S. Ser. No. 60/199,957, filed Apr. 27, 2000; U.S. Ser. No. 60/184,951, filed Feb. 25, 2000; U.S. Ser. No. 60/185,967, filed Mar. 1, 2000; and U.S. Ser. No. 60/197,723, filed Jun. 26, 2000. The contents of these applications are incorporated by reference in their entireties.

### BACKGROUND OF THE INVENTION

The invention relates generally to nucleic acids and polypeptides.

### SUMMARY OF THE INVENTION

The present invention is based, in part, upon the discovery of novel human nucleic acid sequences encoding polypeptides. The NOV-X nucleic acids, polynucleotides, proteins, and polypeptides or fragments thereof described herein collectively include NOV-1, NOV-2a, and NOV-2b, which are novel KIAA1233-like polypeptides; NOV-3a, NOV-3b, NOV-3c, and NOV-3d, which are novel STE20-like polypeptides; NOV-4a, NOV-4b, NOV-4c, NOV-4d, and NOV-4e, which are novel trypsin inhibitor-like polypeptides.

In one aspect, the invention includes an isolated NOV-X nucleic acid molecule which includes a nucleotide sequence encoding a polypeptide that includes the amino acid sequence of SEQ ID NO: 2, 4, 5, 7, 9, 11, 13, 15, 17, 19, 21, or 23. For example, in various embodiments, the nucleic acid can include a nucleotide sequence that includes SEQ ID NO: 1, 3, 6, 8, 10, 12, 14, 16, 18, 20, 22, or 57. Alternatively, the encoded NOV-X polypeptide may have a variant amino acid sequence, e.g., have an identity or similarity less than 100% to the disclosed amino acid sequences, as described herein.

The invention also includes an isolated polypeptide that includes the amino acid sequence of SEQ ID NO: 2, 4, 5, 7, 9, 11, 13, 15, 17, 19, 21, or 23, or a fragment having at least 6 amino acids of these amino acid sequences. Also included is a naturally occurring polypeptide variant of a NOV-X polypeptide, wherein the polypeptide is encoded by a nucleic acid molecule which hybridizes under stringent conditions to a nucleic acid molecule consisting of a NOV-X nucleic acid molecule.

Also included in the invention is an antibody that selectively binds to a NOV-X polypeptide. The antibody is preferably a monoclonal antibody, and most preferably is a human antibody. Such antibodies are useful, for example, in the treatment of a pathological state in a subject wherein the treatment includes administering the antibody to the subject.

The invention further includes a method for producing a NOV-X polypeptide by culturing a host cell expressing one of the herein described NOV-X nucleic acids under conditions in which the nucleic acid molecule is expressed.

The invention also includes methods for detecting the presence of a NOV-X polypeptide or nucleic acid in a sample from a mammal, e.g., a human, by contacting a sample from the mammal with an antibody which selectively binds to one of the herein described polypeptides, and detecting the formation of reaction complexes including the antibody and the polypeptide in the sample. Detecting the formation of complexes in the sample indicates the presence of the polypeptide in the sample.

The invention further includes a method for detecting or diagnosing the presence of a disease, e.g., a pathological condition, associated with altered levels of a polypeptide having an amino acid sequence at least 80% identical to a NOV-X polypeptide in a sample. The method includes measuring the level of the polypeptide in a biological sample from the mammalian subject, e.g., a human, and comparing the level detected to a level of the polypeptide present in normal subjects, or in the same subject at a different time, e.g., prior to onset of a condition. An increase or decrease in the level of the polypeptide as compared to normal levels indicates a disease condition.

Also included in the invention is a method of detecting the presence of a NOV-X nucleic acid molecule in a sample from a mammal, e.g., a human. The method includes contacting the sample with a nucleic acid probe or primer which selectively hybridizes to the nucleic acid molecule and determining whether the nucleic acid probe or primer binds to a nucleic acid molecule in the sample. Binding of the nucleic acid probe or primer indicates the nucleic acid molecule is present in the sample.

The invention further includes a method for detecting or diagnosing the presence of a disease associated with altered levels of a NOV-X nucleic acid in a sample from a mammal, e.g. a human. The method includes measuring the level of the nucleic acid in a biological sample from the mammalian subject and comparing the level detected to a level of the nucleic acid present in normal subjects, or in the same subject at a different time. An increase or decrease in the level of the nucleic acid as compared to normal levels indicates a disease condition.

The invention also includes a method of treating a pathological state in a mammal, e.g,. a human, by administering to the subject a NOV-X polypeptide to the subject in an amount sufficient to alleviate the pathological condition. The polypeptide has an amino acid sequence at least 80% identical to a NOV-X polypeptide.

Alternatively, the mammal may be treated by administering an antibody as herein described in an amount sufficient to alleviate the pathological condition.

Pathological states for which the methods of treatment of the invention are envisioned include hematopoietic, immunological, tumor, cancer, neurodegenerative (e.g. Alzheimer's and Parkinson's disease) and fertility disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention is based, in part, upon the discovery of novel human nucleic acid sequences and of polypeptides encoded by these nucleic acids. The nucleic acids have been named “NOV 1-4”, or collectively, “NOV-X”. Representative NOV-X sequences, and representative examples of uses of these sequences, are briefly discussed below.

Table 1 provides a summary of the NOV-X nucleic acids, their encoded polypeptides and homology.

TABLE 1
Sequences and Corresponding SEQ ID Numbers

| | | SEQ ID | SEQ ID | |
|---|---|---|---|---|
| NOVX | | NO | NO | |
| Assign- | Internal | (nucleic | (poly- | |
| ment | Identification | acid) | peptide) | Homology |
| 1 | 10132038.0.67 | 1 | 2 | KIAA1233 protein |
| 2a | 10132038.0.139 | 3 | 4 | KIAA1233 protein |
| 2b | 10132038.0.136 | 3 | 5 | KIAA1233 protein |
| 3a | 18552586_EXT1 | 6 | 7 | STE20 protein kinase |
| 3b | 18552586_EXT2 | 8 | 9 | STE20 protein kinase |
| 3c | 18552586_EXT3 | 10 | 11 | STE20 protein kinase |
| 3d | 18552586_EXT4 | 12 | 13 | STE20 protein kinase |
| 4a | 10093872.0.107 | 14 | 15 | Trypsin inhibitor |
| 4b | 10093872.1 | 16 | 17 | Trypsin inhibitor |
| 4c | 10093872.0.38 | 18 | 19 | Trypsin inhibitor |
| 4d | 10093872.2 | 20 | 21 | Trypsin inhibitor |
| 4e | 10093872.3 | 22 | 23 | Trypsin inhibitor |

NOV-1: A Novel KIAA1233-Like Polypeptide

A NOV-1 sequence according to the invention is a nucleotide sequence encoding a polypeptide related to KIAA1233 proteins, which bear sequence similarity to lacunin, thrombospondins, proteinases, semaphorins, ADAM-TS, and properdin family members. This invention maps to Unigene cluster Hs.18705. This cluster has been mapped to Chromosome 15 Marker stSG35204, Interval D15S115-D15S152. By integrating information from the Online Mendelian Inheritance in Man (OMIM), this region is identified as 15q22-qter. Therefore, the chromosomal location of the invention is Chromosome 15 Marker stSG35204, Interval D15S115-D15S152, which corresponds to 15q22-qter.

The nucleic acid of the invention, NOV-1, encoding a KIAA1233-like protein originating from chromosome 15, is shown in TABLE 2. The disclosed nucleic acid (SEQ ID NO: 1) is a full-length clone of 1281 nucleotides and contains an open reading framne (ORF) that begins with an ATG initiation codon at nucleotide 416 and ends with a TAA stop codon at nucleotides 4259. A representative ORF encodes a 1281 amino acid polypeptide (SEQ ID NO: 2). The initiation and stop codons of SEQ ID NO: 1 are shown in bold font. Putative untranslated regions are upstream of the initiation codon and downstream of the stop codon in SEQ ID NO: 1.

TABLE 2

| | |
|---|---|
| TAATAGAGACCTTTCAAAGGACAAATTCTGTGAAATAAAGTGGTTTTCTGAAGAGCCTAC | (SEQ ID NO:1) |
| TAATAGGACAGTGTGTTAATATCACTAATAAGAGAGTAATGATTATAAAAAGGAATAAAT | |
| TTATTGAAATTGCAAGATACTTTTCTCCTTTGATTAATATACTGCTAGTTTAGTTTTCTA | |
| CATTTTCAAATAGAACTGGGGAATTTGTGTCGTAGATATTCTTGACAACTAAAGAGATGG | |
| TGGCTGAATTTTTGGGAATGGTTGATAACACTTGATATTTTTAGTTTCCAATTTGGAAGA | |
| GCTCTGTCTCTTGGGATGTCAAATATTATATTCGTCAATTAATGAATGTGTTAATTTATT | |
| ATAGAAATGATATTCTCACAATGATTTCATTTGTAGTGATGGATTTAAAGAGATAATGCC | |
| CTATGACCACTTCCAACCTCTTCCTCGCTGGGAACATAATCCTTGGACTGCATGTTCCGT | |
| GTCCTGTGGAGGAGGGATTCAGAGACGGAGCTTTGTGTGTGTAGAGGTAACCATGCATGG | |
| AGAGATATTGCAGGTGGAAGAATGGAAGTGCATGTACGCACCCAAACCCAAGGTTATGCA | |
| AACTTGTAATCTGTTTGATTGCCCCAAGTGGATTGCCATGGAGTGGTCTCAGTGCACAGT | |
| GACTTGTGGCCGAGGGTTACGGTACCGGGTTGTTCTGTGTATTAACCACCGCGGAGAGCA | |
| TGTTGGGGGCTGCAATCCACAACTGAAGTTACACATCAAAGAAGAATGTGTCATTCCCAT | |
| CCCGTGTTATAAACCAAAAGAAAAAAGTCCAGTGGAAGCAAAATTGCCTTGGCTGAAACA | |
| AGCACAAGAACTAGAAGAGACCAGAATAGCAACAGAAGAACCAACGTTCATTCCAGAACC | |
| CTGGTCAGCCTGCAGTACCACGTGTGGGCCGGGTGTGCAGGTCCGTGAGGTGAAGTGCCG | |
| TGTGCTCCTCACATTCACGCAGACTGAGACTGAGCTGCCCGAGGAAGAGTGTGAAGGCCC | |
| CAAGCTGCCCACCGAACGGCCCTGCCTCCTGGAACGATGTGATGAGAGCCCGGCCTCCCG | |
| AGAGCTAGACATCCCTCTCCCTGAGGACAGTGAGACGACTTACGACTGGGAGTACGCTGG | |
| GTTCACCCCTTGCACAGCAACATGCGTGGGAGGCCATCAAGAAGCCATAGCAGTGTGCTT | |
| ACATATCCAGACCCAGCAGACAGTCAATGACAGCTTGTGTGATATGGTCCACCGTCCTCC | |
| AGCCATGAGCCAGGCCTGTAACACAGAGCCCTGTCCCCCCAGGTGGCATGTGGGCTCTTG | |
| GGGGCCCTGCTCAGCTACCTGTGGAGTTGGAATTCAGACCCGAGATGTGTACTGCCTGCA | |
| CCCAGGGGAGACCCCTGCCCCTCCTGAGGAGTGCCGAGATGAAAAGCCCCATGCTTTACA | |
| AGCATGCAATCAGTTTGACTGCCCTCCTGGCTGGCACATTGAAGAATGGCAGCAGTGTTC | |
| CAGGACTTGTGGCGGGGGAACTCAGAACAGAAGAGTCACCTGTCGGCAGCTGCTAACGGA | |
| TGGCAGCTTTTTGAATCTCTCAGATGAATTGTGCCAAGGACCCAAGGCATCGTCTCACAA | |
| GTCCTGTGCCAGGACAGACTGTCCTCCACATTTAGCTGTGGGAGACTGGTCGAAGTGTTC | |
| TGTCAGTTGTGGTGTTGGAATCCAGAGAAGAAAGCAGGTGTGTCAAAGGCTGGCAGCCAA | |
| AGGTCGGCGCATCCCCCTCAGTGAGATGATGTGCAGGGATCTACCAGGGCTCCCTCTTGT | |
| AAGATCTTGCCAGATGCCTGAGTGCAGTAAAATCAAATCAGAGATGAAGACAAAACTTGG | |
| TGAGCAGGGTCCGCAGATCCTCAGTGTCCAGAGAGTCTACATTCAGACAAGGGAAGAGAA | |
| GCGTATTAACCTGACCATTGGTAGCAGAGCCTATTTGCTGCCCAACACATCCGTGATTAT | |
| TAAGTGCCCAGTGCGACGATTCCAGAAATCTCTGATCCAGTGGGAGAAGGATGGCCGTTG | |
| CCTGCAGAACTCCAAACGGCTTGGCATCACCAAGTCAGGCTCACTAAAAATCCACGGTCT | |
| TGCTGCCCCCGACATCGGCGTGTACCGGTGCATTGCAGGCTCTGCACAGGAAACAGTTGT | |
| GCTCAAGCTCATTGGTACTGACAACCGGCTCATCGCACGCCCAGCCCTCAGGGAGCCTAT | |
| GAGGGAATATCCTGGGATGGACCACAGCGAAGCCAATAGTTTGGGAGTCACATGGCACAA | |
| AATGAGGCAAATGTGGAATAACAAAAATGACCTTTATCTGGATGATGACCACATTAGTAA | |
| CCAGCCTTTCTTGAGAGCTCTGTTAGGCCACTGCAGCAATTCTGCAGGAAGCACCAACTC | |
| CTGGGAGTTGAAGAATAAGCAGTTTGAAGCAGCAGTTAAACAAGGAGCATATAGCATGGA | |
| TACAGCCCAGTTTGATGAGCTGATAAGAAACATGAGTCAGCTCATGGAAACCGGAGAGGT | |
| CAGCGATGATCTTGCGTCCCAGCTGATATATCAGCTGGTGGCCGAATTAGCCAAGGCACA | |
| GCCAACACACATGCAGTGGCGGGGCATCCAGGAAGAGACACCTCCTGCTGCTCAGCTCAG | |
| AGGGGAAACAGGGAGTGTGTCCCAAAGCTCGCATGCAAAAAACTCAGGCAAGCTGACATT | |
| CAAGCCGAAAGGACCTGTTCTCATGAGGCAAAGCCAACCTCCCTCAATTTCATTTAATAA | |
| AACAATAAATTCCAGGATTGGAAATACAGTATACATTACAAAAAGGACAGAGGTCATCAA | |
| TATACTGTGTGACCTTATTACCCCCAGTGAGGCCACATATACATGGACCAAGGATGGAAC | |
| CTTGTTACAGCCCTCAGTAAAAATAATTTTGGATGGAACTGGGAAGATACAGATACAGAA | |
| TCCTACAAGGAAAGAACAAGGCATATATGAATGTTCTGTAGCTAATCATCTTGGTTCAGA | |
| TGTGGAAAGTTCTTCTGTGCTGTATGCAGAGGCACCTGTCATCTTGTCTGTTGAAAGAAA | |
| TATCACCAAACCAGAGCACAACCATCTGTCTGTTGTGGTTGGAGGCATCGTGGAGGCAGC | |
| CCTTGGAGCAAACGTGACAATCCGATGTCCTGTAAAAGGTGTCCCTCAGCCTAATATAAC | |
| TTGGTTGAAGAGAGGAGGATCTCTGAGTGGCAATGTTTCCTTGCTTTTCAATGGATCCCT | |
| GTTGTTGCAGAATGTTTCCCTTGAAAATGAAGGAACCTACGTCTGCATAGCCACCAATGC | |
| TCTTGGAAAGGCAGTGGCAACATCTGTACTCCACTTGCTGGAACGAAGATGGCCAGAGAG | |
| TAGAATCGTATTTCTGCAAGGACATAAAAAGTACATTCTCCAGGCAACCAACACTAGAAC | |
| CAACAGCAATGACCCAACAGGAGAACCCCCGCCTCAAGAGCCTTTTTGGGAGCCTGGTAA | |
| CTGGTCACATTGTTCTGCCACCTGTGGTCATTTGGGAGCCCGCATTCAGAGACCCCAGTG | |
| TGTGATGGCCAATGGGCAGGAAGTGAGTGAGGCCCTGTGTGATCACCTCCAGAAGCCACT | |
| GGCTGGGTTTGAGCCCTGTAACATCCGGGACTGCCCAGCGAGGTGGTTCACAAGTGTGTG | |
| GTCACAGTGCTCTGTGTCTTGCGGTGAAGGATACCACAGTCGGCAGGTGACGTGCAAGCG | |
| GACAAAAGCCAATGGAACTGTGCAGGTGGTGTCTCCAAGAGCATGTGCCCCTAAAGACCG | |
| GCCTCTGGGAAGAAAACCATGTTTTGGTCATCCATGTGTTCAGTGGGAACCAGGGAACCG | |
| GTGTCCTGGACGTTGCATGGGCCGTGCTGTGAGGATGCAGCAGCGTCACACAGCTTGTCA | |
| ACACAACAGCTCTGACTCCAACTGTGATGACAGAAAGAGACCCACCTTAAGAAGGAACTG | |
| CACATCAGGGGCCTGTGATGTGTGTTGGCACACAGGCCCTTGGAAGCCCTGTACAGCAGC | |
| CTGTGGCAGGGGTTTCCAGTCTCGGAAAGTCGACTGTATCCACACAAGGAGTTGCAAACC | |
| TGTGGCCAAGAGACACTGTGTACAGAAAAAGAAACCAATTTCCTGGCGGCACTGTCTTGG | |
| GCCCTCCTGTGATAGAGACTGCACAGACACAACTCACTACTGTATGTTTGTAAAACATCT | |
| TAATTTGTGTTCTCTAGACCGCTACAAACAAAGGTGCTGCCAGTCATGTCAAGAGGGATA | |
| AACCTTTGGAGGGGTCATGATGCTGCTGTGAAGATAAAAGTAGAATATAAAAGCTCTTTT | |
| CCCCATGTCGCTGATTCAAAAACATGTATTTCTTAAAAGACTAGATTCTATGGATCAAAC | |
| AGAGGTTGATGCAAAAACACCACTGTTAAGGTGTAAAGTGAAATTTTCCAATGGTAGTTT | |
| TATATTCCAATTTTTTAAAATGATGTATTCAAGGATGAACAAAATACTATAGCATGCATG | |
| CCACTGCACTTGGGACCTCATCATGTCAGTTGAATCGAGAAATCACCAAGATTATGAGTG | |
| CATCCTCACGTGCTGCCTCTTTCCTGTGATATGTAGACTAGCACAGAGTGGTACATCCTA | |
| AAAACTTGGGAAACACAGCAACCCATGACTTCCTCTTCTCTCAAGTTGCAGGTTTTCAAC | |
| AGTTTTATAAGGTATTTGCATTTTAGAAGCTCTGGCCAGTAGTTGTTAAGATGTTGGCAT | |
| TAATGGCATTTTCATAGATCCTTGGTTTAGTCTGTGAAAAAGAAACCATCTCTCTGGATA | |
| GGCTGTCACACTGACTGACCTAAGGGTTCATGGAAGCATGGCATCTTGTCCTTGCTTTTA | |
| GAACACCCATGGAAGAAAACACAGAGTAGATATTGCTGTCATTTATACAACTACAGAAAT | |
| TTATCTATGACCTAATGAGGCATCTCGGAAGTCAAAGAAGAGGGAAAGTTAACCTTTTCT | |
| ACTGATTTCGTAGTATATTCAGAGCTTTCTTTTAAGAGCTGTGAATGAAACTTTTTCTAA | |
| GCACTATTCTATTGCACACAAACAGAAAACCAAAGCCTTATTAGACCTAATTTATGCATA | |
| AAGTAGTATTCCTGAGAACTTTATTTTGGAAAATTTATAAGAAAGTAATCCAAATAAGAA | |
| ACACGATAGTTGAAAATAATTTTTATAGTAAATAATTGTTTTGGGCTGATTTTTCAGTAA | |
| ATCCAAAGTGACTTAGGTTAGAAGTTACACTAAGGACCAGGGGTTGGAATCAGAATTTAG | |
| TTTAAGATTTGAGGAAAAGGGTAAGGGTTAGTTTCAGTTTTAGGATTAGAGCTAGAATTG | |
| GGTTAGGTGAGAAAGAAAGTTAAGGTTAAGGCTAGAGTTGTCTTTAAGGGTTAGGGTTAG | |
| GACCAGGTTAGGTCAGGGTTGGATTGGGTTTAGATTGGGGCCAGTGCTGGTGTTAGTGAT | |
| AGTGTCAGGATGGAGGTTAGGTTTGGAGTAAGCGTTGTTGCTGAAGTGAGTTCAGGCTAG | |
| CATTAAATTGTAAGTTCTGAAGCTGATTTGGTTATGGGGTCTTTCCCCTGTATACTACCA | |
| GTTGTGTCTTTAGATGGCACACAAGTCCAAATAAGTGGTCATACTTCTTTATTCAGGGTC | |
| TCAGCTGCCTGTACACCTGCTGCCTACATCTTCTTGGCAACAAAGTTACCTGCCACAGGC | |
| TCTGCTGAGCCTAGTTCCTGGTCAGTAATAACTGAACAGTGCATTTTGGCTTTGGATGTG | |
| TCTGTGGACAAGCTTGCTGAGTTTCTCTACCATATTCTGAGCACACGGTCTCTTTTGTTC | |
| TAATTTCAGCTTCACTGACACTGGGTTGAGCACTACTGTATGTGGAGGGTTTGGTGATTG | |
| GGAATGGATGGGGGACAGTGAGGAGGACACACCAGCCCATTAGTTGTTAATCATCAATCA | |
| CATCTGATTGTTGAAGGTTATTAAATTAAAAGAAAGATCATTTGTAACATACTCTTTGTA | |
| TATATTTATTATATGAAAGGTGCAATATTTTATTTTGTACAGTATGTAATAAAGACATGG | |
| GACATATATTTTTCTTATTAACAAAATTTCATATTAAATTGCTTCACTTTGTATTTAAAG | |
| TTAAAAGTTACTATTTTTCATTTGCTATTGTACTTTCATTGTTGTCATTCAATTGACATT | |
| CCTGTGTACTGTATTTTACTACTGTTTTTATAACATGAGAGTTAATGTTTCTGTTTCATG | |
| ATCCTTATGTAATTCAGAAATAAATTTACTTTGATTATTCAGTGGCATCCTTAT | |
| MPYDHFQPLPRWEHNPWTACSVSCGGGIQRRSFVCVEESMHGEILQVEEWKCMYAPKPKVMQTCNLFDCPKWIAMEW | (SEQ ID NO:2) |
| SQCTVTCGRGLRYRVVLCINHRGEHVGGCNPQLKLHIKEECVIPIPCYKPKEKSPVEAKLPWLKQAQELEETRIATE | |
| EPTFIPEPWSACSTTCGPGVQVREVKCRVLLTFTQTETELPEEECEGPKLPTERPCLLEACDESPASRELDIPLPED | |
| SETTYDWEYAGFTPCTATCVGGHQEAIAVCLHIQTQQTVNDSLCDMVHRPPAMSQACNTEPCPPRWHVGSWGPCSAT | |
| CGVGIQTRDVYCLHPGETPAPPEECRDEKPHALQACNQFDCPPGWHIEEWQQCSRTCGGGTQNRRVTCRQLLTDGSF | |
| LNLSDELCQGPKASSHKSCARTDCPPHLAVGDWSKCSVSCGVGIQRRKQVCQRLAAKGRRIPLSEMMCRDLPGLPLV | |
| RSCQMPECSKIKSEMKTKLGEQGPQILSVQRVYIQTREEKRINLTIGSRAYLLPNTSVIIKCPVRRFQKSLIQWEKD | |
| GRCLQNSKRLGITKSGSLKIHGLAAPDIGVYRCIAGSAQETVVLKLIGTDNRLIARPALREPMREYPGMDHSEANSL | |
| GVTWHKMRQMWNNKNDLYLDDDHISNQPFLRALLGHCSNSAGSTNSWELKNKQFEAAVKQGAYSMDTAQFDELIRNM | |
| SQLMETGEVSDDLASQLIYQLVAELAKAQPTHMQWRGIQEETPPAAQLRGETGSVSQSSHAKNSGKLTFKPKGPVLM | |
| RQSQPPSISFNKTINSRIGNTVYITKRTEVINILCDLITPSEATYTWTKDGTLLQPSVKIILDGTGKIQIQNPTRKE | |
| QGIYECSVANHLGSDVESSSVLYAEAPVILSVERNITKPEHNHLSVVVGGIVEAALGANVTIRCPVKGVPQPNITWL | |
| KRGGSLSGNVSLLFNGSLLLQNVSLENEGTYVCIATNALGKAVATSVLHLLERRWPESRIVFLQGHKKYILQATNTR | |
| TNSNDPTGEPPPQEPFWEPGNWSHCSATCGHLGARIQRPQCVMANGQEVSEALCDHLQKPLAGFEPCNIRDCPARWF | |
| TSVWSQCSVSCGEGYHSRQVTCKRTKANGTVQVVSPRACAPKDRPLGRKPCFGHPCVQWEPGNRCPGRCMGRAVRMQ | |
| QRHTACQHNSSDSNCDDRKRPTLRRNCTSGACDVCWHTGPWKPCTAACGRGFQSRKVDCIHTRSCKPVAKRHCVQKK | |
| KPISWRHCLGPSCDRDCTDTTHYCMFVKHLNLCSLDRYKQRCCQSCQEG | |

In a search of sequence databases, it was found, for example, that the disclosed NOV-1 nucleotide sequence has 5106 of 5107 bases (99%) identical to a human mRNA for a KIAA1233 protein (SECR) (GenBank Accession No: ABO33059), as shown in Table 3. In all sequence alignments, identical residues are depicted as “|”. As indicated by the “Expect” value, the probability of this alignment occurring by chance alone is 0.0, the lowest probability.

Furthermore, the encoded amino acid sequence has 1023 of 1023 amino acid residues (100%) identical to, and 1023 of 1023 residues (100%) positive with, a 1023 amino acid residue human KIAA1233 protein (GenBank Accession No: BAA86547), as shown in Table 4. As indicated by the “Expect” value, the probability of this alignment occuring by chance alone is 0, the lowest probability.

TABLE 3
Score = 1.012e+04 bits (5103), Expect = 0.0
Identities = 5106/5107 (99%)
Strand = Plus/Plus

| | | | | |
|---|---|---|---|---|
| NOV1: | 1188 | tagcagtgtgcttacatatccagacccagcagacagtcaatgacagcttgtgtgatatgg | 1247 | (SEQ ID NO:58) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1 | tagcagtgtgcttacatatccagacccagcagacagtcaatgacagcttgtgtgatatgg | 60 | (SEQ ID NO:24) |
| NOV1: | 1248 | tccaccgtcctccagccatgagccaggcctgtaacacagagccctgtccccccaggtggc | 1307 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 61 | tccaccgtcctccagccatgagccaggcctgtaacacagagccctgtccccccaggtggc | 120 | |
| NOV1: | 1308 | atgtgggctcttgggggccctgctcagctacctgtggagttggaattcagacccgagatg | 1367 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 121 | atgtgggctcttgggggccctgctcagctacctgtggagttggaattcagacccgagatg | 180 | |
| NOV1: | 1368 | tgtactgcctgcacccaggggagacccctgcccctcctgaggagtgccgagatgaaaagc | 1427 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 181 | tgtactgcctgcacccaggggagacccctgcccctcctgaggagtgccgagatgaaaagc | 240 | |
| NOV1: | 1428 | cccatgctttacaagcatgcaatcagtttgactgccctcctggctggcacattgaagaat | 1487 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 241 | cccatgctttacaagcatgcaatcagtttgactgccctcctggctggcacattgaagaat | 300 | |
| NOV1: | 1488 | ggcagcagtgttccaggacttgtggcgggggaactcagaacagaagagtcacctgtcggc | 1547 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 301 | ggcagcagtgttccaggacttgtggcgggggaactcagaacagaagagtcacctgtcggc | 360 | |
| NOV1: | 1548 | agctgctaacggatggcagctttttgaatctctcagatgaattgtgccaaggacccaagg | 1607 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 361 | agctgctaacggatggcagctttttgaatctctcagatgaattgtgccaaggacccaagg | 420 | |
| NOV1: | 1608 | catcgtctcacaagtcctgtgccaggacagactgtcctccacatttagctgtgggagact | 1667 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 421 | catcgtctcacaagtcctgtgccaggacagactgtcctccacatttagctgtgggagact | 480 | |
| NOV1: | 1668 | ggtcgaagtgttctgtcagttgtggtgttggaatccagagaagaaagcaggtgtgtcaaa | 1727 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 481 | ggtcgaagtgttctgtcagttgtggtgttggaatccagagaagaaagcaggtgtgtcaaa | 540 | |
| NOV1: | 1728 | ggctggcagccaaaggtcggcgcatccccctcagtgagatgatgtgcagggatctaccag | 1787 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 541 | ggctggcagccaaaggtcggcgcatccccctcagtgagatgatgtgcagggatctaccag | 600 | |
| NOV1: | 1788 | ggctccctcttgtaagatcttgccagatgcctgagtgcagtaaaatcaaatcagagatga | 1847 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 601 | ggctccctcttgtaagatcttgccagatgcctgagtgcagtaaaatcaaatcagagatga | 660 | |
| NOV1: | 1848 | agacaaaacttggtgagcagggtccgcagatcctcagtgtccagagagtctacattcaga | 1907 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 661 | agacaaaacttggtgagcagggtccgcagatcctcagtgtccagagagtctacattcaga | 720 | |
| NOV1: | 1908 | caagggaagagaagcgtattaacctgaccattggtagcagagcctatttgctgcccaaca | 1967 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 721 | caagggaagagaagcgtattaacctgaccattggtagcagagcctatttgctgcccaaca | 780 | |
| NOV1: | 1968 | catccgtgattattaagtgcccagtgcgacgattccagaaatctctgatccagtgggaga | 2027 | |
| | | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\| | | |
| SECR: | 781 | catccgtgattattaagtgccccgtgcgacgattccagaaatctctgatccagtgggaga | 840 | |
| NOV1: | 2028 | aggatggccgttgcctgcagaactccaaacggcttggcatcaccaagtcaggctcactaa | 2087 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 841 | aggatggccgttgcctgcagaactccaaacggcttggcatcaccaagtcaggctcactaa | 900 | |
| NOV1: | 2088 | aaatccacggtcttgctgcccccgacatcggcgtgtaccggtgcattgcaggctctgcac | 2147 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 901 | aaatccacggtcttgctgcccccgacatcggcgtgtaccggtgcattgcaggctctgcac | 960 | |
| NOV1: | 2148 | aggaaacagttgtgctcaagctcattggtactgacaaccggctcatcgcacgcccagccc | 2207 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 961 | aggaaacagttgtgctcaagctcattggtactgacaaccggctcatcgcacgcccagccc | 1020 | |
| NOV1: | 2208 | tcagggagcctatgagggaatatcctgggatggaccacagcgaagccaatagtttgggag | 2267 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1021 | tcagggagcctatgagggaatatcctgggatggaccacagogaagccaatagtttgggag | 1080 | |
| NOV1: | 2268 | tcacatggcacaaaatgaggcaaatgtggaataacaaaaatgacctttatctggatgatg | 2327 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1081 | tcacatggcacaaaatgaggcaaatgtggaataacaaaaatgacctttatctggatgatg | 1140 | |
| NOV1: | 2328 | accacattagtaaccagcctttcttgagagctctgttaggccactgcagcaattctgcag | 2387 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1141 | accacattagtaaccagcctttcttgagagctctgttaggccactgcagcaattctgcag | 1200 | |
| NOV1: | 2388 | gaagcaccaactcctgggagttgaagaataagcagtttgaagcagcagttaaacaaggag | 2447 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1201 | gaagcaccaactcctgggagttgaagaataagcagtttgaagcagcagttaaacaaggag | 1260 | |
| NOV1: | 2448 | catatagcatggatacagcccagtttgatgagctgataagaaacatgagtcagctcatgg | 2507 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1261 | catatagcatggatacagcccagtttgatgagctgataagaaacatgagtcagctcatgg | 1320 | |
| N0V1: | 2508 | aaaccggagaggtcagcgatgatcttgcgtcccagctgatatatcagctggtggccgaat | 2567 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1321 | aaaccggagaggtcagcgatgatcttgcgtcccagctgatatatcagctggtggccgaat | 1380 | |
| NOV1: | 2568 | tagccaaggcacagccaacacacatgcagtggcggggcatccaggaagagacacctcctg | 2627 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1381 | tagccaaggcacagccaacacacatgcagtggcggggcatccaggaagagacacctcctg | 1440 | |
| NOV1: | 2628 | ctgctcagctcagaggggaaacagggagtgtgtcccaaagctcgcatgcaaaaaactcag | 2687 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1441 | ctgctcagctcagaggggaaacagggagtgtgtcccaaagctcgcatgcaaaaaactcag | 1500 | |
| NOV1: | 2688 | gcaagctgacattcaagccgaaaggacctgttctcatgaggcaaagccaacctccctcaa | 2747 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1501 | gcaagctgacattcaagccgaaaggacctgttctcatgaggcaaagccaacctccctcaa | 1560 | |
| NOV1: | 2748 | tttcatttaataaaacaataaattccaggattggaaatacagtatacattacaaaaagga | 2807 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1561 | tttcatttaataaaacaataaattccaggattggaaatacagtatacattacaaaaagga | 1620 | |
| NOV1: | 2808 | cagaggtcatcaatatactgtgtgaccttattacccccagtgaggccacatatacatgga | 2867 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1621 | cagaggtcatcaatatactgtgtgaccttattacccccagtgaggccacatatacatgga | 1680 | |
| NOV1: | 2868 | ccaaggatggaaccttgttacagccctcagtaaaaataattttggatggaactgggaaga | 2927 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1681 | ccaaggatggaaccttgttacagccctcagtaaaaataattttggatggaactgggaaga | 1740 | |
| NOV1: | 2928 | tacagatacagaatcctacaaggaaagaacaaggcatatatgaatgttctgtagctaatc | 2987 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1741 | tacagatacagaatcctacaaggaaagaacaaggcatatatgaatgttctgtagctaatc | 1800 | |
| NOV1: | 2988 | atcttggttcagatgtggaaagttcttctgtgctgtatgcagaggcacctgtcatcttgt | 3047 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1801 | atcttggttcagatgtggaaagttcttctgtgctgtatgcagaggcacctgtcatcttgt | 1860 | |
| NOV1: | 3048 | ctgttgaaagaaatatcaccaaaccagagcacaaccatctgtctgttgtggttggaggca | 3107 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1861 | ctgttgaaagaaatatcaccaaaccagagcacaaccatctgtctgttgtggttggaggca | 1920 | |
| NOV1: | 3108 | tcgtggaggcagcccttggagcaaacgtgacaatccgatgtcctgtaaaaggtgtccctc | 3167 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1921 | tcgtggaggcagcccttggagcaaacgtgacaatccgatgtcctgtaaaaggtgtccctc | 1980 | |
| NOV1: | 3168 | agcctaatataacttggttgaagagaggaggatctctgagtggcaatgtttccttgcttt | 3227 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1982 | agcctaatataacttggttgaagagaggaggatctctgagtggcaatgtttccttgcttt | 2040 | |
| NOV1: | 3228 | tcaatggatccctgttgttgcagaatgtttcccttgaaaatgaaggaacctacgtctgca | 3287 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2041 | tcaatggatccctgttgttgcagaatgtttcccttgaaaatgaaggaacctacgtctgca | 2100 | |
| NOV1: | 3288 | tagccaccaatgctcttggaaaggcagtggcaacatctgtactccacttgctggaacgaa | 3347 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2101 | tagccaccaatgctcttggaaaggcagtggcaacatctgtactccacttgctggaacgaa | 2160 | |
| NOV1: | 3348 | gatggccagagagtagaatcgtatttctgcaaggacataaaaagtacattctccaggcaa | 3407 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2161 | gatggccagagagtagaatcgtatttctgcaaggacataaaaagtacattctccaggcaa | 2220 | |
| NOV1: | 3408 | ccaacactagaaccaacagcaatgacccaacaggagaacccccgcctcaagagccttttt | 3467 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2221 | ccaacactagaaccaacagcaatgacccaacaggagaacccccgcctcaagagccttttt | 2280 | |
| NOV1: | 3468 | gggagcctggtaactggtcacattgttctgccacctgtggtcatttgggagcccgcattc | 3527 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2281 | gggagcctggtaactggtcacattgttctgccacctgtggtcatttgggagcccgcattc | 2340 | |
| NOV1: | 3528 | agagaccccagtgtgtgatggccaatgggcaggaagtgagtgaggccctgtgtgatcacc | 3587 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2341 | agagaccccagtgtgtgatggccaatgggcaggaagtgagtgaggccctgtgtgatcacc | 2400 | |
| NOV1: | 3588 | tccagaagccactggctgggtttgagccctgtaacatccgggactgcccagcgaggtggt | 3647 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2401 | tccagaagccactggctgggtttgagccctgtaacatccgggactgcccagcgaggtggt | 2460 | |
| NOV1: | 3648 | tcacaagtgtgtggtcacagtgctctgtgtcttgcggtgaaggataccacagtcggcagg | 3707 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2461 | tcacaagtgtgtggtcacagtgctctgtgtcttgcggtgaaggataccacagtcggcagg | 2520 | |
| NOV1: | 3708 | tgacgtgcaagcggacaaaagccaatggaactgtgcaggtggtgtctccaagagcatgtg | 3767 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2521 | tgacgtgcaagcggacaaaagccaatggaactgtgcaggtggtgtctccaagagcatgtg | 2580 | |
| NOV1: | 3768 | cccctaaagaccggcctctgggaagaaaaccatgttttggtcatccatgtgttcagtggg | 3827 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2581 | cccctaaagaccggcctctgggaagaaaaccatgttttggtcatccatgtgttcagtggg | 2640 | |
| NOV1: | 3828 | aaccagggaaccggtgtcctggacgttgcatgggccgtgctgtgaggatgcagcagcgtc | 3887 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2641 | aaccagggaaccggtgtcctggacgttgcatgggccgtgctgtgaggatgcagcagcgtc | 2700 | |
| NOV1: | 3888 | acacagcttgtcaacacaacagctctgactccaactgtgatgacagaaagagacccacct | 3947 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2701 | acacagcttgtcaacacaacagctctgactccaactgtgatgacagaaagagacccacct | 2760 | |
| NOV1: | 3948 | taagaaggaactgcacatcaggggcctgtgatgtgtgttggcacacaggcccttggaagc | 4007 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2761 | taagaaggaactgcacatcaggggcctgtgatgtgtgttggcacacaggcccttggaagc | 2820 | |
| NOV1: | 4008 | cctgtacagcagcctgtggcaggggtttccagtctcggaaagtcgactgtatccacacaa | 4067 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2821 | cctgtacagcagcctgtggcaggggtttccagtctcggaaagtcgactgtatccacacaa | 2880 | |
| NOV1: | 4068 | ggagttgcaaacctgtggccaagagacactgtgtacagaaaaagaaaccaatttcctggc | 4127 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2881 | ggagttgcaaacctgtggccaagagacactgtgtacagaaaaagaaaccaatttcctggc | 2940 | |
| NOV1: | 4128 | ggcactgtcttgggccctcctgtgatagagactgcacagacacaactcactactgtatgt | 4187 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2941 | ggcactgtcttgggccctcctgtgatagagactgcacagacacaactcactactgtatgt | 3000 | |
| NOV1: | 4188 | ttgtaaaacatcttaatttgtgttctctagaccgctacaaacaaaggtgctgccagtcat | 4247 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3001 | ttgtaaaacatcttaatttgtgttctctagaccgctacaaacaaaggtgctgccagtcat | 3060 | |
| NOV1: | 4248 | gtcaagagggataaacctttggaggggtcatgatgctgctgtgaagataaaagtagaata | 4307 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3061 | gtcaagagggataaacctttggaggggtcatgatgctgctgtgaagataaaagtagaata | 3120 | |
| NOV1: | 4308 | taaaagctcttttccccatgtcgctgattcaaaaacatgtatttcttaaaagactagatt | 4367 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3121 | taaaagctcttttccccatgtcgctgattcaaaaacatgtatttcttaaaagactagatt | 3180 | |
| NOV1: | 4368 | ctatggatcaaacagaggttgatgcaaaaacaccactgttaaggtgtaaagtgaaatttt | 4427 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3181 | ctatggatcaaacagaggttgatgcaaaaacaccactgttaaggtgtaaagtgaaatttt | 3240 | |
| NOV1: | 4428 | ccaatggtagttttatattccaattttttaaaatgatgtattcaaggatgaacaaaatac | 4487 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3241 | ccaatggtagttttatattccaattttttaaaatgatgtattcaaggatgaacaaaatac | 3300 | |
| NOV1: | 4488 | tatagcatgcatgccactgcacttgggacctcatcatgtcagttgaatcgagaaatcacc | 4547 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3301 | tatagcatgcatgccactgcacttgggacctcatcatgtcagttgaatcgagaaatcacc | 3360 | |
| NOV1: | 4548 | aagattatgagtgcatcctcacgtgctgcctctttcctgtgatatgtagactagcacaga | 4607 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3361 | aagattatgagtgcatcctcacgtgctgcctctttcctgtgatatgtagactagcacaga | 3420 | |
| NOV1: | 4608 | gtggtacatcctaaaaacttgggaaacacagcaacccatgacttcctcttctctcaagtt | 4667 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3421 | gtggtacatcctaaaaacttgggaaacacagcaacccatgacttcctcttctctcaagtt | 3480 | |
| NOV1: | 4668 | gcaggttttcaacagttttataaggtatttgcattttagaagctctggccagtagttgtt | 4727 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3481 | gcaggttttcaacagttttataaggtatttgcattttagaagctctggccagtagttgtt | 3540 | |
| NOV1: | 4728 | aagatgttggcattaatggcattttcatagatccttggtttagtctgtgaaaaagaaacc | 4787 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3541 | aagatgttggcattaatggcattttcatagatccttggtttagtctgtgaaaaagaaacc | 3600 | |
| NOV1: | 4788 | atctctctggataggctgtcacactgactgacctaagggttcatggaagcatggcatctt | 4847 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3601 | atctctctggataggctgtcacactgactgacctaagggttcatggaagcatggcatctt | 3660 | |
| NOV1: | 4848 | gtccttgcttttagaacacccatggaagaaaacacagagtagatattgctgtcatttata | 4907 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3661 | gtccttgcttttagaacacccatggaagaaaacacagagtagatattgctgtcatttata | 3720 | |
| NOV1: | 4908 | caactacagaaatttatctatgacctaatgaggcatctcggaagtcaaagaagagggaaa | 4967 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3721 | caactacagaaatttatctatgacctaatgaggcatctcggaagtcaaagaagagggaaa | 3780 | |
| NOV1: | 4968 | gttaaccttttctactgatttcgtagtatattcagagctttcttttaagagctgtgaatg | 5027 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3781 | gttaaccttttctactgatttcgtagtatattcagagctttcttttaagagctgtgaatg | 3840 | |
| NOV1: | 5028 | aaactttttctaagcactattctattgcacacaaacagaaaaccaaagccttattagacc | 5087 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3841 | aaactttttctaagcactattctattgcacacaaacagaaaaccaaagccttattagacc | 3900 | |
| NOV1: | 5088 | taatttatgcataaagtagtattcctgagaactttattttggaaaatttataagaaagta | 5147 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3901 | taatttatgcataaagtagtattcctgagaactttattttggaaaatttataagaaagta | 3960 | |
| NOV1: | 5148 | atccaaataagaaacacgatagttgaaaataatttttatagtaaataattgttttgggct | 5207 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3961 | atccaaataagaaacacgatagttgaaaataatttttatagtaaataattgttttgggct | 4020 | |
| NOV1: | 5208 | gatttttcagtaaatccaaagtgacttaggttagaagttacactaaggaccaggggttgg | 5267 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4021 | gatttttcagtaaatccaaagtgacttaggttagaagttacactaaggaccaggggttgg | 4080 | |
| NOV1: | 5268 | aatcagaatttagtttaagatttgaggaaaagggtaagggttagtttcagttttaggatt | 5327 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4081 | aatcagaatttagtttaagatttgaggaaaagggtaagggttagtttcagttttaggatt | 4140 | |
| NOV1: | 5328 | agagctagaattgggttaggtgagaaagaaagttaaggttaaggctagagttgtctttaa | 5387 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4141 | agagctagaattgggttaggtgagaaagaaagttaaggttaaggctagagttgtctttaa | 4200 | |
| NOV1: | 5388 | gggttagggttaggaccaggttaggtcagggttggattgggtttagattggggccagtgc | 5447 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4201 | gggttagggttaggaccaggttaggtcagggttggattgggtttagattggggccagtgc | 4260 | |
| NOV1: | 5448 | tggtgttagtgatagtgtcaggatggaggttaggtttggagtaagcgttgttgctgaagt | 5507 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4261 | tggtgttagtgatagtgtcaggatggaggttaggtttggagtaagcgttgttgctgaagt | 4320 | |
| NOV1: | 5508 | gagttcaggctagcattaaattgtaagttctgaagctgatttggttatggggtctttccc | 5567 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4321 | gagttcaggctagcattaaattgtaagttctgaagctgatttggttatggggtctttccc | 4380 | |
| NOV1: | 5568 | ctgtatactaccagttgtgtctttagatggcacacaagtccaaataagtggtcatacttc | 5627 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4381 | ctgtatactaccagttgtgtctttagatggcacacaagtccaaataagtggtcatacttc | 4440 | |
| NOV1: | 5628 | tttattcagggtctcagctgcctgtacacctgctgcctacatcttcttggcaacaaagtt | 5687 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4441 | tttattcagggtctcagctgcctgtacacctgctgcctacatcttcttggcaacaaagtt | 4500 | |
| NOV1: | 5688 | acctgccacaggctctgctgagcctagttcctggtcagtaataactgaacagtgcatttt | 5747 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4501 | acctgccacaggctctgctgagcctagttcctggtcagtaataactgaacagtgcatttt | 4560 | |
| NOV1: | 5748 | ggctttggatgtgtctgtggacaagcttgctgagtttctctaccatattctgagcacacg | 5807 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4561 | ggctttggatgtgtctgtggacaagcttgctgagtttctctaccatattctgagcacacg | 4620 | |
| NOV1: | 5808 | gtctcttttgttctaatttcagcttcactgacactgggttgagcactactgtatgtggag | 5867 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4621 | gtctcttttgttctaatttcagcttcactgacactgggttgagcactactgtatgtggag | 4680 | |
| NOV1: | 5868 | ggtttggtgattgggaatggatgggggacagtgaggaggacacaccagcccattagttgt | 5927 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4681 | ggtttggtgattgggaatggatgggggacagtgaggaggacacaccagcccattagttgt | 4740 | |
| NOV1: | 5928 | taatcatcaatcacatctgattgttgaaggttattaaattaaaagaaagatcatttgtaa | 5987 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4741 | taatcatcaatcacatctgattgttgaaggttattaaattaaaagaaagatcatttgtaa | 4800 | |
| NOV1: | 5988 | catactctttgtatatatttattatatgaaaggtgcaatattttattttgtacagtatgt | 6047 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4801 | catactctttgtatatatttattatatgaaaggtgcaatattttattttgtacagtatgt | 4860 | |
| NOV1: | 6048 | aataaagacatgggacatatatttttcttattaacaaaatttcatattaaattgcttcac | 6107 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4861 | aataaagacatgggacatatatttttcttattaacaaaatttcatattaaattgcttcac | 4920 | |
| NOV1: | 6108 | tttgtatttaaagttaaaagttactatttttcatttgctattgtactttcattgttgtca | 6167 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4921 | tttgtatttaaagttaaaagttactatttttcatttgctattgtactttcattgttgtca | 4980 | |
| NOV1: | 6168 | ttcaattgacattcctgtgtactgtattttactactgtttttataacatgagagttaatg | 6227 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4981 | ttcaattgacattcctgtgtactgtattttactactgtttttataacatgagagttaatg | 5040 | |
| NOV1: | 6228 | tttctgtttcatgatccttatgtaattcagaaataaatttactttgattattcagtggca | 6287 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 5041 | tttctgtttcatgatccttatgtaattcagaaataaatttactttgattattcagtggca | 5100 | |
| NOV1: | 6288 | tccttat | 6294 | |
| | | \|\|\|\|\|\|\| | | |
| SECR: | 5101 | tccttat | 5107 | |

TABLE 4
Score = 2027 bits (5253), Expect = 0.0
Identities = 1023/1023 (100%), Positives = 1023/1023 (100%)

| | | | | |
|---|---|---|---|---|
| NOV1: | 259 | AVCLHIQTQQTVNDSLCDMVHRPPAMSQACNTEPCPPRWHVGSWGPCSATCGVGIQTRDV | 318 | (SEQ ID NO:59) |
| | | AVCLHIQTQQTVNDSLCDMVHRPPAMSQACNTEPCPPRWHVGSWGPCSATCGVGIQTRDV | | |
| SECR: | 1 | AVCLHIQTQQTVNDSLCDMVHRPPAMSQACNTEPCPPRWHVGSWGPCSATCGVGIQTRDV | 60 | (SEQ ID NO:25) |
| NOV1: | 319 | YCLHPGETPAPPEECRDEKPHALQACNQFDCPPGWHIEEWQQCSRTCGGGTQNRRVTCRQ | 378 | |
| | | YCLHPGETPAPPEECRDEKPHALQACNQFDCPPGWHIEEWQQCSRTCGGGTQNRRVTCRQ | | |
| SECR: | 61 | YCLHPGETPAPPEECRDEKPHALQACNQFDCPPGWHIEEWQQCSRTCGGGTQNRRVTCRQ | 120 | |
| NOV1: | 379 | LLTDGSFLNLSDELCQGPKASSHKSCARTDCPPHLAVGDWSKCSVSCGVGIQRRKQVCQR | 438 | |
| | | LLTDGSFLNLSDELCQGPKASSHKSCARTDCPPHLAVGDWSKCSVSCGVGIQRRKQVCQR | | |
| SECR: | 121 | LLTDGSFLNLSDELCQGPKASSHKSCARTDCPPHLAVGDWSKCSVSCGVGIQRRKQVCQR | 180 | |
| NOV1: | 439 | LAAKGRRIPLSEMMCRDLPGLPLVRSCQMPECSKIKSEMKTKLGEQGPQILSVQRVYIQT | 498 | |
| | | LAAKGRRIPLSEMMCRDLPGLPLVRSCQMPECSKIKSEMKTKLGEQGPQILSVQRVYIQT | | |
| SECR: | 181 | LAAKGRRIPLSEMMCRDLPGLPLVRSCQMPECSKIKSEMKTKLGEQGPQILSVQRVYIQT | 240 | |
| NOV1: | 499 | REEKRINLTIGSRAYLLPNTSVIIKCPVRRFQKSLIQWEKDGRCLQNSKRLGITKSGSLK | 558 | |
| | | REEKRINLTIGSRAYLLPNTSVIIKCPVRRFQKSLIQWEKDGRCLQNSKRLGITKSGSLK | | |
| SECR: | 241 | REEKRINLTIGSRAYLLPNTSVIIKCPVRRFQKSLIQWEKDGRCLQNSKRLGITKSGSLK | 300 | |
| NOV1: | 559 | IHGLAAPDIGVYRCIAGSAQETVVLKLIGTDNRLIARPALREPMREYPGMDHSEANSLGV | 618 | |
| | | IHGLAAPDIGVYRCIAGSAQETVVLKLIGTDNRLIARPALREPMREYPGMDHSEANSLGV | | |
| SECR: | 301 | IHGLAAPDIGVYRCIAGSAQETVVLKLIGTDNRLIARPALREPMREYPGMDHSEANSLGV | 360 | |
| NOV1: | 619 | TWHKMRQMWNNKNDLYLDDDHISNQPFLRALLGHCSNSAGSTNSWELKNKQFEAAVKQGA | 678 | |
| | | TWHKMRQMWNNKNDLYLDDDHISNQPFLRALLGHCSNSAGSTNSWELKNKQFEAAVKQGA | | |
| SECR: | 361 | TWHKMRQMWNNKNDLYLDDDHISNQPFLRALLGHCSNSAGSTNSWELKNKQFEAAVKQGA | 420 | |
| NOV1: | 679 | YSMDTAQFDELIRNMSQLMETGEVSDDLASQLIYQLVAELAKAQPTHMQWRGIQEETPPA | 738 | |
| | | YSMDTAQFDELIRNNSQLMETGEVSDDLASQLIYQLVAELAKAQPTHMQWRGIQEETPPA | | |
| SECR: | 421 | YSMDTAQFDELIRNNSQLMETGEVSDDLASQLIYQLVAELAKAQPTHMQWRGIQEETPPA | 480 | |
| NOV1: | 739 | AQLRGETGSVSQSSHAKNSGKLTFKPKGPVLMRQSQPPSISFNKTINSRIGNTVYITKRT | 798 | |
| | | AQLRGETGSVSQSSHAKNSGKLTFKPKGPVLMRQSQPPSISFNKTINSRIGNTVYITKRT | | |
| SECR: | 481 | AQLRGETGSVSQSSHAKNSGKLTFKPKGPVLMRQSQPPSISFNKTINSRIGNTVYITKRT | 540 | |
| NOV1: | 799 | EVINILCDLITPSEATYTWTKDGTLLQPSVKIILDGTGKIQIQNPTRKEQGIYECSVANH | 858 | |
| | | EVINILCDLITPSEATYTWTKDGTLLQPSVKIILDGTGKIQIQNPTRKEQGIYECSVANH | | |
| SECR: | 541 | EVINILCDLITPSEATYTWTKDGTLLQPSVKIILDGTGKIQIQNPTRKEQGIYECSVANH | 600 | |
| NOV1: | 859 | LGSDVESSSVLYAEAPVILSVERNITKPEHNHLSVVVGGIVEAALGANVTIRCPVKGVPQ | 918 | |
| | | LGSDVESSSVLYAEAPVILSVERNITKPEHNHLSVVVGGIVEAALGANVTIRCPVKGVPQ | | |
| SECR: | 601 | LGSDVESSSVLYAEAPVILSVERNITKPEHNHLSVVVGGIVEAALGANVTIRCPVKGVPQ | 660 | |
| NOV1: | 919 | PNITWLKRGGSLSGNVSLLFNGSLLLQNVSLENEGTYVCIATNALGKAVATSVLHLLERR | 978 | |
| | | PNITWLKRGGSLSGNVSLLFNGSLLLQNVSLENEGTYVCIATNALGKAVATSVLHLLERR | | |
| SECR: | 661 | PNITWLKRGGSLSGNVSLLFNGSLLLQNVSLENEGTYVCIATNALGKAVATSVLHLLERR | 720 | |
| NOV1: | 979 | WPESRIVFLQGHKKYILQATNTRTNSNDPTGEPPPQEPFWEPGNWSHCSATCGHLGARIQ | 1038 | |
| | | WPESRIVFLQGHKKYILQATNTRTNSNDPTGEPPPQEPFWEPGNWSHCSATCGHLGARIQ | | |
| SECR: | 721 | WPESRIVFLQGHKKYILQATNTRTNSNDPTGEPPPQEPFWEPGNWSHCSATCGHLGARIQ | 780 | |
| NOV1: | 1039 | RPQCVMANGQEVSEALCDHLQKPLAGFEPCNIRDCPARWFTSVWSQCSVSCGEGYHSRQV | 1098 | |
| | | RPQCVMANGQEVSEALCDHLQKPLAGFEPCNIRDCPARWFTSVWSQCSVSCGEGYHSRQV | | |
| SECR: | 781 | RPQCVMANGQEVSEALCDHLQKPLAGFEPCNIRDCPARWFTSVWSQCSVSCGEGYHSRQV | 840 | |
| NOV1: | 1099 | TCKRTKANGTVQVVSPRACAPKDRPLGRKPCFGHPCVQWEPGNRCPGRCMGRAVRMQQRH | 1158 | |
| | | TCKRTKANGTVQVVSPRACAPKDRPLGRKPCFGHPCVQWEPGNRCPGRCMGRAVRMQQRH | | |
| SECR: | 841 | TCKRTKANGTVQVVSPRACAPKDRPLGRKPCFGHPCVQWEPGNRCPGRCMGRAVRMQQRH | 900 | |
| NOV1: | 1159 | TACQHNSSDSNCDDRKRPTLRRNCTSGACDVCWHTGPWKPCTAACGRGFQSRKVDCIHTR | 1218 | |
| | | TACQHNSSDSNCDDRKRPTLRRNCTSGACDVCWHTGPWKPCTAACGRGFQSRKVDCIHTR | | |
| SECR: | 901 | TACQHNSSDSNCDDRKRPTLRRNCTSGACDVCWHTGPWKPCTAACGRGFQSRKVDCIHTR | 960 | |
| NOV1: | 1219 | SCKPVAKRHCVQKKKPISWRHCLGPSCDRDCTDTTHYCMFVKHLNLCSLDRYKQRCCQSC | 1278 | |
| | | SCKPVAKRHCVQKKKPISWRHCLGPSCDRDCTDTTHYCMFVKHLNLCSLDRYKQRCCQSC | | |
| SECR: | 961 | SCKPVAKRHCVQKKKPISWRHCLGPSCDRDCTDTTHYCMFVKHLNLCSLDRYKQRCCQSC | 1020 | |
| NOV1: | 1279 | QEG | 1281 | |
| | | QEG | | |
| SECR: | 1021 | QEG | 1023 | |

Based the relatedness of NOV-1 to KIAA1233 sequences, which are related to lacunin, thrombospondins, proteinases, semaphorins, ADAM-TS and properdin family members, nucleic acids and proteins according to the invention likely have similar functions as proteins belonging to these families. Thus, the NOV-1 of the invention is implicated in the following diseases and processes and has therapeutic uses in these diseases and processes: (i) inflammation, (ii) cancer, (iii) neuronal development and axonal guidance, (iv) angiogenesis and vasculogenesis—in cancer as well as for ischemia, and (v) tissue regeneration in vivo and in vitro, (vi) and other diseases and disorders.

Functional roles attributed to this family of proteins include cell attachment, spreading, motility, and proliferation, cytoskeletal organization, wound healing, and angiogenesis. Moreover, these proteins are expressed in the nervous systems during development and are thought to play roles in neuronal growth and patterning. In particular, the thrombospondin, METH-1 and ADAMTS families of proteins are potent inhibitors of angiogenesis. The ADAMTS proteins have also been implicated in cleavage of proteglycans and the control of organ shape during development. In addition, the thrombospondins have been implicated in the activation of both transforming growth factor-beta (TGF-β) precursors and TGF-β in a variety of disease states. Furthermore, semaphorin proteins have shown expression in undifferentiated neuroepithelium, suggesting that these proteins are actors in axonal guidance.

NOV 2: A Novel KIAA12334-Like Protein

The NOV-2 sequences according to the invention include nucleotide sequences encoding a polypeptide related to KIAA1233 proteins, which bear sequence similarity to lacunin, thrombospondins, proteinases, semaphorins, ADAM-TS, and properdin family members.

NOV2a and NOV2b are splice variants. Splice variants are sequences that occur naturally within the cells and tissues of individuals. The physiological activity of splice variant products and the original protein, from which they are varied, may be the same (although perhaps at a different level), opposite, or completely different and unrelated. In addition, variants may have no activity at all. When a variant and the original sequence have the same or opposite activity, they may differ in various properties not directly connected to biological activity, such as stability, clearance rate, tissue and cellular localization, temporal pattern of expression, up or down regulation mechanisms, and responses to agonists or antagonists. The presence or level of specific splice variants may be the cause, and/or indicative of, a disease, disorder, pathological or normal condition.

Because a drug may be effective against one variant but not another, or may cause side effects because it targets all splice variants, an effective drug needs to target the particular splice variant. Because soluble variants with therapeutic or disease-related functions may be naturally occurring in specific tissues, they may be optimal candidates for drug targets or protein therapeutics. Variants may have no activity at all and may thus serve as dominant negative natural inhibitors. Thus, splice variants useful in generating new drug targets, protein therapeutics and markers for diagnostics.

NOV-2 maps to Unigene cluster Hs.18705. This cluster has been mapped to Chromosome 15 Marker stSG35204, Interval D15S115-D15S152. By integrating information from the Online Mendelian Inheritance in Man (OMIM), this region is identified as 15q22-qter. Therefore, the chromosomal location of the invention is Chromosome 15 Marker stSG35204, Interval D15S115-D15S152 which corresponds to 15q22-qter.

NOV-2a

A NOV-2a nucleic acid of the invention, encoding a KIAA1233-like protein originating from chromosome 15 is shown in TABLE 5. The disclosed nucleic acid (SEQ ID NO: 3) is 7260 nucleotides and contains an open reading frame (ORF) that begins with an ATG initiation codon at nucleotide 136 and ends with a TAA stop codon at nucleotides 5209. The representative ORF encodes a 1691 amino acid polypeptide (SEQ ID NO: 4). The initiation and stop codons of SEQ ID NO: 3 are shown in bold font. The protein has a predicted molecular weight of 188743.8 daltons. Putative untranslated regions are upstream of the initiation codon and downstream of the stop codon in SEQ ID NO: 3.

TABLE 5

| | |
|---|---|
| CGCACGAGGTGTTGACGGGCGGCTTCTGCCAACTTCTCCCCAGCGCGCGCCGAGCCCGCGCGGCCCCGGGGCTGCACGTC | (SEQ ID NO:3) |
| CCAGATACTTCTGCGGCGCAAGGCTACAACTGAGACCCGGAGGAGACTAGACCCCATGGCTTCCTGGACGAGCCCCTGGT | |
| GGGTGCTGATAGGGATGGTCTTCATGCACTCTCCCCTCCCGCAGACCACAGCTGAGAAATCTCCTGGAGCCTATTTCCTT | |
| CCCGAGTTTGCACTTTCTCCTCAGGGAAGTTTTCTGGAAGACACAACAGGGGAGCAGTTCCTCACTTATCGCTATGATGA | |
| CCAGACCTCAAGAAACACTCGTTCAGATGAAGACAAAGATGGCAACTGGGATGCTTGGGGCGACTGGAGTGACTGCTCCC | |
| GGACCTGTGGGGGAGGAGCATCATATTCTCTGCGGAGATGTTTGACTGGAAGGAATTGTGAAGGGCAGAACATTCGGTAC | |
| AAGACATGCAGCAATCATGACTGCCCTCCAGATGCAGAAGATTTCAGAGCCCAGCAGTGCTCAGCCTACAATGATGTCCA | |
| GTATCAGGGGCATTACTATGAATGGCTTCCACGATATAATGATCCTGCTGCCCCGTGTGCACTCAAGTGTCATGCACAAG | |
| GACAAAACTTGGTGGTGGAGCTGGCACCTAAGGTACTGGATGGAACTCGTTGCAACACGGACTCCTTGGACATGTGTATC | |
| AGTGGCATCTGTCAGGCAGTGGGCTGCGATCGGCAACTGGGAAGCAATGCCAAGGAGGACAACTGTGGAGTCTGTGCCGG | |
| CGATGGCTCCACCTGCAGGCTTGTACGGGGACAATCAAAGTCACACGTTTCTCCTGAAAAAAGAGAAGAAAATGTAATTG | |
| CTGTTCCTTTGGGAAGTCGAAGTGTGAGAATTACAGTGAAAGGACCTGCCCACCTCTTTATTGAATCAAAAACACTTCAA | |
| GGAAGCAAAGGAGAACACAGCTTTAACAGCCCCGGCGTCTTTGTCGTAGAAAACACAACAGTGGAATTTCAGAGGGGCTC | |
| CGAGAGGCAAACTTTTAAGATTCCAGGACCTCTGATGGCTGATTTCATCTTCAAGACCAGGTACACTGCAGCCAAAGACA | |
| GCGTGGTTCAGTTCTTCTTTTACCAGCCCATCAGTCATCAGTGGAGACAAACTGACTTCTTTCCCTGCACTGTGACGTGT | |
| GGAGGAGGTTATCAGCTCAATTCTGCTGAATGTGTGGATATCCGCTTGAAGAGGGTAGTTCCTGACCATTATTGTCACTA | |
| CTACCCTGAAAATGTAAAACCAAAACCAAAACTGAAGGAATGCAGCATGGATCCCTGCCCATCAAGTGATGGATTTAAAG | |
| AGATAATGCCCTATGACCACTTCCAACCTCTTCCTCGCTGGGAACATAATCCTTGGACTGCATGTTCCGTGTCCTGTGGA | |
| GGAGGGATTCAGAGACGGAGCTTTGTGTGTGTAGAGGAATCCATGCATGGAGAGATATTGCAGGTGGAAGAATGGAAGTG | |
| CATGTACGCACCCAAACCCAAGGTTATGCAAACTTGTAATCTGTTTGATTGCCCCAAGTGGATTGCCATGGAGTGGTCTC | |
| AGTGCACAGTGACTTGTGGCCGAGGGTTACGGTACCGGGTTGTTCTGTGTATTAACCACCGCGGAGAGCATGTTGGGGGC | |
| TGCAATCCACAACTGAAGTTACACATCAAAGAAGAATGTGTCATTCCCATCCCGTGTTATAAACCAAAAGAAAAAAGTCC | |
| AGTGGAAGCAAAATTGCCTTGGCTGAAACAAGCACAAGAACTAGAAGAGACCAGAATAGCAACAGAAGAACCAACGTTCA | |
| TTCCAGAACCCTGGTCAGCCTGCAGTACCACGTGTGGGCCGGGTGTGCAGGTCCGTGAGGTGAAGTGCCGTGTGCTCCTC | |
| ACATTCACGCAGACTGAGACTGAGCTGCCCGAGGAAGAGTGTGAAGGCCCCAAGCTGCCCACCGAACGGCCCTGCCTCCT | |
| GGAAGCATGTGATGAGAGCCCGGCCTCCCGAGAGCTAGACATCCCTCTCCCTGAGGACAGTGAGACGACTTACGACTGGG | |
| AGTACGCTGGGTTCACCCCTTGCACAGCAACATGCGTGGGAGGCCATCAAGAAGCCATAGCAGTGTGCTTACATATCCAG | |
| ACCCAGCAGACAGTCAATGACAGCTTGTGTGATATGGTCCACCGTCCTCCAGCCATGAGCCAGGCCTGTAACACAGAGCC | |
| CTGTCCCCCCAGGTGGCATGTGGGCTCTTGGGGGCCCTGCTCAGCTACCTGTGGAGTTGGAATTCAGACCCGAGATGTGT | |
| ACTGCCTGCACCCAGGGGAGACCCCTGCCCCTCCTGAGGAGTGCCGAGATGAAAAGCCCCATGCTTTACAAGCATGCAAT | |
| CAGTTTGACTGCCCTCCTGGCTGGCACATTGAAGAATGGCAGCAGTGTTCCAGGACTTGTGGCGGGGGAACTCAGAACAG | |
| AAGAGTCACCTGTCGGCAGCTGCTAACGGATGGCAGCTTTTTGAATCTCTCAGATGAATTGTGCCAAGGACCCAAGGCAT | |
| CGTCTCACAAGTCCTGTGCCAGGACAGACTGTCCTCCACATTTAGCTGTGGGAGACTGGTCGAAGTGTTCTGTCAGTTGT | |
| GGTGTTGGAATCCAGAGAAGAAAGCAGGTGTGTCAAAGGCTGGCAGCCAAAGGTCGGCGCATCCCCCTCAGTGAGATGAT | |
| GTGCAGGGATCTACCAGGGTTCCCTCTTGTAAGATCTTGCCAGATGCCTGAGTGCAGTAAAATCAAATCAGAGATGAAGA | |
| CAAAACTTGGTGAGCAGGGTCCGCAGATCCTCAGTGTCCAGAGAGTCTACATTCAGACAAGGGAAGAGAAGCGTATTAAC | |
| CTGACCATTGGTAGCAGAGCCTATTTGCTGCCCAACACATCCGTGATTATTAAGTGCCCCGTGCGACGATTCCAGAAATC | |
| TCTGATCCAGTGGGAGAAGGATGGCCGTTGCCTGCAGAACTCCAAACGGCTTGGCATCACCAAGTCAGGCTCACTAAAAA | |
| TCCACGGTCTTGCTGCCCCCGACATCGGCGTGTACCGGTGCATTGCAGGCTCTGCACAGGAAACAGTTGTGCTCAAGCTC | |
| ATTGGTACTGACAACCGGCTCATCGCACGCCCAGCCCTCAGGGAGCCTATGAGGGAATATCCTGGGATGGACCACAGCGA | |
| AGCCAATAGTTTGGGAGTCACATGGCACAAAATGAGGCAAATGTGGAATGGCAAAAATGACCTTTATCTGGATGATGACC | |
| ACATTAGTAACCAGCCTTTCTTGAGAGCTCTGTTAGGCCACTGCAGCAATTCTGCAGGAAGCACCAACTCCTGGGAGTTG | |
| AAGAATAAGCAGTTTGAAGCAGCAGTTAAACAAGGAGCATATAGCATGGATACAGCCCAGTTTGATGAGCTGATAAGAAA | |
| CATGAGTCAGCTCATGGAAACCGGAGAGGTCAGCGATGATCTTGCGTCCCAGCTGATATATCAGCTGGTGGCCGAATTAG | |
| CCAAGGCACAGCCAACACACATGCAGTGGCGGGGCATCCAGGAAGAGACACCTCCTGCTGCTCAGCTCAGAGGGGAAACA | |
| GGGAGTGTGTCCCAAAGCTCGCATGCAAAAAACTCAGGCAAGCTGACATTCAAGCCGAAAGGACCTGTTCTCATGAGGCA | |
| AAGCCAACCTCCCTCAATTTCATTTAATAAAACAATAAATTCCAGGATTGGAAATACAGTATACATTACAAAAAGGACAG | |
| AGGTCATCAATATACTGTGTGACCTTATTACCCCCAGTGAGGCCACATATACATGGACCAAGGATGGAACCTTGTTACAG | |
| CCCTCAGTAAAAATAATTTTGGATGGAACTGGGAAGATACAGATACAGAATCCTACAAGGAAAGAACAAGGCATATATGA | |
| ATGTTCTGTAGCTAATCATCTTGGTTCAGATGTGGAAAGTTCTTCTGTGCTGTATGCAGAGGCACCTGTCATCTTGTCTG | |
| TTGAAAGAAATATCACCAAACCAGAGCACAACCATCTGTCTGTTGTGGTTGGAGGCATCGTGGAGGCAGCCCTTGGAGCA | |
| AACGTGACAATCCGATGTCCTGTAAAAGGTGTCCCTCAGCCTAATATAACTTGGTTGAAGAGAGGAGGATCTCTGAGTGG | |
| CAATGTTTCCTTGCTTTTCAATGGATCCCTGTTGTTGCAGAATGTTTCCCTTGAAAATGAAGGAACCTACGTCTGCATAG | |
| CCACCAATGCTCTTGGAAAGGCAGTGGCAACATCTGTATTCCACTTGCTGGAACGAAGATGGCCAGAGAGTAGAATCGTA | |
| TTTCTGCAAGGACATAAAAAGTACATTCTCCAGGCAACCAACACTAGAACCAACAGCAATGACCCAACAGGAGAACCCCC | |
| GCCTCAAGAGCCTTTTTGGGAGCCTGGTAACTGGTCACATTGTTCTGCCACCTGTGGTCATTTGGGAGCCCGCATTCAGA | |
| GACCCCAGTGTGTGATGGCCAATGGGCAGGAAGTGAGTGAGGCCCTGTGTGATCACCTCCAGAAGCCACTGGCTGGGTTT | |
| GAGCCCTGTAACATCCGGGACTGCCCAGCGAGGTGGTTCACAAGTGTGTGGTCACAGTGCTCTGTGTCTTGCGGTGAAGG | |
| ATACCACAGTCGGCAGGTGACGTGCAAGCGGACAAAAGCCAATGGAACTGTGCAGGTGGTGTCTCCAAGAGCATGTGCCC | |
| CTAAAGACCGGCCTCTGGGAAGAAAACCATGTTTTGGTCATCCATGTGTTCAGTGGGAACCAGGGAACCGGTGTCCTGGA | |
| CGTTGCATGGGCCGTGCTGTGAGGATGCAGCAGCGTCACACAGCTTGTCAACACAACAGCTCTGACTCCAACTGTGATGA | |
| CAGAAAGAGACCCACCTTAAGAAGGAACTGCACATCAGGGGCCTGTGATGTGTGTTGGCACACAGGCCCTTGGAAGCCCT | |
| CAGAAAGAGACCCACCTTAAGAAGGAACTGCACATCAGGGGCCTGTGATGTGTGTTGGCACACAGGCCCTTGGAAGCCCT | |
| GTACAGCAGCCTGTGGCAGGGGTTTCCAGTCTCGGAAAGTCGACTGTATCCACACAAGGAGTTGCAAACCTGTGGCCAAG | |
| AGACACTGTGTACAGAAAAAGAAACCAATTTCCTGGCGGCACTGTCTTGGGCCCTCCTGTGATAGAGACTGCACAGACAC | |
| AACTCACTACTGTATGTTTGTAAAACATCTTAATTTGTGTTCTCTAGACCGCTACAAACAAAGGTGCTGCCAGTCATGTC | |
| AAGAGGGATAAACCTTTGGAGGGGTCATGATGCTGCTGTGAAGATAAAAGTAGAATATAAAAGCTCTTTTCCCCATGTCG | |
| CTGATTCAAAAACATGTATTTCTTAAAAGACTAGATTCTATGGATCAAACAGAGGTTGATGCAAAAACACCACTGTTAAG | |
| GTGTAAAGTGAAATTTTCCAATGGTAGTTTTATATTCCAATTTTTTAAAATGATGTATTCAAGGATGAACAAAATACTAT | |
| AGCATGCATGCCACTGCACTTGGGACCTCATCATGTCAGTTGAATCGAGAAATCACCAAGATTATGAGTGCATCCTCACG | |
| TGCTGCCTCTTTCCTGTGATATGTAGACTAGCACAGAGTGGTACATCCTAAAAACTTGGGAAACACAGCAACCCATGACT | |
| TCCTCTTCTCTCAAGTTGCAGGTTTTCAACAGTTTTATAAGGTATTTGCATTTTAGAAGCTCTGGCCAGTAGTTGTTAAG | |
| ATGTTGGCATTAATGGCATTTTCATAGATCCTTGGTTTAGTCTGTGAAAAAGAAACCATCTCTCTGGATAGGCTGTCACA | |
| CTGACTGACCTAAGGGTTCATGGAAGCATGGCATCTTGTCCTTGCTTTTAGAACACCCATGGAAGAAAACACAGAGTAGA | |
| TATTGCTGTCATTTATACAACTACAGAAATTTATCTATGACCTAATGAGGCATCTCGGAAGTCAAAGAAGAGGGAAAGTT | |
| AACCTTTTCTACTGATTTCGTAGTATATTCAGAGCTTTCTTTTAAGAGCTGTGAATGAAACTTTTTCTAAGCACTATTCT | |
| ATTGCACACAAACAGAAAACCAAAGCCTTATTAGACCTAATTTATGCATAAAGTAGTATTCCTGAGAACTTTATTTTGGA | |
| AAATTTATAAGAAAGTAATCCAAATAAGAAACACGATAGTTGAAAATAATTTTTATAGTAAATAATTGTTTTGGGCTGAT | |
| TTTTCAGTAAATCCAAAGTGACTTAGGTTAGAAGTTACACTAAGGACCAGGGGTTGGAATCAGAATTTAGTTTAAGATTT | |
| GAGGAAAAGGGTAAGGGTTAGTTTCAGTTTTAGGATTAGAGCTAGAATTGGGTTAGGTGAGAAAGAAAGTTAAGGTTAAG | |
| GCTAGAGTTGTCTTTAAGGGTTAGGGTTAGGACCAGGTTAGGTCAGGGTTGGATTGGGTTTAGATTGGGGCCAGTGCTGG | |
| TGTTAGTGATAGTGTCAGGATGGAGGTTAGGTTTGGAGTAAGCGTTGTTGCTGAAGTGAGTTCAGGCTAGCATTAAATTG | |
| TAAGTTCTGAAGCTGATTTGGTTATGGGGTCTTTCCCCTGTATACTACCAGTTGTGTCTTTAGATGGCACACAAGTCCAA | |
| ATAAGTGGTCATACTTCTTTATTCAGGGTCTCAGCTGCCTGTACACCTGCTGCCTACATCTTCTTGGCAACAAAGTTACC | |
| TGCCACAGGCTCTGCTGAGCCTAGTTCCTGGTCAGTAATAACTGAACAGTGCATTTTGGCTTTGGATGTGTCTGTGGACA | |
| AGCTTGCTGAGTTTCTCTACCATATTCTGAGCACACGGTCTCTTTTGTTCTAACTTCAGCTTCACTGACACTGGGTTGAG | |
| CACTACTGTATGTGGAGGGTTTGGTGATTGGGAATGGATGGGGGACAGTGAGGAGGACACACCAGCCCATTAGTTGTTAA | |
| TCATCAATCACATCTGATTGTTGAAGGTTATTAAATTAAAAGAAAGATCATTTGTAACATACTCTTTGTATATATTTATT | |
| ATATGAAAGGTGCAATATTTTATTTTGTACAGTATGTAATAAAGACATGGGACATATATTTTTCTTATTAACAAAATTTC | |
| ATATTAAATTGCTTCACTTTGTATTTAAAGTTAAAAGTTACTATTTTTCATTTGCTATTGTACTTTCATTGTTGTCATTC | |
| AATTGACATTCCTGTGTACTGTATTTTACTACTGTTTTTATAACATGAGAGTTAATGTTTCTGTTTCATGATCCTTATGT | |
| AATTCAGAAATAAATTTACTTTGATTATTCAGTGGCATCCTTATAAAAAAAAAAAAAAAA | |
| MASWTSPWWVLIGMVFMHSPLPQTTAEKSPGAYFLPEFALSPQGSFLEDTTGEQFLTYRYDDQTSRNTRSDEDKDGN | (SEQ ID NO:4) |
| WDAWGDWSDCSRTCGGGASYSLRRCLTGRNCEGQNIRYKTCSNHDCPPDAEDFRAQQCSAYNDVQYQGHYYEWLPRY | |
| NDPAAPCALKCHAQGQNLVVELAPKVLDGTRCNTDSLDMCISGICQAVGCDRQLGSNAKEDNCGVCAGDGSTCRLVR | |
| GQSKSHVSPEKREENVIAVPLGSRSVRITVKGPAHLFIESKTLQGSKGEHSFNSPGVFVVENTTVEFQRGSERQTFK | |
| IPGPLMADFIFKTRYTAAKDSVVQFFFYQPISHQWRQTDFFPCTVTCGGGYQLNSAECVDIRLKRVVPDHYCHYYPE | |
| NVKPKPKLKECSMDPCPSSDGFKEIMPYDHFQPLPRWEHNPWTACSVSCGGGIQRRSFVCVEESMHGEILQVEEWKC | |
| MYAPKPKVMQTCNLFDCPKWIAMEWSQCTVTCGRGLRYRVVLCINHRGEHVGGCNPQLKLHIKEECVIPIPCYKPKE | |
| KSPVEAKLPWLKQAQELEETRIATEEPTFIPEPWSACSTTCGPGVQVREVKCRVLLTFTQTETELPEEECEGPKLPT | |
| ERPCLLEACDESPASRELDIPLPEDSETTYDWEYAGFTPCTATCVGGHQEAIAVCLHIQTQQTVNDSLCDMVHRPPA | |
| MSQACNTEPCPPRWHVGSWGPCSATCGVGIQTRDVYCLHPGETPAPPEECRDEKPHALQACNQFDCPPGWHIEEWQQ | |
| CSRTCGGGTQNRRVTCRQLLTDGSFLNLSDELCQGPKASSHKSCARTDCPPHLAVGDWSKCSVSCGVGIQRRKQVCQ | |
| RLAAKGRRIPLSEMMCRDLPGFPLVRSCQMPECSKIKSEMKTKLGEQGPQILSVQRVYIQTREEKRINLTIGSRAYL | |
| LPNTSVIIKCPVRRFQKSLIQWEKDGRCLQNSKRLGITKSGSLKIHGLAAPDIGVYRCIAGSAQETVVLKLIGTDNR | |
| LIARPALREPMREYPGMDHSEANSLGVTWHKMRQMWNNKNDLYLDDDHISNQPFLRALLGHCSNSAGSTNSWELKNK | |
| QFEAAVKQGAYSMDTAQFDELIRNMSQLMETGEVSDDLASQLIYQLVAELAKAQPTHMQWRGIQEETPPAAQLRGET | |
| GSVSQSSHAKNSGKLTFKPKGPVLMRQSQPPSISFNKTINSRIGNTVYITKRTEVINILCDLITPSEATYTWTKDGT | |
| LLQPSVKIILDGTGKIQIQNPTRKEQGIYECSVANHLGSDVESSSVLYAEAPVILSVERNITKPEHNHLSVVVGGIV | |
| EAALGANVTIRCPVKGVPQPNITWLKRGGSLSGNVSLLFNGSLLLQNVSLENEGTYVCIATNALGKAVATSVFHLLE | |
| RRWPESRIVFLQGHKKYILQATNTRTNSNDPTGEPPPQEPFWEPGNWSHCSATCGHLGARIQRPQCVMANGQEVSEA | |
| LCDHLQKPLAGFEPCNIRDCPARWFTSVWSQCSVSCGEGYHSRQVTCKRTKANGTVQVVSPRACAPKDRPLGRKPCF | |
| GHPCVQWEPGNRCPGRCMGRAVRMQQRHTACQHNSSDSNCDDRKRPTLRRNCTSGACDVCWHTGPWKPCTAACGRGF | |
| QSRKVDCIHTRSCKPVAKRHCVQKKKPISWRHCLGPSCDRDCTDTTHYCMFVKHLNLCSLDRYKQRCCQSCQEG | |

In a search of sequence databases, it was found, for example, that the disclosed NOV-2a nucleotide sequence has 5104 of 5107 bases (99%) identical to a human mRNA for a KIAA1233 protein (GenBank Accession No: ABO33059), as shown in Table 6. In all sequence alignments, identical residues are depicted as “|”. As indicated by the “Expect” value, the probability of this alignment occurring by chance alone is 0.0, the lowest probability.

Furthermore, the encoded amino acid sequence has 1023 of 1023 amino acid residues (100%) identical to, and 1021 of 1023 residues (100%) positive with, a 1023 amino acid residue human KIAA1233 protein (GenBank Accession No: BAA86547), as shown in Table 7. As indicated by the “Expect” value, the probability of this alignment occurring by chance alone is 0.0, the lowest probability.

TABLE 6
Score = 1.010e+04 bits (5095), Expect = 0.0
Identities = 5104/5107 (99%)
Strand = Plus/Plus

| | | | | |
|---|---|---|---|---|
| NOV2a: | 2138 | tagcagtgtgcttacatatccagacccagcagacagtcaatgacagcttgtgtgatatgg | 2197 | (SEQ ID NO:60) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1 | tagcagtgtgcttacatatccagacccagcagacagtcaatgacagcttgtgtgatatgg | 60 | (SEQ ID NO:26) |
| NOV2a: | 2198 | tccaccgtcctccagccatgagccaggcctgtaacacagagccctgtccccccaggtggc | 2257 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 61 | tccaccgtcctccagccatgagccaggcctgtaacacagagccctgtccccccaggtggc | 120 | |
| NOV2a: | 2258 | atgtgggctcttgggggccctgctcagctacctgtggagttggaattcagacccgagatg | 2317 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 121 | atgtgggctcttgggggccctgctcagctacctgtggagttggaattcagacccgagatg | 180 | |
| NOV2a: | 2318 | tgtactgcctgcacccaggggagacccctgcccctcctgaggagtgccgagatgaaaagc | 2377 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 181 | tgtactgcctgcacccaggggagacccctgcccctcctgaggagtgccgagatgaaaagc | 240 | |
| NOV2a: | 2378 | cccatgctttacaagcatgcaatcagtttgactgccctcctggctggcacattgaagaat | 2437 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 241 | cccatgctttacaagcatgcaatcagtttgactgccctcctggctggcacattgaagaat | 300 | |
| NOV2a: | 2438 | ggcagcagtgttccaggacttgtggcgggggaactcagaacagaagagtcacctgtcggc | 2497 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 301 | ggcagcagtgttccaggacttgtggcgggggaactcagaacagaagagtcacctgtcggc | 360 | |
| NOV2a: | 2498 | agctgctaacggatggcagctttttgaatctctcagatgaattgtgccaaggacccaagg | 2557 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 361 | agctgctaacggatggcagctttttgaatctctcagatgaattgtgccaaggacccaagg | 420 | |
| NOV2a: | 2558 | catcgtctcacaagtcctgtgccaggacagactgtcctccacatttagctgtgggagact | 2617 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 421 | catcgtctcacaagtcctgtgccaggacagactgtcctccacatttagctgtgggagact | 480 | |
| NOV2a: | 2618 | ggtcgaagtgttctgtcagttgtggtgttggaatccagagaagaaagcaggtgtgtcaaa | 2677 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 481 | ggtcgaagtgttctgtcagttgtggtgttggaatccagagaagaaagcaggtgtgtcaaa | 540 | |
| NOV2a: | 2678 | ggctggcagccaaaggtcggcgcatccccctcagtgagatgatgtgcagggatctaccag | 2737 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 541 | ggctggcagccaaaggtcggcgcatccccctcagtgagatgatgtgcagggatctaccag | 600 | |
| NOV2a: | 2738 | ggttccctcttgtaagatcttgccagatgcctgagtgcagtaaaatcaaatcagagatga | 2797 | |
| | | \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 601 | ggctccctcttgtaagatcttgccagatgcctgagtgcagtaaaatcaaatcagagatga | 660 | |
| NOV2a: | 2798 | agacaaaacttggtgagcagggtccgcagatcctcagtgtccagagagtctacattcaga | 2857 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 661 | agacaaaacttggtgagcagggtccgcagatcctcagtgtccagagagtctacattcaga | 720 | |
| NOV2a: | 2858 | caagggaagagaagcgtattaacctgaccattggtagcagagcctatttgctgcccaaca | 2917 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 721 | caagggaagagaagcgtattaacctgaccattggtagcagagcctatttgctgcccaaca | 780 | |
| NOV2a: | 2918 | catccgtgattattaagtgccccgtgcgacgattccagaaatctctgatccagtgggaga | 2977 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 781 | catccgtgattattaagtgccccgtgcgacgattccagaaatctctgatccagtgggaga | 840 | |
| NOV2a: | 2978 | aggatggccgttgcctgcagaactccaaacggcttggcatcaccaagtcaggctcactaa | 3037 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 841 | aggatggccgttgcctgcagaactccaaacggcttggcatcaccaagtcaggctcaotaa | 900 | |
| NOV2a: | 3038 | aaatccacggtcttgctgcccccgacatcggcgtgtaccggtgcattgcaggctctgcac | 3097 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 901 | aaatccacggtcttgctgcccccgacatcggcgtgtaccggtgcattgcaggctctgcac | 960 | |
| NOV2a: | 3098 | aggaaacagttgtgctcaagctcattggtactgacaaccggctcatcgcacgcccagccc | 3157 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 961 | aggaaacagttgtgctcaagctcattggtactgacaaccggctcatcgcacgcccagccc | 1020 | |
| NOV2a: | 3158 | tcagggagcctatgagggaatatcctgggatggaccacagcgaagccaatagtttgggag | 3217 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1021 | tcagggagcctatgagggaatatcctgggatggaccacagcgaagccaatagtttgggag | 1080 | |
| NOV2a: | 3218 | tcacatggcacaaaatgaggcaaatgtggaataacaaaaatgacctttatctggatgatg | 3277 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1081 | tcacatggcacaaaatgaggcaaatgtggaataacaaaaatgacctttatctggatgatg | 1140 | |
| NOV2a: | 3278 | accacattagtaaccagcctttcttgagagctctgttaggccactgcagcaattctgcag | 3337 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1141 | accacattagtaaccagcctttcttgagagctctgttaggccactgcagcaattctgcag | 1200 | |
| NOV2a: | 3338 | gaagcaccaactcctgggagttgaagaataagcagtttgaagcagcagttaaacaaggag | 3397 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1201 | gaagcaccaactcctgggagttgaagaataagcagtttgaagcagcagttaaacaaggag | 1260 | |
| NOV2a: | 3398 | catatagcatggatacagcccagtttgatgagctgataagaaacatgagtcagctcatgg | 3457 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1261 | catatagcatggatacagcccagtttgatgagctgataagaaacatgagtcagctcatgg | 1320 | |
| NOV2a: | 3458 | aaaccggagaggtcagcgatgatcttgcgtcccagctgatatatcagctggtggccgaat | 3517 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1321 | aaaccggagaggtcagcgatgatcttgcgtcccagctgatatatcagctggtggccgaat | 1380 | |
| NOV2a: | 3518 | tagccaaggcacagccaacacacatgcagtggcggggcatccaggaagagacacctcctg | 3577 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1381 | tagccaaggcacagccaacacacatgcagtggcggggcatccaggaagagacacctcctg | 1440 | |
| NOV2a: | 3578 | ctgctcagctcagaggggaaacagggagtgtgtcccaaagctcgcatgcaaaaaactcag | 3637 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1441 | ctgctcagctcagaggggaaacagggagtgtgtcccaaagctcgcatgcaaaaaactcag | 1500 | |
| NOV2a: | 3638 | gcaagctgacattcaagccgaaaggacctgttctcatgaggcaaagccaacctccctcaa | 3697 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1501 | gcaagctgacattcaagccgaaaggacctgttctcatgaggcaaagccaacctccctcaa | 1560 | |
| NOV2a: | 3698 | tttcatttaataaaacaataaattccaggattggaaatacagtatacattacaaaaagga | 3757 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1561 | tttcatttaataaaacaataaattccaggattggaaatacagtatacattacaaaaagga | 1620 | |
| NOV2a: | 3758 | cagaggtcatcaatatactgtgtgaccttattacccccagtgaggccacatatacatgga | 3817 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1621 | cagaggtcatcaatatactgtgtgaccttattacccccagtgaggccacatatacatgga | 1680 | |
| NOV2a: | 3818 | ccaaggatggaaccttgttacagccctcagtaaaaataattttggatggaactgggaaga | 3877 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1681 | ccaaggatggaaccttgttacagccctcagtaaaaataattttggatggaactgggaaga | 1740 | |
| NOV2a: | 3878 | tacagatacagaatcctacaaggaaagaacaaggcatatatgaatgttctgtagctaatc | 3937 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1741 | tacagatacagaatcctacaaggaaagaacaaggcatatatgaatgttctgtagctaatc | 1800 | |
| NOV2a: | 3938 | atcttggttcagatgtggaaagttcttctgtgctgtatgcagaggcacctgtcatcttgt | 3997 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1801 | atcttggttcagatgtggaaagttcttctgtgctgtatgcagaggcacctgtcatcttgt | 1860 | |
| NOV2a: | 3998 | ctgttgaaagaaatatcaccaaaccagagcacaaccatctgtctgttgtggttggaggca | 4057 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1861 | ctgttgaaagaaatatcaccaaaccagagcacaaccatctgtctgttgtggttggaggca | 1920 | |
| NOV2a: | 4058 | tcgtggaggcagcccttggagcaaacgtgacaatccgatgtcctgtaaaaggtgtccctc | 4117 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1921 | tcgtggaggcagcccttggagcaaacgtgacaatccgatgtcctgtaaaaggtgtccctc | 1980 | |
| NOV2a: | 4118 | agcctaatataacttggttgaagagaggaggatctctgagtggcaatgtttccttgcttt | 4177 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 1981 | agcctaatataacttggttgaagagaggaggatctctgagtggcaatgtttccttgcttt | 2040 | |
| NOV2a: | 4178 | tcaatggatccctgttgttgcagaatgtttcccttgaaaatgaaggaacctacgtctgca | 4237 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2041 | tcaatggatccctgttgttgcagaatgtttcccttgaaaatgaaggaacctacgtctgca | 2100 | |
| NOV2a: | 4238 | tagccaccaatgctcttggaaaggcagtggcaacatctgtattccacttgctggaacgaa | 4297 | |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| | | |
| SECR: | 2101 | tagccaccaatgctcttggaaaggcagtggcaacatctgtactccacttgctggaacgaa | 2160 | |
| NOV2a: | 4298 | gatggccagagagtagaatcgtatttctgcaaggacataaaaagtacattctccaggcaa | 4357 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2161 | gatggccagagagtagaatcgtatttctgcaaggacataaaaagtacattctccaggcaa | 2220 | |
| NOV2a: | 4358 | ccaacactagaaccaacagcaatgacccaacaggagaacccccgcctcaagagccttttt | 4417 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2221 | ccaacactagaaccaacagcaatgacccaacaggagaacccccgcctcaagagccttttt | 2280 | |
| NOV2a: | 4418 | gggagcctggtaactggtcacattgttctgccacctgtggtcatttgggagcccgcattc | 4477 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2281 | gggagcctggtaactggtcacattgttctgccacctgtggtcatttgggagcccgcattc | 2340 | |
| NOV2a: | 4478 | agagaccccagtgtgtgatggccaatgggcaggaagtgagtgaggccctgtgtgatcacc | 4537 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2341 | agagaccccagtgtgtgatggccaatgggcaggaagtgagtgaggccctgtgtgatcacc | 2400 | |
| NOV2a: | 4538 | tccagaagccactggctgggtttgagccctgtaacatccgggactgcccagcgaggtggt | 4597 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2401 | tccagaagccactggctgggtttgagccctgtaacatccgggactgcccagcgaggtggt | 2460 | |
| NOV2a: | 4598 | tcacaagtgtgtggtcacagtgctctgtgtcttgcggtgaaggataccacagtcggcagg | 4657 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2461 | tcacaagtgtgtggtcacagtgctctgtgtcttgcggtgaaggataccacagtcggcagg | 2520 | |
| NOV2a: | 4658 | tgacgtgcaagcggacaaaagccaatggaactgtgcaggtggtgtctccaagagcatgtg | 4717 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2521 | tgacgtgcaagcggacaaaagccaatggaactgtgcaggtggtgtctccaagagcatgtg | 2580 | |
| NOV2a: | 4718 | cccctaaagaccggcctctgggaagaaaaccatgttttggtcatccatgtgttcagtggg | 4777 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2581 | cccctaaagaccggcctctgggaagaaaaccatgttttggtcatccatgtgttcagtggg | 2640 | |
| NOV2a: | 4778 | aaccagggaaccggtgtcctggacgttgcatgggccgtgctgtgaggatgcagcagcgtc | 4837 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2641 | aaccagggaaccggtgtcctggacgttgcatgggccgtgctgtgaggatgcagcagcgtc | 2700 | |
| NOV2a: | 4838 | acacagcttgtcaacacaacagctctgactccaactgtgatgacagaaagagacccacct | 4897 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2701 | acacagcttgtcaacacaacagctctgactccaactgtgatgacagaaagagacccacct | 2760 | |
| NOV2a: | 4898 | taagaaggaactgcacatcaggggcctgtgatgtgtgttggcacacaggcccttggaagc | 4957 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2761 | taagaaggaactgcacatcaggggcctgtgatgtgtgttggcacacaggcccttggaagc | 2820 | |
| NOV2a: | 4958 | cctgtacagcagcctgtggcaggggtttccagtctcggaaagtcgactgtatccacacaa | 5017 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2821 | cctgtacagcagcctgtggcaggggtttccagtctcggaaagtcgactgtatccacacaa | 2880 | |
| NOV2a: | 5018 | ggagttgcaaacctgtggccaagagacactgtgtacagaaaaagaaaccaatttcctggc | 5077 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2881 | ggagttgcaaacctgtggccaagagacactgtgtacagaaaaagaaaccaatttcctggc | 2940 | |
| NOV2a: | 5078 | ggcactgtcttgggccctcctgtgatagagactgcacagacacaactcactactgtatgt | 5137 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 2941 | ggcactgtcttgggccctcctgtgatagagactgcacagacacaactcactactgtatgt | 3000 | |
| NOV2a: | 5138 | ttgtaaaacatcttaatttgtgttctctagaccgctacaaacaaaggtgctgccagtcat | 5197 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3001 | ttgtaaaacatcttaatttgtgttctctagaccgctacaaacaaaggtgctgccagtcat | 3060 | |
| NOV2a: | 5198 | gtcaagagggataaacctttggaggggtcatgatgctgctgtgaagataaaagtagaata | 5257 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3061 | gtcaagagggataaacctttggaggggtcatgatgctgctgtgaagataaaagtagaata | 3120 | |
| NOV2a: | 5258 | taaaagctcttttccccatgtcgctgattcaaaaacatgtatttcttaaaagactagatt | 5317 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3121 | taaaagctcttttccccatgtcgctgattcaaaaacatgtatttcttaaaagactagatt | 3180 | |
| NOV2a: | 5318 | ctatggatcaaacagaggttgatgcaaaaacaccactgttaaggtgtaaagtgaaatttt | 5377 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3181 | ctatggatcaaacagaggttgatgcaaaaacaccactgttaaggtgtaaagtgaaatttt | 3240 | |
| NOV2a: | 5378 | ccaatggtagttttatattccaattttttaaaatgatgtattcaaggatgaacaaaatac | 5437 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3241 | ccaatggtagttttatattccaattttttaaaatgatgtattcaaggatgaacaaaatac | 3300 | |
| NOV2a: | 5438 | tatagcatgcatgccactgcacttgggacctcatcatgtcagttgaatcgagaaatcacc | 5497 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3301 | tatagcatgcatgccactgcacttgggacctcatcatgtcagttgaatcgagaaatcacc | 3360 | |
| NOV2a: | 5498 | aagattatgagtgcatcctcacgtgctgcctctttcctgtgatatgtagactagcacaga | 5557 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3361 | aagattatgagtgcatcctcacgtgctgcctctttcctgtgatatgtagactagcacaga | 3420 | |
| NOV2a: | 5558 | gtggtacatcctaaaaacttgggaaacacagcaacccatgacttcctcttctctcaagtt | 5617 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3421 | gtggtacatcctaaaaacttgggaaacacagcaacccatgacttcctcttctctcaagtt | 3480 | |
| NOV2a: | 5618 | gcaggttttcaacagttttataaggtatttgcattttagaagctctggccagtagttgtt | 5677 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3481 | gcaggttttcaacagttttataaggtatttgcattttagaagctctggccagtagttgtt | 3540 | |
| NOV2a: | 5678 | aagatgttggcattaatggcattttcatagatccttggtttagtctgtgaaaaagaaacc | 5737 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3541 | aagatgttggcattaatggcattttcatagatccttggtttagtctgtgaaaaagaaacc | 3600 | |
| NOV2a: | 5738 | atctctctggataggctgtcacactgactgacctaagggttcatggaagcatggcatctt | 5797 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3601 | atctctctggataggctgtcacactgactgacctaagggttcatggaagcatggcatctt | 3660 | |
| NOV2a: | 5798 | gtccttgcttttagaacacccatggaagaaaacacagagtagatattgctgtcatttata | 5857 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3661 | gtccttgcttttagaacacccatggaagaaaacacagagtagatattgctgtcatttata | 3720 | |
| NOV2a: | 5858 | caactacagaaatttatctatgacctaatgaggcatctcggaagtcaaagaagagggaaa | 5917 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3721 | caactacagaaatttatctatgacctaatgaggcatctcggaagtcaaagaagagggaaa | 3780 | |
| NOV2a: | 5918 | gttaaccttttctactgatttcgtagtatattcagagctttcttttaagagctgtgaatg | 5977 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3781 | gttaaccttttctactgatttcgtagtatattcagagctttcttttaagagctgtgaatg | 3840 | |
| NOV2a: | 5978 | aaactttttctaagcactattctattgcacacaaacagaaaaccaaagccttattagacc | 6037 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3841 | aaactttttctaagcactattctattgcacacaaacagaaaaccaaagccttattagacc | 3900 | |
| NOV2a: | 6038 | taatttatgcataaagtagtattcctgagaactttattttggaaaatttataagaaagta | 6097 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3901 | taatttatgcataaagtagtattcctgagaactttattttggaaaatttataagaaagta | 3960 | |
| NOV2a: | 6098 | atccaaataagaaacacgatagttgaaaataatttttatagtaaataattgttttgggct | 6157 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 3961 | atccaaataagaaacacgatagttgaaaataatttttatagtaaataattgttttgggct | 4020 | |
| NOV2a: | 6158 | gatttttcagtaaatccaaagtgacttaggttagaagttacactaaggaccaggggttgg | 6217 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4021 | gatttttcagtaaatccaaagtgacttaggttagaagttacactaaggaccaggggttgg | 4080 | |
| NOV2a: | 6218 | aatcagaatttagtttaagatttgaggaaaagggtaagggttagtttcagttttaggatt | 6277 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4081 | aatcagaatttagtttaagatttgaggaaaagggtaagggttagtttcagttttaggatt | 4140 | |
| NOV2a: | 6278 | agagctagaattgggttaggtgagaaagaaagttaaggttaaggctagagttgtctttaa | 6337 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4141 | agagctagaattgggttaggtgagaaagaaagttaaggttaaggctagagttgtctttaa | 4200 | |
| NOV2a: | 6338 | gggttagggttaggaccaggttaggtcagggttggattgggtttagattggggccagtgc | 6397 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4201 | gggttagggttaggaccaggttaggtcagggttggattgggtttagattggggccagtgc | 4260 | |
| NOV2a: | 6398 | tggtgttagtgatagtgtcaggatggaggttaggtttggagtaagcgttgttgctgaagt | 6457 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4261 | tggtgttagtgatagtgtcaggatggaggttaggtttggagtaagcgttgttgctgaagt | 4320 | |
| NOV2a: | 6458 | gagttcaggctagcattaaattgtaagttctgaagctgatttggttatggggtctttccc | 6517 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4321 | gagttcaggctagcattaaattgtaagttctgaagctgatttggttatggggtctttccc | 4380 | |
| NOV2a: | 6518 | ctgtatactaccagttgtgtctttagatggcacacaagtccaaataagtggtcatacttc | 6577 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4381 | ctgtatactaccagttgtgtctttagatggcacacaagtccaaataagtggtcatacttc | 4440 | |
| NOV2a: | 6578 | tttattcagggtctcagctgcctgtacacctgctgcctacatcttcttggcaacaaagtt | 6637 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4441 | tttattcagggtctcagctgcctgtacacctgctgcctacatcttcttggcaacaaagtt | 4500 | |
| NOV2a: | 6638 | acctgccacaggctctgctgagcctagttcctggtcagtaataactgaacagtgcatttt | 6697 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4501 | acctgccacaggctctgctgagcctagttcctggtcagtaataactgaacagtgcatttt | 4560 | |
| NOV2a: | 6698 | ggctttggatgtgtctgtggacaagcttgctgagtttctctaccatattctgagcacacg | 6757 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4561 | ggctttggatgtgtctgtggacaagcttgctgagtttctctaccatattctgagcacacg | 4620 | |
| NOV2a: | 6758 | gtctcttttgttctaacttcagcttcactgacactgggttgagcactactgtatgtggag | 6817 | |
| | | \|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4621 | gtctcttttgttctaatttcagcttcactgacactgggttgagcactactgtatgtggag | 4680 | |
| NOV2a: | 6818 | ggtttggtgattgggaatggatgggggacagtgaggaggacacaccagcccattagttgt | 6877 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4681 | ggtttggtgattgggaatggatgggggacagtgaggaggacacaccagcccattagttgt | 4740 | |
| NOV2a: | 6878 | taatcatcaatcacatctgattgttgaaggttattaaattaaaagaaagatcatttgtaa | 6937 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4741 | taatcatcaatcacatctgattgttgaaggttattaaattaaaagaaagatcatttgtaa | 4800 | |
| NOV2a: | 6938 | catactctttgtatatatttattatatgaaaggtgcaatattttattttgtacagtatgt | 6997 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4801 | catactctttgtatatatttattatatgaaaggtgcaatattttattttgtacagtatgt | 4860 | |
| NOV2a: | 6998 | aataaagacatgggacatatatttttcttattaacaaaatttcatattaaattgcttcac | 7057 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4861 | aataaagacatgggacatatatttttcttattaacaaaatttcatattaaattgcttcac | 4920 | |
| NOV2a: | 7058 | tttgtatttaaagttaaaagttactatttttcatttgctattgtactttcattgttgtca | 7117 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4921 | tttgtatttaaagttaaaagttactatttttcatttgctattgtactttcattgttgtca | 4980 | |
| NOV2a: | 7118 | ttcaattgacattcctgtgtactgtattttactactgtttttataacatgagagttaatg | 7177 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 4981 | ttcaattgacattcctgtgtactgtattttactactgtttttataacatgagagttaatg | 5040 | |
| NOV2a: | 7178 | tttctgtttcatgatccttatgtaattcagaaataaatttactttgattattcagtggca | 7237 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SECR: | 5041 | tttctgtttcatgatccttatgtaattcagaaataaatttactttgattattcagtggaa | 5100 | |
| NOV2a: | 7238 | tccttat | 7244 | |
| | | \|\|\|\|\|\|\| | | |
| SECR: | 5101 | tccttat | 5107 | |

TABLE 7
Score = 2045 bits (5300), Expect = 0.0
Identities = 1021/1023 (99%), Positives = 1021/1023 (99%)

| | | | |
|---|---|---|---|
| NOV2A: | 669 | AVCLHIQTQQTVNDSLCDMVHRPPAMSQACNTEPCPPRWHVGSWGPCSATCGVGIQTRDV | 728 |
| | | AVCLHIQTQQTVNDSLCDMVHRPPAMSQACNTEPCPPRWHVGSWGPCSATCGVGIQTRDV | |
| SECR: | 1 | AVCLHIQTQQTVNDSLCDMVHRPPAMSQACNTEPCPPRWHVGSWGPCSATCGVGIQTRDV | 60 |
| NOV2A: | 729 | YCLHPGETPAPPEECRDEKPHALQACNQFDCPPGWHIEEWQQCSRTCGGGTQNRRVTCRQ | 788 |
| | | YCLHPGETPAPPEECRDEKPHALQACNQFDCPPGWHIEEWQQCSRTCGGGTQNRRVTCRQ | |
| SECR: | 61 | YCLHPGETPAPPEECRDEKPHALQACNQFDCPPGWHIEEWQQCSRTCGGGTQNRRVTCRQ | 120 |
| NOV2A: | 789 | LLTDGSFLNLSDELCQGPKASSHKSCARTDCPPHLAVGDWSKCSVSCGVGIQRRKQVCQR | 848 |
| | | LLTDGSFLNLSDELCQGPKASSHKSCARTDCPPHLAVGDWSKCSVSCGVGIQRRKQVCQR | |
| SECR: | 121 | LLTDGSFLNLSDELCQGPKASSHKSCARTDCPPHLAVGDWSKCSVSCGVGIQRRKQVCQR | 180 |
| NOV2A: | 849 | LAAKGRRIPLSEMMCRDLPGFPLVRSCQMPECSKIKSEMKTKLGEQGPQILSVQRVYIQT | 908 |
| | | LAAKGRRIPLSEMMCRDLPG PLVRSCQMPECSKIKSEMKTKLGEQGPQILSVQRVYIQT | |
| SECR: | 181 | LAAKGRRIPLSEMMCRDLPGLPLVRSCQMPECSKIKSEMKTKLGEQGPQILSVQRVYIQT | 240 |
| NOV2A: | 909 | REEKRINLTIGSRAYLLPNTSVIIKCPVRRFQKSLIQWEKDGRCLQNSKRLGITKSGSLK | 968 |
| | | REEKRINLTIGSRAYLLPNTSVIIKCPVRRFQKSLIQWEKDGRCLQNSKRLGITKSGSLK | |
| SECR: | 241 | REEKRINLTIGSRAYLLPNTSVIIKCPVRRFQKSLIQWEKDGRCLQNSKRLGITKSGSLK | 300 |
| NOV2A: | 969 | IHGLAAPDIGVYRCIAGSAQETVVLKLIGTDNRLIARPALREPMREYPGMDHSEANSLGV | 1028 |
| | | IHGLAAPDIGVYRCIAGSAQETVVLKLIGTDNRLIARPALREPMREYPGMDHSEANSLGV | |
| SECR: | 301 | IHGLAAPDIGVYRCIAGSAQETVVLKLIGTDNRLIARPALREPMREYPGMDHSEANSLGV | 360 |
| NOV2A: | 1029 | TWHKMRQMWNNKNDLYLDDDHISNQPFLRALLGHCSNSAGSTNSWELKNKQFEAAVKQGA | 1088 |
| | | TWHKMRQMWNNKNDLYLDDDHISNQPFLRALLGHCSNSAGSTNSWELKNKQFEAAVKQGA | |
| SECR: | 361 | TWHKMRQMWNNKNDLYLDDDHISNQPFLRALLGHCSNSAGSTNSWELKNKQFEAAVKQGA | 420 |
| NOV2A: | 1089 | YSMDTAQFDELIRNMSQLMETGEVSDDLASQLIYQLVAELAKAQPTHMQWRGIQEETPPA | 1148 |
| | | YSMDTAQFDELIRNMSQLMETGEVSDDLASQLIYQLVAELAKAQPTHMQWRGIQEETPPA | |
| SEC: | 421 | YSMDTAQFDELIRNMSQLMETGEVSDDLASQLIYQLVAELAKAQPTHMQWRGIQEETPPA | 480 |
| NOV2A: | 1149 | AQLRGETGSVSQSSHAKNSGKLTFKPKGPVLMRQSQPPSISFNKTINSRIGNTVYITKRT | 1208 |
| | | AQLRGETGSVSQSSHAKNSGKLTFKPKGPVLMRQSQPPSISFNKTINSRIGNTVYITKRT | |
| SECR: | 481 | AQLRGETGSVSQSSHAKNSGKLTFKPKGPVLMRQSQPPSISFNKTINSRIGNTVYITKRT | 540 |
| NOV2A: | 1209 | EVINILCDLITPSEATYTWTKDGTLLQPSVKIILDGTGKIQIQNPTRKEQGIYECSVANH | 1268 |
| | | EVINILCDLITPSEATYTWTKDGTLLQPSVKIILDGTGKIQIQNPTRKEQGIYECSVANH | |
| SECR: | 541 | EVINILCDLITPSEATYTWTKDGTLLQPSVKIILDGTGKIQIQNPTRKEQGIYECSVANH | 600 |
| NOV2A: | 1269 | LGSDVESSSVLYAEAPVILSVERNITKPEHNHLSVVVGGIVEAALGANVTIRCPVKGVPQ | 1328 |
| | | LGSDVESSSVLYAEAPVILSVERNITKPEHNHLSVVVGGIVEAALGANVTIRCPVKGVPQ | |
| SECR: | 601 | LGSDVESSSVLYAEAPVILSVERNITKPEHNHLSVVVGGIVEAALGANVTIRCPVKGVPQ | 660 |
| NOV2A: | 1329 | PNITWLKRGGSLSGNVSLLFNGSLLLQNVSLENEGTYVCIATNALGKAVATSVFHLLERR | 1388 |
| | | PNITWLKRGGSLSGNVSLLFNGSLLLQNVSLENEGTYVCIATNALGKAVATSV HLLERR | |
| SECR: | 661 | PNITWLKRGGSLSGNVSLLFNGSLLLQNVSLENEGTYVCIATNALGKAVATSVLHLLERR | 720 |
| NOV2A: | 1389 | WPESRIVFLQGHKKYILQATNTRTNSNDPTGEPPPQEPFWEPGNWSHCSATCGHLGARIQ | 1448 |
| | | WPESRIVFLQGHKKYILQATNTRTNSNDPTGEPPPQEPFWEPGNWSHCSATCGHLGARIQ | |
| SECR: | 721 | WPESRIVFLQGHKKYILQATNTRTNSNDPTGEPPPQEPFWEPGNWSHCSATCGHLGARIQ | 780 |
| NOV2A: | 1449 | RPQCVMANGQEVSEALCDHLQKPLAGFEPCNIRDCPARWFTSVWSQCSVSCGEGYHSRQV | 1508 |
| | | RPQCVMANGQEVSEALCDHLQKPLAGFEPCNIRDCPARWFTSVWSQCSVSCGEGYHSRQV | |
| SECR: | 781 | RPQCVMANCQEVSEALCDHLQKPLAGFEPCNIRDCPARWFTSVWSQCSVSCGEGYHSRQV | 840 |
| NOV2A: | 1509 | TCKRTKANGTVQVVSPRACAPKDRPLGRKPCFGHPCVQWEPGNRCPGRCMGRAVRMQQRH | 1568 |
| | | TCKRTKANGTVQVVSPRACAPKDRPLGRKPCFGHPCVQWEPGNRCPGRCMGRAVRMQQRH | |
| SECR: | 841 | TCKRTKANGTVQVVSPRACAPKDRPLGRKPCFGHPCVQWEPGNRCPGRCMGRAVRNQQRH | 900 |
| NOV2A: | 1569 | TACQHNSSDSNCDDRKRPTLRRNCTSGACDVCWHTGPWKPCTAACGRGFQSRKVDCIHTR | 1628 |
| | | TACQHNSSDSNCDDRKRPTLRRNCTSGACDVCWHTGPWKPCTAACGRGFQSRKVDCIHTR | |
| SECR: | 901 | TACQHNSSDSNCDDRKRPTLRRNCTSGACDVCWHTGPWKPCTAACGRGFQSRKVDCIHTR | 960 |
| NOV2A: | 1629 | SCKPVAKRHCVQKKKPISWRHCLGPSCDRDCTDTTHYCMFVKHLNLCSLDRYKQRCCQSC | 1688 |
| | | SCKPVAKRHCVQKKKPISWRHCLGPSCDRDCTDTTHYCMFVKHLNLCSLDRYKQRCCQSC | |
| SECR: | 961 | SCKPVAKRHCVQKKKPISWRHCLGPSCDRDCTDTTHYCMFVKHLNLCSLDRYKQRCCQSC | 1020 |
| NOV2A: | 1689 | QEG 1691 (SEQ ID NO: 61) | |
| | | QEG | |
| SECR: | 1021 | QEG 1023 (SEQ ID NO: 27) | |

SignalP and PSORT analysis indicate that NOV-2 may be localized in the endoplasmic reticulum, with likely cleavage sites between positions 26 and 27. Thus, it is likely that NOV-2a protein is available at the appropriate sub-cellular localization for the therapeutic uses described in this application.

Based the relatedness of the disclosed NOV-2a to KIAA1233 sequences, which are related to lacunin, thrombospondins, proteinases, semaphorins, ADAM-TS and properdin family members, the nucleic acids and proteins of the invention can have similar finctions as proteins belonging to these families.

Functional roles attributed to this family of proteins include cell attachment, spreading, motility, and proliferation, cytoskeletal organization, wound healing, and angiogenesis. Moreover, these proteins are expressed in the nervous systems during development and are thought to play roles in neuronal growth and patterning. In particular, the thrombospondin, METH-1 and ADAMTS families of proteins are potent inhibitors of angiogenesis. The ADAMTS proteins have also been implicated in cleavage of proteglycans and the control of organ shape during development. In addition, the thrombospondins have been implicated in the activation of both transforming growth factor-beta (TGF-β) precursors and TGF-β in a variety of disease states. Furthermore, semaphorin proteins have shown expression in undifferentiated neuroepithelium, suggesting that these proteins are actors in axonal guidance. Thus, the NOV-2a sequences of the invention is implicated in the following diseases and processes and has therapeutic uses in these diseases and processes: (i) inflammation, (ii) cancer, (iii) neuronal development and axonal guidance, (iv) angiogenesis and vasculogenesis—in cancer as well as for ischemia, and (v) tissue regeneration in vivo and in vitro, (vi) and other diseases and disorders.

NOV 2b:

A NOV-2b nucleic acid of the invention, encoding a KIAA1233-like protein, is found within the nucleotide sequence of NOV-2a (SEQ ID NO: 3) in Table 5. The disclosed nucleic acid is 6303 nucleotides in length and contains an open reading frame (ORF) that begins with an ATG initiation codon at nucleotide 425 and ends with a TAA stop codon at nucleotides 4268 (SEQ ID NO: 57). The initiation and stop codons of NOV-2b are shown in bold font in SEQ ID NO: 4. The representative ORF encodes a 406 amino acid polypeptide (SEQ ID NO: 5), which is shown below in Table 8. Putative untranslated regions are upstream of the initiation codon and downstream of the stop codon in SEQ ID NO: 57.

TABLE 8

| | |
|---|---|
| TATAATTATTAATAGAGACCTTTCAAAGGACAAATTCTGTGAAATAAAGTGGTTTTCTGA | (SEQ ID NO:57) |
| AGAGCCTACTAATAGGACAGTGTGTTAATATCACTAATAAGAGAGTAATGATTATAAAAA | |
| GGAATAAATTTATTGAAATTGCAAGATACTTTTCTCCTTTGATTAATATACTGCTAGTTT | |
| AGTTTTCTACATTTTCAAATAGAACTGGGGAATTTGTGTCGTAGATATTCTTGACAACTA | |
| AAGAGATGGTGGCTGAATTTTTGGGAATGGTTGATAACACTTGATATTTTTAGTTTCCAA | |
| TTTGGAAGAGCTCTGTCTCTTGGGATGTCAAATATTATATTCGTCAATTAATGAATGTGT | |
| TAATTTATTATAGAAATGATATTCTCACAATGATTTCATTTGTAGTGATGGATTTAAAGA | |
| GATAATGCCCTATGACCACTTCCAACCTCTTCCTCGCTGGGAACATAATCCTTGGACTGC | |
| ATGTTCCGTGTCCTGTGGAGGAGGGATTCAGAGACGGAGCTTTGTGTGTGTAGAGGAATC | |
| CATGCATGGAGAGATATTGCAGGTGGAAGAATGGAAGTGCATGTACGCACCCAAACCCAA | |
| GGTTATGCAAACTTGTAATCTGTTTGATTGCCCCAAGTGGATTGCCATGGAGTGGTCTCA | |
| GTGCACAGTGACTTGTGGCCGAGGGTTACGGTACCGGGTTGTTCTGTGTATTAACCACCG | |
| CGGAGAGCATGTTGGGGGCTGCAATCCACAACTGAAGTTACACATCAAAGAAGAATGTGT | |
| CATTCCCATCCCGTGTTATAAACCAAAAGAAAAAAGTCCAGTGGAAGCAAAATTGCCTTG | |
| GCTGAAACAAGCACAAGAACTAGAAGAGACCAGAATAGCAACAGAAGAACCAACGTTCAT | |
| TCCAGAACCCTGGTCAGCCTGCAGTACCACGTGTGGGCCGGGTGTGCAGGTCCGTGAGGT | |
| GAAGTGCCGTGTGCTCCTCACATTCACGCAGACTGAGACTGAGCTGCCCGAGGAAGAGTG | |
| TGAAGGCCCCAAGCTGCCCACCGAACGGCCCTGCCTCCTGGAAGCATGTGATGAGAGCCC | |
| GGCCTCCCGAGAGCTAGACATCCCTCTCCCTGAGGACAGTGAGACGACTTACGACTGGGA | |
| GTACGCTGGGTTCACCCCTTGCACAGCAACATGCGTGGGAGGCCATCAAGAAGCCATAGC | |
| AGTGTGCTTACATATCCAGACCCAGCAGACAGTCAATGACAGCTTGTGTGATATGGTCCA | |
| CCGTCCTCCAGCCATGAGCCAGGCCTGTAACACAGAGCCCTGTCCCCCCAGGTGGCATGT | |
| GGGCTCTTGGGGGCCCTGCTCAGCTACCTGTGGAGTTGGAATTCAGACCCGAGATGTGTA | |
| CTGCCTGCACCCAGGGGAGACCCCTGCCCCTCCTGAGGAGTGCCGAGATGAAAAGCCCCA | |
| TGCTTTACAAGCATGCAATCAGTTTGACTGCCCTCCTGGCTGGCACATTGAAGAATGGCA | |
| GCAGTGTTCCAGGACTTGTGGCGGGGGAACTCAGAACAGAAGAGTCACCTGTCGGCAGCT | |
| GCTAACGGATGGCAGCTTTTTGAATCTCTCAGATGAATTGTGCCAAGGACCCAAGGCATC | |
| GTCTCACAAGTCCTGTGCCAGGACAGACTGTCCTCCACATTTAGCTGTGGGAGACTGGTC | |
| GAAGTGTTCTGTCAGTTGTGGTGTTGGAATCCAGAGAAGAAAGCAGGTGTGTCAAAGGCT | |
| GGCAGCCAAAGGTCGGCGCATCCCCCTCAGTGAGATGATGTGCAGGGATCTACCAGGGTT | |
| CCCTCTTGTAAGATCTTGCCAGATGCCTGAGTGCAGTAAAATCAAATCAGAGATGAAGAC | |
| AAAACTTGGTGAGCAGGGTCCGCAGATCCTCAGTGTCCAGAGAGTCTACATTCAGACAAG | |
| GGAAGAGAAGCGTATTAACCTGACCATTGGTAGCAGAGCCTATTTGCTGCCCAACACATC | |
| CGTGATTATTAAGTGCCCCGTGCGACGATTCCAGAAATCTCTGATCCAGTGGGAGAAGGA | |
| TGGCCGTTGCCTGCAGAACTCCAAACGGCTTGGCATCACCAAGTCAGGCTCACTAAAAAT | |
| CCACGGTCTTGCTGCCCCCGACATCGGCGTGTACCGGTGCATTGCAGGCTCTGCACAGGA | |
| AACAGTTGTGCTCAAGCTCATTGGTACTGACAACCGGCTCATCGCACGCCCAGCCCTCAG | |
| GGAGCCTATGAGGGAATATCCTGGGATGGACCACAGCGAAGCCAATAGTTTGGGAGTCAC | |
| ATGGCACAAAATGAGGCAAATGTGGAATAACAAAAATGACCTTTATCTGGATGATGACCA | |
| CATTAGTAACCAGCCTTTCTTGAGAGCTCTGTTAGGCCACTGCAGCAATTCTGCAGGAAG | |
| CACCAACTCCTGGGAGTTGAAGAATAAGCAGTTTGAAGCAGCAGTTAAACAAGGAGCATA | |
| TAGCATGGATACAGCCCAGTTTGATGAGCTGATAAGAAACATGAGTCAGCTCATGGAAAC | |
| CGGAGAGGTCAGCGATGATCTTGCGTCCCAGCTGATATATCAGCTGGTGGCCGAATTAGC | |
| CAAGGCACAGCCAACACACATGCAGTGGCGGGGCATCCAGGAAGAGACACCTCCTGCTGC | |
| TCAGCTCAGAGGGGAAACAGGGAGTGTGTCCCAAAGCTCGCATGCAAAAAACTCAGGCAA | |
| GCTGACATTCAAGCCGAAAGGACCTGTTCTCATGAGGCAAAGCCAACCTCCCTCAATTTC | |
| ATTTAATAAAACAATAAATTCCAGGATTGGAAATACAGTATACATTACAAAAAGGACAGA | |
| GGTCATCAATATACTGTGTGACCTTATTACCCCCAGTGAGGCCACATATACATGGACCAA | |
| GGATGGAACCTTGTTACAGCCCTCAGTAAAAATAATTTTGGATGGAACTGGGAAGATACA | |
| GATACAGAATCCTACAAGGAAAGAACAAGGCATATATGAATGTTCTGTAGCTAATCATCT | |
| TGGTTCAGATGTGGAAAGTTCTTCTGTGCTGTATGCAGAGGCACCTGTCATCTTGTCTGT | |
| TGAAAGAAATATCACCAAACCAGAGCACAACCATCTGTCTGTTGTGGTTGGAGGCATCGT | |
| GGAGGCAGCCCTTGGAGCAAACGTGACAATCCGATGTCCTGTAAAAGGTGTCCCTCAGCC | |
| TAATATAACTTGGTTGAAGAGAGGAGGATCTCTGAGTGGCAATGTTTCCTTGCTTTTCAA | |
| TGGATCCCTGTTGTTGCAGAATGTTTCCCTTGAAAATGAAGGAACCTACGTCTGCATAGC | |
| CACCAATGCTCTTGGAAAGGCAGTGGCAACATCTGTACTCCACTTGCTGGAACGAAGATG | |
| GCCAGAGAGTAGAATCGTATTTCTGCAAGGACATAAAAAGTACATTCTCCAGGCAACCAA | |
| CACTAGAACCAACAGCAATGACCCAACAGGAGAACCCCCGCCTCAAGAGCCTTTTTGGGA | |
| GCCTGGTAACTGGTCACATTGTTCTGCCACCTGTGGTCATTTGGGAGCCCGCATTCAGAG | |
| ACCCCAGTGTGTGATGGCCAATGGGCAGGAAGTGAGTGAGGCCCTGTGTGATCACCTCCA | |
| GAAGCCACTGGCTGGGTTTGAGCCCTGTAACATCCGGGACTGCCCAGCGAGGTGGTTCAC | |
| AAGTGTGTGGTCACAGTGCTCTGTGTCTTGCGGTGAAGGATACCACAGTCGGCAGGTGAC | |
| GTGCAAGCGGACAAAAGCCAATGGAACTGTGCAGGTGGTGTCTCCAAGAGCATGTGCCCC | |
| TAAAGACCGGCCTCTGGGAAGAAAACCATGTTTTGGTCATCCATGTGTTCAGTGGGAACC | |
| AGGGAACCGGTGTCCTGGACGTTGCATGGGCCGTGCTGTGAGGATGCAGCAGCGTCACAC | |
| AGCTTGTCAACACAACAGCTCTGACTCCAACTGTGATGACAGAAAGAGACCCACCTTAAG | |
| AAGGAACTGCACATCAGGGGCCTGTGATGTGTGTTGGCACACAGGCCCTTGGAAGCCCTG | |
| TACAGCAGCCTGTGGCAGGGGTTTCCAGTCTCGGAAAGTCGACTGTATCCACACAAGGAG | |
| TTGCAAACCTGTGGCCAAGAGACACTGTGTACAGAAAAAGAAACCAATTTCCTGGCGGCA | |
| CTGTCTTGGGCCCTCCTGTGATAGAGACTGCACAGACACAACTCACTACTGTATGTTTGT | |
| AAAACATCTTAATTTGTGTTCTCTAGACCGCTACAAACAAAGGTGCTGCCAGTCATGTCA | |
| AGAGGGATAAACCTTTGGAGGGGTCATGATGCTGCTGTGAAGATAAAAGTAGAATATAAA | |
| AGCTCTTTTCCCCATGTCGCTGATTCAAAAACATGTATTTCTTAAAAGACTAGATTCTAT | |
| GGATCAAACAGAGGTTGATGCAAAAACACCACTGTTAAGGTGTAAAGTGAAATTTTCCAA | |
| TGGTAGTTTTATATTCCAATTTTTTAAAATGATGTATTCAAGGATGAACAAAATACTATA | |
| GCATGCATGCCACTGCACTTGGGACCTCATCATGTCAGTTGAATCGAGAAATCACCAAGA | |
| TTATGAGTGCATCCTCACGTGCTGCCTCTTTCCTGTGATATGTAGACTAGCACAGAGTGG | |
| TACATCCTAAAAACTTGGGAAACACAGCAACCCATGACTTCCTCTTCTCTCAAGTTGCAG | |
| GTTTTCAACAGTTTTATAAGGTATTTGCATTTTAGAAGCTCTGGCCAGTAGTTGTTAAGA | |
| TGTTGGCATTAATGGCATTTTCATAGATCCTTGGTTTAGTCTGTGAAAAAGAAACCATCT | |
| CTCTGGATAGGCTGTCACACTGACTGACCTAAGGGTTCATGGAAGCATGGCATCTTGTCC | |
| TTGCTTTTAGAACACCCATGGAAGAAAACACAGAGTAGATATTGCTGTCATTTATACAAC | |
| TACAGAAATTTATCTATGACCTAATGAGGCATCTCGGAAGTCAAAGAAGAGGGAAAGTTA | |
| ACCTTTTCTACTGATTTCGTAGTATATTCAGAGCTTTCTTTTAAGAGCTGTGAATGAAAC | |
| TTTTTCTAAGCACTATTCTATTGCACACAAACAGAAAACCAAAGCCTTATTAGACCTAAT | |
| TTATGCATAAAGTAGTATTCCTGAGAACTTTATTTTGGAAAATTTATAAGAAAGTAATCC | |
| AAATAAGAAACACGATAGTTGAAAATAATTTTTATAGTAAATAATTGTTTTGGGCTGATT | |
| TTTCAGTAAATCCAAAGTGACTTAGGTTAGAAGTTACACTAAGGACCAGGGGTTGGAATC | |
| AGAATTTAGTTTAAGATTTGAGGAAAAGGGTAAGGGTTAGTTTCAGTTTTAGGATTAGAG | |
| CTAGAATTGGGTTAGGTGAGAAAGAAAGTTAAGGTTAAGGCTAGAGTTGTCTTTAAGGGT | |
| TAGGGTTAGGACCAGGTTAGGTCAGGGTTGGATTGGGTTTAGATTGGGGCCAGTGCTGGT | |
| GTTAGTGATAGTGTCAGGATGGAGGTTAGGTTTGGAGTAAGCGTTGTTGCTGAAGTGAGT | |
| TCAGGCTAGCATTAAATTGTAAGTTCTGAAGCTGATTTGGTTATGGGGTCTTTCCCCTGT | |
| ATACTACCAGTTGTGTCTTTAGATGGCACACAAGTCCAAATAAGTGGTCATACTTCTTTA | |
| TTCAGGGTCTCAGCTGCCTGTACACCTGCTGCCTACATCTTCTTGGCAACAAAGTTACCT | |
| GCCACAGGCTCTGCTGAGCCTAGTTCCTGGTCAGTAATAACTGAACAGTGCATTTTGGCT | |
| TTGGATGTGTCTGTGGACAAGCTTGCTGAGTTTCTCTACCATATTCTGAGCACACGGTCT | |
| CTTTTGTTCTAATTTCAGCTTCACTGACACTGGGTTGAGCACTACTGTATGTGGAGGGTT | |
| TGGTGATTGGGAATGGATGGGGGACAGTGAGGAGGACACACCAGCCCATTAGTTGTTAAT | |
| CATCAATCACATCTGATTGTTGAAGGTTATTAAATTAAAAGAAAGATCATTTGTAACATA | |
| CTCTTTGTATATATTTATTATATGAAAGGTGCAATATTTTATTTTGTACAGTATGTAATA | |
| AAGACATGGGACATATATTTTTCTTATTAACAAAATTTCATATTAAATTGCTTCACTTTG | |
| TATTTAAAGTTAAAAGTTACTATTTTTCATTTGCTATTGTACTTTCATTGTTGTCATTCA | |
| ATTGACATTCCTGTGTACTGTATTTTACTACTGTTTTTATAACATGAGAGTTAATGTTTC | |
| TGTTTCATGATCCTTATGTAATTCAGAAATAAATTTACTTTGATTATTCAGTGGCATCCT | |
| TAT | |
| MPYDHFQPLPRWEHNPWTACSVSCGGGIQRRSFVCVEESMHGEILQVEEWKCMYAPKPKVMQTCNLFDCPKWIAMEW | (SEQ ID NO: 5) |
| SQCTVTCGRGLRYRVVLCINHRGEHVGGCNPQLKLHIKEECVIPIPCYKPKEKSPVEAKLPWLKQAQELEETRIATE | |
| EPTFIPEPWSACSTTCGPGVQVREVKCRVLLTFTQTETELPEEECEGPKLPTERPCLLEACDESPASRELDIPLPED | |
| SETTYDWEYAGFTPCTATCVGGHQEAIAVCLHIQTQQTVNDSLCDMVHRPPAMSQACNTEPCPPRWHVGSWGPCSAT | |
| CGVGIQTRDVYCLHPGETPAPPEECRDEKPHALQACNQFDCPPGWHIEEWQQCSRTCGGGTQNRRVTCRQLLTDGSF | |
| LNLSDELCQGPKASSHKSCARTDCPPHLAVGDWSKCSVSCGVGIQRRKQVCQRLAAKGRRIPLSEMMCRDLPGFPLV | |
| RSCQMPECSKIKSEMKTKLGEQGPQILSVQRVYIQTREEKRINLTIGSRAYLLPNTSVIIKCPVRRFQKSLIQWEKD | |
| GRCLQNSKRLGITKSGSLKIHGLAAPDIGVYRCIAGSAQETVVLKLIGTDNRLIARPALREPMREYPGMDHSEANSL | |
| GVTWHKMRQMWNNKNDLYLDDDHISNQPFLRALLGHCSNSAGSTNSWELKNKQFEAAVKQGAYSMDTAQFDELIRNM | |
| SQLMETGEVSDDLASQLIYQLVAELAKAQPTHMQWRGIQEETPPAAQLRGETGSVSQSSHAKNSGKLTFKPKGPVLM | |
| RQSQPPSISFNKTINSRIGNTVYITKRTEVINILCDLITPSEATYTWTKDGTLLQPSVKIILDGTGKIQIQNPTRKE | |
| QGIYECSVANHLGSDVESSSVLYAEAPVILSVERNITKPEHNHLSVVVGGIVEAALGANVTIRCPVKGVPQPNITWL | |
| KRGGSLSGNVSLLFNGSLLLQNVSLENEGTYVCIATNALGKAVATSVLHLLERRWPESRIVFLQGHKKYILQATNTR | |
| TNSNDPTGEPPPQEPFWEPGNWSHCSATCGHLGARIQRPQCVMANGQEVSEALCDHLQKPLAGFEPCNIRDCPARWF | |
| TSVWSQCSVSCGEGYHSRQVTCKRTKANGTVQVVSPRACAPKDRPLGRKPCFGHPCVQWEPGNRCPGRCMGRAVRMQ | |
| QRHTACQHNSSDSNCDDRKRPTLRRNCTSGACDVCWHTGPWKPCTAACGRGFQSRKVDCIHTRSCKPVAKRHCVQKK | |
| KPISWRHCLGPSCDRDCTDTTHYCMFVKHLNLCSLDRYKQRCCQSCQEG | |

Table 9 shows a multiple sequence alignment of NOV-1, NOV-2a, and NOV-2b polypeptides with a KIAA1233 protein (GenBank Accession No: BAA86547), that demonstrates the homology between disclosed sequences according to the invention and a known member of the protein family.

TABLE 9

| | |
|---|---|
| KIAA1233 | ------------------------------------------------------------ |
| NOV1 | ------------------------------------------------------------ |
| NOV2b | ------------------------------------------------------------ |
| NOV2a | MASWTSPWWVLIGMVFMHSPLPQTTAEKSPGAYFLPEFALSPQGSFLEDTTGEQFLTYRY |
| KIAA1233 | ------------------------------------------------------------ |
| NOV1 | ------------------------------------------------------------ |
| NOV2b | ------------------------------------------------------------ |
| NOV2a | DDQTSRNTRSDEDKDGNWDAWGDWSDCSRTCGGGASYSLRRCLTGRNCEGQNIRYKTCSN |
| KIAA1233 | ------------------------------------------------------------ |
| NOV1 | ------------------------------------------------------------ |
| NOV2b | ------------------------------------------------------------ |
| NOV2a | HDCPPDAEDFRAQQCSAYNDVQYQGHYYEWLPRYNDPAAPCALKCHAQGQNLVVELAPKV |
| KIAA1233 | ------------------------------------------------------------ |
| NOV1 | ------------------------------------------------------------ |
| NOV2b | ------------------------------------------------------------ |
| NOV2a | LDGTRCNTDSLDMCISGICQAVGCDRQLGSNAKEDNCGVCAGDGSTCRLVRGQSKSHVSP |
| KIAA1233 | ------------------------------------------------------------ |
| NOV1 | ------------------------------------------------------------ |
| NOV2b | ------------------------------------------------------------ |
| NQV2a | EKREENVIAVPLGSRSVRITVKGPAHLFIESKTLQGSKGEHSFNSPGVFVVENTTVEFQR |
| KIAA1233 | ------------------------------------------------------------ |
| NOV1 | ------------------------------------------------------------ |
| NOV2b | ------------------------------------------------------------ |
| NOV2a | GSERQTFKIPGPLMADFIFKTRYTAAKDSVVQFFFYQPISHQWRQTDFFPCTVTCGGGYQ |
| KIAA1233 | ------------------------------------------------------------ |
| NOV1 | --------------------------------------------------MPYDHFQPLP |
| NOV2b | --------------------------------------------------MPYDHFQPLP |
| NOV2a | LNSAECVDIRLKRVVPDHYCHYYPENVKPKPKLKECSMDPCPSSDGFKEIMPYDHFQPLP |
| KIAA1233 | ------------------------------------------------------------ |
| NOV1 | RWEHNPWTACSVSCGGGIQRRSFVCVEESMHGEILQVEEWKCMYAPKPKVMQTCNLFDCP |
| NOV2b | RWEHNPWTACSVSCGGGIQRRSFVCVEESMHGEILQVEEWKCMYAPKPKVMQTCNLFDCP |
| NOV2a | RWEHNPWTACSVSCGGGIQRRSFVCVEESMHGEILQVEEWKCMYAPKPKVMQTCNLFDCP |
| KIAA1233 | ------------------------------------------------------------ |
| NOV1 | KWIAMEWSQCTVTCGRGLRYRVVLCINHRGEHVGGCNPQLKLHIKEECVIPIPCYKPKEK |
| NOV2b | KWIAMEWSQCTVTCGRGLRYRVVLCINHRGEHVGGCNPQLKLHIKEECVIPIPCYKPKEK |
| NOV2a | KWIAMEWSQCTVTCGRGLRYRVVLCINHRGEHVGGCNPQLKLHIKEECVIPIPCYKPKEK |
| KIAA1233 | ------------------------------------------------------------ |
| NOV1 | SPVEAKLPWLKQAQELEETRIATEEPTFIPEPWSACSTTCGPGVQVREVKCRVLLTFTQT |
| NOV2b | SPVEAKLPWLKQAQELEETRIATEEPTFIPEPWSACSTTCGPGVQVREVKCRVLLTFTQT |
| NOV2a | SPVEAKLPWLKQAQELEETRIATEEPTFIPEPWSACSTTCGPGVQVREVKCRVLLTFTQT |
| KIAA1233 | ------------------------------------------------------------ |
| Nov1 | ETELPEEECEGPKLPTERPCLLEACDESPASRELDIPLPEDSETTYDWEYAGFTPCTATC |
| NOV2b | ETELPEEECEGPKLPTERPCLLEACDESPASRELDIPLPEDSETTYDWEYAGFTPCTATC |
| NOV2a | ETELPEEECEGPKLPTERPCLLEACDESPASRELDIPLPEDSETTYDWEYAGFTPCTATC |
| KIAA1233 | --------AVCLHIQTQQTVNDSLCDMVHRPPAMSQACNTEPCPPRWHVGSWGPCSATCG |
| NOV1 | VGGHQEAIAVCLHIQTQQTVNDSLCDMVHRPPAMSQACNTEPCPPRWHVGSWGPCSATCG |
| NOV2b | VGGHQEAIAVCLHIQTQQTVNDSLCDMVHRPPAMSQACNTEPCPPRWHVGSWGPCSATCG |
| NOV2a | VGGHQEAIAVCLHIQTQQTVNDSLCDMVHRPPAMSQACNTEPCPPRWHVGSWGPCSATCG |
| | **************************************************** |
| KIAA1233 | VGIQTRDVYCLHPGETPAPPEECRDEKPHALQACNQFDCPPGWHIEEWQQCSRTCGGGTQ |
| NOV1 | VGIQTRDVYCLHPGETPAPPEECRDEKPHALQACNQFDCPPGWHIEEWQQCSRTCGGGTQ |
| NOV2b | VGIQTRDVYCLHPGETPAPPEECRDEKPHALQACNQFDCPPGWHIEEWQQCSRTCGGGTQ |
| NOV2a | VGIQTRDVYCLHPGETPAPPEECRDEKPHALQACNQFDCPPGWHIEEWQQCSRTCGGGTQ |
| | ************************************************************ |
| KIAA1233 | NRRVTCRQLLTDGSFLNLSDELCQGPKASSHKSCARTDCPPHLAVGDWSKCSVSCGVGIQ |
| NOV1 | NRRVTCRQLLTDGSFLNLSDELCQGPKASSHKSCARTDCPPHLAVGDWSKCSVSCGVGIQ |
| NOV2b | NRRVTCRQLLTDGSFLNLSDELCQGPKASSHKSCARTDCPPHLAVGDWSKCSVSCGVGIQ |
| NOV2a | NRRVTCRQLLTDGSFLNLSDELCQGPKASSHKSCARTDCPPHLAVGDWSKCSVSCGVGIQ |
| | ************************************************************ |
| KIAA1233 | RRKQVCQRLAAKGRRIPLSEMMCRDLPGLPLVRSCQMPECSKIKSEMKTKLGEQGPQILS |
| Nov1 | RRKQVCQRLAAKGRRIPLSEMMCRDLPGLPLVRSCQMPECSKIKSEMKTKLGEQGPQILS |
| NOV2b | RRKQVCQRLAAKGRRIPLSEMMCRDLPGFPLVRSCQMPECSKIKSEMKTKLGEQGPQILS |
| NOV2a | RRKQVCQRLAAKGRRIPLSEMMCRDLPGFPLVRSCQMPECSKIKSEMKTKLGEQGPQILS |
| | ****************************:******************************* |
| KIAA1233 | VQRVYIQTREEKRINLTIGSRAYLLPNTSVIIKCPVRRFQKSLIQWEKDGRCLQNSKRLG |
| NOV1 | VQRVYIQTREEKRINLTIGSRAYLLPNTSVIIKCPVRRFQKSLIQWEKDGRCLQNSKRLG |
| NOV2b | VQRVYIQTREEKRINLTIGSRAYLLPNTSVIIKCPVRRFQKSLIQWEKDGRCLQNSKRLG |
| NOV2a | VQRVYIQTREEKRINLTIGSRAYLLPNTSVIIKCPVRRFQKSLIQWEKDGRCLQNSKRLG |
| | ************************************************************ |
| KIAA1233 | ITKSGSLKIHGLAAPDIGVYRCIAGSAQETVVLKLIGTDNRLIARPALREPMREYPGMDH |
| NOV1 | ITKSGSLKIHGLAAPDIGVYRCIAGSAQETVVLKLIGTDNRLIARPALREPMREYPGMDH |
| NOV2b | ITKSGSLKIHGLAAPDIGVYRCIAGSAQETVVLKLIGTDNRLIARPALREPMREYPGMDH |
| NOV2a | ITKSGSLKIHGLAAPDIGVYRCIAGSAQETVVLKLIGTDNRLIARPALREPMREYPGMDH |
| | ************************************************************ |
| KIAA1233 | SEANSLGVTWHKMRQMWNNKNDLYLDDDHISNQPFLRALLGHCSNSAGSTNSWELKNKQF |
| NOV1 | SEANSLGVTWHKMRQMWNNKNDLYLDDDHISNQPFLRALLGHCSNSAGSTNSWELKNKQF |
| NOV2b | SEANSLGVTWHKMRQMWNNKNDLYLDDDHISNQPFLRALLGHCSNSAGSTNSWELKNKQF |
| NOV2a | SEANSLGVTWHKMRQMWNNKNDLYLDDDHISNQPFLRALLGHCSNSAGSTNSWELKNKQF |
| | ************************************************************ |
| KIAA1233 | EAAVKQGAYSMDTAQFDELIRNMSQLMETGEVSDDLASQLIYQLVAELAKAQPTHMQWRG |
| NOV1 | EAAVKQGAYSMDTAQFDELIRNMSQLMETGEVSDDLASQLIYQLVAELAKAQPTHMQWRG |
| NOV2b | EAAVKQGAYSMDTAQFDELIRNMSQLMETGEVSDDLASQLIYQLVAELAKAQPTHMQWRG |
| NOV2a | EAAVKQGAYSMDTAQFDELIRNMSQLMETGEVSDDLASQLIYQLVAELAKAQPTHMQWRG |
| | ************************************************************ |
| KIAA1233 | IQEETPPAAQLRGETGSVSQSSHAKNSGKLTFKPKGPVLMRQSQPPSISFNKTINSRIGN |
| NOV1 | IQEETPPAAQLRGETGSVSQSSHAKNSGKLTFKPKGPVLMRQSQPPSISFNKTINSRIGN |
| NOV2b | IQEETPPAAQLRGETGSVSQSSHAKNSGKLTFKPKGPVLMRQSQPPSISFNKTINSRIGN |
| NOV2a | IQEETPPAAQLRGETGSVSQSSHAKNSGKLTFKPKGPVLMRQSQPPSISFNKTINSRIGN |
| | ************************************************************ |
| KIAA1233 | TVYITKRTEVINILCDLITPSEATYTWTKDGTLLQPSVKIILDGTGKIQIQNPTRKEQGI |
| NOV1 | TVYITKRTEVINILCDLITPSEATYTWTKDGTLLQPSVKIILDGTGKIQIQNPTRKEQGI |
| NOV2b | TVYITKRTEVINILCDLITPSEATYTWTKDGTLLQPSVKIILDGTGKIQIQNPTRKEQGI |
| NOV2a | TVYITKRTEVINILCDLITPSEATYTWTKDGTLLQPSVKIILDGTGKIQIQNPTRKEQGI |
| | ************************************************************ |
| KIAA1233 | YECSVANHLGSDVESSSVLYAEAPVILSVERNITKPEHNHLSVVVGGIVEAALGANVTIR |
| NOV1 | YECSVANHLGSDVESSSVLYAEAPVILSVERNITKPEHNHLSVVVGGIVEAALGANVTIR |
| NOV2b | YECSVANHLGSDVESSSVLYAEAPVILSVERNITKPEHNHLSVVVGGIVEAALGANVTIR |
| NOV2a | YECSVANHLGSDVESSSVLYAEAPVILSVERNITKPEHNHLSVVVGGIVEAALGANVTIR |
| | ************************************************************ |
| KIAA1233 | CPVKGVPQPNITWLKRGGSLSGNVSLLFNGSLLLQNVSLENEGTYVCIATNALGKAVATS |
| NOV1 | CPVKGVPQPNITWLKRGGSLSGNVSLLFNGSLLLQNVSLENEGTYVCIATNALGKAVATS |
| NOV2b | CPVKGVPQPNITWLKRGGSLSGNVSLLFNGSLLLQNVSLENEGTYVCIATNALGKAVATS |
| NOV2a | CPVKGVPQPNITWLKRGGSLSGNVSLLFNGSLLLQNVSLENEGTYVCIATNALGKAVATS |
| | ************************************************************ |
| KIAA1233 | VLHLLERRWPESRIVFLQGHKKYILQATNTRTNSNDPTGEPPPQEPFWEPGNWSHCSATC |
| NOV1 | VLHLLERRWPESRIVFLQGHKKYILQATNTRTNSNDPTGEPPPQEPFWEPGNWSHCSATC |
| NOV2b | VLHLLERRWPESRIVFLQGHKKYILQATNTRTNSNDPTGEPPPQEPFWEPGNWSHCSATC |
| NOV2a | VFHLLERRWPESRIVFLQGHKKYILQATNTRTNSNDPTGEPPPQEPFWEPGNWSHCSATC |
| | *:********************************************************** |
| KIAA1233 | GHLGARIQRPQCVMANGQEVSEALCDHLQKPLAGFEPCNIRDCPARWFTSVWSQCSVSCG |
| NOV1 | GHLGARIQRPQCVMANGQEVSEALCDHLQKPLAGFEPCNIRDCPARWFTSVWSQCSVSCG |
| NOV2b | GHLGARIQRPQCVMANGQEVSEALCDHLQKPLAGFEPCNIRDCPARWFTSVWSQCSVSCG |
| NOV2a | GHLGARIQRPQCVMANGQEVSEALCDHLQKPLAGFEPCNIRDCPARWFTSVWSQCSVSCG |
| | ************************************************************ |
| KIAA1233 | EGYHSRQVTCKRTKANGTVQVVSPRACAPKDRPLGRKPCFGHPCVQWEPGNRCPGRCMGR |
| NOV1 | EGYHSRQVTCKRTKANGTVQVVSPRACAPKDRPLGRKPCFGHPCVQWEPGNRCPGRCMGR |
| NOV2b | EGYHSRQVTCKRTKANGTVQVVSPRACAPKDRPLGRKPCFGHPCVQWEPGNRCPGRCMGR |
| NOV2a | EGYHSRQVTCKRTKANGTVQVVSPRACAPKDRPLGRKPCFGHPCVQWEPGNRCPGRCMGR |
| | ************************************************************ |
| KIAA1233 | AVRMQQRHTACQHNSSDSNCDDRKRPTLRRNCTSGACDVCWHTGPWKPCTAACGRGFQSR |
| NOV1 | AVRMQQRHTACQHNSSDSNCDDRKRPTLRRNCTSGACDVCWHTGPWKPCTAACGRGFQSR |
| NOV2b | AVRMQQRHTACQHNSSDSNCDDRKRPTLRRNCTSGACDVCWHTGPWKPCTAACGRGFQSR |
| NOV2a | AVRMQQRHTACQHNSSDSNCDDRKRPTLRRNCTSGACDVCWHTGPWKPCTAACGRGFQSR |
| | ************************************************************ |
| KIAA1233 | KVDCIHTRSCKPVAKRHCVQKKKPISWRHCLGPSCDRDCTDTTHYCMFVKHLNLCSLDRY |
| NOV1 | KVDCIHTRSCKPVAKRHCVQKKKPISWRHCLGPSCDRDCTDTTHYCMFVKHLNLCSLDRY |
| NOV2b | KVDCIHTRSCKPVAKRHCVQKKKPISWRHCLGPSCDRDCTDTTHYCMFVKHLNLCSLDRY |
| NOV2a | KVDCIHTRSCKPVAKRHCVQKKKPISWRHCLGPSCDRDCTDTTHYCMFVKHLNLCSLDRY |
| | ************************************************************ |
| KIAA1233 | KQRCCQSCQEG (SEQ ID NO: 28) |
| NOV1 | KQRCCQSCQEG (SEQ ID NO: 2) |
| NOV2b | KQRCCQSCQEG (SEQ ID NO: 5) |
| NOV2a | KQRCCQSCQEG (SEQ ID NO: 4) |
| | ********** |

Consensus key
* - single, fully conserved residue
: - conservation of strong groups
. - conservation of weak groups - no consensus

Based the relatedness of the disclosed NOV-2b to the disclosed NOV-1, the disclosed NOV-2a, and KIAA1233 sequences, which as noted are related to lacunin, thrombospondins, proteinases, semaphorins, ADAM-TS and properdin family members, the nucleic acids and proteins of the invention can have similar functions as proteins belonging to these families. Thus, the invention is implicated in the following diseases and processes and has therapeutic uses in these diseases and processes: (i) inflammation, (ii) cancer, (iii) neuronal development and axonal guidance, (iv) angiogenesis and vasculogenesis—in cancer as well as for ischemia, and (v) tissue regeneration in vivo and in vitro, and (vi) and other diseases and disorders.

Functional roles attributed to this family of proteins include cell attachment, spreading, motility, and proliferation, cytoskeletal organization, wound healing, and angiogenesis. Moreover, these proteins are expressed in the nervous systems during development and are thought to play roles in neuronal growth and patterning. In particular, the thrombospondin, METH-1 and ADAMTS families of proteins are potent inhibitors of angiogenesis. The ADAMTS proteins have also been implicated in cleavage of proteglycans and the control of organ shape during development. In addition, the thrombospondins have been implicated in the activation of both transforming growth factor-beta (TGF-β) precursors and TGF-β in a variety of disease states. Furthermore, semaphorin proteins have shown expression in undifferentiated neuroepithelium, suggesting that these proteins are actors in axonal guidance.

The novel nucleic acids of the invention encoding human proteins includes the nucleic acids whose sequences are provided as NOV-1, NOV-2a, and NOV-2b, respectively, or fragments thereof. The invention also includes mutant or variant nucleic acids any of whose bases may be changed from the corresponding bases shown as NOV-1, NOV-2a, and NOV-2b, while still encoding a protein that maintains its human KIAA1233-like proteins activities and physiological functions, or a fragment of such nucleic acids. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as anti-sense binding nucleic acids in therapeutic applications in a subject.

The novel proteins of the invention includes the human KIAA1233-like proteins whose sequences are provided as NOV-1, NOV-2a, and NOV-2b, respectively. The invention also includes a mutant or variant protein any of whose residues may be changed from the corresponding residues shown as NOV-1, NOV-2a, and NOV-2b, while still encoding a protein that maintains its human KIAA1233-like protein activities and physiological functions, or a functional fragment thereof.

The invention further encompasses antibodies and antibody fragments, such as F_{ab} or (F_{ab})₂, that bind immunospecifically to any of the proteins of the invention.

The expression pattern, and protein similarity information for the invention suggest that NOV-1, NOV-2a and NOV-2b may function as human KIAA1233-like proteins. Therefore, the nucleic acid and protein of the invention are useful in potential therapeutic applications implicated, for example but not limited to, (i) inflammation, (ii) cancer, (iii) neuronal development and axonal guidance, (iv) angiogenesis and vasculogenesis—in cancer as well as for ischemia, and (v) tissue regeneration in vivo and in vitro, (vi) and other diseases and disorders. The homology to antigenic secreted and membrane proteins also suggests that antibodies directed against the novel genes may be useful in treatment and prevention of (i) inflammation, (ii) cancer, (iii) neuronal development and axonal guidance, (iv) angiogenesis and vasculogenesis—in cancer as well as for ischemia, and (v) tissue regeneration in vivo and in vitro, and (vi) other diseases and disorders.

Potential therapeutic uses for the invention(s) are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

NOV-3: A Novel STE20 Protein Kinase

The NOV-3 sequences (NOV-3a, NOV-3b, NOV-3c, and NOV-3d) according to the invention are splice variants related to STE20 protein kinases. The differences between the four sequences relate to the four ways of independently combining two deletions arising from two splice variants in the mRNAs.

Splice variants are sequences that occur naturally within the cells and tissues of individuals. The physiological activity of splice variant products and the original protein, from which they are varied, may be the same (although perhaps at a different level), opposite, or completely different and unrelated. In addition, variants may have no activity at all. When a variant and the original sequence have the same or opposite activity, they may differ in various properties not directly connected to biological activity, such as stability, clearance rate, tissue and cellular localization, temporal pattern of expression, up or down regulation mechanisms, and responses to agonists or antagonists. The presence or level of specific splice variants may be the cause, and/or indicative of, a disease, disorder, pathological or normal condition.

Because a drug may be effective against one variant but not another, or may cause side effects because it targets all splice variants, an effective drug needs to target the particular splice variant. Because soluble variants with therapeutic or disease-related functions may be naturally occurring in specific tissues, they may be optimal candidates for drug targets or protein therapeutics. Variants may have no activity at all and may thus serve as dominant negative natural inhibitors. Thus, splice variants useful in generating new drug targets, protein therapeutics and markers for diagnostics.

NOV-3 sequences according to the invention encode polypeptides related to STE20 protein kinases, whose subgroups include GCK, SLK, and PSK proteins. Therefore, the nucleic acids and proteins of the invention can have similar functions as proteins belonging to these subgroups.

Functional roles attributed to STE20 proteins include cytoskeletal organization, apoptosis, and signal transduction pathways. Thus, the NOV-3 nucleic acids and polypeptides, antibodies and related compounds according to the invention will be useful in therapeutic and diagnostic applications in disorders associated with, e.g., metabolic and endocrine disorders, cancer, bone disorders, and tissue/cell growth regulation disorders.

NOV-3 sequences were initially identified by searching CuraGen's Human SeqCalling database for DNA sequences that translate into proteins with similarity to the STE20 protein kinase family. The SeqCalling assembly for NOV-3 was analyzed further to identify open reading frame(s) encoding for novel full length protein(s) and novel splice variants of these genes. This was done by extending the SeqCalling assembly using additional SeqCalling assemblies, publicly available EST sequences and public genomic sequence. Public ESTs and additional CuraGen SeqCalling assemblies were identified by the CuraTools program SeqExtend. They were included in the DNA sequence extension for SeqCalling assembly 18552586 when extended overlaps were found.

SeqCalling is a differential expression and sequencing procedure that normalizes mRNA species in a sample, and is disclosed in U.S. Ser. No. 09/417,386 filed Oct. 13, 1999, which is incorporated herein by reference in its entirety.

A genomic clone of NOV-3 was analyzed by Genscan™ and Grail™ to identify exons and putative coding sequences/open reading frames. The NOV-3 clone was also analyzed by TblastN, BlastX and other homology programs to identify regions translating to proteins with similarity to the original protein/protein family of interest.

The results of these analyses were integrated and manually corrected for apparent inconsistencies, thereby obtaining the sequences encoding the full-length proteins. When necessary, the process to identify and analyse cDNAs/ESTs and genomic clones was reiterated to derive the full-length sequence. The full-length DNA sequences as well as their splice forms, and the full-length protein sequences that they encode, are disclosed herein.

NOV-3 was mapped to chromosome 17.

Based on the CuraGen SeqCalling database information, the NOV-3 is expressed in heart tissue. Moreover, based on the expression of STE-20 family members, the following tissues are also likely to express the invention: brain (especially hippocampus and cerebral cortex), prostate, and blood hematopoetic cell lines. The patterns of expression for this gene and its family members, combined with its similarity to the STE20 kinase family of genes, suggests that the NOV-3 proteins function as kinases in the tissues of expression. Thus, NOV-3 is implicated in disorders involving these tissues. Some of these disorders include: cardiovascular disorders, diabetes, leukemia/lymphoma, cancer, musculoskeletal disorders, muscular generation, reproductive health, metabolic and endocrine disorders, gastrointestinal disorders, immune and autoinumune disorders, respiratory disorders, bone disorders, and tissue/cell growth regulation disorders.

Additional utilities for NOV-3 nucleic acids and polypeptides according to the invention are also disclosed herein.

NOV-3a

A NOV-3a sequence according to the invention is a nucleic acid sequence encoding a polypeptide related to STE20 family of protein kinases. A disclosed NOV-3a nucleic acid and its encoded polypeptide includes the sequences shown in Table 10. The disclosed nucleic acid (SEQ ID NO: 6) is 3999 nucleotides in length and contains an open reading frame (ORF) that begins with an ATG initiation codon at nucleotides 1-3 and ends with a TGA stop codon at nucleotides 3996-3999. The start and stop codons are shown in bold font. The respective ORF encodes a 1332 amino acid polypeptide (SEQ ID NO: 7).

TABLE 10

| | |
|---|---|
| ATGGGCGACCCAGCCCCCGCCCGCAGCCTGGACGACATCGACCTGTCCGCCCTGCGGGACCCTGCTGGGATCTTTGAGCT | (SEQ ID NO: 6) |
| TGTGGAGGTGGTCGGCAATGGAACCTACGGACAGGTGTACAAGGGTCGGCATGTCAAGACGGGGCAGCTGGCTGCCATCA | |
| AGGTCATGGATGTCACGGAGGACGAGGAGGAAGAGATCAAACAGGAGATCAACATGCTGAAAAAGTACTCTCACCACCGC | |
| AACATCGCCACCTACTACGGAGCCTTCATCAAGAAGAGCCCCCCGGGAAACGATGACCAGCTCTGGCTGGTGATGGAGTT | |
| CTGTGGTGCTGGTTCAGTGACTGACCTGGTAAAGAACACAAAAGGCAACGCCCTGAAGGAGGACTGTATCGCCTATATCT | |
| GCAGGGAGATCCTCAGGGGTCTGGCCCATCTCCATGCCCACAAGGTGATCCATCGAGACATCAAGGGGCAGAATGTGCTG | |
| CTGACAGAGAATGCTGAGGTCAAGCTAGTGGATTTTGGGGTGAGTGCTCAGCTGGACCGCACCGTGGGCAGACGGAACAC | |
| TTTCATTGGGACTCCCTACTGGATGGCTCCAGAGGTCATCGCCTGTGATGAGAACCCTGATGCCACCTATGATTACAGGA | |
| GTGATATTTGGTCTCTAGGAATCACAGCCATCGAGATGGCAGAGGGAGCCCCCCCTCTGTGTGACATGCACCCCATGCGA | |
| GCCCTCTTCCTCATTCCTCGGAACCCTCCGCCCAGGCTCAAGTCCAAGAAGTGGTCTAAGAAGTTCATTGACTTCATTGA | |
| CACATGTCTCATCAAGACTTACCTGAGCCGCCCACCCACGGAGCAGCTACTGAAGTTTCCCTTCATCCGGGACCAGCCCA | |
| CGGAGCGGCAGGTCCGCATCCAGCTTAAGGACCACATTGACCGATCCCGGAAGAAGCGGGGTGAGAAAGAGGAGACAGAA | |
| TATGAGTACAGCGGCAGCGAGGAGGAAGATGACAGCCATGGAGAGGAAGGAGAGCCAAGCTCCATCATGAACGTGCCTGG | |
| AGAGTCGACTCTACGCCGGGAGTTTCTCCGGCTCCAGCAGGAAAATAAGAGCAACTCAGAGGCTTTAAAACAGCAGCAGC | |
| AGCTGCAGCAGCAGCAGCAGCGAGACCCCGAGGCACACATCAAACACCTGCTGCACCAGCGGCAGCGGCGCATAGAGGAG | |
| CAGAAGGAGGAGCGGCGCCGCGTGGAGGAGCAACAGCGGCGGGAGCGGGAGCAGCGGAAGCTGCAGGAGAAGGAGCAGCA | |
| GCGGCGGCTGGAGGACATGCAGGCTCTGCGGCGGGAGGAGGAGCGGCGGCAGGCGGAGCGCGAGCAGGAATATATTCGTC | |
| ACAGGCTAGAGGAGGAGCAGCGACAGCTCGAGATCCTTCAGCAACAGCTGCTCCAGGAACAGGCCCTGCTGCTGGAATAC | |
| AAGCGGAAGCAGCTGGAGGAGCAGCGGCAGTCAGAACGTCTCCAGAGGCAGCTGCAGCAGGAGCATGCCTACCTCAAGTC | |
| CCTGCAGCAGCAGCAACAGCAGCAGCAGCTTCAGAAACAGCAGCAGCAGCAGCTCCTGCCTGGGGACAGGAAGCCCCTGT | |
| ACCATTATGGTCGGGGCATGAATCCCGCTGACAAACCAGCCTGGGCCCGAGAGGTAGAAGAGAGAACAAGGATGAACAAG | |
| CAGCAGAACTCTCCCTTGGCCAAGAGCAAGCCAGGCAGCACGGGGCCTGAGCCCCCCATCCCCCAGGCCTCCCCAGGGCC | |
| CCCAGGACCCCTTTCCCAGACTCCTCCTATGCAGAGGCCGGTGGAGCCCCAGGAGGGACCGCACAAGAGCCTGGTGGCAC | |
| ACCGGGTCCCACTGAAGCCATATGCAGCACCTGTACCCCGATCCCAGTCCCTGCAGGACCAGCCCACCCGAAACCTGGCT | |
| GCCTTCCCAGCCTCCCATGACCCCGACCCTGCCATCCCCGCACCCACTGCCACGCCCAGTGCCCGAGGAGCTGTCATCCG | |
| CCAGAATTCAGACCCCACCTCTGAAGGACCTGGCCCCAGCCCGAATCCCCCAGCCTGGGTCCGCCCAGATAACGAGGCCC | |
| CACCCAAGGTGCCTCAGAGGACCTCATCTATCGCCACTGCCCTTAACACCAGTGGGGCCGGAGGGTCCCGGCCAGCCCAG | |
| GCAGTCCGTGCCAGTAACCCCGACCTCAGGAGGAGCGACCCTGGCTGGGAACGCTCGGACAGCGTCCTTCCAGCCTCTCA | |
| CGGGCACCTCCCCCAGGCTGGCTCACTGGAGCGGAACCGCGTGGGAGTCTCCTCCAAACCGGACAGCTCCCCTGTGCTCT | |
| CCCCTGGGAATAAAGCCAAGCCCGACGACCACCGCTCACGGCCAGGCCGGCCCGCAAGCTATAAGCGAGCAATTGGTGAG | |
| GACTTTGTGTTGCTGAAAGAGCGGACTCTGGACGAGGCCCCTCGGCCTCCCAAGAAGGCCATGGACTACTCGTCGTCCAG | |
| CGAGGAGGTGGAAAGCAGTGAGGACGACGAGGAGGAAGGCGAAGGCGGGCCAGCAGAGGGGAGCAGAGATACCCCTGGGG | |
| GCCGCAGCGATGGGGATACAGACAGCGTCAGCACCATGGTGGTCCACGACGTCGAGGAGATCACCGGGACCCAGCCCCCA | |
| TACGGGGGCGGCACCATGGTGGTCCAGCGCACCCCTGAAGAGGAGCGGAACCTGCTGCATGCTGACAGCAATGGGTACAC | |
| AAACCTGCCTGACGTGGTCCAGCCCAGCCACTCACCCACCGAGAACAGCAAAGGCCAAAGCCCACCCTCGAAGGATGGGA | |
| GTGGTGACTACCAGTCTCGTGGGCTGGTAAAGGCCCCTGGCAAGAGCTCGTTCACGATGTTTGTGGATCTAGGGATCTAC | |
| CAGCCTGGAGGCAGTGGGGACAGCATCCCCATCACAGCCCTAGTGGGTGGAGAGGGCACTCGGCTCGACCAGCTGCAGTA | |
| CGACGTGAGGAAGGGTTCTGTGGTCAACGTGAATCCCACCAACACCCGGGCCCACAGTGAGACCCCTGAGATCCGGAAGT | |
| ACAAGAAGCGATTCAACTCCGAGATCCTCTGTGCAGCCCTTTGGGGGGTCAACCTGCTGGTGGGCACGGAGAACGGGCTG | |
| ATGTTGCTGGACCGAAGTGGGCAGGGCAAGGTGTATGGACTCATTGGGCGGCGACGCTTCCAGCAGATGGATGTGCTGGA | |
| GGGGCTCAACCTGCTCATCACCATCTCAGGGAAAAGGAACAAACTGCGGGTGTATTACCTGTCCTGGCTCCGGAACAAGA | |
| TTCTGCACAATGACCCAGAAGTGGAGAAGAAGCAGGGCTGGACCACCGTGGGGGACATGGAGGGCTGCGGGCACTACCGT | |
| GTTGTGAAATACGAGCGGATTAAGTTCCTGGTCATCGCCCTCAAGAGCTCCGTGGAGGTGTATGCCTGGGCCCCCAAACC | |
| CTACCACAAATTCATGGCCTTCAAGTCCTTTGCCGACCTCCCCCACCGCCCTCTGCTGGTCGACCTGACAGTAGAGGAGG | |
| GGCAGCGGCTCAAGGTCATCTATGGCTCCAGTGCTGGCTTCCATGCTGTGGATGTCGACTCGGGGAACAGCTATGACATC | |
| TACATCCCTGTGCACATCCAGAGCCAGATCACGCCCCATGCCATCATCTTCCTCCCCAACACCGACGGCATGGAGATGCT | |
| GCTGTGCTACGAGGACGAGGGTGTCTACGTCAACACGTACGGGCGCATCATTAAGGATGTGGTGCTGCAGTGGGGGGAGA | |
| TGCCTACTTCTGTGGCCTACATCTGCTCCAACCAGATAATGGGCTGGGGTGAGAAAGCCATTGAGATCCGCTCTGTGGAG | |
| ACGGGCCACCTCGACGGGGTCTTCATGCACAAACGAGCTCAGAGGCTCAAGTTCCTGTGTGAGCGGAATGACAAGGTGTT | |
| TTTTGCCTCAGTCCGCTCTGGGGGCAGCAGCCAAGTTTACTTCATGACTCTGAACCGTAACTGCATCATGAACTGGTGA | |
| MGDPAPARSLDDIDLSALRDPAGIFELVEVVGNGTYGQVYKGRHVKTGQLAAIKVMDVTEDEEEEIKQEINMLKKYSHHR | (SEQ ID NO: 7) |
| NIATYYGAFIKKSPPGNDDQLWLVMEFCGAGSVTDLVKNTKGNALKEDCIAYICREILRGLAHLHAHKVIHRDIKGQNVL | |
| LTENAEVKLVDFGVSAQLDRTVGRRNTFIGTPYWMAPEVIACDENPDATYDYRSDIWSLGITAIEMAEGAPPLCDMHPMR | |
| ALFLIPRNPPPRLKSKKWSKKFIDFIDTCLIKTYLSRPPTEQLLKFPFIRDQPTERQVRIQLKDHIDRSRKKRGEKEETE | |
| YEYSGSEEEDDSHGEEGEPSSIMNVPGESTLRREFLRLQQENKSNSEALKQQQQLQQQQQRDPEAHIKHLLHQRQRRIEE | |
| QKEERRRVEEQQRREREQRKLQEKEQQRRLEDMQALRREEERRQAEREQEYIRHRLEEEQRQLEILQQQLLQEQALLLEY | |
| KRKQLEEQRQSERLQRQLQQEHAYLKSLQQQQQQQQLQKQQQQQLLPGDRKPLYHYGRGMNPADKPAWAREVEERTRMNK | |
| QQNSPLAKSKPGSTGPEPPIPQASPGPPGPLSQTPPMQRPVEPQEGPHKSLVAHRVPLKPYAAPVPRSQSLQDQPTRNLA | |
| AFPASHDPDPAIPAPTATPSARGAVIRQNSDPTSEGPGPSPNPPAWVRPDNEAPPKVPQRTSSIATALNTSGAGGSRPAQ | |
| AVRASNPDLRRSDPGWERSDSVLPASHGHLPQAGSLERNRVGVSSKPDSSPVLSPGNKAKPDDHRSRPGRPASYKRAIGE | |
| DFVLLKERTLDEAPRPPKKAMDYSSSSEEVESSEDDEEEGEGGPAEGSRDTPGGRSDGDTDSVSTMVVHDVEEITGTQPP | |
| YGGGTMVVQRTPEEERNLLHADSNGYTNLPDVVQPSHSPTENSKGQSPPSKDGSGDYQSRGLVKAPGKSSFTMFVDLGIY | |
| QPGGSGDSIPITALVGGEGTRLDQLQYDVRKGSVVNVNPTNTRAHSETPEIRKYKKRFNSEILCAALWGVNLLVGTENGL | |
| MLLDRSGQGKVYGLIGRRRFQQMDVLEGLNLLITISGKRNKLRVYYLSWLRNKILHNDPEVEKKQGWTTVGDMEGCGHYR | |
| VVKYERIKFLVIALKSSVEVYAWAPKPYHKFMAFKSFADLPHRPLLVDLTVEEGQRLKVIYGSSAGFHAVDVDSGNSYDI | |
| YIPVHIQSQITPHAIIFLPNTDGMEMLLCYEDEGVYVNTYGRIIKDVVLQWGEMPTSVAYICSNQIMGWGEKAIEIRSVE | |
| TGHLDGVFMHKRAQRLKFLCERNDKVFFASVRSGGSSQVYFMTLNRNCIMNW | |

The disclosed NOV-3a nucleic acid sequence has homology (73% identity) to a mouse mRNA for a NIK protein (NIK) (GenBank Accession No: MMU88984), as shown in Table 11. NIK proteins are a subgroup of the STE20 family of protein kinases. As indicated by the “Expect” value, the probability of this alignment occurring by chance alone is 4.3e-298, which is an incredibly low probability score. Moreover, the disclosed, encoded amino acid sequence has 1095 of 1332 amino acid residues (82%) identical to a human NIK-related protein (GenBank Accession No: BAA90753), as shown in Table 12. As indicated by the “Expect” value, the probability of this alignment occurring by chance alone is 0, the lowest probability score.

TABLE 11
Score = 3892 (584.0 bits), Expect = 4.3e-298, Sum P(2) = 4.3e-298
Identities = 1224/1657 (73%), Positives = 1224/1657 (73%), Strand = Plus /
Plus

| | | | |
|---|---|---|---|
| NOV3a: | 4 | GGCGACCCAGCC-CCCGCCCGCAGCCTGGACGACATCGACCTGTCCGCCCTGCGGGACCC | 62 |
| | | GGCGA C A C CCCGC AGCCTGG GACAT GACCTGTC CCCTGCGGGACCC | |
| NIK: | 3 | GGCGAACGACTCTCCCGCGAAGAGCCTGGTGGACATTGACCTGTCGTCCCTGCGGGACCC | 62 |
| NOV3a: | 63 | TGCTGGGATCTTTGAGCTTGTGGAGGTGGTCGGCAATGGAACCTACGGACAGGTGTACAA | 122 |
| | | TGCTGGGAT TTTGAGCT GTGGA GTGGT GG AATGG ACCTA GGACA GT TA AA | |
| NIK: | 63 | TGCTGGGATTTTTGAGCTGGTGGAAGTGGTTGGAAATGGCACCTATGGACAAGTCTATAA | 122 |
| NOV3a: | 123 | GGGTCGGCATGTCAAGACGGGGCAGCTGGCTGCCATCAAGGTCATGGATGTCACGGAGGA | 182 |
| | | GGGTCG CATGT AA ACGG CA CTG C GCCATCAAGGT ATGGA GTCAC GAGGA | |
| NIK: | 123 | GGGTCGACATGTTAAAACGGT-CA-CTGCC-GCCATCAAGGTTATGGACGTCACCGAGGA | 179 |
| NOV3a: | 183 | CGAGGAGGAAGAGATCAAACAGGAGATCAACATGCTGAAAAAGTACTCTCACCACCGCAA | 242 |
| | | GA GAGGAAGA ATCA AC GGAGAT AA ATGCTGAA AAGTA TCTCA CA CG AA | |
| NIK: | 180 | TGAAGAGGAAGAAATCACACTGGAGATAAATATGCTGAAGAAGTATTCTCATCATCGAAA | 239 |
| NOV3a: | 243 | CATCGCCACCTACTACGGAGCCTTCATCAAGAAGAGCCCCCCGGGAAACGATGACCAGCT | 302 |
| | | AT GCCAC TACTA GG GC TTCAT AAGAAGAGCCC CC GGA A GATGACCA CT | |
| NIK: | 240 | TATTGCCACGTACTATGGTGCTTTCATTAAGAAGAGCCCTCCAGGACATGATGACCAACT | 299 |
| NOV3a: | 303 | CTGGCTGGTGATGGAGTTCTGTGGTGCTGGTTCAGTGACTGACCTGGTAAAGAACACAAA | 362 |
| | | CTGGCT GT ATGGAGTT TGTGG GCTGG TC T AC GACCT GT AAGAACAC AA | |
| NIK: | 300 | CTGGCTTGTTATGGAGTTTTGTGGGGCTGGGTCCATCACAGACCTTGTGAAGAACACCAA | 359 |
| NOV3a: | 363 | AGGCAACGCCCTGAAGGAGGACTGTATCGCCTATATCTGCAGGGAGATCCTCAGGGGTCT | 422 |
| | | AGG AAC C CT AA GA GACTG AT GC TA ATCT CAGGGA ATCCTCAGGGG T | |
| NIK: | 360 | AGGGAACACTCTCAAAGAAGACTGGATTGCTTACATCTCCAGGGAAATCCTCAGGGGATT | 419 |
| NOV3a: | 423 | GGCCCATCTCCATGCCCACAAGGTGATCCATCGAGACATCAAGGGGCAGAATGTGCTGCT | 482 |
| | | GGC CATCTCCAT CAC A GT AT CA CGAGA ATCAAGGG CA AATGTGCTGCT | |
| NIK: | 420 | GGCACATCTCCATATTCACCACGTTATTCACCGAGATATCAAGGGCCAAAATGTGCTGCT | 479 |
| NOV3a: | 483 | GACAGAGAATGCTGAGGTCAAGCTAGTGGATTTTGGGGTGAGTGCTCAGCTGGACCGCAC | 542 |
| | | GAC GAGAATGCTGAGGT AA CT GT GATTTTGG GT AG GCTCAGCTGGAC G AC | |
| NIK: | 480 | GACCGAGAATGCTGAGGTGAAACTTGTTGATTTTGGTGTAAGCGCTCAGCTGGACAGGAC | 539 |
| NOV3a: | 543 | CGTGGG-CAGACGGAACACTTTCATTGGGACTCCCTACTGGATGGCTCCAGAGGTCATCG | 601 |
| | | GT GG C GA G AA AC TTCAT GG AC CCCTACTGGATGGCTCCAGAGGTCATCG | |
| NIK: | 540 | GGTTGGACGGA-GAAATACGTTCATAGGCACACCCTACTGGATGGCTCCAGAGGTCATCG | 598 |
| NOV3a: | 602 | CCTGTGATGAGAACCCTGATGCCACCTATGATTACAGGAGTGATATTTGGTCTCTA-GGA | 660 |
| | | CCTGTGATGAGAACCC GA GCCAC TA GA TACAG AGTGA T TGGTC CT GG | |
| NIK: | 599 | CCTGTGATGAGAACCCAGACGCCACTTACGACTACAGAAGTGACCTCTGGTC-CTGTGGC | 657 |
| NOV3a: | 661 | ATCACAGCCATCGAGATGGCAGAGGGAGCCCCCCCTCTGTGTGACATGCACCCCATGCGA | 720 |
| | | ATCACAGCCATCGAGATGGC GA GG G CCCCCCTCT TGTGACATGCA CC ATG GA | |
| NIK: | 658 | ATCACAGCCATCGAGATGGCTGAAGGGGGCCCCCCTCTCTGTGACATGCATCCAATGAGA | 717 |
| NOV3a: | 721 | GCCCTCTTCCTCATTCCTCGGAACCCTCCGCCCAGGCTCAAGTCCAAGAAGTGGTCTAAG | 780 |
| | | GC CT TT CTCAT CC G AACCCTCC CCCAGGCT AAGTC AA AA TGGTC AAG | |
| NIK: | 718 | GCGCTGTTTCTCATCCCCAGAAACCCTCCTCCCAGGCTGAAGTCAAAAAAATGGTCAAAG | 777 |
| NOV3a: | 781 | AAGTTCATTGA-CTTCATTGACACATGTCTCATCAAGACTTACCTG-AGCCGCCCACCCA | 838 |
| | | AA TT TT A CTT AT GA TGTCT T AAGA TTAC TG AGC GCCC C A | |
| NIK: | 778 | AAATTT-TTCAGCTTTATAGAAGGCTGTCTGGTGAAGAATTACATGCAGCGGCCCTCT-A | 835 |
| NOV3a: | 839 | CGGAGCAGCTACTGAAGTTTCCCTTCATCCGGGACCAGCCCACGGAGCGGCAGGTCCGCA | 898 |
| | | C GAGCA CT T AA CC TTCAT GGGA CAGCCCA GA GGCAGGT CG A | |
| NIK: | 836 | CAGAGCAACTTTTAAAACACCCTTTCATAAGGGATCAGCCCAATGAAAGGCAGGTTCGAA | 895 |
| NOV3a: | 899 | TCCAGCTTAAGGACCACATTGACCGATCCCGGAAGAAGCGGGGTGAGAAAGAGGAGACAG | 958 |
| | | TCCAGCTTAAGGA CACAT GACCG CC G AAGAAG G GG GAGAAAGA GAGAC G | |
| NIK: | 896 | TCCAGCTTAAGGATCACATAGACCGGACCAGAAAGAAGAGAGGCGAGAAAGATGAGACGG | 955 |
| NOV3a: | 959 | AATATGAGTACAGCGGCAGCGAGGAGGAAGATGAC-A-GC-CATGGAG-AGGAAGGAGAG | 1014 |
| | | A TA GAGTACAGCGG AGCGAGGAGGA GA GA A G C TG AG AGGA GGAGAG | |
| NIK: | 956 | AGTACGAGTACAGCGGGAGCGAGGAGGAGGAGGAGGAAGTGCCTG-AGCAGGAGGGAGAG | 1014 |
| NOV3a: | 1015 | CCAAGCTCCATCATGAACGTGCCTGGAGAGTCGACTCTACGCCGGGAGTTTCTCCGGCTC | 1074 |
| | | CCAAG TCCATC T AA GTGCCTGGAGAGTC ACTCT CG CG GA TT CT G CT | |
| NIK: | 1015 | CCAAGTTCCATCGTCAATGTGCCTGGAGAGTCAACTCTGCGACGTGATTTCCTGAGACTG | 1074 |
| NOV3a: | 1075 | CAGCAGGAAAATAAG-AGCAACTCAGAGGCTTTAAAACAG-CAGCAGCAGCTGCAGCAGC | 1132 |
| | | CAGCAGGA AA AAG AGC TC GAGGCT T AG CAGCAGC CTGCAG AGC | |
| NIK: | 1075 | CAGCAGGAGAACAAGGAGCGG-TCTGAGGCTCTGCGG-AGACAGCAGCTTCTGCAGGAGC | 1132 |
| NOV3a: | 1133 | AGCAGCAGCGAGACCCCGAGGCACACATCAAACACCTGCTGCACCAGCGGCAGCGGCGCA | 1192 |
| | | AGCAGC CG GA C GAGG A A A CA CTGCTG AG GGCAG GCG A | |
| NIK: | 1133 | AGCAGCTCCGGGAGCAGGAGGAGTATAAGAGGCAGCTGCTGGCTGAGAGGCAGAAGCGGA | 1192 |
| NOV3a: | 1193 | TAGAGGAGCAGAAGGAGGAGCGGCGCCGCGTGGAGGAGCAACAGCGGCGGGAGCGGGA-G | 1251 |
| | | T GA AGCAGAA GA AG GG G CG TGGA GAGCAACA G G GA CGGGA G | |
| NIK: | 1193 | TTGAACAGCAGAAAGAACAGAGGAGGCGGCTGGAAGAGCAACAAAGAAGAGAACGGGAAG | 1252 |
| NOV3a: | 1252 | CAGCGGAAGCTGCAGGAGAAGGAGCAGCAGCGGCG-G--CTGGAGGACATGCAGGC-TCT | 1307 |
| | | C GGA GC GCAGGAG GAGCAGC GCGGCG G C GAGGA A G AGGC TCT | |
| NIK: | 1253 | CCA-GGAGGCAGCAGGAGCGTGAGCAGCGGCGGCGTGAACAAGAGGAGAAG-AGGCGTCT | 1310 |
| NOV3a: | 1308 | GCGGCGGGA--GGAGGAGCGGCGGCAGGCGGAGCGCGAGCAGGAATATATTCGTCACAGG | 1365 |
| | | CG GG A GGA GCGGCG A G GAG GAG AGGA A C A AGG | |
| NIK: | 1311 | -CGA-GGAACTGGAAAGGCGGCGTAAAGAAGAGGAAGAG-AGGAG-ACGGGCAGAAGAGG | 1366 |
| NOV3a: | 1366 | CTA-GAGGAG-GAGCAGCGACAGCTCGAGATCCTTCAGCAACAGCTGCTCCAGGAACAGG | 1423 |
| | | A GAGGAG G G AG G A C GAG T C TCAG C GC GCT AGGA AG | |
| NIK: | 1367 | AGAAGAGGAGAGTGGAGAGGGAACAGGAG-TACATCAGG--CGGCAGCTAGAGGAGGAGC | 1423 |
| NOV3a: | 1424 | CCCTGCTGCTGGA-ATACA--AGCGGAAGCAGCTGGAGGAGCAGCGGCA-GTCAGAACGT | 1479 |
| | | C GC CTGGA AT C AGC G AGC GCT AGGAGCAG G CA GT A C | |
| NIK: | 1424 | AGCGGCACCTGGAGATCCTGCAGCAGCAGCTGCTCCAGGAGCAG-GCCATGTTACTGCAC | 1482 |
| NOV3a: | 1480 | CTCCAGAGGCAGCTGCA-GCAGGAGCATGCCTACCTCAAGTCCCTGCAGCAGCAGCAACA | 1538 |
| | | CCA AGG GC GCA GCA AGCA GC CC C G CCC GCAGCAGCAG A CA | |
| NIK: | 1483 | GACCACAGGAGGCCGCACGCAC-AGCA-GCAG-CCGCC-GCCCCCGCAGCAGCAGGA-CA | 1537 |
| NOV3a: | 1539 | GCAGCAG--C-AGCTT-CA-GAAACAGCAGCAGCAGCAGCTCC-TG-CC-TGGGGACAGG | 1590 |
| | | G AGCA C AGCTT CA G CAG AGC AGC C C TG CC TG GACAG | |
| NIK: | 1538 | GGAGCAAACCGAGCTTTCATGCTCCAG-AGCCCAAGCCTCACTATGACCCTGCTGACAG- | 1595 |
| NOV3a: | 1591 | AAGCCCCTGTACCATTATGGTCGGGGCATGAATCCCGCT-GA-CAAAC-CAGCCTGGGCC | 1647 |
| | | AGC C G A TGGTC C G ATC C C GA CAA C CC G C | |
| NIK: | 1596 | -AGCTCGGGAGGTACAGTGGTCCCACCTGGCATCTCTCAAGAACAATGTCTCCCCTGTCT | 1654 |
| NOV3a: | 1648 | CGAGA 1652 (SEQ ID NO:62) | |
| | | CGAGA | |
| NIK: | 1655 | CGAGA 1659 (SEQ ID NO: 29) | |

TABLE 12
Score = 2104 bits (5451), Expect = 0.0
Identities = 1095/1332 (82%), Positives = 1095/1332 (82%), Gaps = 37/1332
(2%)

| | | | |
|---|---|---|---|
| NOV3a: | 1 | MGDPAPARSLDDIDLSALRDPAGIFELVEVVGNGTYGQVYKGRHVKTGQLAAIKVMDVTX | 60 |
| | | MGDPAPARSLDDIDLSALRDPAGIFELVEVVGNGTYGQVYKGRHVKTGQLAAIKVMDVT | |
| NIK: | 1 | MGDPAPARSLDDIDLSALRDPAGIFELVEVVGNGTYGQVYKGRHVKTGQLAAIKVMDVTE | 60 |
| NOV3a: | 61 | XXXXXIKQEINMLKKYSHHRNIATYYGAFIKKSPPGNDDQLWLVMEFCGAGSVTDLVKNT | 120 |
| | | IKQEINMLKKYSHHRNIATYYGAFIKKSPPGNDDQLWLVMEFCGAGSVTDLVKNT | |
| NIK: | 61 | DEEEEIKQEINMLKKYSHHRNIATYYGAFIKKSPPGNDDQLWLVMEFCGAGSVTDLVKNT | 120 |
| NOV3a: | 121 | KGNALKEDCIAYICREILRGLAHLHAHKVIHRDIKGQNVLLTENAEVKLVDFGVSAQLDR | 180 |
| | | KGNALKEDCIAYICREILRGLAHLHAHKVIHRDIKGQNVLLTENAEVKLVDFGVSAQLDR | |
| NIK: | 121 | KGNALKEDCIAYICREILRGLAHLHAHKVIHRDIKGQNVLLTENAEVKLVDFGVSAQLDR | 180 |
| NOV3a: | 181 | TVGRRNTFIGTPYWMAPEVIACDENPDATYDYRSDIWSLGITAIEMAEGAPPLCDMHPMR | 240 |
| | | TVGRRNTFIGTPYWMAPEVIACDENPDATYDYRSDIWSLGITAIEMAEGAPPLCDMHPMR | |
| NIK: | 181 | TVGRRNTFIGTPYWMAPEVIACDENPDATYDYRSDIWSLGITAIEMAEGAPPLCDMHPMR | 240 |
| NOV3a: | 241 | ALFLIPRNPPPRLKSKKWSKKFIDFIDTCLIKTYLSRPPTEQLLKFPFIRDQPTERQVRI | 300 |
| | | ALFLIPRNPPPRLKSKKWSKKFIDFIDTCLIKTYLSRPPTEQLLKFPFIRDQPTERQVRI | |
| NIK: | 241 | ALFLIPRNPPPRLKSKKWSKKFIDFIDTCLIKTYLSRPPTEQLLKFPFIRDQPTERQVRI | 300 |
| NOV3a: | 301 | QLKDHIXXXXXXXXXXXXXXXXXXXXXXXXXXXXXXXXPSSIMNVPGESTLRREFLRLQQ | 360 |
| | | QLKDHI PSSIMNVPGESTLRREFLRLQQ | |
| NIK: | 301 | QLKDHIDRSRKKRGEKEETEYEYSGSEEEDDSHGEEGEPSSIMNVPGESTLRREFLRLQQ | 360 |
| NOV3a: | 361 | ENKSNSEALKXXXXXXXXXXRDPEAHIKHLLHXXXXXXXXXXXXXXXXXXXXXXXXXXXX | 420 |
| | | ENKSNSEALK RDPEAHIKHLLH | |
| NIK: | 361 | ENKSNSEALKQQQQLQQQQQRDPEAHIKHLLHQRQRRIEEQKEERRRVEEQQRREREQRK | 420 |
| NOV3a: | 421 | XXXXXXXXXXXDMQALXXXXXXXXXXXXXXYIRHRXXXXXXXXXXXXXXXXXXXXXXXXY | 480 |
| | | DMQAL Y | |
| NIK: | 421 | LQEKEQQRRLEDMQAL-----------------------------RREEERRQAEREQEY | 451 |
| NOV3a: | 481 | KRKXXXXXXXXXXXXXXXXXXHAYLKSXXXXXXXXXXXXXXXXXXXPGDRKPLYHYGRGM | 540 |
| | | KRK HAYLKS PGDRKPLYHYGRGM | |
| NIK: | 452 | KRKQLEEQRQSERLQRQLQQEHAYLKSLQQQQQQQQLQKQQQQQLLPGDRKPLYHYGRGM | 511 |
| NOV3a: | 541 | NPADKPAWAREVEERTRMNKQQNSPLAKSKPGSTXXXXXXXXXXXXXXXXXXXXXXMQRP | 600 |
| | | NPADKPAWAREVEERTRMNKQQNSPLAKSKPGST MQRP | |
| NIK: | 512 | NPADKPAWAREVEERTRMNKQQNSPLAKSKPGSTGPEPPIPQASPGPPGPLSQTPPMQRP | 571 |
| NOV3a: | 601 | VEPQEGPHKSLVAHRVPLKPYAAPVPRSQSLQDQPTRNLAAFPASHXXXXXXXXXXXXXX | 660 |
| | | VEPQEGPHKSLVAHRVPLKPYAAPVPRSQSLQDQPTRNLAAFPASH | |
| NIK: | 572 | VEPQEGPHKSLVAHRVPLKPYAAPVPRSQSLQDQPTRNLAAFPASHDPDPAIPAPTATPS | 631 |
| NOV3a: | 661 | XRGAVIRQNSDPTSEGPGPSPNPPAWVRPDNEAPPKVPQRTSSIATALNTSGAGGSRPAQ | 720 |
| | | RGAVIRQNSDPTSEGPGPSPNPPAWVRPDNEAPPKVPQRTSSIATALNTSGAGGSRPAQ | |
| NIK: | 632 | ARGAVIRQNSDPTSEGPGPSPNPPAWVRPDNEAPPKVPQRTSSIATALNTSGAGGSRPAQ | 691 |
| NOV3a: | 721 | AVRASNPDLRRSDPGWERSDSVLPASHGHLPQAGSLERNRVGVSSKPDSSPVLSPGNKAK | 780 |
| | | AVRASNPDLRRSDPGWERSDSVLPASHGHLPQAGSLERNRVGVSSKPDSSPVLSPGNKAK | |
| NIK: | 692 | AVRASNPDLRRSDPGWERSDSVLPASHGHLPQAGSLERNRVGVSSKPDSSPVLSPGNKAK | 751 |
| NOV3a: | 781 | PDDHRSRPGRPASYKRAIGEDFVLLKERTLDEAPRPPKKAMDYXXXXXXXXXXXXXXXXX | 840 |
| | | PDDHRSRPGRPA DFVLLKERTLDEAPRPPKKAMDY | |
| NIK: | 752 | PDDHRSRPGRPA--------DFVLLKERTLDEAPRPPKKAMDYSSSSEEVESSEDDEEEG | 803 |
| NOV3a: | 841 | XXXXXXXXRDTPGGRSDGDTDSVSTMVVHDVEEITGTQPPYGGGTMVVQRTPEEERNLLH | 900 |
| | | RDTPGGRSDGDTDSVSTMVVHDVEEITGTQPPYGGGTMVVQRTPEEERNLLH | |
| NIK: | 804 | EGGPAEGSRDTPGGRSDGDTDSVSTMVVHDVEEITGTQPPYGGGTMVVQRTPEEERNLLH | 863 |
| NOV3a: | 901 | ADSNGYTNLPDVVQPSHSPTENSKGQSPPSKDGSGDYQSRGLVKAPGKSSFTMFVDLGIY | 960 |
| | | ADSNGYTNLPDVVQPSHSPTENSKGQSPPSKDGSGDYQSRGLVKAPGKSSFTMFVDLGIY | |
| NIK: | 864 | ADSNGYTNLPDVVQPSHSPTENSKGQSPPSKDGSGDYQSRGLVKAPGKSSFTMFVDLGIY | 923 |
| NOV3a: | 961 | QPGGSGDSIPITALVGGEGTRLDQLQYDVRKGSVVNVNPTNTRAHSETPEIRKYKKRFNS | 1020 |
| | | QPGGSGDSIPITALVGGEGTRLDQLQYDVRKGSVVNVNPTNTRAHSETPEIRKYKKRFNS | |
| NIK: | 924 | QPGGSGDSIPITALVGGEGTRLDQLQYDVRKGSVVNVNPTNTRAHSETPEIRKYKKRFNS | 983 |
| NOV3a: | 1021 | EILCAALWGVNLLVGTENGLMLLDRSGQGKVYGLIGRRRFQQMDVLEGLNLLITISGKRN | 1080 |
| | | EILCAALWGVNLLVGTENGLMLLDRSGQGKVYGLIGRRRFQQMDVLEGLNLLITISGKRN | |
| NIK: | 984 | EILCAALWGVNLLVGTENGLMLLDRSGQGKVYGLIGRRRFQQMDVLEGLNLLITISGKRN | 1043 |
| NOV3a: | 1081 | KLRVYYLSWLRNKILHNDPEVEKKQGWTTVGDMEGCGHYRVVKYERIKFLVIALKSSVEV | 1140 |
| | | KLRVYYLSWLRNKILHNDPEVEKKQGWTTVGDMEGCGHYRVVKYERIKFLVIALKSSVEV | |
| NIK: | 1044 | KLRVYYLSWLRNKILHNDPEVEKKQGWTTVGDMEGCGHYRVVKYERIKFLVIALKSSVEV | 1103 |
| NOV3a: | 1141 | YAWAPKPYHKFMAFKSFADLPHRPLLVDLTVEEGQRLKVIYGSSAGFHAVDVDSGNSYDI | 1200 |
| | | YAWAPKPYHKFMAFKSFADLPHRPLLVDLTVEEGQRLKVIYGSSAGFHAVDVDSGNSYDI | |
| NIK: | 1104 | YAWAPKPYHKFMAFKSFADLPHRPLLVDLTVEEGQRLKVIYGSSAGFHAVDVDSGNSYDI | 1163 |
| NOV3a: | 1201 | YIPVHIQSQITPHAIIFLPNTDGMEMLLCYEDEGVYVNTYGRIIKDVVLQWGEMPTSVAY | 1260 |
| | | YIPVHIQSQITPHAIIFLPNTDGMEMLLCYEDEGVYVNTYGRIIKDVVLQWGEMPTSVAY | |
| NIK: | 1164 | YIPVHIQSQITPHAIIFLPNTDGMEMLLCYEDEGVYVNTYGRIIKDVVLQWGEMPTSVAY | 1223 |
| NOV3a: | 1261 | ICSNQIMGWGEKAIEIRSVETGHLDGVFMHKRAQRLKFLCERNDKVFFASVRSGGSSQVY | 1320 |
| | | ICSNQIMGWGEKAIEIRSVETGHLDGVFMHKRAQRLKFLCERNDKVFFASVRSGGSSQVY | |
| NIK: | 1224 | ICSNQIMGWGEKAIEIRSVETGHLDGVFMHKRAQRLKFLCERNDKVFFASVRSGGSSQVY | 1283 |
| NOV3a: | 1321 | FMTLNRNCIMNW 1332 (SEQ ID NO: 63) | |
| | | FMTLNRNCIMNW | |
| NIK: | 1284 | FMTLNRNCIMNW 1295 (SEQ ID NO: 30) | |

Based on its relatedness to known members of the STE20 family of protein kinases, NOV3a provides new diagnostic and therapeutic compositions useful in the treatment of disorders associated with alterations in the expression of members of the STE20 family of protein kinases. Nucleic acids, polypeptides, antibodies, and other compositions of the present invention are useful in the treatrnent and diagnosis of a variety of diseases and pathologies, including, by way of nonlimiting example, those involving metabolic and endocrine disorders, cancer, bone disorders, and tissue/cell growth regulation disorders-.

NOV-3b

A NOV-3b sequence according to the invention is a nucleic acid sequence encoding a polypeptide related to STE20 family of protein kinases. A disclosed NOV-3b nucleic acid and its encoded polypeptide includes the sequences shown in Table 13. The disclosed nucleic acid (SEQ ID NO: 8) is 3912 nucleotides in length and contains an open reading frame (ORF) that begins with an ATG initiation codon at nucleotides 1-3 and ends with a TGA stop codon at nucleotides 3910-3912. The start and stop codons are shown in bold font. The respective ORF encodes a 1303 amino acid polypeptide (SEQ ID NO: 9).

TABLE 13

| | |
|---|---|
| ATGGGCGACCCAGCCCCCGCCCGCAGCCTGGACGACATCGACCTGTCCGCCCTGCGGGACCCTGCTGGGATCTTTGAGCT | (SEQ ID NO: 8) |
| TGTGGAGGTGGTCGGCAATGGAACCTACGGACAGGTGTACAAGGGTCGGCATGTCAAGACGGGGCAGCTGGCTGCCATCA | |
| AGGTCATGGATGTCACGGAGGACGAGGAGGAAGAGATCAAACAGGAGATCAACATGCTGAAAAAGTACTCTCACCACCGC | |
| AACATCGCCACCTACTACGGAGCCTTCATCAAGAAGAGCCCCCCGGGAAACGATGACCAGCTCTGGCTGGTGATGGAGTT | |
| CTGTGGTGCTGGTTCAGTGACTGACCTGGTAAAGAACACAAAAGGCAACGCCCTGAAGGAGGACTGTATCGCCTATATCT | |
| GCAGGGAGATCCTCAGGGGTCTGGCCCATCTCCATGCCCACAAGGTGATCCATCGAGACATCAAGGGGCAGAATGTGCTG | |
| CTGACAGAGAATGCTGAGGTCAAGCTAGTGGATTTTGGGGTGAGTGCTCAGCTGGACCGCACCGTGGGCAGACGGAACAC | |
| TTTCATTGGGACTCCCTACTGGATGGCTCCAGAGGTCATCGCCTGTGATGAGAACCCTGATGCCACCTATGATTACAGGA | |
| GTGATATTTGGTCTCTAGGAATCACAGCCATCGAGATGGCAGAGGGAGCCCCCCCTCTGTGTGACATGCACCCCATGCGA | |
| GCCCTCTTCCTCATTCCTCGGAACCCTCCGCCCAGGCTCAAGTCCAAGAAGTGGTCTAAGAAGTTCATTGACTTCATTGA | |
| CACATGTCTCATCAAGACTTACCTGAGCCGCCCACCCACGGAGCAGCTACTGAAGTTTCCCTTCATCCGGGACCAGCCCA | |
| CGGAGCGGCAGGTCCGCATCCAGCTTAAGGACCACATTGACCGATCCCGGAAGAAGCGGGGTGAGAAAGAGGAGACAGAA | |
| TATGAGTACAGCGGCAGCGAGGAGGAAGATGACAGCCATGGAGAGGAAGGAGAGCCAAGCTCCATCATGAACGTGCCTGG | |
| AGAGTCGACTCTACGCCGGGAGTTTCTCCGGCTCCAGCAGGAAAATAAGAGCAACTCAGAGGCTTTAAAACAGCAGCAGC | |
| AGCTGCAGCAGCAGCAGCAGCGAGACCCCGAGGCACACATCAAACACCTGCTGCACCAGCGGCAGCGGCGCATAGAGGAG | |
| CAGAAGGAGGAGCGGCGCCGCGTGGAGGAGCAACAGCGGCGGGAGCGGGAGCAGCGGAAGCTGCAGGAGAAGGAGCAGCA | |
| GCGGCGGCTGGAGGACATGCAGGCTCTGCGGCGGGAGGAGGAGCGGCGGCAGGCGGAGCGCGAGCAGGAATATATTCGTC | |
| ACAGGCTAGAGGAGCAGCGGCAGTCAGAACGTCTCCAGAGGCAGCTGCAGCAGGAGCATGCCTACCTCAAGTCCCTGCAG | |
| CAGCAGCAACAGCAGCAGCAGCTTCAGAAACAGCAGCAGCAGCAGCTCCTGCCTGGGGACAGGAAGCCCCTGTACCATTA | |
| TGGTCGGGGCATGAATCCCGCTGACAAACCAGCCTGGGCCCGAGAGGTAGAAGAGAGAACAAGGATGAACAAGCAGCAGA | |
| ACTCTCCCTTGGCCAAGAGCAAGCCAGGCAGCACGGGGCCTGAGCCCCCCATCCCCCAGGCCTCCCCAGGGCCCCCAGGA | |
| CCCCTTTCCCAGACTCCTCCTATGCAGAGGCCGGTGGAGCCCCAGGAGGGACCGCACAAGAGCCTGGTGGCACACCGGGT | |
| CCCACTGAAGCCATATGCAGCACCTGTACCCCGATCCCAGTCCCTGCAGGACCAGCCCACCCGAAACCTGGCTGCCTTCC | |
| CAGCCTCCCATGACCCCGACCCTGCCATCCCCGCACCCACTGCCACGCCCAGTGCCCGAGGAGCTGTCATCCGCCAGAAT | |
| TCAGACCCCACCTCTGAAGGACCTGGCCCCAGCCCGAATCCCCCAGCCTGGGTCCGCCCAGATAACGAGGCCCCACCCAA | |
| GGTGCCTCAGAGGACCTCATCTATCGCCACTGCCCTTAACACCAGTGGGGCCGGAGGGTCCCGGCCAGCCCAGGCAGTCC | |
| GTGCCAGTAACCCCGACCTCAGGAGGAGCGACCCTGGCTGGGAACGCTCGGACAGCGTCCTTCCAGCCTCTCACGGGCAC | |
| CTCCCCCAGGCTGGCTCACTGGAGCGGAACCGCGTGGGAGTCTCCTCCAAACCGGACAGCTCCCCTGTGCTCTCCCCTGG | |
| GAATAAAGCCAAGCCCGACGACCACCGCTCACGGCCAGGCCGGCCCGCAAGCTATAAGCGAGCAATTGGTGAGGACTTTG | |
| TGTTGCTGAAAGAGCGGACTCTGGACGAGGCCCCTCGGCCTCCCAAGAAGGCCATGGACTACTCGTCGTCCAGCGAGGAG | |
| GTGGAAAGCAGTGAGGACGACGAGGAGGAAGGCGAAGGCGGGCCAGCAGAGGGGAGCAGAGATACCCCTGGGGGCCGCAG | |
| CGATGGGGATACAGACAGCGTCAGCACCATGGTGGTCCACGACGTCGAGGAGATCACCGGGACCCAGCCCCCATACGGGG | |
| GCGGCACCATGGTGGTCCAGCGCACCCCTGAAGAGGAGCGGAACCTGCTGCATGCTGACAGCAATGGGTACACAAACCTG | |
| CCTGACGTGGTCCAGCCCAGCCACTCACCCACCGAGAACAGCAAAGGCCAAAGCCCACCCTCGAAGGATGGGAGTGGTGA | |
| CTACCAGTCTCGTGGGCTGGTAAAGGCCCCTGGCAAGAGCTCGTTCACGATGTTTGTGGATCTAGGGATCTACCAGCCTG | |
| GAGGCAGTGGGGACAGCATCCCCATCACAGCCCTAGTGGGTGGAGAGGGCACTCGGCTCGACCAGCTGCAGTACGACGTG | |
| AGGAAGGGTTCTGTGGTCAACGTGAATCCCACCAACACCCGGGCCCACAGTGAGACCCCTGAGATCCGGAAGTACAAGAA | |
| GCGATTCAACTCCGAGATCCTCTGTGCAGCCCTTTGGGGGGTCAACCTGCTGGTGGGCACGGAGAACGGGCTGATGTTGC | |
| TGGACCGAAGTGGGCAGGGCAAGGTGTATGGACTCATTGGGCGGCGACGCTTCCAGCAGATGGATGTGCTGGAGGGGCTC | |
| AACCTGCTCATCACCATCTCAGGGAAAAGGAACAAACTGCGGGTGTATTACCTGTCCTGGCTCCGGAACAAGATTCTGCA | |
| CAATGACCCAGAAGTGGAGAAGAAGCAGGGCTGGACCACCGTGGGGGACATGGAGGGCTGCGGGCACTACCGTGTTGTGA | |
| AATACGAGCGGATTAAGTTCCTGGTCATCGCCCTCAAGAGCTCCGTGGAGGTGTATGCCTGGGCCCCCAAACCCTACCAC | |
| AAATTCATGGCCTTCAAGTCCTTTGCCGACCTCCCCCACCGCCCTCTGCTGGTCGACCTGACAGTAGAGGAGGGGCAGCG | |
| GCTCAAGGTCATCTATGGCTCCAGTGCTGGCTTCCATGCTGTGGATGTCGACTCGGGGAACAGCTATGACATCTACATCC | |
| CTGTGCACATCCAGAGCCAGATCACGCCCCATGCCATCATCTTCCTCCCCAACACCGACGGCATGGAGATGCTGCTGTGC | |
| TACGAGGACGAGGGTGTCTACGTCAACACGTACGGGCGCATCATTAAGGATGTGGTGCTGCAGTGGGGGGAGATGCCTAC | |
| TTCTGTGGCCTACATCTGCTCCAACCAGATAATGGGCTGGGGTGAGAAAGCCATTGAGATCCGCTCTGTGGAGACGGGCC | |
| ACCTCGACGGGGTCTTCATGCACAAACGAGCTCAGAGGCTCAAGTTCCTGTGTGAGCGGAATGACAAGGTGTTTTTTGCC | |
| TCAGTCCGCTCTGGGGGCAGCAGCCAAGTTTACTTCATGACTCTGAACCGTAACTGCATCATGAACTGGTGA | |
| MGDPAPARSLDDIDLSALRDPAGIFELVEVVGNGTYGQVYKGRHVKTGQLAAIKVMDVTEDEEEEIKQEINMLKKYSHHR | (SEQ ID NO: 9) |
| NIATYYGAFIKKSPPGNDDQLWLVMEFCGAGSVTDLVKNTKGNALKEDCIAYICREILRGLAHLHAHKVIHRDIKGQNVL | |
| LTENAEVKLVDFGVSAQLDRTVGRRNTFIGTPYWMAPEVIACDENPDATYDYRSDIWSLGITAIEMAEGAPPLCDMHPMR | |
| ALFLIPRNPPPRLKSKKWSKKFIDFIDTCLIKTYLSRPPTEQLLKFPFIRDQPTERQVRIQLKDHIDRSRKKRGEKEETE | |
| YEYSGSEEEDDSHGEEGEPSSIMNVPGESTLRREFLRLQQENKSNSEALKQQQQLQQQQQRDPEAHIKHLLHQRQRRIEE | |
| QKEERRRVEEQQRREREQRKLQEKEQQRRLEDMQALRREEERRQAEREQEYIRHRLEEQRQSERLQRQLQQEHAYLKSLQ | |
| QQQQQQQLQKQQQQQLLPGDRKPLYHYGRGMNPADKPAWAREVEERTRMNKQQNSPLAKSKPGSTGPEPPIPQASPGPPG | |
| PLSQTPPMQRPVEPQEGPHKSLVAHRVPLKPYAAPVPRSQSLQDQPTRNLAAFPASHDPDPAIPAPTATPSARGAVIRQN | |
| SDPTSEGPGPSPNPPAWVRPDNEAPPKVPQRTSSIATALNTSGAGGSRPAQAVRASNPDLRRSDPGWERSDSVLPASHGH | |
| LPQAGSLERNRVGVSSKPDSSPVLSPGNKAKPDDHRSRPGRPASYKRAIGEDFVLLKERTLDEAPRPPKKAMDYSSSSEE | |
| VESSEDDEEEGEGGPAEGSRDTPGGRSDGDTDSVSTMVVHDVEEITGTQPPYGGGTMVVQRTPEEERNLLHADSNGYTNL | |
| PDVVQPSHSPTENSKGQSPPSKDGSGDYQSRGLVKAPGKSSFTMFVDLGIYQPGGSGDSIPITALVGGEGTRLDQLQYDV | |
| RKGSVVNVNPTNTRAHSETPEIRKYKKRFNSEILCAALWGVNLLVGTENGLMLLDRSGQGKVYGLIGRRRFQQMDVLEGL | |
| NLLITISGKRNKLRVYYLSWLRNKILHNDPEVEKKQGWTTVGDMEGCGHYRVVKYERIKFLVIALKSSVEVYAWAPKPYH | |
| KFMAFKSFADLPHRPLLVDLTVEEGQRLKVIYGSSAGFHAVDVDSGNSYDIYIPVHIQSQITPHAIIFLPNTDGMEMLLC | |
| YEDEGVYVNTYGRIIKDVVLQWGEMPTSVAYICSNQIMGWGEKAIEIRSVETGHLDGVFMHKRAQRLKFLCERNDKVFFA | |
| SVRSGGSSQVYFMTLNRNCIMNW | |

The disclosed NOV-3b nucleic acid sequence has homology (75% identity) to a mouse mRNA for a NIK protein (NIK) (GenBank Accession No: MMU88984), as shown in Table 14. NIK proteins are a subgroup of the STE20 family of protein kinases. As indicated by the “Expect” value, the probability of this alignment occurring by chance alone is 3.3e-295, which is an incredibly low probability score. Moreover, the disclosed, encoded amino acid sequence has 1093 of 1303 amino acid residues (83%) identical to a human NIK-related protein (GenBank Accession No: BAA90753), as shown in Table 15. As indicated by the “Expect” value, the probability of this alignment occurring by chance alone is 0.0, the lowest probability score.

TABLE 14
Score = 3828 (574.4 bits), Expect = 3.3e-295, Sum P(2) = 3.3e-295
Identities = 1128/1488 (75%), Positives = 1128/1488 (75%), Strand = Plus /
Plus

| | | | |
|---|---|---|---|
| NOV3b: | 4 | GGCGACCCAGCC-CCCGCCCGCAGCCTGGACGACATCGACCTGTCCGCCCTGCGGGACCC | 62 |
| | | GGCGA C A C CCCGC AGCCTGG GACAT GACCTGTC CCCTGCGGGACCC | |
| NIK: | 3 | GGCGAACGACTCTCCCGCGAAGAGCCTGGTGGACATTGACCTGTCGTCCCTGCGGGACCC | 62 |
| NOV3b: | 63 | TGCTGGGATCTTTGAGCTTGTGGAGGTGGTCGGCAATGGAACCTACGGACAGGTGTACAA | 122 |
| | | TGCTGGGAT TTTGAGCT GTGGA GTGGT GG AATGG ACCTA GGACA GT TA AA | |
| NIK: | 63 | TGCTGGGATTTTTGAGCTGGTGGAAGTGGTTGGAAATGGCACCTATGGACAAGTCTATAA | 122 |
| NOV3b: | 123 | GGGTCGGCATGTCAAGACGGGGCAGCTGGCTGCCATCAAGGTCATGGATGTCACGGAGGA | 182 |
| | | GGGTCG CATGT AA ACGG CA CTG C GCCATCAAGGT ATGGA GTCAC GAGGA | |
| NIK: | 123 | GGGTCGACATGTTAAAACGGT-CA-CTGCC-GCCATCAAGGTTATGGACGTCACCGAGGA | 179 |
| NOV3b: | 183 | CGAGGAGGAAGAGATCAAACAGGAGATCAACATGCTGAAAAAGTACTCTCACCACCGCAA | 242 |
| | | GA GAGGAAGA ATCA AC GGAGAT AA ATGCTGAA AAGTA TCTCA CA CG AA | |
| NIK: | 180 | TGAAGAGGAAGAAATCACACTGGAGATAAATATGCTGAAGAAGTATTCTCATCATCGAAA | 239 |
| NOV3b: | 243 | CATCGCCACCTACTACGGAGCCTTCATCAAGAAGAGCCCCCCGGGAAACGATGACCAGCT | 302 |
| | | AT GCCAC TACTA GG GC TTCAT AAGAAGAGCCC CC GGA A GATGACCA CT | |
| NIK: | 240 | TATTGCCACGTACTATGGTGCTTTCATTAAGAAGAGCCCTCCAGGACATGATGACCAACT | 299 |
| NOV3b: | 303 | CTGGCTGGTGATGGAGTTCTGTGGTGCTGGTTCAGTGACTGACCTGGTAAAGAACACAAA | 362 |
| | | CTGGCT GT ATGGAGTT TGTGG GCTGG TC T AC GACCT GT AAGAACAC AA | |
| NIK: | 300 | CTGGCTTGTTATGGAGTTTTGTGGGGCTGGGTCCATCACAGACCTTGTGAAGAACACCAA | 359 |
| NOV3b: | 363 | AGGCAACGCCCTGAAGGAGGACTGTATCGCCTATATCTGCAGGGAGATCCTCAGGGGTCT | 422 |
| | | AGG AAC C CT AA GA GACTG AT GC TA ATCT CAGGGA ATCCTCAGGGG T | |
| NIK: | 360 | AGGGAACACTCTCAAAGAAGACTGGATTGCTTACATCTCCAGGGAAATCCTCAGGGGATT | 419 |
| NOV3b: | 423 | GGCCCATCTCCATGCCCACAAGGTGATCCATCGAGACATCAAGGGGCAGAATGTGCTGCT | 482 |
| | | GGC CATCTCCAT CAC A GT AT CA CGAGA ATCAAGGG CA AATGTGCTGCT | |
| NIK: | 420 | GGCACATCTCCATATTCACCACGTTATTCACCGAGATATCAAGGGCCAAAATGTGCTGCT | 479 |
| NOV3b: | 483 | GACAGAGAATGCTGAGGTCAAGCTAGTGGATTTTGGGGTGAGTGCTCAGCTGGACCGCAC | 542 |
| | | GAC GAGAATGCTGAGGT AA CT GT GATTTTGG GT AG GCTCAGCTGGAC G AC | |
| NIK: | 480 | GACCGAGAATGCTGAGGTGAAACTTGTTGATTTTGGTGTAAGCGCTCAGCTGGACAGGAC | 539 |
| NOV3b: | 543 | CGTGGG-CAGACGGAACACTTTCATTGGGACTCCCTACTGGATGGCTCCAGAGGTCATCG | 601 |
| | | GT GG C GA G AA AC TTCAT GG AC CCCTACTGGATGGCTCCAGAGGTCATCG | |
| NIK: | 540 | GGTTGGACGGA-GAAATACGTTCATAGGCACACCCTACTGGATGGCTCCAGAGGTCATCG | 598 |
| NOV3b: | 602 | CCTGTGATGAGAACCCTGATGCCACCTATGATTACAGGAGTGATATTTGGTCTCTA-GGA | 660 |
| | | CCTGTGATGAGAACCC GA GCCAC TA GA TACAG AGTGA T TGGTC CT GG | |
| NIK: | 599 | CCTGTGATGAGAACCCAGACGCCACTTACGACTACAGAAGTGACCTCTGGTC-CTGTGGC | 657 |
| NOV3b: | 661 | ATCACAGCCATCGAGATGGCAGAGGGAGCCCCCCCTCTGTGTGACATGCACCCCATGCGA | 720 |
| | | ATCACAGCCATCGAGATGGC GA GG G CCCCCCTCT TGTGACATGCA CC ATG GA | |
| NIK: | 658 | ATCACAGCCATCGAGATGGCTGAAGGGGGCCCCCCTCTCTGTGACATGCATCCAATGAGA | 717 |
| NOV3b: | 721 | GCCCTCTTCCTCATTCCTCGGAACCCTCCGCCCAGGCTCAAGTCCAAGAAGTGGTCTAAG | 780 |
| | | GC CT TT CTCAT CC G AACCCTCC CCCAGGCT AAGTC AA AA TGGTC AAG | |
| NIK: | 718 | GCGCTGTTTCTCATCCCCAGAAACCCTCCTCCCAGGCTGAAGTCAAAAAAATGGTCAAAG | 777 |
| NOV3b: | 781 | AAGTTCATTGA-CTTCATTGACACATGTCTCATCAAGACTTACCTG-AGCCGCCCACCCA | 838 |
| | | AA TT TT A CTT AT GA TGTCT T AAGA TTAC TG AGC GCCC C A | |
| NIK: | 778 | AAATTT-TTCAGCTTTATAGAAGGCTGTCTGGTGAAGAATTACATGCAGCGGCCCTCT-A | 835 |
| NOV3b: | 839 | CGGAGCAGCTACTGAAGTTTCCCTTCATCCGGGACCAGCCCACGGAGCGGCAGGTCCGCA | 898 |
| | | C GAGCA CT T AA CC TTCAT GGGA CAGCCCA GA GGCAGGT CG A | |
| NIK: | 836 | CAGAGCAACTTTTAAAACACCCTTTCATAAGGGATCAGCCCAATGAAAGGCAGGTTCGAA | 895 |
| NOV3b: | 899 | TCCAGCTTAAGGACCACATTGACCGATCCCGGAAGAAGCGGGGTGAGAAAGAGGAGACAG | 958 |
| | | TCCAGCTTAAGGA CACAT GACCG CC G AAGAAG G GG GAGAAAGA GAGAC G | |
| NIK: | 896 | TCCAGCTTAAGGATCACATAGACCGGACCAGAAAGAAGAGAGGCGAGAAAGATGAGACGG | 955 |
| NOV3b: | 959 | AATATGAGTACAGCGGCAGCGAGGAGGAAGATGAC-A-GC-CATGGAG-AGGAAGGAGAG | 1014 |
| | | A TA GAGTACAGCGG AGCGAGGAGGA GA GA A G C TG AG AGGA GGAGAG | |
| NIK: | 956 | AGTACGAGTACAGCGGGAGCGAGGAGGAGGAGGAGGAAGTGCCTG-AGCAGGAGGGAGAG | 1014 |
| NOV3b: | 1015 | CCAAGCTCCATCATGAACGTGCCTGGAGAGTCGACTCTACGCCGGGAGTTTCTCCGGCTC | 1074 |
| | | CCAAG TCCATC T AA GTGCCTGGAGAGTC ACTCT CG CG GA TT CT G CT | |
| NIK: | 1015 | CCAAGTTCCATCGTCAATGTGCCTGGAGAGTCAACTCTGCGACGTGATTTCCTGAGACTG | 1074 |
| NOV3b: | 1075 | CAGCAGGAAAATAAG-AGCAACTCAGAGGCTTTAAAACAG-CAGCAGCAGCTGCAGCAGC | 1132 |
| | | CAGCAGGA AA AAG AGC TC GAGGCT T AG CAGCAGC CTGCAG AGC | |
| NIK: | 1075 | CAGCAGGAGAACAAGGAGCGG-TCTGAGGCTCTGCGG-AGACAGCAGCTTCTGCAGGAGC | 1132 |
| NOV3b: | 1133 | AGCAGCAGCGAGACCCCGAGGCACACATCAAACACCTGCTGCACCAGCGGCAGCGGCGCA | 1192 |
| | | AGCAGC CG GA C GAGG A A A CA CTGCTG AG GGCAG GCG A | |
| NIK: | 1133 | AGCAGCTCCGGGAGCAGGAGGAGTATAAGAGGCAGCTGCTGGCTGAGAGGCAGAAGCGGA | 1192 |
| NOV3b: | 1193 | TAGAGGAGCAGAAGGAGGAGCGGCGCCGCGTGGAGGAGCAACAGCGGCGGGAGCGGGA-G | 1251 |
| | | T GA AGCAGAA GA AG GG G CG TGGA GAGCAACA G G GA CGGGA G | |
| NIK: | 1193 | TTGAACAGCAGAAAGAACAGAGGAGGCGGCTGGAAGAGCAACAAAGAAGAGAACGGGAAG | 1252 |
| NOV3b: | 1252 | CAGCGGAAGCTGCAGGAGAAGGAGCAGCAGCGGCG-G--CTGGAGGACATGCAGGC-TCT | 1307 |
| | | C GGA GC GCAGGAG GAGCAGC GCGGCG G C GAGGA A G AGGC TCT | |
| NIK: | 1253 | CCA-GGAGGCAGCAGGAGCGTGAGCAGCGGCGGCGTGAACAAGAGGAGAAG-AGGCGTCT | 1310 |
| NOV3b: | 1308 | GCGGCGGGA--GGAGGAGCGGCGGCAGGCGGAGCGCGAGCAGGAATATATTCGTCACAGG | 1365 |
| | | CG GG A GGA GCGGCG A G GAG GAG AGGA A C A AGG | |
| NIK: | 1311 | -CGA-GGAACTGGAAAGGCGGCGTAAAGAAGAGGAAGAG-AGGAG-ACGGGCAGAAGAGG | 1366 |
| NOV3b: | 1366 | CTA-GAGGAGCAGCGGC-AGT---CAGAACGT-CTCCAGA-GGCAGCTGCAGCAGGAGCA | 1418 |
| | | A GAGGAG AG GG AG CAG A GT C CAG GGCAGCT AG AGGAGCA | |
| NIK: | 1367 | AGAAGAGGAG-AGTGGAGAGGGAACAGGA-GTACATCAGGCGGCAGCTAGAGGAGGAGCA | 1424 |
| NOV3b: | 1419 | T-GCCTACCTCAAGTCCCTGCAGCAGCAGCAACAGCAGCAGCAGCTTCA-GAAACAGCAG | 1476 |
| | | G C ACCT AG CCTGCAGCAGCAGC C CAG AGCAG CA G AC GCA | |
| NIK: | 1425 | GCGGC-ACCTGGAGATCCTGCAGCAGCAGCTGCTCCAGGAGCAGGC-CATGTTACTGCAC | 1482 |
| NOV3b: | 1477 | CAGCAGCAG 1485 (SEQ ID NO: 64) | |
| | | A CA CAG | |
| NIK: | 1483 | GACCA-CAG 1490 (SEQ ID NO: 31) | |

TABLE 15
Score = 2114 bits (5478), Expect = 0.0
Identities = 1093/1303 (83%), Positives = 1093/1303 (83%), Gaps = 8/1303 (0%)

| | | | |
|---|---|---|---|
| NOV3b: | 1 | MGDPAPARSLDDIDLSALRDPAGIFELVEVVGNGTYGQVYKGRHVKTGQLAAIKVMDVTX | 60 |
| | | MGDPAPARSLDDIDLSALRDPAGIFELVEVVGNGTYGQVYKGRHVKTGQLAAIKVMDVT | |
| NIK: | 1 | MGDPAPARSLDDIDLSALRDPAGIFELVEVVGNGTYGQVYKGRHVKTGQLAAIKVMDVTE | 60 |
| NOV3b: | 61 | XXXXXIKQEINMLKKYSHHRNIATYYGAFIKKSPPGNDDQLWLVMEFCGAGSVTDLVKNT | 120 |
| | | IKQEINMLKKYSHHRNIATYYGAFIKKSPPGNDDQLWLVMEFCGAGSVTDLVKNT | |
| NIK: | 61 | DEEEEIKQEINMLKKYSHHRNIATYYGAFIKKSPPGNDDQLWLVMEFCGAGSVTDLVKNT | 120 |
| NOV3b: | 121 | KGNALKEDCIAYICREILRGLAHLHAHKVIHRDIKGQNVLLTENAEVKLVDFGVSAQLDR | 180 |
| | | KGNALKEDCIAYICREILRGLAHLHAHKVIHRDIKGQNVLLTENAEVKLVDFGVSAQLDR | |
| NIK: | 121 | KGNALKEDCIAYICREILRGLAHLHAHKVIHRDIKGQNVLLTENAEVKLVDFGVSAQLDR | 180 |
| NOV3b: | 181 | TVGRRNTFIGTPYWMAPEVIACDENPDATYDYRSDIWSLGITAIEMAEGAPPLCDMHPMR | 240 |
| | | TVGRRNTFIGTPYWMAPEVIACDENPDATYDYRSDIWSLGITAIEMAEGAPPLCDMHPMR | |
| NIK: | 181 | TVGRRNTFIGTPYWMAPEVIACDENPDATYDYRSDIWSLGITAIEMAEGAPPLCDMHPMR | 240 |
| NOV3b: | 241 | ALFLIPRNPPPRLKSKKWSKKFIDFIDTCLIKTYLSRPPTEQLLKFPFIRDQPTERQVRI | 300 |
| | | ALFLIPRNPPPRLKSKKWSKKFIDFIDTCLIKTYLSRPPTEQLLKFPFIRDQPTERQVRI | |
| NIK: | 241 | ALFLIPRNPPPRLKSKKWSKKFIDFIDTCLIKTYLSRPPTEQLLKFPFIRDQPTERQVRI | 300 |
| NOV3b: | 301 | QLKDHIXXXXXXXXXXXXXXXXXXXXXXXXXXXXXXXXPSSIMNVPGESTLRREFLRLQQ | 360 |
| | | QLKDHI PSSIMNVPGESTLRREFLRLQQ | |
| NIK: | 301 | QLKDHIDRSRKKRGEKEETEYEYSGSEEEDDSHGEEGEPSSIMNVPGESTLRREFLRLQQ | 360 |
| NOV3b: | 361 | ENKSNSEALKXXXXXXXXXXRDPEAHIKHLLHXXXXXXXXXXXXXXXXXXXXXXXXXXXX | 420 |
| | | ENKSNSEALK RDPEAHIKHLLH | |
| NIK: | 361 | ENKSNSEALKQQQQLQQQQQRDPEAHIKHLLHQRQRRIEEQKEERRRVEEQQRREREQRK | 420 |
| NOV3b: | 421 | XXXXXXXXXXXDMQALXXXXXXXXXXXXXXYIRHXXXXXXXXXXXXXXXXXXHAYLKSXX | 480 |
| | | DMQAL Y R HAYLKS | |
| NIK: | 421 | LQEKEQQRRLEDMQALRREEERRQAEREQEYKRKQLEEQRQSERLQRQLQQEHAYLKSLQ | 480 |
| NOV3b: | 481 | XXXXXXXXXXXXXXXXXPGDRKPLYHYGRGMNPADKPAWAREVEERTRMNKQQNSPLAKS | 540 |
| | | PGDRKPLYHYGRGMNPADKPAWAREVEERTRMNKQQNSPLAKS | |
| NIK: | 481 | QQQQQQQLQKQQQQQLLPGDRKPLYHYGRGMNPADKPAWAREVEERTRMNKQQNSPLAKS | 540 |
| NOV3b: | 541 | KPGSTXXXXXXXXXXXXXXXXXXXXXXMQRPVEPQEGPHKSLVAHRVPLKPYAAPVPRSQ | 600 |
| | | KPGST MQRPVEPQEGPHKSLVAHRVPLKPYAAPVPRSQ | |
| NIK: | 541 | KPGSTGPEPPIPQASPGPPGPLSQTPPMQRPVEPQEGPHKSLVAHRVPLKPYAAPVPRSQ | 600 |
| NOV3b: | 601 | SLQDQPTRNLAAFPASHXXXXXXXXXXXXXXXRGAVIRQNSDPTSEGPGPSPNPPAWVRP | 660 |
| | | SLQDQPTRNLAAFPASH RGAVIRQNSDPTSEGPGPSPNPPAWVRP | |
| NIK: | 601 | SLQDQPTRNLAAFPASHDPDPAIPAPTATPSARGAVIRQNSDPTSEGPGPSPNPPAWVRP | 660 |
| NOV3b: | 661 | DNEAPPKVPQRTSSIATALNTSGAGGSRPAQAVRASNPDLRRSDPGWERSDSVLPASHGH | 720 |
| | | DNEAPPKVPQRTSSIATALNTSGAGGSRPAQAVRASNPDLRRSDPGWERSDSVLPASHGH | |
| NIK: | 661 | DNEAPPKVPQRTSSIATALNTSGAGGSRPAQAVRASNPDLRRSDPGWERSDSVLPASHGH | 720 |
| NOV3b: | 721 | LPQAGSLERNRVGVSSKPDSSPVLSPGNKAKPDDHRSRPGRPASYKRAIGEDFVLLKERT | 780 |
| | | LPQAGSLERNRVGVSSKPDSSPVLSPGNKAKPDDHRSRPGRPA DFVLLKERT | |
| NIK: | 721 | LPQAGSLERNRVGVSSKPDSSPVLSPGNKAKPDDHRSRPGRPA--------DFVLLKERT | 772 |
| NOV3b: | 781 | LDEAPRPPKKAMDYXXXXXXXXXXXXXXXXXXXXXXXXXRDTPGGRSDGDTDSVSTMVVH | 840 |
| | | LDEAPRPPKKAMDY RDTPGGRSDGDTDSVSTMVVH | |
| NIK: | 773 | LDEAPRPPKKAMDYSSSSEEVESSEDDEEEGEGGPAEGSRDTPGGRSDGDTDSVSTMVVH | 832 |
| NOV3b: | 841 | DVEEITGTQPPYGGGTMVVQRTPEEERNLLHADSNGYTNLPDVVQPSHSPTENSKGQSPP | 900 |
| | | DVEEITGTQPPYGGGTMVVQRTPEEERNLLHADSNGYTNLPDVVQPSHSPTENSKGQSPP | |
| NIK: | 833 | DVEEITGTQPPYGGGTMVVQRTPEEERNLLHADSNGYTNLPDVVQPSHSPTENSKGQSPP | 892 |
| NOV3b: | 901 | SKDGSGDYQSRGLVKAPGKSSFTMFVDLGIYQPGGSGDSIPITALVGGEGTRLDQLQYDV | 960 |
| | | SKDGSGDYQSRGLVKAPGKSSFTMFVDLGIYQPGGSGDSIPITALVGGEGTRLDQLQYDV | |
| NIK: | 893 | SKDGSGDYQSRGLVKAPGKSSFTMFVDLGIYQPGGSGDSIPITALVGGEGTRLDQLQYDV | 952 |
| NOV3b: | 961 | RKGSVVNVNPTNTRAHSETPEIRKYKKRFNSEILCAALWGVNLLVGTENGLMLLDRSGQG | 1020 |
| | | RKGSVVNVNPTNTRAHSETPEIRKYKKRFNSEILCAALWGVNLLVGTENGLMLLDPSGQG | |
| NIK: | 953 | RKGSVVNVNPTNTRAHSETPEIRKYKKRFNSEILCAALWGVNLLVGTENGLMLLDRSGQG | 1012 |
| NOV3b: | 1021 | KVYGLIGRRRFQQMDVLEGLNLLITISGKRNKLRVYYLSWLRNKILHNDPEVEKKQGWTT | 1080 |
| | | KVYGLIGRRRFQQMDVLEGLNLLITISGKRNKLRVYYLSWLRNKILHNDPEVEKKQGWTT | |
| NIK: | 1013 | KVYGLIGRRRFQQMDVLEGLNLLITISGKRNKLRVYYLSWLRNKILHNDPEVEKKQGWTT | 1072 |
| NOV3b: | 1081 | VGDMEGCGHYRVVKYERIKFLVIALKSSVEVYAWAPKPYHKFMAFKSFADLPHRPLLVDL | 1140 |
| | | VGDMEGCGHYRVVKYERIKFLVIALKSSVEVYAWAPKPYHKFMAFKSFADLPHRPLLVDL | |
| NIK: | 1073 | VGDMEGCGHYRVVKYERIKFLVIALKSSVEVYAWAPKPYHKFMAFKSFADLPHRPLLVDL | 1132 |
| NOV3b: | 1141 | TVEEGQRLKVIYGSSAGFHAVDVDSGNSYDIYIPVHIQSQITPHAIIFLPNTDGMEMLLC | 1200 |
| | | TVEEGQRLKVIYGSSAGFHAVDVDSGNSYDIYIPVHIQSQITPHAIIFLPNTDGMEMLLC | |
| NIK: | 1133 | TVEEGQRLKVIYGSSAGFHAVDVDSGNSYDIYIPVHIQSQITPHAIIFLPNTDGMEMLLC | 1192 |
| NOV3b: | 1201 | YEDEGVYVNTYGRIIKDVVLQWGEMPTSVAYICSNQIMGWGEKAIEIRSVETGHLDGVFM | 1260 |
| | | YEDEGVYVNTYGRIIKDVVLQWGEMPTSVAYICSNQIMGWGEKAIEIRSVETGHLDGVFM | |
| NIK: | 1193 | YEDEGVYVNTYGRIIKDVVLQWGEMPTSVAYICSNQIMGWGEKAIEIRSVETGHLDGVFM | 1252 |

| | | |
|---|---|---|
| NOV3b: | 1261 | HKRAQRLKFLCERNDKVFFASVRSGGSSQVYFMTLNRNCIMNW 1303 (SEQ ID NO: 65) |
| | | HKRAQRLKFLCERNDKVFFASVRSGGSSQVYFMTLNPNCIMNW |
| NIK: | 1253 | HKRAQRLKFLCERNDKVFFASVRSGGSSQVYFMTLNRNCIMNW 1295 (SEQ ID NO: 32) |

Based on its relatedness to known members of the STE20 family of protein kinases, NOV3b provides new diagnostic and therapeutic compositions useful in the treatment of disorders associated with alterations in the expression of members of the STE20 family of protein kinases. Nucleic acids, polypeptides, antibodies, and other compositions of the present invention are useful in the treatment and diagnosis of a variety of diseases and pathologies, including, by way of nonlimiting example, those involving metabolic and endocrine disorders, cancer, bone disorders, and tissue/cell growth regulation disorders.

NOV-3c

A NOV-3c sequence according to the invention is a nucleic acid sequence encoding a polypeptide related to STE20 family of protein kinases. A disclosed NOV-3c nucleic acid and its encoded polypeptide includes the sequences shown in Table 16. The disclosed nucleic acid (SEQ ID NO: 10) is 3822 nucleotides in length and contains an open reading frame (ORF) that begins with an ATG initiation codon at nucleotides 1-3 and ends with a TGA stop codon at nucleotides 3820-3822. The start and stop codons are shown in bold font. A respective ORF encodes a 1273 amino acid polypeptide (SEQ ID NO: 11).

TABLE 16

| | |
|---|---|
| ATGGGCGACCCAGCCCCCGCCCGCAGCCTGGACGACATCGACCTGTCCGCCCTGCGGGACCCTGCTGGGATCTTTGAGCT | (SEQ ID NO: 10) |
| TGTGGAGGTGGTCGGCAATGGAACCTACGGACAGGTGTACAAGGGTCGGCATGTCAAGACGGGGCAGCTGGCTGCCATCA | |
| AGGTCATGGATGTCACGGAGGACGAGGAGGAAGAGATCAAACAGGAGATCAACATGCTGAAAAAGTACTCTCACCACCGC | |
| AACATCGCCACCTACTACGGAGCCTTCATCAAGAAGAGCCCCCCGGGAAACGATGACCAGCTCTGGCTGGTGATGGAGTT | |
| CTGTGGTGCTGGTTCAGTGACTGACCTGGTAAAGAACACAAAAGGCAACGCCCTGAAGGAGGACTGTATCGCCTATATCT | |
| GCAGGGAGATCCTCAGGGGTCTGGCCCATCTCCATGCCCACAAGGTGATCCATCGAGACATCAAGGGGCAGAATGTGCTG | |
| CTGACAGAGAATGCTGAGGTCAAGCTAGTGGATTTTGGGGTGAGTGCTCAGCTGGACCGCACCGTGGGCAGACGGAACAC | |
| TTTCATTGGGACTCCCTACTGGATGGCTCCAGAGGTCATCGCCTGTGATGAGAACCCTGATGCCACCTATGATTACAGGA | |
| GTGATATTTGGTCTCTAGGAATCACAGCCATCGAGATGGCAGAGGGAGCCCCCCCTCTGTGTGACATGCACCCCATGCGA | |
| GCCCTCTTCCTCATTCCTCGGAACCCTCCGCCCAGGCTCAAGTCCAAGAAGTGGTCTAAGAAGTTCATTGACTTCATTGA | |
| CACATGTCTCATCAAGACTTACCTGAGCCGCCCACCCACGGAGCAGCTACTGAAGTTTCCCTTCATCCGGGACCAGCCCA | |
| CGGAGCGGCAGGTCCGCATCCAGCTTAAGGACCACATTGACCGATCCCGGAAGAAGCGGGGTGAGAAAGAGGAGACAGAA | |
| TATGAGTACAGCGGCAGCGAGGAGGAAGATGACAGCCATGGAGAGGAAGGAGAGCCAAGCTCCATCATGAACGTGCCTGG | |
| AGAGTCGACTCTACGCCGGGAGTTTCTCCGGCTCCAGCAGGAAAATAAGAGCAACTCAGAGGCTTTAAAACAGCAGCAGC | |
| AGCTGCAGCAGCAGCAGCAGCGAGACCCCGAGGCACACATCAAACACCTGCTGCACCAGCGGCAGCGGCGCATAGAGGAG | |
| CAGAAGGAGGAGCGGCGCCGCGTGGAGGAGCAACAGCGGCGGGAGCGGGAGCAGCGGAAGCTGCAGGAGAAGGAGCAGCA | |
| GCGGCGGCTGGAGGACATGCAGGCTCTGCGGCGGGAGGAGGAGCGGCGGCAGGCGGAGCGCGAGCAGGAATATATTCGTC | |
| ACAGGCTAGAGGAGGAGCAGCGACAGCTCGAGATCCTTCAGCAACAGCTGCTCCAGGAACAGGCCCTGCTGCTGGAATAC | |
| AAGCGGAAGCAGCTGGAGGAGCAGCGGCAGTCAGAACGTCTCCAGAGGCAGCTGCAGCAGGAGCATGCCTACCTCAAGTC | |
| CCTGCAGCAGCAGCAACAGCAGCAGCAGCTTCAGAAACAGCAGCAGCAGCAGCTCCTGCCTGGGGACAGGAAGCCCCTGT | |
| ACCATTATGGTCGGGGCATGAATCCCGCTGACAAACCAGCCTGGGCCCGAGAGGTAGTGGCACACCGGGTCCCACTGAAG | |
| CCATATGCAGCACCTGTACCCCGATCCCAGTCCCTGCAGGACCAGCCCACCCGAAACCTGGCTGCCTTCCCAGCCTCCCA | |
| TGACCCCGACCCTGCCATCCCCGCACCCACTGCCACGCCCAGTGCCCGAGGAGCTGTCATCCGCCAGAATTCAGACCCCA | |
| CCTCTGAAGGACCTGGCCCCAGCCCGAATCCCCCAGCCTGGGTCCGCCCAGATAACGAGGCCCCACCCAAGGTGCCTCAG | |
| AGGACCTCATCTATCGCCACTGCCCTTAACACCAGTGGGGCCGGAGGGTCCCGGCCAGCCCAGGCAGTCCGTGCCAGTAA | |
| CCCCGACCTCAGGAGGAGCGACCCTGGCTGGGAACGCTCGGACAGCGTCCTTCCAGCCTCTCACGGGCACCTCCCCCAGG | |
| CTGGCTCACTGGAGCGGAACCGCGTGGGAGTCTCCTCCAAACCGGACAGCTCCCCTGTGCTCTCCCCTGGGAATAAAGCC | |
| AAGCCCGACGACCACCGCTCACGGCCAGGCCGGCCCGCAAGCTATAAGCGAGCAATTGGTGAGGACTTTGTGTTGCTGAA | |
| AGAGCGGACTCTGGACGAGGCCCCTCGGCCTCCCAAGAAGGCCATGGACTACTCGTCGTCCAGCGAGGAGGTGGAAAGCA | |
| GTGAGGACGACGAGGAGGAAGGCGAAGGCGGGCCAGCAGAGGGGAGCAGAGATACCCCTGGGGGCCGCAGCGATGGGGAT | |
| ACAGACAGCGTCAGCACCATGGTGGTCCACGACGTCGAGGAGATCACCGGGACCCAGCCCCCATACGGGGGCGGCACCAT | |
| GGTGGTCCAGCGCACCCCTGAAGAGGAGCGGAACCTGCTGCATGCTGACAGCAATGGGTACACAAACCTGCCTGACGTGG | |
| TCCAGCCCAGCCACTCACCCACCGAGAACAGCAAAGGCCAAAGCCCACCCTCGAAGGATGGGAGTGGTGACTACCAGTCT | |
| CGTGGGCTGGTAAAGGCCCCTGGCAAGAGCTCGTTCACGATGTTTGTGGATCTAGGGATCTACCAGCCTGGAGGCAGTGG | |
| GGACAGCATCCCCATCACAGCCCTAGTGGGTGGAGAGGGCACTCGGCTCGACCAGCTGCAGTACGACGTGAGGAAGGGTT | |
| CTGTGGTCAACGTGAATCCCACCAACACCCGGGCCCACAGTGAGACCCCTGAGATCCGGAAGTACAAGAAGCGATTCAAC | |
| TCCGAGATCCTCTGTGCAGCCCTTTGGGGGGTCAACCTGCTGGTGGGCACGGAGAACGGGCTGATGTTGCTGGACCGAAG | |
| TGGGCAGGGCAAGGTGTATGGACTCATTGGGCGGCGACGCTTCCAGCAGATGGATGTGCTGGAGGGGCTCAACCTGCTCA | |
| TCACCATCTCAGGGAAAAGGAACAAACTGCGGGTGTATTACCTGTCCTGGCTCCGGAACAAGATTCTGCACAATGACCCA | |
| GAAGTGGAGAAGAAGCAGGGCTGGACCACCGTGGGGGACATGGAGGGCTGCGGGCACTACCGTGTTGTGAAATACGAGCG | |
| GATTAAGTTCCTGGTCATCGCCCTCAAGAGCTCCGTGGAGGTGTATGCCTGGGCCCCCAAACCCTACCACAAATTCATGG | |
| CCTTCAAGTCCTTTGCCGACCTCCCCCACCGCCCTCTGCTGGTCGACCTGACAGTAGAGGAGGGGCAGCGGCTCAAGGTC | |
| ATCTATGGCTCCAGTGCTGGCTTCCATGCTGTGGATGTCGACTCGGGGAACAGCTATGACATCTACATCCCTGTGCACAT | |
| CCAGAGCCAGATCACGCCCCATGCCATCATCTTCCTCCCCAACACCGACGGCATGGAGATGCTGCTGTGCTACGAGGACG | |
| AGGGTGTCTACGTCAACACGTACGGGCGCATCATTAAGGATGTGGTGCTGCAGTGGGGGGAGATGCCTACTTCTGTGGCC | |
| TACATCTGCTCCAACCAGATAATGGGCTGGGGTGAGAAAGCCATTGAGATCCGCTCTGTGGAGACGGGCCACCTCGACGG | |
| GGTCTTCATGCACAAACGAGCTCAGAGGCTCAAGTTCCTGTGTGAGCGGAATGACAAGGTGTTTTTTGCCTCAGTCCGCT | |
| CTGGGGGCAGCAGCCAAGTTTACTTCATGACTCTGAACCGTAACTGCATCATGAACTGGTGA | |
| MGDPAPARSLDDIDLSALRDPAGIFELVEVVGNGTYGQVYKGRHVKTGQLAAIKVMDVTEDEEEEIKQEINMLKKYSHHR | (SEQ ID NO: 11) |
| NIATYYGAFIKKSPPGNDDQLWLVMEFCGAGSVTDLVKNTKGNALKEDCIAYICREILRGLAHLHAHKVIHRDIKGQNVL | |
| LTENAEVKLVDFGVSAQLDRTVGRRNTFIGTPYWMAPEVIACDENPDATYDYRSDIWSLGITAIEMAEGAPPLCDMHPMR | |
| ALFLIPRNPPPRLKSKKWSKKFIDFIDTCLIKTYLSRPPTEQLLKFPFIRDQPTERQVRIQLKDHIDRSRKKRGEKEETE | |
| YEYSGSEEEDDSHGEEGEPSSIMNVPGESTLRREFLRLQQENKSNSEALKQQQQLQQQQQRDPEAHIKHLLHQRQRRIEE | |
| QKEERRRVEEQQRREREQRKLQEKEQQRRLEDMQALRREEERRQAEREQEYIRHRLEEEQRQLEILQQQLLQEQALLLEY | |
| KRKQLEEQRQSERLQRQLQQEHAYLKSLQQQQQQQQLQKQQQQQLLPGDRKPLYHYGRGMNPADKPAWAREVVAHRVPLK | |
| PYAAPVPRSQSLQDQPTRNLAAFPASHDPDPAIPAPTATPSARGAVIRQNSDPTSEGPGPSPNPPAWVRPDNEAPPKVPQ | |
| RTSSIATALNTSGAGGSRPAQAVRASNPDLRRSDPGWERSDSVLPASHGHLPQAGSLERNRVGVSSKPDSSPVLSPGNKA | |
| KPDDHRSRPGRPASYKRAIGEDFVLLKERTLDEAPRPPKKAMDYSSSSEEVESSEDDEEEGEGGPAEGSRDTPGGRSDGD | |
| TDSVSTMVVHDVEEITGTQPPYGGGTMVVQRTPEEERNLLHADSNGYTNLPDVVQPSHSPTENSKGQSPPSKDGSGDYQS | |
| RGLVKAPGKSSFTMFVDLGIYQPGGSGDSIPITALVGGEGTRLDQLQYDVRKGSVVNVNPTNTRAHSETPEIRKYKKRFN | |
| SEILCAALWGVNLLVGTENGLMLLDRSGQGKVYGLIGRRRFQQMDVLEGLNLLITISGKRNKLRVYYLSWLRNKILHNDP | |
| EVEKKQGWTTVGDMEGCGHYRVVKYERIKFLVIALKSSVEVYAWAPKPYHKFMAFKSFADLPHRPLLVDLTVEEGQRLKV | |
| IYGSSAGFHAVDVDSGNSYDIYIPVHIQSQITPHAIIFLPNTDGMEMLLCYEDEGVYVNTYGRIIKDVVLQWGEMPTSVA | |
| YICSNQIMGWGEKAIEIRSVETGHLDGVFMHKRAQRLKFLCERNDKVFFASVRSGGSSQVYFMTLNRNCIMNW | |

The disclosed NOV-3c nucleic acid sequence has homology (72% identity) to a mouse mRNA for a NIK protein (NIK) (GenBank Accession No: MMU88984), as shown in Table 17. NIK proteins are a subgroup of the STE20 family of protein kinases. As indicated by the “Expect” value, the probability of this alignment occurring by chance alone is 9.1e-299. Moreover, the disclosed, encoded amino acid sequence has 1048 of 1332 amino acid residues (78%) identical to a human NIK-related protein (GenBank Accession No: BAA90753), shown in Table 18. Furthermore, the encoded amino acid sequence also has homology (79% identity) to a human GCK kinase (GenBank Accession No: BAA94838), another subgroup of the STE20 kinase family, as shown in Table 19. As indicated by the “Expect” value, the probability of these amino acid alignments occurring by chance alone are both 0.0, the lowest probability score.

TABLE 17
Score = 3907 (586.2 bits), Expect = 9.1e-299, Sum P(2) = 9.1e-299
Identities = 1297/1788 (72%), Positives = 1297/1788 (72%), Strand = Plus /
Plus

| | | | |
|---|---|---|---|
| NOV3c: | 4 | GGCGACCCAGCC-CCCGCCCGCAGCCTGGACGACATCGACCTGTCCGCCCTGCGGGACCC | 62 |
| | | GGCGA C A C CCCGC AGCCTGG GACAT GACCTGTC CCCTGCGGGACCC | |
| NIK: | 3 | GGCGAACGACTCTCCCGCGAAGAGCCTGGTGGACATTGACCTGTCGTCCCTGCGGGACCC | 62 |
| NOV3c: | 63 | TGCTGGGATCTTTGAGCTTGTGGAGGTGGTCGGCAATGGAACCTACGGACAGGTGTACAA | 122 |
| | | TGCTGGGAT TTTGAGCT GTGGA GTGGT GG AATGG ACCTA GGACA GT TA AA | |
| NIK: | 63 | TGCTGGGATTTTTGAGCTGGTGGAAGTGGTTGGAAATGGCACCTATGGACAAGTCTATAA | 122 |
| NOV3c: | 123 | GGGTCGGCATGTCAAGACGGGGCAGCTGGCTGCCATCAAGGTCATGGATGTCACGGAGGA | 182 |
| | | GGGTCG CATGT AA ACGG CA CTG C GCCATCAAGGT ATGGA GTCAC GAGGA | |
| NIK: | 123 | GGGTCGACATGTTAAAACGGT-CA-CTGCC-GCCATCAAGGTTATGGACGTCACCGAGGA | 179 |
| NOV3c: | 183 | CGAGGAGGAAGAGATCAAACAGGAGATCAACATGCTGAAAAAGTACTCTCACCACCGCAA | 242 |
| | | GA GAGGAAGA ATCA AC GGAGAT AA ATGCTGAA AAGTA TCTCA CA CG AA | |
| NIK: | 180 | TGAAGAGGAAGAAATCACACTGGAGATAAATATGCTGAAGAAGTATTCTCATCATCGAAA | 239 |
| NOV3c: | 243 | CATCGCCACCTACTACGGAGCCTTCATCAAGAAGAGCCCCCCGGGAAACGATGACCAGCT | 302 |
| | | AT GCCAC TACTA GG GC TTCAT AAGAAGAGCCC CC GGA A GATGACCA CT | |
| NIK: | 240 | TATTGCCACGTACTATGGTGCTTTCATTAAGAAGAGCCCTCCAGGACATGATGACCAACT | 299 |
| NOV3c: | 303 | CTGGCTGGTGATGGAGTTCTGTGGTGCTGGTTCAGTGACTGACCTGGTAAAGAACACAAA | 362 |
| | | CTGGCT GT ATGGAGTT TGTGG GCTGG TC T AC GACCT GT AAGAACAC AA | |
| NIK: | 300 | CTGGCTTGTTATGGAGTTTTGTGGGGCTGGGTCCATCACAGACCTTGTGAAGAACACCAA | 359 |
| NOV3c: | 363 | AGGCAACGCCCTGAAGGAGGACTGTATCGCCTATATCTGCAGGGAGATCCTCAGGGGTCT | 422 |
| | | AGG AAC C CT AA GA GACTG AT GC TA ATCT CAGGGA ATCCTCAGGGG T | |
| NIK: | 360 | AGGGAACACTCTCAAAGAAGACTGGATTGCTTACATCTCCAGGGAAATCCTCAGGGGATT | 419 |
| NOV3c: | 423 | GGCCCATCTCCATGCCCACAAGGTGATCCATCGAGACATCAAGGGGCAGAATGTGCTGCT | 482 |
| | | GGC CATCTCCAT CAC A GT AT CA CGAGA ATCAAGGG CA AATGTGCTGCT | |
| NIK: | 420 | GGCACATCTCCATATTCACCACGTTATTCACCGAGATATCAAGGGCCAAAATGTGCTGCT | 479 |
| NOV3c: | 483 | GACAGAGAATGCTGAGGTCAAGCTAGTGGATTTTGGGGTGAGTGCTCAGCTGGACCGCAC | 542 |
| | | GAC GAGAATGCTGAGGT AA CT GT GATTTTGG GT AG GCTCAGCTGGAC G AC | |
| NIK: | 480 | GACCGAGAATGCTGAGGTGAAACTTGTTGATTTTGGTGTAAGCGCTCAGCTGGACAGGAC | 539 |
| NOV3c: | 543 | CGTGGG-CAGACGGAACACTTTCATTGGGACTCCCTACTGGATGGCTCCAGAGGTCATCG | 601 |
| | | GT GG C GA G AA AC TTCAT GG AC CCCTACTGGATGGCTCCAGAGGTCATCG | |
| NIK: | 540 | GGTTGGACGGA-GAAATACGTTCATAGGCACACCCTACTGGATGGCTCCAGAGGTCATCG | 598 |
| NOV3c: | 602 | CCTGTGATGAGAACCCTGATGCCACCTATGATTACAGGAGTGATATTTGGTCTCTA-GGA | 660 |
| | | CCTGTGATGAGAACCC GA GCCAC TA GA TACAG AGTGA T TGGTC CT GG | |
| NIK: | 599 | CCTGTGATGAGAACCCAGACGCCACTTACGACTACAGAAGTGACCTCTGGTC-CTGTGGC | 657 |
| NOV3c: | 661 | ATCACAGCCATCGAGATGGCAGAGGGAGCCCCCCCTCTGTGTGACATGCACCCCATGCGA | 720 |
| | | ATCACAGCCATCGAGATGGC GA GG G CCCCCCTCT TGTGACATGCA CC ATG GA | |
| NIK: | 658 | ATCACAGCCATCGAGATGGCTGAAGGGGGCCCCCCTCTCTGTGACATGCATCCAATGAGA | 717 |
| NOV3c: | 721 | GCCCTCTTCCTCATTCCTCGGAACCCTCCGCCCAGGCTCAAGTCCAAGAAGTGGTCTAAG | 780 |
| | | GC CT TT CTCAT CC G AACCCTCC CCCAGGCT AAGTC AA AA TGGTC AAG | |
| NIK: | 718 | GCGCTGTTTCTCATCCCCAGAAACCCTCCTCCCAGGCTGAAGTCAAAAAAATGGTCAAAG | 777 |
| NOV3c: | 781 | AAGTTCATTGA-CTTCATTGACACATGTCTCATCAAGACTTACCTG-AGCCGCCCACCCA | 838 |
| | | AA TT TT A CTT AT GA TGTCT T AAGA TTAC TG AGC GCCC C A | |
| NIK: | 778 | AAATTT-TTCAGCTTTATAGAAGGCTGTCTGGTGAAGAATTACATGCAGCGGCCCTCT-A | 835 |
| NOV3c: | 839 | CGGAGCAGCTACTGAAGTTTCCCTTCATCCGGGACCAGCCCACGGAGCGGCAGGTCCGCA | 898 |
| | | C GAGCA CT T AA CC TTCAT GGGA CAGCCCA GA GGCAGGT CG A | |
| NIK: | 836 | CAGAGCAACTTTTAAAACACCCTTTCATAAGGGATCAGCCCAATGAAAGGCAGGTTCGAA | 895 |
| NOV3c: | 899 | TCCAGCTTAAGGACCACATTGACCGATCCCGGAAGAAGCGGGGTGAGAAAGAGGAGACAG | 958 |
| | | TCCAGCTTAAGGA CACAT GACCG CC G AAGAAG G GG GAGAAAGA GAGAC G | |
| NIK: | 896 | TCCAGCTTAAGGATCACATAGACCGGACCAGAAAGAAGAGAGGCGAGAAAGATGAGACGG | 955 |
| NOV3c: | 959 | AATATGAGTACAGCGGCAGCGAGGAGGAAGATGAC-A-GC-CATGGAG-AGGAAGGAGAG | 1014 |
| | | A TA GAGTACAGCGG AGCGAGGAGGA GA GA A G C TG AG AGGA GGAGAG | |
| NIK: | 956 | AGTACGAGTACAGCGGGAGCGAGGAGGAGGAGGAGGAAGTGCCTG-AGCAGGAGGGAGAG | 1014 |
| NOV3c: | 1015 | CCAAGCTCCATCATGAACGTGCCTGGAGAGTCGACTCTACGCCGGGAGTTTCTCCGGCTC | 1074 |
| | | CCAAG TCCATC T AA GTGCCTGGAGAGTC ACTCT CG CG GA TT CT G CT | |
| NIK: | 1015 | CCAAGTTCCATCGTCAATGTGCCTGGAGAGTCAACTCTGCGACGTGATTTCCTGAGACTG | 1074 |
| NOV3c: | 1075 | CAGCAGGAAAATAAG-AGCAACTCAGAGGCTTTAAAACAG-CAGCAGCAGCTGCAGCAGC | 1132 |
| | | CAGCAGGA AA AAG AGC TC GAGGCT T AG CAGCAGC CTGCAG AGC | |
| NIK: | 1075 | CAGCAGGAGAACAAGGAGCGG-TCTGAGGCTCTGCGG-AGACAGCAGCTTCTGCAGGAGC | 1132 |
| NOV3c: | 1133 | AGCAGCAGCGAGACCCCGAGGCACACATCAAACACCTGCTGCACCAGCGGCAGCGGCGCA | 1192 |
| | | AGCAGC CG GA C GAGG A A A CA CTGCTG AG GGCAG GCG A | |
| NIK: | 1133 | AGCAGCTCCGGGAGCAGGAGGAGTATAAGAGGCAGCTGCTGGCTGAGAGGCAGAAGCGGA | 1192 |
| NOV3c: | 1193 | TAGAGGAGCAGAAGGAGGAGCGGCGCCGCGTGGAGGAGCAACAGCGGCGGGAGCGGGA-G | 1251 |
| | | T GA AGCAGAA GA AG GG G CG TGGA GAGCAACA G G GA CGGGA G | |
| NIK: | 1193 | TTGAACAGCAGAAAGAACAGAGGAGGCGGCTGGAAGAGCAACAAAGAAGAGAACGGGAAG | 1252 |
| NOV3c: | 1252 | CAGCGGAAGCTGCAGGAGAAGGAGCAGCAGCGGCG-G--CTGGAGGACATGCAGGC-TCT | 1307 |
| | | C GGA GC GCAGGAG GAGCAGC GCGGCG G C GAGGA A G AGGC TCT | |
| NIK: | 1253 | CCA-GGAGGCAGCAGGAGCGTGAGCAGCGGCGGCGTGAACAAGAGGAGAAG-AGGCGTCT | 1310 |
| NOV3c: | 1308 | GCGGCGGGA--GGAGGAGCGGCGGCAGGCGGAGCGCGAGCAGGAATATATTCGTCACAGG | 1365 |
| | | CG GG A GGA GCGGCG A G GAG GAG AGGA A C A AGG | |
| NIK: | 1311 | -CGA-GGAACTGGAAAGGCGGCGTAAAGAAGAGGAAGAG-AGGAG-ACGGGCAGAAGAGG | 1366 |
| NOV3c: | 1366 | CTA-GAGGAG-GAGCAGCGACAGCTCTCGAGATCCTTCAGCAACAGCTGCTCCAGGAACAGG | 1423 |
| | | A GAGGAG G G AG G A C GAG T C TCAG C GC GCT AGGA AG | |
| NIK: | 1367 | AGAAGAGGAGAGTGGAGAGGGAACAGGAG-TACATCAGG--CGGCAGCTAGAGGAGGAGC | 1423 |
| NOV3c: | 1424 | CCCTGCTGCTGGA-ATACA--AGCGGAAGCAGCTGGAGGAGCAGCGGCA-GTCAGAACGT | 1479 |
| | | C GC CTGGA AT C AGC G AGC GCT AGGAGCAG G CA GT A C | |
| NIK: | 1424 | AGCGGCACCTGGAGATCCTGCAGCAGCAGCTGCTCCAGGAGCAG-GCCATGTTACTGCAC | 1482 |
| NOV3c: | 1480 | CTCCAGAGGCAGCTGCA-GCAGGAGCATGCCTACCTCAAGTCCCTGCAGCAGCAGCAACA | 1538 |
| | | CCA AGG GC GCA GCA AGCA GC CC C G CCC GCAGCAGCAG A CA | |
| NIK: | 1483 | GACCACAGGAGGCCGCACGCAC-AGCA-GCAG-CCGCC-GCCCCCGCAGCAGCAGGA-CA | 1537 |
| NOV3c: | 1539 | GCAGCAG--C-AGCTT-CA-GAAACAGCAGCAGCAGCAGCTCC-TG-CC-TGGGGACAGG | 1590 |
| | | G AGCA C AGCTT CA G CAG AGC AGC C C TG CC TG GACAG | |
| NIK: | 1538 | GGAGCAAACCGAGCTTTCATGCTCCAG-AGCCCAAGCCTCACTATGACCCTGCTGACAG- | 1595 |
| NOV3c: | 1591 | AAGCCCCTGTACCATTATGGTCGGGGCATGAATCCCGCT-GA-CAAAC-CAGCCTGGGCC | 1647 |
| | | AGC C G A TGGTC C G ATC C C GA CAA C CC G C | |
| NIK: | 1596 | -AGCTCGGGAGGTACAGTGGTCCCACCTGGCATCTCTCAAGAACAATGTCTCCCCTGTCT | 1654 |
| NOV3c: | 1648 | CGAGAGGTAGTGGCACACCGGGTCCCACTGAAGCCATAT--GCAGCACCTGTACC-CCGA | 1704 |
| | | CGAGA T C C G G CCC T CCA AT GCA CACC A C CCG | |
| NIK: | 1655 | CGAGATCCCATTCCTT-CAGTGACCCT-TCT-CCAAATTCGCA-CACCACCATCTCCGC | 1710 |
| NOV3c: | 1705 | TCCCAGTCCCTGCAGGACCAGCCCACCCGAAACCTGGCTGCCTTCCCAGCCTCCCATGAC | 1764 |
| | | TC CAG CC CA G CCA CC CCCG A GG GC CAG C C TGAC | |
| NIK: | 1711 | TCTCAGGACC--CATGTCCA-CCTTCCCGCAGTGAGGG-GCTCAGTCAGAG-CTC-TGAC | 1764 |
| NOV3c: | 1765 | CCCGACCCTGCCATCCCCGCACCCAC 1790 (SEQ ID NO: 66) | |
| | | C A C G T CCCG CCCAC | |
| NIK: | 1765 | TCTAAGTCGGAGGTGCCCGAGCCCAC 1790 (SEQ ID NO: 33) | |

TABLE 18
Score = 1985 bits (5143), Expect = 0.0
Identities = 1048/1332 (78%), Positives = 1051/1332 (78%) , Gaps = 96/1332
(7%)

| | | | |
|---|---|---|---|
| NOV3c: | 1 | MGDPAPARSLDDIDLSALRDPAGIFELVEVVGNGTYGQVYKGRHVKTGQLAAIKVMDVTX | 60 |
| | | MGDPAPARSLDDIDLSALRDPAGIFELVEVVGNGTYGQVYKGRHVKTGQLAAIKVMDVT | |
| NIK: | 1 | MGDPAPARSLDDIDLSALRDPAGIFELVEVVGNGTYGQVYKGRHVKTGQLAAIKVMDVTE | 60 |
| NOV3c: | 61 | XXXXXIKQEINMLKKYSHHRNIATYYGAFIKKSPPGNDDQLWLVMEFCGAGSVTDLVKNT | 120 |
| | | IKQEINMLKKYSHHRNIATYYGAFIKKSPPGNDDQLWLVMEFCGAGSVTDLVKNT | |
| NIK: | 61 | DEEEEIKQEINMLKKYSHHRNIATYYGAFIKKSPPGNDDQLWLVMEFCGAGSVTDLVKNT | 120 |
| NOV3c: | 121 | KGNALKEDCIAYICREILRGLAHLHAHKVIHRDIKGQNVLLTENAEVKLVDFGVSAQLDR | 180 |
| | | KGNALKEDCIAYICREILRGLAHLHAHKVIHRDIKGQNVLLTENAEVKLVDFGVSAQLDR | |
| NIK: | 121 | KGNALKEDCIAYICREILRGLAHLHAHKVIHRDIKGQNVLLTENAEVKLVDFGVSAQLDR | 180 |
| NOV3c: | 181 | TVGRRNTFIGTPYWMAPEVIACDENPDATYDYRSDIWSLGITAIEMAEGAPPLCDMHPMR | 240 |
| | | TVGRRNTFIGTPYWMAPEVIACDENPDATYDYRSDIWSLGITAIEMAEGAPPLCDMHPMR | |
| NIK: | 181 | TVGRRNTFIGTPYWMAPEVIACDENPDATYDYRSDIWSLGITAIEMAEGAPPLCDMHPMR | 240 |
| NOV3c: | 241 | ALFLIPRNPPPRLKSKKWSKKFIDFIDTCLIKTYLSRPPTEQLLKFPFIRDQPTERQVRI | 300 |
| | | ALFLIPRNPPPRLKSKKWSKKFIDFIDTCLIKTYLSRPPTEQLLKFPFIRDQPTERQVRI | |
| NIK: | 241 | ALFLIPRNPPPRLKSKKWSKKFIDFIDTCLIKTYLSRPPTEQLLKFPFIRDQPTERQVRI | 300 |
| NOV3c: | 301 | QLKDHIXXXXXXXXXXXXXXXXXXXXXXXXXXXXXXXXPSSIMNVPGESTLRREFLRLQQ | 360 |
| | | QLKDHI PSSIMNVPGESTLRREFLRLQQ | |
| NIK: | 301 | QLKDHIDRSRKKRGEKEETEYEYSGSEEEDDSHGEEGEPSSIMNVPGESTLRREFLRLQQ | 360 |
| NOV3c: | 361 | ENKSNSEALKXXXXXXXXXXRDPEAHIKHLLHXXXXXXXXXXXXXXXXXXXXXXXXXXXX | 420 |
| | | ENKSNSEALK RDPEAHIKHLLH | |
| NIK: | 361 | ENKSNSEALKQQQQLQQQQQRDPEAHIKHLLHQRQRRIEEQKEERRRVEEQQRREREQRK | 420 |
| NOV3c: | 421 | XXXXXXXXXXXDMQALXXXXXXXXXXXXXXYIRHRXXXXXXXXXXXXXXXXXXXXXXXXY | 480 |
| | | DMQAL Y | |
| NIK: | 421 | LQEKEQQRRLEDMQAL-----------------------------RREEERRQAEREQEY | 451 |
| NOV3c: | 481 | KRKXXXXXXXXXXXXXXXXXXHAYLKSXXXXXXXXXXXXXXXXXXXPGDRKPLYHYGRGM | 540 |
| | | KRK HAYLKS PGDRKPLYHYGRGM | |
| NIK: | 452 | KRKQLEEQRQSERLQRQLQQEHAYLKSLQQQQQQQQLQKQQQQQLLPGDRKPLYHYGRGM | 511 |
| NOV3c: | 541 | NPADKPAWAREVVAH------------------------------------------RVP | 558 |
| | | NPADKPAWAREV P | |
| NIK: | 512 | NPADKPAWAREVEERTRMNKQQNSPLAKSKPGSTGPEPPIPQASPGPPGPLSQTPPMQRP | 571 |
| NOV3c: | 559 | LKPYAAP-----------------VPRSQSLQDQPTRNLAAFPASHXXXXXXXXXXXXXX | 601 |
| | | ++P P VPRSQSLQDQPTRNLAAFPASH | |
| NIK: | 572 | VEPQEGPHKSLVAHRVPLKPYAAPVPRSQSLQDQPTRNLAAFPASHDPDPAIPAPTATPS | 631 |
| NOV3c: | 602 | XRGAVIRQNSDPTSEGPGPSPNPPAWVRPDNEAPPKVPQRTSSIATALNTSGAGGSRPAQ | 661 |
| | | RGAVIRQNSDPTSEGPGPSPNPPAWVRPDNEAPPKVPQRTSSIATALNTSGAGGSRPAQ | |
| NIK: | 632 | ARGAVIRQNSDPTSEGPGPSPNPPAWVRPDNEAPPKVPQRTSSIATALNTSGAGGSRPAQ | 691 |
| NOV3c: | 662 | AVRASNPDLRRSDPGWERSDSVLPASHGHLPQAGSLERNRVGVSSKPDSSPVLSPGNKAK | 721 |
| | | AVRASNPDLRRSDPGWERSDSVLPASHGHLPQAGSLERNRVGVSSKPDSSPVLSPGNKAK | |
| NIK: | 692 | AVRASNPDLRRSDPGWERSDSVLPASHGHLPQAGSLERNRVGVSSKPDSSPVLSPGNKAK | 751 |
| NOV3c: | 722 | PDDHRSRPGRPASYKRAIGEDFVLLKERTLDEAPRPPKKAMDYXXXXXXXXXXXXXXXXX | 781 |
| | | PDDHRSRPGRPA DFVLLKERTLDEAPRPPKKAMDY | |
| NIK: | 752 | PDDHRSRPGRPA--------DFVLLKERTLDEAPRPPKKAMDYSSSSEEVESSEDDEEEG | 803 |
| NOV3c: | 782 | XXXXXXXXRDTPGGRSDGDTDSVSTMVVHDVEEITGTQPPYGGGTMVVQRTPEEERNLLH | 841 |
| | | RDTPGGRSDGDTDSVSTMVVHDVEEITGTQPPYGGGTMVVQRTPEEERNLLH | |
| NIK: | 804 | EGGPAEGSRDTPGGRSDGDTDSVSTMVVHDVEEITGTQPPYGGGTMVVQRTPEEERNLLH | 863 |
| NOV3c: | 842 | ADSNGYTNLPDVVQPSHSPTENSKGQSPPSKDGSGDYQSRGLVKAPGKSSFTMFVDLGIY | 901 |
| | | ADSNGYTNLPDVVQPSHSPTENSKGQSPPSKDGSGDYQSRGLVKAPGKSSFTMFVDLGIY | |
| NIK: | 864 | ADSNGYTNLPDVVQPSHSPTENSKGQSPPSKDGSGDYQSRGLVKAPGKSSFTMFVDLGIY | 923 |
| NOV3c: | 902 | QPGGSGDSIPITALVGGEGTRLDQLQYDVRKGSVVNVNPTNTRAHSETPEIRKYKKRFNS | 961 |
| | | QPGGSGDSIPITALVGGEGTRLDQLQYDVRKGSVVNVNPTNTRAHSETPEIRKYKKRFNS | |
| NIK: | 924 | QPGGSGDSIPITALVGGEGTRLDQLQYDVRKGSVVNVNPTNTRAHSETPEIRKYKKRFNS | 983 |
| NOV3c: | 962 | EILCAALWGVNLLVGTENGLMLLDRSGQGKVYGLIGRRRFQQMDVLEGLNLLITISGKRN | 1021 |
| | | EILCAALWGVNLLVGTENGLMLLDRSGQGKVYGLIGRRRFQQMDVLEGLNLLITISGKRN | |
| NIK: | 984 | EILCAALWGVNLLVGTENGLMLLDRSGQGKVYGLIGRRRFQQMDVLEGLNLLITISGKRN | 1043 |
| NOV3c: | 1022 | KLRVYYLSWLRNKILHNDPEVEKKQGWTTVGDMEGCGHYRVVKYERIKFLVIALKSSVEV | 1081 |
| | | KLRVYYLSWLRNKILHNDPEVEKKQGWTTVGDMEGCGHYRVVKYERIKFLVIALKSSVEV | |
| NIK: | 1044 | KLRVYYLSWLRNKILHNDPEVEKKQGWTTVGDMEGCGHYRVVKYERIKFLVIALKSSVEV | 1103 |
| NOV3c: | 1082 | YAWAPKPYHKFMAFKSFADLPHRPLLVDLTVEEGQRLKVIYGSSAGFHAVDVDSGNSYDI | 1141 |
| | | YAWAPKPYHKFMAFKSFADLPHRPLLVDLTVEEGQRLKVIYGSSAGFHAVDVDSGNSYDI | |
| NIK: | 1104 | YAWAPKPYHKFMAFKSFADLPHRPLLVDLTVEEGQRLKVIYGSSAGFHAVDVDSGNSYDI | 1163 |
| NOV3c: | 1142 | YIPVHIQSQITPHAIIFLPNTDGMEMLLCYEDEGVYVNTYGRIIKDVVLQWGEMPTSVAY | 1201 |
| | | YIPVHIQSQITPHAIIFLPNTDGMEMLLCYEDEGVYVNTYGRIIKDVVLQWGEMPTSVAY | |
| NIK: | 1164 | YIPVHIQSQITPHAIIFLPNTDGMEMLLCYEDEGVYVNTYGRIIKDVVLQWGEMPTSVAY | 1223 |
| NOV3c: | 1202 | ICSNQIMGWGEKAIEIRSVETGHLDGVFMHKRAQRLKFLCERNDKVFFASVRSGGSSQVY | 1261 |
| | | ICSNQIMGWGEKAIEIRSVETGHLDGVFMHKRAQRLKFLCERNDKVFFASVRSGGSSQVY | |
| NIK: | 1224 | ICSNQIMGWGEKAIEIRSVETGHLDGVFMHKRAQRLKFLCERNDKVFFASVRSGGSSQVY | 1283 |
| NOV3c: | 1262 | FMTLNRNCIMNW 1273 (SEQ ID NO: 67) | |
| | | FMTLNRNCIMNW | |
| NIK: | 1284 | FMTLNRNCIMNW 1295 (SEQ ID NO: 34) | |

TABLE 19
Score = 2007 bits (5201), Expect = 0.0
Identities = 1056/1332 (79%) , Positives = 1059/1332 (79%), Gaps = 88/1332
(6%)

| | | | |
|---|---|---|---|
| NOV3c: | 1 | MGDPAPARSLDDIDLSALRDPAGIFELVEVVGNGTYGQVYKGRHVKTGQLAAIKVMDVTX | 60 |
| | | MGDPAPARSLDDIDLSALRDPAGIFELVEVVGNGTYGQVYKGRHVKTGQLAAIKVMDVT | |
| GCK: | 1 | MGDPAPARSLDDIDLSALRDPAGIFELVEVVGNGTYGQVYKGRHVKTGQLAAIKVMDVTE | 60 |
| NOV3c: | 61 | XXXXXIKQEINMLKKYSHHRNIATYYGAFIKKSPPGNDDQLWLVMEFCGAGSVTDLVKNT | 120 |
| | | IKQEINMLKKYSHHRNIATYYGAFIKKSPPGNDDQLWLVMEFCGAGSVTDLVKNT | |
| GCK: | 61 | DEEEEIKQEINMLKKYSHHRNIATYYGAFIKKSPPGNDDQLWLVMEFCGAGSVTDLVKNT | 120 |
| NOV3c: | 121 | KGNALKEDCIAYICREILRGLAHLHAHKVIHRDIKGQNVLLTENAEVKLVDFGVSAQLDR | 180 |
| | | KGNALKEDCIAYICREILRGLAHLHAHKVIHRDIKGQNVLLTENAEVKLVDFGVSAQLDR | |
| GCK: | 121 | KGNALKEDCIAYICREILRGLAHLHAHKVIHRDIKGQNVLLTENAEVKLVDFGVSAQLDR | 180 |
| NOV3c: | 181 | TVGRRNTFIGTPYWMAPEVIACDENPDATYDYRSDIWSLGITAIEMAEGAPPLCDMHPMR | 240 |
| | | TVGRRNTFIGTPYWMAPEVIACDENPDATYDYRSDIWSLGITAIEMAEGAPPLCDMHPMR | |
| GCK: | 181 | TVGRRNTFIGTPYWMAPEVIACDENPDATYDYRSDIWSLGITAIEMAEGAPPLCDMHPMR | 240 |
| NOV3c: | 241 | ALFLIPRNPPPRLKSKKWSKKFIDFIDTCLIKTYLSRPPTEQLLKFPFIRDQPTERQVRI | 300 |
| | | ALFLIPRNPPPRLKSKKWSKKFIDFIDTCLIKTYLSRPPTEQLLKFPFIRDQPTERQVRI | |
| GCK: | 241 | ALFLIPRNPPPRLKSKKWSKKFIDFIDTCLIKTYLSRPPTEQLLKFPFIRDQPTERQVRI | 300 |
| NOV3c: | 301 | QLKDHIXXXXXXXXXXXXXXXXXXXXXXXXXXXXXXXXPSSIMNVPGESTLRREFLRLQQ | 360 |
| | | QLKDHI PSSIMNVPGESTLRREFLRLQQ | |
| GCK: | 301 | QLKDHIDRSRKKRGEKEETEYEYSGSEEEDDSHGEEGEPSSIMNVPGESTLRREFLRLQQ | 360 |
| NOV3c: | 361 | ENKSNSEALKXXXXXXXXXXRDPEAHIKHLLHXXXXXXXXXXXXXXXXXXXXXXXXXXXX | 420 |
| | | ENKSNSEALK RDPEAHIKHLLH | |
| GCK: | 361 | ENKSNSEALKQQQQLQQQQQRDPEAHIKHLLHQRQRRIEEQKEERRRVEEQQRREREQRK | 420 |
| NOV3c: | 421 | XXXXXXXXXXDMQALXXXXXXXXXXXXXXYIRHRXXXXXXXXXXXXXXXXXXXXXXXXY | 480 |
| | | DMQAL Y | |
| GCK: | 421 | LQEKEQQRRLEDMQAL-----------------------------RREEERRQAEREQEY | 451 |
| NOV3c: | 481 | KRKXXXXXXXXXXXXXXXXXXHAYLKSXXXXXXXXXXXXXXXXXXXPGDRKPLYHYGRGM | 540 |
| | | KRK HAYLKS PGDRKPLYHYGRGM | |
| GCK: | 452 | KRKQLEEQRQSERLQRQLQQEHAYLKSLQQQQQQQQLQKQQQQQLLPGDRKPLYHYGRGM | 511 |
| NOV3c: | 541 | NPADKPAWAREVVAH------------------------------------------RVP | 558 |
| | | NPADKPAWAREV + P | |
| GCK: | 512 | NPADKPAWAREVEERTRMNKQQNSPLAKSKPGSTGPEPPIPQASPGPPGPLSQTPPMQRP | 571 |
| NOV3c: | 559 | LKPYAAP-----------------VPRSQSLQDQPTRNLAAFPASHXXXXXXXXXXXXXX | 601 |
| | | ++P P VPRSQSLQDQPTRNLAAFPASH | |
| GCK: | 572 | VEPQEGPHKSLVAHRVPLKPYAAPVPRSQSLQDQPTRNLAAFPASHDPDPAIPAPTATPS | 631 |
| NOV3c: | 602 | XRGAVIRQNSDPTSEGPGPSPNPPAWVRPDNEAPPKVPQRTSSIATALNTSGAGGSRPAQ | 661 |
| | | RGAVIRQNSDPTSEGPGPSPNPPAWVRPDNEAPPKVPQRTSSIATALNTSGAGGSRPAQ | |
| GCK: | 632 | ARGAVIRQNSDPTSEGPGPSPNPPAWVRPDNEAPPKVPQRTSSIATALNTSGAGGSRPAQ | 691 |
| NOV3c: | 662 | AVRASNPDLRRSDPGWERSDSVLPASHGHLPQAGSLERNRVGVSSKPDSSPVLSPGNKAK | 721 |
| | | AVRASNPDLRRSDPGWERSDSVLPASHGHLPQAGSLERNRVGVSSKPDSSPVLSPGNKAK | |
| GCK: | 692 | AVRASNPDLRRSDPGWERSDSVLPASHGHLPQAGSLERNRVGVSSKPDSSPVLSPGNKAK | 751 |
| NOV3c: | 722 | PDDHRSRPGRPASYKRAIGEDFVLLKERTLDEAPRPPKKAMDYXXXXXXXXXXXXXXXXX | 781 |
| | | PDDHRSRPGRPASYKRAIGEDFVLLKERTLDEAPRPPKKAMDY | |
| GCK: | 752 | PDDHRSRPGRPASYKRAIGEDFVLLKERTLDEAPRPPKKAMDYSSSSEEVESSEDDEEEG | 811 |
| NOV3c: | 782 | XXXXXXXXRDTPGGRSDGDTDSVSTMVVHDVEEITGTQPPYGGGTMVVQRTPEEERNLLH | 841 |
| | | RDTPGGRSDGDTDSVSTMVVHDVEEITGTQPPYGGGTMVVQRTPEEERNLLH | |
| GCK: | 812 | EGGPAEGSRDTPGGRSDGDTDSVSTMVVHDVEEITGTQPPYGGGTMVVQRTPEEERNLLH | 871 |
| NOV3c: | 842 | ADSNGYTNLPDVVQPSHSPTENSKGQSPPSKDGSGDYQSRGLVKAPGKSSFTMFVDLGIY | 901 |
| | | ADSNGYTNLPDVVQPSHSPTENSKGQSPPSKDGSGDYQSRGLVKAPGKSSFTMFVDLGIY | |
| GCK: | 872 | ADSNGYTNLPDVVQPSHSPTENSKGQSPPSKDGSGDYQSRGLVKAPGKSSFTMFVDLGIY | 931 |
| NOV3c: | 902 | QPGGSGDSIPITALVGGEGTRLDQLQYDVRKGSVVNVNPTNTRAHSETPEIRKYKKRFNS | 961 |
| | | QPGGSGDSIPITALVGGEGTRLDQLQYDVRKGSVVNVNPTNTRAHSETPEIRKYKKRFNS | |
| GCK: | 932 | QPGGSGDSIPITALVGGEGTRLDQLQYDVRKGSVVNVNPTNTRAHSETPEIRKYKKRFNS | 991 |
| NOV3c: | 962 | EILCAALWGVNLLVGTENGLMLLDRSGQGKVYGLIGRRRFQQMDVLEGLNLLITISGKRN | 1021 |
| | | EILCAALWGVNLLVGTENGLMLLDRSGQGKVYGLIGRRRFQQMDVLEGLNLLITISGKRN | |
| GCK: | 992 | EILCAALWGVNLLVGTENGLMLLDRSGQGKVYGLIGRRRFQQMDVLEGLNLLITISGKRN | 1051 |
| NOV3c: | 1022 | KLRVYYLSWLRNKILHNDPEVEKKQGWTTVGDMEGCGHYRVVKYERIKFLVIALKSSVEV | 1081 |
| | | KLRVYYLSWLRNKILHNDPEVEKKQGWTTVGDMEGCGHYRVVKYERIKFLVIALKSSVEV | |
| GCK: | 1052 | KLRVYYLSWLRNKILHNDPEVEKKQGWTTVGDMEGCGHYRVVKYERIKFLVIALKSSVEV | 1111 |
| NOV3c: | 1082 | YAWAPKPYHKFMAFKSFADLPHRPLLVDLTVEEGQRLKVIYGSSAGFHAVDVDSGNSYDI | 1141 |
| | | YAWAPKPYHKFMAFKSFADLPHRPLLVDLTVEEGQRLKVIYGSSAGFHAVDVDSGNSYDI | |
| GCK: | 1112 | YAWAPKPYHKFMAFKSFADLPHRPLLVDLTVEEGQRLKVIYGSSAGFHAVDVDSGNSYDI | 1171 |
| NOV3c: | 1142 | YIPVHIQSQITPHAIIFLPNTDGMEMLLCYEDEGVYVNTYGRIIKDVVLQWGEMPTSVAY | 1201 |
| | | YIPVHIQSQITPHAIIFLPNTDGMEMLLCYEDEGVYVNTYGRIIKDVVLQWGEMPTSVAY | |
| GCK: | 1172 | YIPVHIQSQITPHAIIFLPNTDGMEMLLCYEDEGVYVNTYGRIIKDVVLQWGEMPTSVAY | 1231 |
| NOV3c: | 1202 | ICSNQIMGWGEKAIEIRSVETGHLDGVFMHKRAQRLKFLCERNDKVFFASVRSGGSSQVY | 1261 |
| | | ICSNQIMGWGEKAIEIRSVETGHLDGVFMHKRAQRLKFLCERNDKVFFASVRSGGSSQVY | |
| GCK: | 1232 | ICSNQIMGWGEKAIEIRSVETGHLDGVFMHKRAQRLKFLCERNDKVFFASVRSGGSSQVY | 1291 |
| NOV3c: | 1262 | FMTLNRNCIMNW 1273 (SEQ ID No: 68) | |
| | | FMTLNRNCIMNW | |
| GCK: | 1292 | FMTLNRNCIMNW 1303 (SEQ ID NO: 35) | |

Based on its relatedness to known members of the STE20 family of protein kinases, NOV3b provides new diagnostic and therapeutic compositions useful in the treatment of disorders associated with alterations in the expression of members of the STE20 family of protein kinases. Nucleic acids, polypeptides, antibodies, and other compositions of the present invention are useful in the treatment and diagnosis of a variety of diseases and pathologies, including, by way of nonlimiting example, those involving metabolic and endocrine disorders, cancer, bone disorders, and tissue/cell growth regulation disorders.

NOV-3d

A NOV-3d sequence according to the invention is a nucleic acid sequence encoding a polypeptide related to STE20 family of protein kinases. A disclosed NOV-3d nucleic acid and its encoded polypeptide includes the sequences shown in Table 20. The disclosed nucleic acid (SEQ ID NO: 12) is 3735 nucleotides in length and contains an open reading frame (ORF) that begins with an ATG initiation codon at nucleotides 1-3 and ends with a TGA stop codon at nucleotides 3733-3735. The start and stop codons are shown in bold font. The disclosed, respective ORF encodes a 1244 amino acid polypeptide (SEQ ID NO: 13).

TABLE 20

| | |
|---|---|
| ATGGGCGACCCAGCCCCCGCCCGCAGCCTGGACGACATCGACCTGTCCGCCCTGCGGGACCCTGCTGGGATCTTTGAGCT | (SEQ ID NO: 12) |
| TGTGGAGGTGGTCGGCAATGGAACCTACGGACAGGTGTACAAGGGTCGGCATGTCAAGACGGGGCAGCTGGCTGCCATCA | |
| AGGTCATGGATGTCACGGAGGACGAGGAGGAAGAGATCAAACAGGAGATCAACATGCTGAAAAAGTACTCTCACCACCGC | |
| AACATCGCCACCTACTACGGAGCCTTCATCAAGAAGAGCCCCCCGGGAAACGATGACCAGCTCTGGCTGGTGATGGAGTT | |
| CTGTGGTGCTGGTTCAGTGACTGACCTGGTAAAGAACACAAAAGGCAACGCCCTGAAGGAGGACTGTATCGCCTATATCT | |
| GCAGGGAGATCCTCAGGGGTCTGGCCCATCTCCATGCCCACAAGGTGATCCATCGAGACATCAAGGGGCAGAATGTGCTG | |
| CTGACAGAGAATGCTGAGGTCAAGCTAGTGGATTTTGGGGTGAGTGCTCAGCTGGACCGCACCGTGGGCAGACGGAACAC | |
| TTTCATTGGGACTCCCTACTGGATGGCTCCAGAGGTCATCGCCTGTGATGAGAACCCTGATGCCACCTATGATTACAGGA | |
| GTGATATTTGGTCTCTAGGAATCACAGCCATCGAGATGGCAGAGGGAGCCCCCCCTCTGTGTGACATGCACCCCATGCGA | |
| GCCCTCTTCCTCATTCCTCGGAACCCTCCGCCCAGGCTCAAGTCCAAGAAGTGGTCTAAGAAGTTCATTGACTTCATTGA | |
| CACATGTCTCATCAAGACTTACCTGAGCCGCCCACCCACGGAGCAGCTACTGAAGTTTCCCTTCATCCGGGACCAGCCCA | |
| CGGAGCGGCAGGTCCGCATCCAGCTTAAGGACCACATTGACCGATCCCGGAAGAAGCGGGGTGAGAAAGAGGAGACAGAA | |
| TATGAGTACAGCGGCAGCGAGGAGGAAGATGACAGCCATGGAGAGGAAGGAGAGCCAAGCTCCATCATGAACGTGCCTGG | |
| AGAGTCGACTCTACGCCGGGAGTTTCTCCGGCTCCAGCAGGAAAATAAGAGCAACTCAGAGGCTTTAAAACAGCAGCAGC | |
| AGCTGCAGCAGCAGCAGCAGCGAGACCCCGAGGCACACATCAAACACCTGCTGCACCAGCGGCAGCGGCGCATAGAGGAG | |
| CAGAAGGAGGAGCGGCGCCGCGTGGAGGAGCAACAGCGGCGGGAGCGGGAGCAGCGGAAGCTGCAGGAGAAGGAGCAGCA | |
| GCGGCGGCTGGAGGACATGCAGGCTCTGCGGCGGGAGGAGGAGCGGCGGCAGGCGGAGCGCGAGCAGGAATATATTCGTC | |
| ACAGGCTAGAGGAGCAGCGGCAGTCAGAACGTCTCCAGAGGCAGCTGCAGCAGGAGCATGCCTACCTCAAGTCCCTGCAG | |
| CAGCAGCAACAGCAGCAGCAGCTTCAGAAACAGCAGCAGCAGCAGCTCCTGCCTGGGGACAGGAAGCCCCTGTACCATTA | |
| TGGTCGGGGCATGAATCCCGCTGACAAACCAGCCTGGGCCCGAGAGGTAGTGGCACACCGGGTCCCACTGAAGCCATATG | |
| CAGCACCTGTACCCCGATCCCAGTCCCTGCAGGACCAGCCCACCCGAAACCTGGCTGCCTTCCCAGCCTCCCATGACCCC | |
| GACCCTGCCATCCCCGCACCCACTGCCACGCCCAGTGCCCGAGGAGCTGTCATCCGCCAGAATTCAGACCCCACCTCTGA | |
| AGGACCTGGCCCCAGCCCGAATCCCCCAGCCTGGGTCCGCCCAGATAACGAGGCCCCACCCAAGGTGCCTCAGAGGACCT | |
| CATCTATCGCCACTGCCCTTAACACCAGTGGGGCCGGAGGGTCCCGGCCAGCCCAGGCAGTCCGTGCCAGTAACCCCGAC | |
| CTCAGGAGGAGCGACCCTGGCTGGGAACGCTCGGACAGCGTCCTTCCAGCCTCTCACGGGCACCTCCCCCAGGCTGGCTC | |
| ACTGGAGCGGAACCGCGTGGGAGTCTCCTCCAAACCGGACAGCTCCCCTGTGCTCTCCCCTGGGAATAAAGCCAAGCCCG | |
| ACGACCACCGCTCACGGCCAGGCCGGCCCGCAAGCTATAAGCGAGCAATTGGTGAGGACTTTGTGTTGCTGAAAGAGCGG | |
| ACTCTGGACGAGGCCCCTCGGCCTCCCAAGAAGGCCATGGACTACTCGTCGTCCAGCGAGGAGGTGGAAAGCAGTGAGGA | |
| CGACGAGGAGGAAGGCGAAGGCGGGCCAGCAGAGGGGAGCAGAGATACCCCTGGGGGCCGCAGCGATGGGGATACAGACA | |
| GCGTCAGCACCATGGTGGTCCACGACGTCGAGGAGATCACCGGGACCCAGCCCCCATACGGGGGCGGCACCATGGTGGTC | |
| CAGCGCACCCCTGAAGAGGAGCGGAACCTGCTGCATGCTGACAGCAATGGGTACACAAACCTGCCTGACGTGGTCCAGCC | |
| CAGCCACTCACCCACCGAGAACAGCAAAGGCCAAAGCCCACCCTCGAAGGATGGGAGTGGTGACTACCAGTCTCGTGGGC | |
| TGGTAAAGGCCCCTGGCAAGAGCTCGTTCACGATGTTTGTGGATCTAGGGATCTACCAGCCTGGAGGCAGTGGGGACAGC | |
| ATCCCCATCACAGCCCTAGTGGGTGGAGAGGGCACTCGGCTCGACCAGCTGCAGTACGACGTGAGGAAGGGTTCTGTGGT | |
| CAACGTGAATCCCACCAACACCCGGGCCCACAGTGAGACCCCTGAGATCCGGAAGTACAAGAAGCGATTCAACTCCGAGA | |
| TCCTCTGTGCAGCCCTTTGGGGGGTCAACCTGCTGGTGGGCACGGAGAACGGGCTGATGTTGCTGGACCGAAGTGGGCAG | |
| GGCAAGGTGTATGGACTCATTGGGCGGCGACGCTTCCAGCAGATGGATGTGCTGGAGGGGCTCAACCTGCTCATCACCAT | |
| CTCAGGGAAAAGGAACAAACTGCGGGTGTATTACCTGTCCTGGCTCCGGAACAAGATTCTGCACAATGACCCAGAAGTGG | |
| AGAAGAAGCAGGGCTGGACCACCGTGGGGGACATGGAGGGCTGCGGGCACTACCGTGTTGTGAAATACGAGCGGATTAAG | |
| TTCCTGGTCATCGCCCTCAAGAGCTCCGTGGAGGTGTATGCCTGGGCCCCCAAACCCTACCACAAATTCATGGCCTTCAA | |
| GTCCTTTGCCGACCTCCCCCACCGCCCTCTGCTGGTCGACCTGACAGTAGAGGAGGGGCAGCGGCTCAAGGTCATCTATG | |
| GCTCCAGTGCTGGCTTCCATGCTGTGGATGTCGACTCGGGGAACAGCTATGACATCTACATCCCTGTGCACATCCAGAGC | |
| CAGATCACGCCCCATGCCATCATCTTCCTCCCCAACACCGACGGCATGGAGATGCTGCTGTGCTACGAGGACGAGGGTGT | |
| CTACGTCAACACGTACGGGCGCATCATTAAGGATGTGGTGCTGCAGTGGGGGGAGATGCCTACTTCTGTGGCCTACATCT | |
| GCTCCAACCAGATAATGGGCTGGGGTGAGAAAGCCATTGAGATCCGCTCTGTGGAGACGGGCCACCTCGACGGGGTCTTC | |
| ATGCACAAACGAGCTCAGAGGCTCAAGTTCCTGTGTGAGCGGAATGACAAGGTGTTTTTTGCCTCAGTCCGCTCTGGGGG | |
| CAGCAGCCAAGTTTACTTCATGACTCTGAACCGTAACTGCATCATGAACTGGTGA | |
| MGDPAPARSLDDIDLSALRDPAGIFELVEVVGNGTYGQVYKGRHVKTGQLAAIKVMDVTEDEEEEIKQEINMLKKYSHHR | (SEQ ID NO: 13) |
| NIATYYGAFIKKSPPGNDDQLWLVMEFCGAGSVTDLVKNTKGNALKEDCIAYICREILRGLAHLHAHKVIHRDIKGQNVL | |
| LTENAEVKLVDFGVSAQLDRTVGRRNTFIGTPYWMAPEVIACDENPDATYDYRSDIWSLGITAIEMAEGAPPLCDMHPMR | |
| ALFLIPRNPPPRLKSKKWSKKFIDFIDTCLIKTYLSRPPTEQLLKFPFIRDQPTERQVRIQLKDHIDRSRKKRGEKEETE | |
| YEYSGSEEEDDSHGEEGEPSSIMNVPGESTLRREFLRLQQENKSNSEALKQQQQLQQQQQRDPEAHIKHLLHQRQRRIEE | |
| QKEERRRVEEQQRREREQRKLQEKEQQRRLEDMQALRREEERRQAEREQEYIRHRLEEQRQSERLQRQLQQEHAYLKSLQ | |
| QQQQQQQLQKQQQQQLLPGDRKPLYHYGRGMNPADKPAWAREVVAHRVPLKPYAAPVPRSQSLQDQPTRNLAAFPASHDP | |
| DPAIPAPTATPSARGAVIRQNSDPTSEGPGPSPNPPAWVRPDNEAPPKVPQRTSSIATALNTSGAGGSRPAQAVRASNPD | |
| LRRSDPGWERSDSVLPASHGHLPQAGSLERNRVGVSSKPDSSPVLSPGNKAKPDDHRSRPGRPASYKRAIGEDFVLLKER | |
| TLDEAPRPPKKAMDYSSSSEEVESSEDDEEEGEGGPAEGSRDTPGGRSDGDTDSVSTMVVHDVEEITGTQPPYGGGTMVV | |
| QRTPEEERNLLHADSNGYTNLPDVVQPSHSPTENSKGQSPPSKDGSGDYQSRGLVKAPGKSSFTMFVDLGIYQPGGSGDS | |
| IPITALVGGEGTRLDQLQYDVRKGSVVNVNPTNTRAHSETPEIRKYKKRFNSEILCAALWGVNLLVGTENGLMLLDRSGQ | |
| GKVYGLIGRRRFQQMDVLEGLNLLITISGKRNKLRVYYLSWLRNKILHNDPEVEKKQGWTTVGDMEGCGHYRVVKYERIK | |
| FLVIALKSSVEVYAWAPKPYHKFMAFKSFADLPHRPLLVDLTVEEGQRLKVIYGSSAGFHAVDVDSGNSYDIYIPVHIQS | |
| QITPHAIIFLPNTDGMEMLLCYEDEGVYVNTYGRIIKDVVLQWGEMPTSVAYICSNQIMGWGEKAIEIRSVETGHLDGVF | |
| MHKRAQRLKFLCERNDKVFFASVRSGGSSQVYFMTLNRNCIMNW | |

The disclosed NOV-3d nucleic acid sequence has homology (73% identity) to a mouse mRNA for a NIK protein (NIK) (GenBank Accession No: MMU88984), as shown in Table 21. NIK proteins are a subgroup of the STE20 family of protein kinases. As indicated by the “Expect” value, the probability of this alignment occurring by chance alone is 2.2e-295. Moreover, the disclosed, encoded amino acid sequence has 1046 of 1303 amino acid residues (80%) identical to a human NIK-related protein (GenBank Accession No: BAA90753), shown in Table 22. Furthermore, the disclosed, encoded amino acid sequence also has homology (80% identity) to a human GCK kinase (GenBank Accession No: BAA94838), another subgroup of the STE20 kinase family, as shown in Table 23. As indicated by the “Expect” value, the probability of these amino acid alignments occurring by chance alone are both 0.0, the lowest probability score.

TABLE 21
Score = 3832 (575.0 bits), Expect = 2.2e-295, Sum P(2) = 2.2e-295
Identities = 1260/1725 (73%), Positives = 1260/1725 (73%), Strand = Plus /
Plus

| | | | |
|---|---|---|---|
| NOV3d: | 4 | GGCGACCCAGCC-CCCGCCCGCAGCCTGGACGACATCGACCTGTCCGCCCTGCGGGACCC | 62 |
| | | GGCGA C A C CCCGC AGCCTGG GACAT GACCTGTC CCCTGCGGGACCC | |
| NIK: | 3 | GGCGAACGACTCTCCCGCGAAGAGCCTGGTGGACATTGACCTGTCGTCCCTGCGGGACCC | 62 |
| NOV3d: | 63 | TGCTGGGATCTTTGAGCTTGTGGAGGTGGTCGGCAATGGAACCTACGGACAGGTGTACAA | 122 |
| | | TGCTGGGAT TTTGAGCT GTGGA GTGGT GG AATGG ACCTA GGACA GT TA AA | |
| NIK: | 63 | TGCTGGGATTTTTGAGCTGGTGGAAGTGGTTGGAAATGGCACCTATGGACAAGTCTATAA | 122 |
| NOV3d: | 123 | GGGTCGGCATGTCAAGACGGGGCAGCTGGCTGCCATCAAGGTCATGGATGTCACGGAGGA | 182 |
| | | GGGTCG CATGT AA ACGG CA CTG C GCCATCAAGGT ATGGA GTCAC GAGGA | |
| NIK: | 123 | GGGTCGACATGTTAAAACGGT-CA-CTGCC-GCCATCAAGGTTATGGACGTCACCGAGGA | 179 |
| NOV3d: | 183 | CGAGGAGGAAGAGATCAAACAGGAGATCAACATGCTGAAAAAGTACTCTCACCACCGCAA | 242 |
| | | GA GAGGAAGA ATCA AC GGAGAT AA ATGCTGAA AAGTA TCTCA CA CG AA | |
| NIK: | 180 | TGAAGAGGAAGAAATCACACTGGAGATAAATATGCTGAAGAAGTATTCTCATCATCGAAA | 239 |
| NOV3d: | 243 | CATCGCCACCTACTACGGAGCCTTCATCAAGAAGAGCCCCCCGGGAAACGATGACCAGCT | 302 |
| | | AT GCCAC TACTA GG GC TTCAT AAGAAGAGCCC CC GGA A GATGACCA CT | |
| NIK: | 240 | TATTGCCACGTACTATGGTGCTTTCATTAAGAAGAGCCCTCCAGGACATGATGACCAACT | 299 |
| NOV3d: | 303 | CTGGCTGGTGATGGAGTTCTGTGGTGCTGGTTCAGTGACTGACCTGGTAAAGAACACAAA | 362 |
| | | CTGGCT GT ATGGAGTT TGTGG GCTGG TC T AC GACCT GT AAGAACAC AA | |
| NIK: | 300 | CTGGCTTGTTATGGAGTTTTGTGGGGCTGGGTCCATCACAGACCTTGTGAAGAACACCAA | 359 |
| NOV3d: | 363 | AGGCAACGCCCTGAAGGAGGACTGTATCGCCTATATCTGCAGGGAGATCCTCAGGGGTCT | 422 |
| | | AGG AAC C CT AA GA GACTG AT GC TA ATCT CAGGGA ATCCTCAGGGG T | |
| NIK: | 360 | AGGGAACACTCTCAAAGAAGACTGGATTGCTTACATCTCCAGGGAAATCCTCAGGGGATT | 419 |
| NOV3d: | 423 | GGCCCATCTCCATGCCCACAAGGTGATCCATCGAGACATCAAGGGGCAGAATGTGCTGCT | 482 |
| | | GGC CATCTCCAT CAC A GT AT CA CGAGA ATCAAGGG CA AATGTGCTGCT | |
| NIK: | 420 | GGCACATCTCCATATTCACCACGTTATTCACCGAGATATCAAGGGCCAAAATGTGCTGCT | 479 |
| NOV3d: | 483 | GACAGAGAATGCTGAGGTCAAGCTAGTGGATTTTGGGGTGAGTGCTCAGCTGGACCGCAC | 542 |
| | | GAC GAGAATGCTGAGGT AA CT GT GATTTTGG GT AG GCTCAGCTGGAC G AC | |
| NIK: | 480 | GACCGAGAATGCTGAGGTGAAACTTGTTGATTTTGGTGTAAGCGCTCAGCTGGACAGGAC | 539 |
| NOV3d: | 543 | CGTGGG-CAGACGGAACACTTTCATTGGGACTCCCTACTGGATGGCTCCAGAGGTCATCG | 601 |
| | | GT GG C GA G AA AC TTCAT GG AC CCCTACTGGATGGCTCCAGAGGTCATCG | |
| NIK: | 540 | GGTTGGACGGA-GAAATACGTTCATAGGCACACCCTACTGGATGGCTCCAGAGGTCATCG | 598 |
| NOV3d: | 602 | CCTGTGATGAGAACCCTGATGCCACCTATGATTACAGGAGTGATATTTGGTCTCTA-GGA | 660 |
| | | CCTGTGATGAGAACCC GA GCCAC TA GA TACAG AGTGA T TGGTC CT GG | |
| NIK: | 599 | CCTGTGATGAGAACCCAGACGCCACTTACGACTACAGAAGTGACCTCTGGTC-CTGTGGC | 657 |
| NOV3d: | 661 | ATCACAGCCATCGAGATGGCAGAGGGAGCCCCCCCTCTGTGTGACATGCACCCCATGCGA | 720 |
| | | ATCACAGCCATCGAGATGGC GA GG G CCCCCCTCT TGTGACATGCA CC ATG GA | |
| NIK: | 658 | ATCACAGCCATCGAGATGGCTGAAGGGGGCCCCCCTCTCTGTGACATGCATCCAATGAGA | 717 |
| NOV3d: | 721 | GCCCTCTTCCTCATTCCTCGGAACCCTCCGCCCAGGCTCAAGTCCAAGAAGTGGTCTAAG | 780 |
| | | GC CT TT CTCAT CC G AACCCTCC CCCAGGCT AAGTC AA AA TGGTC AAG | |
| NIK: | 718 | GCGCTGTTTCTCATCCCCAGAAACCCTCCTCCCAGGCTGAAGTCAAAAAAATGGTCAAAG | 777 |
| NOV3d: | 781 | AAGTTCATTGA-CTTCATTGACACATGTCTCATCAAGACTTACCTG-AGCCGCCCACCCA | 838 |
| | | AA TT TT A CTT AT GA TGTCT T AAGA TTAC TG AGC GCCC C A | |
| NIK: | 778 | AAATTT-TTCAGCTTTATAGAAGGCTGTCTGGTGAAGAATTACATGCAGCGGCCCTCT-A | 835 |
| NOV3d: | 839 | CGGAGCAGCTACTGAAGTTTCCCTTCATCCGGGACCAGCCCACGGAGCGGCAGGTCCGCA | 898 |
| | | C GAGCA CT T AA CC TTCAT GGGA CAGCCCA GA GGCAGGT CG A | |
| NIK: | 836 | CAGAGCAACTTTTAAAACACCCTTTCATAAGGGATCAGCCCAATGAAAGGCAGGTTCGAA | 895 |
| NOV3d: | 899 | TCCAGCTTAAGGACCACATTGACCGATCCCGGAAGAAGCGGGGTGAGAAAGAGGAGACAG | 958 |
| | | TCCAGCTTAAGGA CACAT GACCG CC G AAGAAG G GG GAGAAAGA GAGAC G | |
| NIK: | 896 | TCCAGCTTAAGGATCACATAGACCGGACCAGAAAGAAGAGAGGCGAGAAAGATGAGACGG | 955 |
| NOV3d: | 959 | AATATGAGTACAGCGGCAGCGAGGAGGAAGATGAC-A-GC-CATGGAG-AGGAAGGAGAG | 1014 |
| | | A TA GAGTACAGCGG AGCGAGGAGGA GA GA A G C TG AG AGGA GGAGAG | |
| NIK: | 956 | AGTACGAGTACAGCGGGAGCGAGGAGGAGGAGGAGGAAGTGCCTG-AGCAGGAGGGAGAG | 1014 |
| NOV3d: | 1015 | CCAAGCTCCATCATGAACGTGCCTGGAGAGTCGACTCTACGCCGGGAGTTTCTCCGGCTC | 1074 |
| | | CCAAG TCCATC T AA GTGCCTGGAGAGTC ACTCT CG CG GA TT CT G CT | |
| NIK: | 1015 | CCAAGTTCCATCGTCAATGTGCCTGGAGAGTCAACTCTGCGACGTGATTTCCTGAGACTG | 1074 |
| NOV3d: | 1075 | CAGCAGGAAAATAAG-AGCAACTCAGAGGCTTTAAAACAG-CAGCAGCAGCTGCAGCAGC | 1132 |
| | | CAGCAGGA AA AAG AGC TC GAGGCT T AG CAGCAGC CTGCAG AGC | |
| NIK: | 1075 | CAGCAGGAGAACAAGGAGCGG-TCTGAGGCTCTGCGG-AGACAGCAGCTTCTGCAGGAGC | 1132 |
| NOV3d: | 1133 | AGCAGCAGCGAGACCCCGAGGCACACATCAAACACCTGCTGCACCAGCGGCAGCGGCGCA | 1192 |
| | | AGCAGC CG GA C GAGG A A A CA CTGCTG AG GGCAG GCG A | |
| NIK: | 1133 | AGCAGCTCCGGGAGCAGGAGGAGTATAAGAGGCAGCTGCTGGCTGAGAGGCAGAAGCGGA | 1192 |
| NOV3d: | 1193 | TAGAGGAGCAGAAGGAGGAGCGGCGCCGCGTGGAGGAGCAACAGCGGCGGGAGCGGGA-G | 1251 |
| | | T GA AGCAGAA GA AG GG G CG TGGA GAGCAACA G G GA CGGGA G | |
| NIK: | 1193 | TTGAACAGCAGAAAGAACAGAGGAGGCGGCTGGAAGAGCAACAAAGAAGAGAACGGGAAG | 1252 |
| NOV3d: | 1252 | CAGCGGAAGCTGCAGGAGAAGGAGCAGCAGCGGCG-G--CTGGAGGACATGCAGGC-TCT | 1307 |
| | | C GGA GC GCAGGAG GAGCAGC GCGGCG G C GAGGA A G AGGC TCT | |
| NIK: | 1253 | CCA-GGAGGCAGCAGGAGCGTGAGCAGCGGCGGCGTGAACAAGAGGAGAAG-AGGCGTCT | 1310 |
| NOV3d: | 1308 | GCGGCGGGA--GGAGGAGCGGCGGCAGGCGGAGCGCGAGCAGGAATATATTCGTCACAGG | 1365 |
| | | CG GG A GGA GCGGCG A G GAG GAG AGGA A C A AGG | |
| NIK: | 1311 | -CGA-GGAACTGGAAAGGCGGCGTAAAGAAGAGGAAGAG-AGGAG-ACGGGCAGAAGAGG | 1366 |
| NOV3d: | 1366 | CTA-GAGGAGCAGCGGC-AGT---CAGAACGT-CTCCAGA-GGCAGCTGCAGCAGGAGCA | 1418 |
| | | A GAGGAG AG GG AG CAG A GT C CAG GGCAGCT AG AGGAGCA | |
| NIK: | 1367 | AGAAGAGGAG-AGTGGAGAGGGAACAGGA-GTACATCAGGCGGCAGCTAGAGGAGGAGCA | 1424 |
| NOV3d: | 1419 | T-GCCTACCTCAAGTCCCTGCAGCAGCAGCAACAGCAGCAGCAGCTTCA-GAAACAGCA- | 1475 |
| | | G C ACCT AG CCTGCAGCAGCAGC C CAG AGCAG CA G AC GCA | |
| NIK: | 1425 | GCGGC-ACCTGGAGATCCTGCAGCAGCAGCTGCTCCAGGAGCAGGC-CATGTTACTGCAC | 1482 |
| NOV3d: | 1476 | G-CAGCAGCAGCTCCTGCCTGGGGA-CAGGAAGCCCCTGTACCATTATGGTCGGGGCATG | 1533 |
| | | G C CAG AG CC GC G A CAG A GCC C G CC A G C G CA G | |
| NIK: | 1483 | GACCACAGGAGG-CC-GCACGCACAGCAGCA-GCCGCCGCCCCCGCA--G-CAG--CAGG | 1534 |
| NOV3d: | 1534 | AATCCCGCTGACAAACC-AGCCTGG--GCCCGAGAGGTAGTGGCACACCGGGTCCCA-CT | 1589 |
| | | A C G G CAAACC AGC T GC C AGAG G C CAC G CCC CT | |
| NIK: | 1535 | A--CAGGA-G-CAAACCGAGCTTTCATGCTCCAGAGCCCAAGCCTCACTATGACCCTGCT | 1590 |
| NOV3d: | 1590 | GA-AGCCATATGCAGC-ACC-TGTACCCCGATCCCAGTCCCTGCAGGACCAGCCCACCCG | 1646 |
| | | GA AG T G AG AC TG CCC T CA TC CT AG AC A C CCC | |
| NIK: | 1591 | GACAGAGCTCGGGAGGTACAGTGGTCCCACCTGGCA-TCTCTCAAGAACAATGTCTCCCC | 1649 |
| NOV3d: | 1647 | AAACCTG-GCTGCC-TTCC--CAGCCTCCCATGACCCCGACCCTGC-CATCCCCGCACCC | 1701 |
| | | C G G T CC TTCC CAG CCC T CCC A C GC CA C CC CC | |
| NIK: | 1650 | TGTCTCGAGATCCCATTCCTTCAGTGACCCTTCTCCCAAATTC-GCACACCACCATCTCC | 1708 |
| NOV3d: | 1702 | ACTGCCACG-CCCAGTGCCC 1720 (SEQ ID NO: 69) | |
| | | CT CA G CCCA TG CC | |
| NIK: | 1709 | GCTCTCAGGACCCA-TGTCC 1727 (SEQ ID NO: 36) | |

TABLE 22
Score = 1995 bits (5170), Expect = 0.0
Identities = 1046/1303 (80%), Positives = 1049/1303 (80%), Gaps = 67/1303
(5%)

| | | | |
|---|---|---|---|
| NOV3d: | 1 | MGDPAPARSLDDIDLSALRDPAGIFELVEVVGNGTYGQVYKGRHVKTGQLAAIKVMDVTX | 60 |
| | | MGDPAPARSLDDIDLSALRDPAGIFELVEVVGNGTYGQVYKGRHVKTGQLAAIKVMDVT | |
| NIK: | 1 | MGDPAPARSLDDIDLSALRDPAGIFELVEVVGNGTYGQVYKGRHVKTGQLAAIKVMDVTE | 60 |
| NOV3d: | 61 | XXXXXIKQEINMLKKYSHHRNIATYYGAFIKKSPPGNDDQLWLVMEFCGAGSVTDLVKNT | 120 |
| | | IKQEINMLKKYSHHRNIATYYGAFIKKSPPGNDDQLWLVMEFCGAGSVTDLVKNT | |
| NIK: | 61 | DEEEEIKQEINMLKKYSHHRNIATYYGAFIKKSPPGNDDQLWLVMEFCGAGSVTDLVKNT | 120 |
| NOV3d: | 121 | KGNALKEDCIAYICREILRGLAHLHAHKVIHRDIKGQNVLLTENAEVKLVDFGVSAQLDR | 180 |
| | | KGNALKEDCIAYICREILRGLAHLHAHKVIHRDIKGQNVLLTENAEVKLVDFGVSAQLDR | |
| NIK: | 121 | KGNALKEDCIAYICREILRGLAHLHAHKVIHRDIKGQNVLLTENAEVKLVDFGVSAQLDR | 180 |
| NOV3d: | 181 | TVGRRNTFIGTPYWMAPEVIACDENPDATYDYRSDIWSLGITAIEMAEGAPPLCDMHPMR | 240 |
| | | TVGRRNTFIGTPYWMAPEVIACDENPDATYDYRSDIWSLGITAIEMAEGAPPLCDMHPMR | |
| NIK: | 181 | TVGRRNTFIGTPYWMAPEVIACDENPDATYDYRSDIWSLGITAIEMAEGAPPLCDMHPMR | 240 |
| NOV3d: | 241 | ALFLIPRNPPPRLKSKKWSKKFIDFIDTCLIKTYLSRPPTEQLLKFPFIRDQPTERQVRI | 300 |
| | | ALFLIPRNPPPRLKSKKWSKKFIDFIDTCLIKTYLSRPPTEQLLKFPFIRDQPTERQVRI | |
| NIK: | 241 | ALFLIPRNPPPRLKSKKWSKKFIDFIDTCLIKTYLSRPPTEQLLKFPFIRDQPTERQVRI | 300 |
| NOV3d: | 301 | QLKDHIXXXXXXXXXXXXXXXXXXXXXXXXXXXXXXXXPSSIMNVPGESTLRREFLRLQQ | 360 |
| | | QLKDHI PSSIMNVPGESTLRREFLRLQQ | |
| NIK: | 301 | QLKDHIDRSRKKRGEKEETEYEYSGSEEEDDSHGEEGEPSSIMNVPGESTLRREFLRLQQ | 360 |
| NOV3d: | 361 | ENKSNSEALKXXXXXXXXXXRDPEAHIKHLLHXXXXXXXXXXXXXXXXXXXXXXXXXXXX | 420 |
| | | ENKSNSEALK RDPEAHIKHLLH | |
| NIK: | 361 | ENKSNSEALKQQQQLQQQQQRDPEAHIKHLLHQRQRRIEEQKEERRRVEEQQRREREQRK | 420 |
| NOV3d: | 421 | XXXXXXXXXXXDMQALXXXXXXXXXXXXXXYIRHXXXXXXXXXXXXXXXXXXHAYLKSXX | 480 |
| | | DMQAL Y R HAYLKS | |
| NIK: | 421 | LQEKEQQRRLEDMQALRREEERRQAEREQEYKRKQLEEQRQSERLQRQLQQEHAYLKSLQ | 480 |
| NOV3d: | 481 | XXXXXXXXXXXXXXXXXPGDRKPLYHYGRGMNPADKPAWAREVVAH-------------- | 526 |
| | | PGDRKPLYHYGRGMNPADKPAWAREV | |
| NIK: | 481 | QQQQQQQLQKQQQQQLLPGDRKPLYHYGRGMNPADKPAWAREVEERTRMNKQQNSPLAKS | 540 |
| NOV3d: | 527 | ----------------------------RVPLKPYAAP-----------------VPRSQ | 541 |
| | | +P++P P VPRSQ | |
| NIK: | 541 | KPGSTGPEPPIPQASPGPPGPLSQTPPMQRPVEPQEGPHKSLVAHRVPLKPYAAPVPRSQ | 600 |
| NOV3d: | 542 | SLQDQPTRNLAAFPASHXXXXXXXXXXXXXXXRGAVIRQNSDPTSEGPGPSPNPPAWVRP | 601 |
| | | SLQDQPTRNLAAFPASH RGAVIRQNSDPTSEGPGPSPNPPAWVRP | |
| NIK: | 601 | SLQDQPTRNLAAFPASHDPDPAIPAPTATPSARGAVIRQNSDPTSEGPGPSPNPPAWVRP | 660 |
| NOV3d: | 602 | DNEAPPKVPQRTSSIATALNTSGAGGSRPAQAVRASNPDLRRSDPGWERSDSVLPASHGH | 661 |
| | | DNEAPPKVPQRTSSIATALNTSGAGGSRPAQAVRASNPDLRRSDPGWERSDSVLPASHGH | |
| NIK: | 661 | DNEAPPKVPQRTSSIATALNTSGAGGSRPAQAVRASNPDLRRSDPGWERSDSVLPASHGH | 720 |
| Nov3d: | 662 | LPQAGSLERNRVGVSSKPDSSPVLSPGNKAKPDDHRSRPGRPASYKRAIGEDFVLLKERT | 721 |
| | | LPQAGSLERNRVGVSSKPDSSPVLSPGNKAKPDDHRSRPGRPA DFVLLKERT | |
| NIK: | 721 | LPQAGSLERNRVGVSSKPDSSPVLSPGNKAKPDDHRSRPGRPA--------DFVLLKERT | 772 |
| NOV3d: | 722 | LDEAPRPPKKAMDYXXXXXXXXXXXXXXXXXXXXXXXXXRDTPGGRSDGDTDSVSTMVVH | 781 |
| | | LDEAPRPPKKAMDY RDTPGGRSDGDTDSVSTMVVH | |
| NIK: | 773 | LDEAPRPPKKAMDYSSSSEEVESSEDDEEEGEGGPAEGSRDTPGGRSDGDTDSVSTMVVH | 832 |
| NOV3d: | 782 | DVEEITGTQPPYGGGTMVVQRTPEEERNLLHADSNGYTNLPDVVQPSHSPTENSKGQSPP | 841 |
| | | DVEEITGTQPPYGGGTMVVQRTPEEERNLLHADSNGYTNLPDVVQPSHSPTENSKGQSPP | |
| NIK: | 833 | DVEEITGTQPPYGGGTMVVQRTPEEEPNLLHADSNGYTNLPDVVQPSHSPTENSKGQSPP | 892 |
| NOV3d: | 842 | SKDGSGDYQSRGLVKAPGKSSFTMFVDLGIYQPGGSGDSIPITALVGGEGTRLDQLQYDV | 901 |
| | | SKDGSGDYQSRGLVKAPGKSSFTMFVDLGIYQPGGSGDSIPITALVGGEGTRLDQLQYDV | |
| NIK: | 893 | SKDGSGDYQSRGLVKAPGKSSFTMFVDLGIYQPGGSGDSIPITALVGGEGTRLDQLQYDV | 952 |
| NOV3d: | 902 | RKGSVVNVNPTNTRAHSETPEIRKYKKRFNSEILCAALWGVNLLVGTENGLMLLDRSGQG | 961 |
| | | RKGSVVNVNPTNTRAHSETPEIRKYKKRFNSEILCAALWGVNLLVGTENGLMLLDRSGQG | |
| NIK: | 953 | RKGSVVNVNPTNTRAHSETPEIRKYKKRFNSEILCAALWGVNLLVGTENGLMLLDRSGQG | 1012 |
| NOV3d: | 962 | KVYGLIGRRRFQQMDVLEGLNLLITISGKRNKLRVYYLSWLRNKILHNDPEVEKKQGWTT | 1021 |
| | | KVYGLIGRRRFQQMDVLEGLNLLITISGKRNKLRVYYLSWLRNKILHNDPEVEKKQGWTT | |
| NIK: | 1013 | KVYGLIGRRRFQQMDVLEGLNLLITISGKRNKLRVYYLSWLRNKILHNDPEVEKKQGWTT | 1072 |
| NOV3d: | 1022 | VGDMEGCGHYRVVKYERIKFLVIALKSSVEVYAWAPKPYHKFMAFKSFADLPHRPLLVDL | 1081 |
| | | VGDMEGCGHYRVVKYERIKFLVIALKSSVEVYAWAPKPYHKFMAFKSFADLPHRPLLVDL | |
| NIK: | 1073 | VGDMEGCGHYRVVKYERIKFLVIALKSSVEVYAWAPKPYHKFMAFKSFADLPHRPLLVDL | 1132 |
| NOV3d: | 1082 | TVEEGQRLKVIYGSSAGFHAVDVDSGNSYDIYIPVHIQSQITPHAIIFLPNTDGMEMLLC | 1141 |
| | | TVEEGQRLKVIYGSSAGFHAVDVDSGNSYDIYIPVHIQSQITPHAIIFLPNTDGMEMLLC | |
| NIK: | 1133 | TVEEGQRLKVIYGSSAGFHAVDVDSGNSYDIYIPVHIQSQITPHAIIFLPNTDGMEMLLC | 1192 |
| NOV3d: | 1142 | YEDEGVYVNTYGRIIKDVVLQWGEMPTSVAYICSNQIMGWGEKAIEIRSVETGHLDGVFM | 1201 |
| | | YEDEGVYVNTYGRIIKDVVLQWGEMPTSVAYICSNQIMGWGEKAIEIRSVETGHLDGVFM | |
| NIK: | 1193 | YEDEGVYVNTYGRIIKDVVLQWGEMPTSVAYICSNQIMGWGEKAIEIRSVETGHLDGVFM | 1252 |

| | | |
|---|---|---|
| NOV3d: | 1202 | HKRAQRLKFLCERNDKVFFASVRSGGSSQVYFMTLNRNCIMNW 1244 (SEQ ID NO: 70) |
| | | HKRAQRLKFLCERNDKVFFASVRSGGSSQVYFMTLNRNCIMNW |
| NIK: | 1253 | HKRAQRLKFLCERNDKVFFASVRSGGSSQVYFMTLNRNCIMNW 1295 (SEQ ID NO: 37) |

TABLE 23
Score = 2018 bits (5228), Expect = 0.0
Identities = 1054/1303 (80%), Positives = 1057/1303 (80%), Gaps = 59/1303
(4%)

| NOV3d: | 1 | MGDPAPARSLDDIDLSALRDPAGIFELVEVVGNGTYGQVYKGRHVKTGQLAAIKVMDVTX | 60 | |
|---|---|---|---|---|
| | | MGDPAPARSLDDIDLSALRDPAGIFELVEVVGNGTYGQVYKGRHVKTGQLAAIKVMDVT | | |
| GCK: | 1 | MGDPAPARSLDDIDLSALRDPAGIFELVEVVGNGTYGQVYKGRHVKTGQLAAIKVMDVTE | 60 | |
| NOV3d: | 61 | XXXXXIKQEINMLKKYSHHRNIATYYGAFIKKSPPGNDDQLWLVMEFCGAGSVTDLVKNT | 120 | |
| | | IKQEINMLKKYSHHRNIATYYGAFIKKSPPGNDDQLWLVMEFCGAGSVTDLVKNT | | |
| GCK: | 61 | DEEEEIKQEINMLKKYSHHRNIATYYGAFIKKSPPGNDDQLWLVMEFCGAGSVTDLVKNT | 120 | |
| NOV3d: | 121 | KGNALKEDCIAYICREILRGLAHLHAHKVIHRDIKGQNVLLTENAEVKLVDFGVSAQLDR | 180 | |
| | | KGNALKEDCIAYICREILRGLAHLHAHKVIHRDIKGQNVLLTENAEVKLVDFGVSAQLDR | | |
| GCK: | 121 | KGNALKEDCIAYICREILRGLAHLHAHKVIHRDIKGQNVLLTENAEVKLVDFGVSAQLDR | 180 | |
| NOV3d: | 181 | TVGRRNTFIGTPYWMAPEVIACDENPDATYDYRSDIWSLGITAIEMAEGAPPLCDMHPMR | 240 | |
| | | TVGRRNTFIGTPYWMAPEVIACDENPDATYDYRSDIWSLGITAIEMAEGAPPLCDMHPMR | | |
| GCK: | 181 | TVGRRNTFIGTPYWMAPEVIACDENPDATYDYRSDIWSLGITAIEMAEGAPPLCDMHPMR | 240 | |
| NOV3d: | 241 | ALFLIPRNPPPRLKSKKWSKKFIDFIDTCLIKTYLSRPPTEQLLKFPFIRDQPTERQVRI | 300 | |
| | | ALFLIPRNPPPRLKSKKWSKKFIDFIDTCLIKTYLSRPPTEQLLKFPFIRDQPTERQVRI | | |
| GCK: | 241 | ALFLIPRNPPPRLKSKKWSKKFIDFIDTCLIKTYLSRPPTEQLLKFPFIRDQPTERQVRI | 300 | |
| NOV3d: | 301 | QLKDHIXXXXXXXXXXXXXXXXXXXXXXXXXXXXXXXXPSSIMNVPGESTLRREFLRLQQ | 360 | |
| | | QLKDHI PSSIMNVPGESTLRREFLRLQQ | | |
| GCK: | 301 | QLKDHIDRSRKKRGEKEETEYEYSGSEEEDDSHGEEGEPSSIMNVPGESTLRREFLRLQQ | 360 | |
| NOV3d: | 361 | ENKSNSEALKXXXXXXXXXXRDPEAHIKHLLHXXXXXXXXXXXXXXXXXXXXXXXXXXXX | 420 | |
| | | ENKSNSEALK RDPEAHIKHLLH | | |
| GCK: | 361 | ENKSNSEALKQQQQLQQQQQRDPEAHIKHLLHQRQRRIEEQKEERRRVEEQQRREREQRK | 420 | |
| NOV3d: | 421 | XXXXXXXXXXXDMQALXXXXXXXXXXXXXXYIRHXXXXXXXXXXXXXXXXXXHAYLKSXX | 480 | |
| | | DMQAL Y R HAYLKS | | |
| GCK: | 421 | LQEKEQQRRLEDMQALRREEERRQAEREQEYKRKQLEEQRQSERLQRQLQQEHAYLKSLQ | 480 | |
| NOV3d: | 481 | XXXXXXXXXXXXXXXXXPGDRKPLYHYGRGMNPADKPAWAREVVAH-------------- | 526 | |
| | | PGDRKPLYHYGRGMNPADKPAWAREV | | |
| GCK: | 481 | QQQQQQQLQKQQQQQLLPGDRKPLYHYGRGMNPADKPAWAREVEERTRMNKQQNSPLAKS | 540 | |
| NOV3d: | 527 | ----------------------------RVPLKPYAAP-----------------VPRSQ | 541 | |
| | | +P++P P VPRSQ | | |
| GCK: | 541 | KPGSTGPEPPIPQASPGPPGPLSQTPPMQRPVEPQEGPHKSLVAHRVPLKPYAAPVPRSQ | 600 | |
| NOV3d: | 542 | SLQDQPTRNLAAFPASHXXXXXXXXXXXXXXXRGAVIRQNSDPTSEGPGPSPNPPAWVRP | 601 | |
| | | SLQDQPTRNLAAFPASH RGAVIRQNSDPTSEGPGPSPNPPAWVRP | | |
| GCK: | 601 | SLQDQPTRNLAAFPASHDPDPAIPAPTATPSARGAVIRQNSDPTSEGPGPSPNPPAWVRP | 660 | |
| NOV3d: | 602 | DNEAPPKVPQRTSSIATALNTSGAGGSRPAQAVRASNPDLRRSDPGWERSDSVLPASHGH | 661 | |
| | | DNEAPPKVPQRTSSIATALNTSGAGGSRPAQAVRASNPDLRRSDPGWERSDSVLPASHGH | | |
| GCK: | 661 | DNEAPPKVPQRTSSIATALNTSGAGGSRPAQAVRASNPDLRRSDPGWERSDSVLPASHGH | 720 | |
| NOV3d: | 662 | LPQAGSLERNRVGVSSKPDSSPVLSPGNKAKPDDHRSRPGRPASYKRAIGEDFVLLKERT | 721 | |
| | | LPQAGSLERNRVGVSSKPDSSPVLSPGNKAKPDDHRSRPGRPASYKRAIGEDFVLLKERT | | |
| GCK: | 721 | LPQAGSLERNRVGVSSKPDSSPVLSPGNKAKPDDHRSRPGRPASYKRAIGEDFVLLKERT | 780 | |
| NOV3d: | 722 | LDEAPRPPKKAMDYXXXXXXXXXXXXXXXXXXXXXXXXXRDTPGGRSDGDTDSVSTMVVH | 781 | |
| | | LDEAPRPPKKAMDY RDTPGGRSDGDTDSVSTMVVH | | |
| GCK: | 781 | LDEAPRPPKKAMDYSSSSEEVESSEDDEEEGEGGPAEGSRDTPGGRSDGDTDSVSTMVVH | 840 | |
| NOV3d: | 782 | DVEEITGTQPPYGGGTMVVQRTPEEERNLLHADSNGYTNLPDVVQPSHSPTENSKGQSPP | 841 | |
| | | DVEEITGTQPPYGGGTMVVQRTPEEERNLLHADSNGYTNLPDVVQPSHSPTENSKGQSPP | | |
| GCK: | 841 | DVEEITGTQPPYGGGTMVVQRTPEEERNLLHADSNGYTNLPDVVQPSHSPTENSKGQSPP | 900 | |
| NOV3d: | 842 | SKDGSGDYQSRGLVKAPGKSSFTMFVDLGIYQPGGSGDSIPITALVGGEGTRLDQLQYDV | 901 | |
| | | SKDGSGDYQSRGLVKAPGKSSFTMFVDLGIYQPGGSGDSIPITALVGGEGTRLDQLQYDV | | |
| GCK: | 901 | SKDGSGDYQSRGLVKAPGKSSFTMFVDLGIYQPGGSGDSIPITALVGGEGTRLDQLQYDV | 960 | |
| NOV3d: | 902 | RKGSVVNVNPTNTRAHSETPEIRKYKKRFNSEILCAALWGVNLLVGTENGLMLLDRSGQG | 961 | |
| | | RKGSVVNVNPTNTRAHSETPEIRKYKKRFNSEILCAALWGVNLLVGTENGLMLLDRSGQG | | |
| GCK: | 961 | RKGSVVNVNPTNTRAHSETPEIRKYKKRFNSEILCAALWGVNLLVGTENGLMLLDRSGQG | 1020 | |
| NOV3d: | 962 | KVYGLIGRRRFQQMDVLEGLNLLITISGKRNKLRVYYLSWLRNKILHNDPEVEKKQGWTT | 1021 | |
| | | KVYGLIGRRRFQQMDVLEGLNLLITISGKRNKLRVYYLSWLRNKILHNDPEVEKKQGWTT | | |
| GCK: | 1021 | KVYGLIGRRRFQQMDVLEGLNLLITISGKRNKLRVYYLSWLRNKILHNDPEVEKKQGWTT | 1080 | |
| NOV3d: | 1022 | VGDMEGCGHYRVVKYERIKFLVIALKSSVEVYAWAPKPYHKFMAFKSFADLPHRPLLVDL | 1081 | |
| | | VGDMEGCGHYRVVKYERIKFLVIALKSSVEVYAWAPKPYHKFMAFKSFADLPHRPLLVDL | | |
| GCK: | 1081 | VGDMEGCGHYRVVKYERIKFLVIALKSSVEVYAWAPKPYHKFMAFKSFADLPHRPLLVDL | 1140 | |
| NOV3d: | 1082 | TVEEGQRLKVIYGSSAGFHAVDVDSGNSYDIYIPVHIQSQITPHAIIFLPNTDGMEMLLC | 1141 | |
| | | TVEEGQRLKVIYGSSAGFHAVDVDSGNSYDIYIPVHIQSQITPHAIIFLPNTDGMEMLLC | | |
| GCK: | 1141 | TVEEGQRLKVIYGSSAGFHAVDVDSGNSYDIYIPVHIQSQITPHAIIFLPNTDGMEMLLC | 1200 | |
| NOV3d: | 1142 | YEDEGVYVNTYGRIIKDVVLQWGEMPTSVAYICSNQIMGWGEKAIEIRSVETGHLDGVFM | 1201 | |
| | | YEDEGVYVNTYGRIIKDVVLQWGEMPTSVAYICSNQIMGWGEKAIEIRSVETGHLDGVFM | | |
| GCK: | 1201 | YEDEGVYVNTYGRIIKDVVLQWGEMPTSVAYICSNQIMGWGEKAIEIRSVETGHLDGVFM | 1260 | |
| NOV3d: | 1202 | HKRAQRLKFLCERNDKVFFASVRSGGSSQVYFMTLNRNCIMNW | 1244 | (SEQ ID NO: 71) |
| | | HKPAQRLKFLCERNDKVFFASVRSGGSSQVYFMTLNRNCIMNW | | |
| GCK: | 1261 | HKRAQRLKFLCERNDKVFFASVRSGGSSQVYFMTLNRNCIMNW 1303 (SEQ ID NO: 38) | | |

Based on its relatedness to known members of the STE20 family of protein kinases, NOV3d provides new diagnostic and therapeutic compositions useful in the treatment of disorders associated with alterations in the expression of members of the STE20 family of protein kinases. Nucleic acids, polypeptides, antibodies, and other compositions of the present invention are useful in the treatment and diagnosis of a variety of diseases and pathologies, including, by way of nonlimiting example, those involving metabolic and endocrine disorders, cancer, bone disorders, and tissue/cell growth regulation disorders.

Table 24 shows a multiple sequence alignment of the disclosed NOV-3 polypeptides with a STE20 protein (GenBank Accession No: BAA90753), indicating the homology between the present invention and a known member of the protein family.

TABLE 24

| | |
|---|---|
| STE20 | MGDPAPARSLDDIDLSALRDPAGIFELVEVVGNGTYGQVYKGRHVKTGQLAAIKVMDVTE |
| NOV3b | MGDPAPARSLDDIDLSALRDPAGIFELVEVVGNGTYGQVYKGRHVKTGQLAAIKVMDVTE |
| NOV3a | MGDPAPARSLDDIDLSALRDPAGIFELVEVVGNGTYGQVYKGRHVKTGQLAAIKVMDVTE |
| NOV3d | MGDPAPARSLDDIDLSALRDPAGIFELVEVVGNGTYGQVYKGRHVKTGQLAAIKVMDVTE |
| NOV3c | MGDPAPARSLDDIDLSALRDPAGIFELVEVVGNGTYGQVYKGRHVKTGQLAAIKVMDVTE |
| | ************************************************************ |
| STE20 | DEEEEIKQEINMLKKYSHHRNIATYYGAFIKKSPPGNDDQLWLVMEFCGAGSVTDLVKNT |
| NOV3b | DEEEEIKQEINMLKKYSHHRNIATYYGAFIKKSPPGNDDQLWLVMEFCGAGSVTDLVKNT |
| NOV3a | DEEEEIKQEINMLKKYSHHRNIATYYGAFIKKSPPGNDDQLWLVMEFCGAGSVTDLVKNT |
| NOV3d | DEEEEIKQEINMLKKYSHHRNIATYYGAFIKKSPPGNDDQLWLVMEFCGAGSVTDLVKNT |
| NOV3c | DEEEEIKQEINMLKKYSHHRNIATYYGAFIKKSPPGNDDQLWLVMEFCGAGSVTDLVKNT |
| | ************************************************************* |
| STE20 | KGNALKEDCIAYICREILRGLAHLHAHKVIHRDIKGQNVLLTENAEVKLVDFGVSAQLDR |
| NOV3b | KGNALKEDCIAYICREILRGLAHLHAHKVIHRDIKGQNVLLTENAEVKLVDFGVSAQLDR |
| NOV3a | KGNALKEDCIAYICREILRGLAHLHAHKVIHRDIKGQNVLLTENAEVKLVDFGVSAQLDR |
| NOV3d | KGNALKEDCIAYICREILRGLAHLHAHKVIHRDIKGQNVLLTENAEVKLVDFGVSAQLDR |
| NOV3c | KGNALKEDCIAYICREILRGLAHLHAHKVIHRDIKGQNVLLTENAEVKLVDFGVSAQLDR |
| | ************************************************************ |
| STE20 | TVGRRNTFIGTPYWMAPEVIACDENPDATYDYRSDIWSLGITAIEMAEGAPPLCDMHPMR |
| NOV3b | TVGRRNTFIGTPYWMAPEVIACDENPDATYDYRSDIWSLGITAIEMAEGAPPLCDMHPMR |
| NOV3a | TVGRRNTFIGTPYWMAPEVIACDENPDATYDYRSDIWSLGITAIEMAEGAPPLCDMHPMR |
| NOV3d | TVGRRNTFIGTPYWMAPEVIACDENPDATYDYRSDIWSLGITAIEMAEGAPPLCDMHPMR |
| NOV3c | TVGRRNTFIGTPYWMAPEVIACDENPDATYDYRSDIWSLGITAIEMAEGAPPLCDMHPMR |
| | ************************************************************ |
| STE20 | ALFLIPRNPPPRLKSKKWSKKFIDFIDTCLIKTYLSRPPTEQLLKFPFIRDQPTERQVRI |
| NOV3b | ALFLIPRNPPPRLKSKKWSKKFIDFIDTCLIKTYLSRPPTEQLLKFPFIRDQPTERQVRI |
| NOV3a | ALFLIPRNPPPRLKSKKWSKKFIDFIDTCLIKTYLSRPPTEQLLKFPFIRDQPTERQVRI |
| NOV3d | ALFLIPRNPPPRLKSKKWSKKFIDFIDTCLIKTYLSRPPTEQLLKFPFIRDQPTERQVRI |
| NOV3c | ALFLIPRNPPPRLKSKKWSKKFIDFIDTCLIKTYLSRPPTEQLLKFPFIRDQPTERQVRI |
| | ************************************************************ |
| STE20 | QLKDHIDRSRKKRGEKEETEYEYSGSEEEDDSHGEEGEPSSIMNVPGESTLRREFLRLQQ |
| NOV3b | QLKDHIDRSRKKRGEKEETEYEYSGSEEEDDSHGEEGEPSSIMNVPGESTLRREFLRLQQ |
| NOV3a | QLKDHIDRSRKKRGEKEETEYEYSGSEEEDDSHGEEGEPSSIMNVPGESTLRREFLRLQQ |
| NOV3d | QLKDHIDRSRKKRGEKEETEYEYSGSEEEDDSHGEEGEPSSIMNVPGESTLRREFLRLQQ |
| NOV3c | QLKDHIDRSRKKRGEKEETEYEYSGSEEEDDSHGEEGEPSSIMNVPGESTLRREFLRLQQ |
| | ************************************************************* |
| STE20 | ENKSNSEALKQQQQLQQQQQRDPEAHIKHLLHQRQRRIEEQKEERRRVEEQQRREREQRK |
| NOV3b | ENKSNSEALKQQQQLQQQQQRDPEAHIKHLLHQRQRRIEEQKEERRRVEEQQRREREQRK |
| NOV3a | ENKSNSEALKQQQQLQQQQQRDPEAHIKHLLHQRQRRIEEQKEERRRVEEQQRREREQRK |
| NOV3d | ENKSNSEALKQQQQLQQQQQRDPEAHIKHLLHQRQRRIEEQKEERRRVEEQQRREREQRK |
| NOV3c | ENKSNSEALKQQQQLQQQQQRDPEAHIKHLLHQRQRRIEEQKEERRRVEEQQRREREQRK |
| | ************************************************************ |
| STE20 | LQEKEQQRRLEDMQALRREEERRQAEREQEYKRKQLEE---------------------- |
| NOV3b | LQEKEQQRRLEDMQALRREEERRQAEREQEYIRHRLEE---------------------- |
| NOV3a | LQEKEQQRRLEDMQALRREEERRQAEREQEYIRHRLEEEQRQLEILQQQLLQEQALLLEY |
| NOV3d | LQEKEQQRRLEDMQALRREEERRQAEREQEYIRHRLEE---------------------- |
| NOV3c | LQEKEQQRRLEDMQALRREEERRQAEREQEYIRHRLEEEQRQLEILQQQLLQEQALLLEY |
| | ******************************* *::*** |
| STE20 | -------QRQSERLQRQLQQEHAYLKSLQQQQQQQQLQKQQQQQLLPGDRKPLYHYGRGM |
| NOV3b | -------QRQSERLQRQLQQEHAYLKSLQQQQQQQQLQKQQQQQLLPGDRKPLYHYGRGM |
| NOV3a | KRKQLEEQRQSERLQRQLQQEHAYLKSLQQQQQQQQLQKQQQQQLLPGDRKPLYHYGRGM |
| NOV3d | -------QRQSERLQRQLQQEHAYLKSLQQQQQQQQLQKQQQQQLLPGDRKPLYHYGRGM |
| NOV3c | KRKQLEEQRQSERLQRQLQQEHAYLKSLQQQQQQQQLQKQQQQQLLPGDRKPLYHYGRGM |
| | ***************************************************** |
| STE20 | NPADKPAWAREVEERTRMNKQQNSPLAKSKPGSTGPEPPIPQASPGPPGPLSQTPPMQRP |
| NOV3b | NPADKPAWAREVEERTRMNKQQNSPLAKSKPGSTGPEPPIPQASPGPPGPLSQTPPMQRP |
| NOV3a | NPADKPAWAREVEERTRMNKQQNSPLAKSKPGSTGPEPPIPQASPGPPGPLSQTPPMQRP |
| NOV3d | NPADKPAWAREV------------------------------------------------ |
| NOV3c | NPADKPAWAREV------------------------------------------------ |
| | ************ |
| STE20 | VEPQEGPHKSLVAHRVPLKPYAAPVPRSQSLQDQPTRNLAAFPASHDPDPAIPAPTATPS |
| NOV3b | VEPQEGPHKSLVAHRVPLKPYAAPVPRSQSLQDQPTRNLAAFPASHDPDPAIPAPTATPS |
| NOV3a | VEPQEGPHKSLVAHRVPLKPYAAPVPRSQSLQDQPTRNLAAFPASHDPDPAIPAPTATPS |
| NOV3d | -----------VAHRVPLKPYAAPVPRSQSLQDQPTRNLAAFPASHDPDPAIPAPTATPS |
| NOV3c | -----------VAHRVPLKPYAAPVPRSQSLQDQPTRNLAAFPASHDPDPAIPAPTATPS |
| | ************************************************* |
| STE20 | ARGAVIRQNSDPTSEGPGPSPNPPAWVRPDNEAPPKVPQRTSSIATALNTSGAGGSRPAQ |
| NOV3b | ARGAVIRQNSDPTSEGPGPSPNPPANVRPDNEAPPKVPQRTSSIATALNTSGAGGSRPAQ |
| NOV3a | ARGAVIRQNSDPTSEGPGPSPNPPAWVRPDNEAPPKVPQRTSSIATALNTSGAGGSRPAQ |
| NOV3d | ARGAVIRQNSDPTSEGPGPSPNPPAWVRPDNEAPPKVPQRTSSIATALNTSGAGGSRPAQ |
| NOV3c | ARGAVIRQNSDPTSEGPGPSPNPPAWVRPDNEAPPKVPQRTSSIATALNTSGAGGSRPAQ |
| | ************************************************************ |
| STE20 | AVRASNPDLRRSDPGWERSDSVLPASHGHLPQAGSLERNRVGVSSKPDSSPVLSPGNKAK |
| NOV3b | AVRASNPDLRRSDPGWERSDSVLPASHGHLPQAGSLERNRVGVSSKPDSSPVLSPGNKAK |
| NOV3a | AVRASNPDLRRSDPGWERSDSVLPASHGHLPQAGSLERNRVGVSSKPDSSPVLSPGNKAK |
| NOV3d | AVRASNPDLRRSDPGWERSDSVLPASHGHLPQAGSLERNRVGVSSKPDSSPVLSPGNKAK |
| NOV3c | AVRASNPDLRRSDPGWERSDSVLPASHGHLPQAGSLERNRVGVSSKPDSSPVLSPGNKAK |
| | ************************************************************ |
| STE20 | PDDHRSRPGRPA--------DFVLLKERTLDEAPRPPKKAMDYSSSSEEVESSEDDEEEG |
| NOV3b | PDDHRSRPGRPASYKRAIGEDFVLLKERTLDEAPRPPKKAMDYSSSSEEVESSEDDEEEG |
| NOV3a | PDDHRSRPGRPASYKRAIGEDFVLLKERTLDEAPRPPKKAMDYSSSSEEVESSEDDEEEG |
| NOV3d | PDDHRSRPGRPASYKRAIGEDFVLLKERTLDEAPRPPKKAMDYSSSSEEVESSEDDEEEG |
| NOV3c | PDDHRSRPGRPASYKRAIGEDFVLLKERTLDEAPRPPKKAMDYSSSSEEVESSEDDEEEG |
| | ************ **************************************** |
| STE20 | EGGPAEGSRDTPGGRSDGDTDSVSTMVVHDVEEITGTQPPYGGGTMVVQRTPEEERNLLH |
| NOV3b | EGGPAEGSRDTPGGRSDGDTDSVSTMVVHDVEEITGTQPPYGGGTMVVQRTPEEERNLLH |
| NOV3a | EGGPAEGSRDTPGGRSDGDTDSVSTMVVHDVEEITGTQPPYGGGTMVVQRTPEEERNLLH |
| NOV3d | EGGPAEGSRDTPGGRSDGDTDSVSTMVVHDVEEITGTQPPYGGGTMVVQRTPEEERNLLH |
| NOV3c | EGGPAEGSRDTPGGRSDGDTDSVSTMVVHDVEEITGTQPPYGGGTMVVQRTPEEERNLLH |
| | ************************************************************ |
| STE20 | ADSNGYTNLPDVVQPSHSPTENSKGQSPPSKDGSGDYQSRGLVKAPGKSSFTMFVDLGIY |
| NOV3b | ADSNGYTNLPDVVQPSHSPTENSKGQSPPSKDGSGDYQSRGLVKAPGKSSFTMFVDLGIY |
| NOV3a | ADSNGYTNLPDVVQPSHSPTENSKGQSPPSKDGSGDYQSRGLVKAPGKSSFTMFVDLGIY |
| NOV3d | ADSNGYTNLPDVVQPSHSPTENSKGQSPPSKDGSGDYQSRGLVKAPGKSSFTMFVDLGIY |
| NOV3c | ADSNGYTNLPDVVQPSHSPTENSKGQSPPSKDGSGDYQSRGLVKAPGKSSFTMFVDLGIY |
| | ************************************************************ |
| STE20 | QPGGSGDSIPITALVGGEGTRLDQLQYDVRKGSVVNVNPTNTRAHSETPEIRKYKKRFNS |
| NOV3b | QPGGSGDSIPITALVGGEGTRLDQLQYDVRKGSVVNVNPTNTRAHSETPEIRKYKKRFNS |
| NOV3a | QPGGSGDSIPITALVGGEGTRLDQLQYDVRKGSVVNVNPTNTRAHSETPEIRKYKKRFNS |
| NOV3d | QPGGSGDSIPITALVGGEGTRLDQLQYDVRKGSVVNVNPTNTRAHSETPEIRKYKKRFNS |
| NOV3c | QPGGSGDSIPITALVGGEGTRLDQLQYDVRKGSVVNVNPTNTRAHSETPEIRKYKKRFNS |
| | ************************************************************ |
| STE20 | EILCAALWGVNLLVGTENGLMLLDRSGQGKVYGLIGRRRFQQMDVLEGLNLLITISGKRN |
| NOV3b | EILCAALWGVNLLVGTENGLMLLDRSGQGKVYGLIGRRRFQQMDVLEGLNLLITISGKRN |
| NOV3a | EILCAALWGVNLLVGTENGLMLLDRSGQGKVYGLIGRRRFQQMDVLEGLNLLITISGKRN |
| NOV3d | EILCAALWGVNLLVGTENGLMLLDRSGQGKVYGLIGRRRFQQMDVLEGLNLLITISGKRN |
| NOV3c | EILCAALWGVNLLVGTENGLMLLDRSGQGKVYGLIGRRRFQQMDVLEGLNLLITISGKRN |
| | ************************************************************ |
| STE20 | KLRVYYLSWLRNKILHNDPEVEKKQGWTTVGDMEGCGHYRVVKYERIKFLVIALKSSVEV |
| NOV3b | KLRVYYLSWLRNKILHNDPEVEKKQGWTTVGDMEGCGHYRVVKYERIKFLVIALKSSVEV |
| NOV3a | KLRVYYLSWLRNKILHNDPEVEKKQGWTTVGDMEGCGHYRVVKYERIFKLVIALKSSVEV |
| NOV3d | KLRVYYLSWLRNKILHNDPEVEKKQGWTTVGDMEGCGHYRVVKYERIKFLVIALKSSVEV |
| NOV3c | KLRVYYLSWLRNKILHNDPEVEKKQGWTTVGDMEGCGHYRVVKYERIKFLVIALKSSVEV |
| | ************************************************************ |
| STE20 | YAWAPKPYHKFMAFKSFADLPHRPLLVDLTVEEGQRLKVIYGSSAGFHAVDVDSGNSYDI |
| NOV3b | YAWAPKPYHKFMAFKSFADLPHRPLLVDLTVEEGQRLKVIYGSSAGFHAVDVDSGNSYDI |
| NOV3a | YAWAPKPYHKFMAFKSFADLPHRPLLVDLTVEEGQRLKVIYGSSAGFHAVDVDSGNSYDI |
| NOV3d | YAWAPKPYHKFMAFKSFADLPHRPLLVDLTVEEGQRLKVIYGSSAGFHAVDVDSGNSYDI |
| NOV3c | YAWAPKPYHKFMAFKSFADLPHRPLLVDLTVEEGQRLKVIYGSSAGFHAVDVDSGNSYDI |
| | ************************************************************ |
| STE20 | YIPVHIQSQITPHAIIFLPNTDGMEMLLCYEDEGVYVNTYGRIIKDVVLQWGEMPTSVAY |
| NOV3b | YIPVHIQSQITPHAIIFLPNTDGMEMLLCYEDEGVYVNTYGRIIKDVVLQWGEMPTSVAY |
| NOV3a | YIPVHIQSQITPHAIIFLPNTDGMEMLLCYEDEGVYVNTYGRIIKDVVLQWGEMPTSVAY |
| NOV3d | YIPVHIQSQITPHAIIFLPNTDGMEMLLCYEDEGVYVNTYGRIIKDVVLQWGEMPTSVAY |
| NOV3c | YIPVHIQSQITPHAIIFLPNTDGMEMLLCYEDEGVYVNTYGRIIKDVVLQWGEMPTSVAY |
| | ************************************************************ |
| STE20 | ICSNQIMGWGEKAIEIRSVETGHLDGVFMHKRAQRLKFLCERNDKVFFASVRSGGSSQVY |
| NOV3b | ICSNQIMGWGEKAIEIRSVETGHLDGVFMHKRAQRLKFLCERNDKVFFASVRSGGSSQVY |
| NOV3a | ICSNQIMGWGEKAIEIRSVETGHLDGVFMHKRAQRLKFLCERNDKVFFASVRSGGSSQVY |
| NOV3d | ICSNQIMGWGEKAIEIRSVETGHLDGVFMHKRAQRLKFLCERNDKVFFASVRSGGSSQVY |
| NOV3c | ICSNQIMGWGEKAIEIRSVETGHLDGVFMHKRAQRLKFLCERNDKVFFASVRSGGSSQVY |
| | ************************************************************ |
| STE20 | FMTLNRNCIMNW (SEQ ID NO: 39) |
| NOV3b | FMTLNRNCIMNW (SEQ ID NO: 9) |
| NOV3a | FMTLNRNCIMNW (SEQ ID NO: 7) |
| NOV3d | FMTLNRNCIMNW (SEQ ID NO: 13) |
| NOV3c | FMTLNRNCIMNW (SEQ ID NO: 11) |
| | ************ |

Consensus key
* - single, fully conserved residue
: - conservation of strong groups
. - conservation of weak groups
- no consensus

Based on the relatedness between NOV-3 and STE20 kinases, the disclosed NOV3 proteins are novel members of the STE20 protein kinase family. Therefore, the nucleic acids and proteins of the inventions are useful in potential therapeutic applications implicated in various pathologies and disorders described and other pathologies and disorders related to aberrant function or aberrant expression of these STE20-protein kinases.

Potential therapeutic uses for the nucleic acids and proteins of the invention include, by way of nonlimiting example, protein therapeutic, small molecule drug target, antibody target (including therapeutic, diagnostic, or drug targeting/cytotoxic antibodies), diagnostic and/or prognostic marker, gene therapy (gene delivery/gene ablation), research tools, and tissue regeneration in vitro and in vivo (regeneration for all these tissues and cell types composing these tissues and cell types derived from these tissues).

The nucleic acids and proteins of the invention are useful in potential therapeutic applications implicated in various names of pathologies/disorders described above, as well as other pathologies or disorders. For example, a cDNA encoding the STE20 protein kinase-like protein may be useful in gene therapy, and the STE20 protein kinase-like protein may be useful when administered to a subject in need thereof. By way of nonlimiting example, the compositions of the present invention will have efficacy for treatment of patients suffering from the pathologies described above. The novel nucleic acids encoding the STE20 protein kinase-like proteins, and the STE20 protein kinase-like proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV-4: A Novel Trypsin Inhibitor-Like Protein

The NOV-4 sequences (NOV-4a, NOV-4b, NOV-4c, NOV-4d, and NOV-4e) according to the invention are nucleotide sequences encoding respective polypeptides related to trypsin inhibitor proteins.

The disclosed NOV-4 sequences are splice variants. Splice variants occur naturally. When a variant and the original sequence have the same or opposite activity, they may differ in various properties not directly connected to biological activity. A certain variant may be expressed mainly in one tissue, while the original sequence from which it has been varied, or another variant derived from the same sequence, may be expressed mainly in another tissue. The presence or level of specific splice variants may be the cause, and/or indicative of, a disease, disorder, pathological or normal condition.

Because a drug may be effective against one variant but not another, or may cause side effects because it targets all splice variants, an effective drug needs to target the particular splice variant. Because soluble variants with therapeutic or disease-related functions may be naturally occurring in specific tissues, they may be optimal candidates for drug targets or protein therapeutics. Variants may have no activity at all and may serve as dominant negative natural inhibitors. Thus, splice variants useful in generating new drug targets, protein therapeutics and markers for diagnostics.

NOV-4 sequences according to the invention encode polypeptides related to trypsin inhibitor proteins that are expressed in brain tumors, polypeptides related to sperm coat glycoproteins, and polypeptides related to glioma pathogenesis related proteins. See Yamakawa et al., 1998, Biochim Biophys Acta 1395(2):202-8; Murphy et al., 1995, Gene 159(1): 131-5. In addition, similarities were found between NOV-4 and insect allergens in wasps, hornets, fire ants, and secreted/membrane proteins in nematode pathogens. See J Allergy Clin Immunol 1990, 85(6):988-96. Therefore, the nucleic acids and proteins of the NOV-4 splice variants described in this invention can have similar functions as these proteins.

NOV-4 proteins are expressed in the following tissues: pituitary gland, mammary gland, adrenal gland, thalamus, and fetal lung.

Functional roles attributed to trypsin inhibitor proteins include sperm coat maturation, immunological responses, glioma pathogenesis, and signal transduction pathways. Thus, NOV-4 nucleic acids and polypeptides, antibodies and related compounds according to the invention will be useful in therapeutic and diagnostic applications in disorders associated with, e.g., reproductive disorders, immunological disorders, cancer, and metabolic disorders. Additional utilities for NOV-4 nucleic acids and polypeptides according to the invention are disclosed herein.

NOV-4a

A NOV-4a sequence according to the invention is a nucleic acid sequence that encodes a polypeptide related to trypsin inhibitor proteins. A disclosed NOV-4a nucleic acid and its encoded polypeptide is included in Table 25. The disclosed nucleic acid (SEQ ID NO: 14) is 2305 nucleotides in length and contains an open reading frame (ORF) that begins with an ATG initiation codon at nucleotide 453, and ends with a TGA stop codon at nucleotide 1602. A disclosed, representative ORF encodes a 383 amino acid polypeptide (SEQ ID NO: 15). NOV-4a is missing one exon in the 5'nucleotide region compared to other splice variants (NOV-4b and NOV-4c), resulting in an alternative methionine start codon and a Kozak sequence.

TABLE 25

| | |
|---|---|
| CTCTGACTGCTCCTATTGAGCTGTCTGCTCGCTGTGCCCGCTGTGCCTGCTGTGCCCGCG | (SEQ ID NO: 14) |
| CTGTCGCCGCTGCTACCGCGTCTACTGGACGCGGGAGACGCCAGCGAGCTGGTGATTGGA | |
| GCCCTGCGGAGAGCTCAAGCGCCCAGCTCTGCCCGAGGAGCCCAGGCTGCCCCGTGAGTC | |
| CCATAGTTGCTACAGGAGTGGAGCCATGAGCTGCGTCCTGGGTGGTGTCATCCCCTTGGG | |
| GCTGCTGTTCCTGGTCCGCGGATCCCAAGGCTACCTCCTGCCCAACGTCACTCTCTTAGA | |
| GGAGCTGCTCAGCAAATACCAGCACAACGAGTCTCACTCCCGGGTCCGCAGAGCCATCCC | |
| CAGGGAGGACAAGGAGGAGATCCTCATGCTGCACAACAAGCTTCGGGGCCAGGTGCAGCC | |
| TCAGGCCTCCAACATGGAGTACATGACCTGGGATGACGAACTGGGGCAGGTATCGCTCTC | |
| CGGGGTTCCATGTGCAGTCCTGGTATGACGAGGTGAAGGACTACACCTACCCCTACCCGA | |
| GCGAGTGCAACCCCTGGTGTCCAGAGAGGTGCTCGGGGCCTATGTGCACGCACTACACAC | |
| AGATAGTTTGGGCCACCACCAACAAGATCGGTTGTGCTGTGAACACCTGCCGGAAGATGA | |
| CTGTCTGGGGAGAAGTTTGGGAGAACGCGGTCTACTTTGTCTGCAATTATTCTCCAAAGG | |
| GGAACTGGATTGGAGAAGCCCCCTACAAGAATGGCCGGCCCTGCTCTGAGTGCCCACCCA | |
| GCTATGGAGGCAGCTGCAGGAACAACTTGTGTTACCGAGAAGAAACCTACACTCCAAAAC | |
| CTGAAACGGACGAGATGAATGAGGTGGAAACGGCTCCCATTCCTGAAGAAAACCATGTTT | |
| GGCTCCAACCGAGGGTGATGAGACCCACCAAGCCCAAGAAAACCTCTGCGGTCAACTACA | |
| TGACCCAAGTCGTCAGATGTGACACCAAGATGAAGGACAGGTGCAAAGGGTCCACGTGTA | |
| ACAGGTACCAGTGCCCAGCAGGCTGCCTGAACCACAAGGCGAAGATCTTTGGAACTCTGT | |
| TCTATGAAAGCTCGTCTAGCATATGCCGCGCCGCCATCCACTACGGGATCCTGGATGACA | |
| AGGGAGGCCTGGTGGATATCACCAGGAACGGGAAGGTCCCCTTCTTCGTGAAGTCTGAGA | |
| GACACGGCGTGCAGTCCCTCAGCAAATACAAACCTTCCAGCTCATTCATGGTGTCAAAAG | |
| TGAAAGTGCAGGATTTGGACTGCTACACGACCGTTGCTCAGCTGTGCCCGTTTGAAAAGC | |
| CAGCAACTCACTGCCCAAGAATCCATTGTCCGGCACACTGCAAAGACGAACCTTCCTACT | |
| GGGCTCCGGTGTTTGGAACCAACATCTATGCAGATACCTCAAGCATCTGCAAGACAGCCG | |
| TGCACGCGGGAGTCATCAGCAACGAGAGTGGGGGTGACGTGGACGTGATGCCCGTGGATA | |
| AAAAGAAGACCTACGTGGGCTCGCTCAGGAATGGAGTTCAGTCTGAAAGCCTGGGGACTC | |
| CTCGGGATGGAAAGGCCTTCCGGATCTTTGCTGTCAGGCAGTGAATTTCCAGCACCAGGG | |
| GAGAAGGGGCGTCTTCAGGAGGGCTTCGGGGTTTTGCTTTTATTTTTATTTTGTCATTGC | |
| GGGGTATATGGAGAGTCAGGAAACTTCCTTTGACTGATGTTCAGTGTCCATCACTTTGTG | |
| GCCTGTGGGTGAGGTGACATCTCATCCCCTCACTGAAGCAACAGCATCCCAAGGTGCTCA | |
| GCCGGACTCCCTGGTGCCTGATCCTGCTGGGGCCCGGGGGTCTCCATCTGGACGTCCTCT | |
| CTCCTTTAGAGATCTGAGCTGTCTCTTAAAGGGGACAGTTGCCCAAAATGTTCCTTGCTA | |
| TGTGTTCTTCTGTTGGTGGAGGAAGTTGATTTCAACCTCCCTGCCAAAAGAACAAACCAT | |
| TTGAAGCTCACAATTGTGAAGCATTCACGGCGTCGGAAGAGGCCTTTTGAGCAAGCGCCA | |
| ATGAGTTTCAGGAATGAAGTAGAAGGTAGTTATTTAAAAATAAAAAACACAGTCCGTCCC | |
| TACCAATAGAGGAAAATGGTTTTAATGTTTGCTGGTCAGACAGACAAATGGGCTAGAGTA | |
| AGAGGGCTGCGGGTATGAGAGACCCCGGCTCCGCCCTGGCACGTGTCCTTGCTGGCGGCC | |
| CGCCACAGGCCCCCTTCAATGGCCGCATTCAGGATGGCTCTATACACAGCAGTGCTGGTT | |
| TATGTAAAGTTCAGCAGTCACTTCA | |
| MTNWGRYRSPGFHVQSWYDEVKDYTYPYPSECNPWCPERCSGPMCTHYTQIVWATTNKI | (SEQ ID NO: 15) |
| GCAVNTCRKMTVWGEVWENAVYFVCNYSPKGNWIGEAPYKNGRPCSECPPSYGGSCRNN | |
| LCYREETYTPKPETDEMNEVETAPIPEENHVWLQPRVMRPTKPKKTSAVNYMTQVVRCD | |
| TKMKDRCKGSTCNRYQCPAGCLNHKAKIFGTLFYESSSSICRAAIHYGILDDKGGLVDI | |
| TRNGKVPFFVKSERHGVQSLSKYKPSSSFMVSKVKVQDLDCYTTVAQLCPFEKPATHCP | |
| RIHCPAHCKDEPSYWAPVFGTNIYADTSSICKTAVHAGVISNESGGDVDVMPVDKKKTY | |
| VGSLRNGVQSESLGTPRDGKAFRIFAVRQ | |

The disclosed NOV-4a amino acid sequence has a high level of homology (99% identity, 99% similarity) to a human trypsin inhibitor-like protein (GenBank Accession No: CAB66795), shown in Table 26. Moreover, the NOV-4a amino acid sequence has homology (72% identity, 82% similarity) to a known human trypsin inhibitor (TREMBL ACC No: 043692), also shown in Table 26. As indicated by the “Expect” values, the probability of these alignments occurring by chance alone is 0.0 and 5.3e-51, respectively.

TABLE 26
Score = 786 bits (2031), Expect = 0.0
Identities = 380/381 (99%), Positives = 381/381 (99%)

| | | | |
|---|---|---|---|
| NOV4a: | 3 | NWGRYRSPGFHVQSWYDEVKDYTYPYPSECNPWCPERCSGPMCTHYTQIVWATTNKIGCA | 62 |
| | | +WGRYRSPGFHVQSWYDEVKDYTYPYPSECNPWCPERCSGPMCTHYTQIVWATTNKIGCA | |
| TRYP: | 117 | HWGRYRSPGFHVQSWYDEVKDYTYPYPSECNPWCPERCSGPMCTHYTQIVWATTNKIGCA | 176 |
| NOV4a: | 63 | VNTCRKMTVWGEVWENAVYFVCNYSPKGNWIGEAPYKNGRPCSECPPSYGGSCRNNLCYR | 122 |
| | | VNTCRKMTVWGEVWENAVYFVCNYSPKGNWIGEAPYKNGRPCSECPPSYGGSCRNNLCYR | |
| TRYP: | 177 | VNTCRKMTVWGEVWENAVYFVCNYSPKGNWIGEAPYKNGRPCSECPPSYGGSCRNNLCYR | 236 |
| NOV4a: | 123 | EETYTPKPETDEMNEVETAPIPEENHVWLQPRVMRPTKPKKTSAVNYMTQVVRCDTKMKD | 182 |
| | | EETYTPKPETDEMNEVETAPIPEENHVWLQPRVMRPTKPKKTSAVNYMTQVVRCDTKMKD | |
| TRYP: | 237 | EETYTPKPETDEMNEVETAPIPEENHVWLQPRVMRPTKPKKTSAVNYMTQVVRCDTKMKD | 296 |
| NOV4a: | 183 | RCKGSTCNRYQCPAGCLNHKAKIFGTLFYESSSSICRAAIHYGILDDKGGLVDITRNGKV | 242 |
| | | RCKGSTCNRYQCPAGCLNHKAKIFGTLFYESSSSICRAAIHYGILDDKGGLVDITRNGKV | |
| TRYP: | 297 | RCKGSTCNRYQCPAGCLNHKAKIFGTLFYESSSSICRAAIHYGILDDKGGLVDITRNGKV | 356 |
| NOV4a: | 243 | PFFVKSERHGVQSLSKYKPSSSFMVSKVKVQDLDCYTTVAQLCPFEKPATHCPRIHCPAH | 302 |
| | | PFFVKSERHGVQSLSKYKPSSSFMVSKVKVQDLDCYTTVAQLCPFEKPATHCPRIHCPAH | |
| TRYP: | 357 | PFFVKSERHGVQSLSKYKPSSSFMVSKVKVQDLDCYTTVAQLCPFEKPATHCPRIHCPAH | 416 |
| NOV4a: | 303 | CKDEPSYWAPVFGTNIYADTSSICKTAVHAGVISNESGGDVDVMPVDKKKTYVGSLRNGV | 362 |
| | | CKDEPSYWAPVFGTNIYADTSSICKTAVHAGVISNESGGDVDVMPVDKKKTYVGSLRNGV | |
| TRYP: | 417 | CKDEPSYWAPVFGTNIYADTSSICKTAVHAGVISNESGGDVDVMPVDKKKTYVGSLRNGV | 476 |
| NOV4a: | 363 | QSESLGTPRDGKAFRIFAVRQ 383 (SEQ ID NO: 72) | |
| | | QSESLGTPRDGKAFRIFAVRQ | |
| TRYP: | 477 | QSESLGTPRDGKAFRIFAVRQ 497 (SEQ ID NO: 40) | |

Score = 530 (186.6 bits), Expect = 5.3e-51, P = 5.3e-51
Identities =85/117 (72%), Positives = 97/117 (82%)

| | | | | |
|---|---|---|---|---|
| NOV4a: | 5 | GRYRSPGFHVQSWYDEVKDYTYPYPSECNPWCPERCSGPMCTHYTQIVWATTNKIGCAVN | 64 | |
| | | GRYRS V+ WYDEVKDY +PYP +CNP CP RC GPMCTHYTQ+VWAT+N+IGCA++ | | |
| TRYP: | 130 | GRYRSILQLVKPWYDEVKDYAFPYPQDCNPRCPMRCFGPMCTHYTQMVWATSNRIGCAIH | 189 | |
| NOV4a: | 65 | TCRKMTVWGEVWENAVYFVCNYSPKGNWIGEAPYKNGRPCSECPPSYGGSCRNNLCY | 121 | (SEQ ID NO: 73) |
| | | TC+ M VWG VW AVY VCNY+PKGNWIGEAPYK G PCS CPPSYGGSC +NLC+ | | |
| TRYP: | 190 | TCQNMNVWGSVWRRAVYLVCNYAPKGNWIGEAPYKVGVPCSSCPPSYGGSCTDNLCF | 246 | (SEQ ID NO: 41) |

Furthermore, a PROSITE database search of protein families and domains confirmed that a NOV-4a polypeptide is a member of the trypsin inhibitor family. One of the conserved regions found in trypsin inhibitors is a SCP domain, located at the C-terminal half. The pattern of this conserved domain is: [LIVMFYH]-[LIVMFY]-x-C-[NQRHS]-Y-x-[PARH]-x-[GL]-N-[LIVMFYWDN] (SEQ ID NO: 56). This pattern is found in amino acids 81-92 of SEQ ID NO: 15.

PSORT analyses indicate that that NOV-4a is likely located in the nucleus (certainty=0.3000). The predicted molecular weight of NOV-4a is 43185.7 daltons.

Based on its relatedness to known members of the trypsin inhibitor family of proteins, NOV4a provides new diagnostic and therapeutic compositions useful in the treatment of disorders associated with alterations in the expression of members of the trypsin inhibitor protein family. Nucleic acids, polypeptides, antibodies, and other compositions of the present invention are useful in the treatment and diagnosis of a variety of diseases and pathologies, including, by way of nonlimiting example, those involving reproductive disorders, immunological disorders, cancer, and metabolic disorders.

NOV-4b

A disclosed NOV-4b sequence according to the invention is a nucleic acid sequence that encodes a polypeptide related to trypsin inhibitor proteins. A disclosed NOV-4b nucleic acid and its encoded polypeptide are included in Table 27. The disclosed nucleic acid (SEQ ID NO: 16) is 2400 nucleotides in length and contains an open reading frame (ORF) that begins with an ATG initiation codon at nucleotide 205, and ends with a TGA stop codon at nucleotide 1697. A disclosed, representative ORF encodes a 497 amino acid polypeptide (SEQ ID NO: 17).

TABLE 27

| | |
|---|---|
| CTCTGACTGCTCCTATTGAGCTGTCTGCTCGCTGTGCCCGCTGTGCCTGCTGTGCCCGCGCTGTCGCCGCTGCTACCGCG | (SEQ ID NO:16) |
| TCTACTGGACGCGGGAGACGCCAGCGAGCTGGTGATTGGAGCCCTGCGGAGAGCTCAAGCGCCCAGCTCTGCCCGAGGAG | |
| CCCAGGCTGCCCCGTGAGTCCCATAGTTGCTGCAGGAGTGGAGCCATGAGCTGCGTCCTGGGTGGTGTCATCCCCTTGGG | |
| GCTGCTGTTCCTGGTCCGCGGATCCCAAGGCTACCTCCTGCCCAACGTCACTCTCTTAGAGGAGCTGCTCAGCAAATACC | |
| AGCACAACGAGTCTCACTCCCGGGTCCGCAGAGCCATCCCCAGGGAGGACAAGGAGGAGATCCTCATGCTGCACAACAAG | |
| CTTCGGGGCCAGGTGCAGCCTCAGGCCTCCAACATGGAGTACATGACCTGGGATGACGAACTGGAGAAGTCTGCTGCAGC | |
| GTGGGCCAGTCAGTGCATCTGGGAGCACGGGCCCACCGGTCTGCTGGTGTCCATCGGGCAGAACCTGGGCGCTCACTGGG | |
| GCAGGTATCGCTCTCCGGGGTTCCATGTGCAGTCCTGGTATGACGAGGTGAAGGACTACACCTACCCCTACCCGAGCGAG | |
| TGCAACCCCTGGTGTCCAGAGAGGTGCTCGGGGCCTATGTGCACGCACTACACACAGATAGTTTGGGCCACCACCAACAA | |
| GATCGGTTGTGCTGTGAACACCTGCCGGAAGATGACTGTCTGGGGAGAAGTTTGGGAGAACGCGGTCTACTTTGTCTGCA | |
| ATTATTCTCCAAAGGGGAACTGGATTGGAGAAGCCCCCTACAAGAATGGCCGGCCCTGCTCTGAGTGCCCACCCAGCTAT | |
| GGAGGCAGCTGCAGGAACAACTTGTGTTACCGAGAAGAAACCTACACTCCAAAACCTGAAACGGACGAGATGAATGAGGT | |
| GGAAACGGCTCCCATTCCTGAAGAAAACCATGTTTGGCTCCAACCGAGGGTGATGAGACCCACCAAGCCCAAGAAAACCT | |
| CTGCGGTCAACTACATGACCCAAGTCGTCAGATGTGACACCAAGATGAAGGACAGGTGCAAAGGGTCCACGTGTAACAGG | |
| TACCAGTGCCCAGCAGGCTGCCTGAACCACAAGGCGAAGATCTTTGGAAGTCTGTTCTATGAAAGCTCGTCTAGCATATG | |
| CCGCGCCGCCATCCACTACGGGATCCTGGATGACAAGGGAGGCCTGGTGGATATCACCAGGAACGGGAAGGTCCCCTTCT | |
| TCGTGAAGTCTGAGAGACACGGCGTGCAGTCCCTCAGCAAATACAAACCTTCCAGCTCATTCATGGTGTCAAAAGTGAAA | |
| GTGCAGGATTTGGACTGCTACACGACCGTTGCTCAGCTGTGCCCGTTTGAAAAGCCAGCAACTCACTGCCCAAGAATCCA | |
| TTGTCCGGCACACTGCAAAGACGAACCTTCCTACTGGGCTCCGGTGTTTGGAACCAACATCTATGCAGATACCTCAAGCA | |
| TCTGCAAGACAGCCGTGCACGCGGGAGTCATCAGCAACGAGAGTGGGGGTGACGTGGACGTGATGCCCGTGGATAAAAAG | |
| AAGACCTACGTGGGCTCGCTCAGGAATGGAGTTCAGTCTGAAAGCCTGGGGACTCCTCGGGATGGAAAGGCCTTCCGGAT | |
| CTTTGCTGTCAGGCAGTGAATTTCCAGCACCAGGGGAGAAGGGGCGTCTTCAGGAGGGCTTCGGGGTTTTGCTTTTATTT | |
| TTATTTTGTCATTGCGGGGTATATGGAGAGTCAGGAAACTTCCTTTGACTGATGTTCAGTGTCCATCACTTTGTGGCCTG | |
| TGGGTGAGGTGACATCTCATCCCCTCACTGAAGCAACAGCATCCCAAGGTGCTCAGCCGGACTCCCTGGTGCCTGATCCT | |
| GCTGGGGCCCGGGGGTCTCCATCTGGACGTCCTCTCTCCTTTAGAGATCTGAGCTGTCTCTTAAAGGGGACAGTTGCCCA | |
| AAATGTTCCTTGCTATGTGTTCTTCTGTTGGTGGAGGAAGTTGATTTCAACCTCCCTGCCAAAAGAACAAACCATTTGAA | |
| GCTCACAATTGTGAAGCATTCACGGCGTCGGAAGAGGCCTTTTGAGCAAGCGCCAATGAGTTTCAGGAATGAAGTAGAAG | |
| GTAGTTATTTAAAAATAAAAAACACAGTCCGTCCCTACCAATAGAGGAAAATGGTTTTAATGTTTGCTGGTCAGACAGAC | |
| AAATGGGCTAGAGTAAGAGGGCTGCGGGTATGAGAGACCCCGGCTCCGCCCTGGCACGTGTCCTTGCTGGCGGCCCGCCA | |
| CAGGCCCCCTTCAATGGCCGCATTCAGGATGGCTCTATACACAGCAGTGCTGGTTTATGTAAAGTTCAGCAGTCACTTCA | |
| MSCVLGGVIPLGLLFLVRGSQGYLLPNVTLLEELLSKYQHNESHSRVRRAIPREDKEEILMLHNKLRGQVQPQASNMEY | (SEQ ID NO: 17) |
| MTWDDELEKSAAAWASQCIWEHGPTGLLVSIGQNLGAHWGRYRSPGFHVQSWYDEVKDYTYPYPSECNPWCPERCSGPM | |
| CTHYTQIVWATTNKIGCAVNTCRKMTVWGEVWENAVYFVCNYSPKGNWIGEAPYKNGRPCSECPPSYGGSCRNNLCYRE | |
| ETYTPKPETDEMNEVETAPIPEENHVWLQPRVMRPTKPKKTSAVNYMTQVVRCDTKMKDRCKGSTCNRYQCPAGCLNHK | |
| AKIFGSLFYESSSSICRAAIHYGILDDKGGLVDITRNGKVPFFVKSERHGVQSLSKYKPSSSFMVSKVKVQDLDCYTTV | |
| AQLCPFEKPATHCPRIHCPAHCKDEPSYWAPVFGTNIYADTSSICKTAVHAGVISNESGGDVDVMPVDKKKTYVGSLRN | |
| GVQSESLGTPRDGKAFRIFAVRQ | |

The disclosed NOV-4b amino acid sequence has 124 of 191 amino acid residues (64%) identical to, and 148 of 191 (77%) similar to, a known human trypsin inhibitor (TREMBL ACC No: 043692), as shown in Table 28. As indicated by the “Expect” value, the probability of this alignment occurring by chance alone is 6.1e-73, which is a very low probability score.

TABLE 28
Score = 737 (259.4 bits), Expect = 6.le-73, P = 6.le-73
Identities = 124/191 (64%), Positives = 148/191 (77%)

| | | | |
|---|---|---|---|
| NOV4b: | 45 | SRVRRAIPREDKEEILMLHNKLRGQVQPQASNMEYMTWDDELEKSAAWASQCIWEHGPT | 104 |
| | | +R +R I +D IL HN++RG+V P A+NMEYM WD+L KSA AWA+CIW+HGP+ | |
| TRYP : | 56 | ARRKRYISQNDMIAILDYHNQVRGKVFPPAANMEYMVWDENLAKSAEAWAATCIWDHGPS | 115 |
| NOV4b: | 105 | GLLVSIGQNLGAHWGRYRSPGFHVQSWYDEVKDYTYPYPSECNPWCPERCSGPMCTHYTQ | 164 |
| | | LL +GQNL GRYRS V+WYDEVKDY +PYP +CNP CP RC GPMCTHYTQ | |
| TRYP : | 116 | YLLRFLGQNLSVRTGRYRSILQLVKPWYDEVKDYAFPYPQDCNPRCPMRCFGPMCTHYTQ | 175 |
| NOV4b: | 165 | IVWATTNKIGCAVNTCRKMTVWGEVWENAVYFVCNYSPKGNWIGEAPYKNGRPCSECPPS | 224 |
| | | +VWAT+N+IGCA++TC+M VWG VW AVY VCNY+PKGNWIGEAPYK G PCS CPPS | |
| TRYP | 176 | MVWATSNRIGCAIHTCQNMNVWGSVWRRAVYLVCNYAPKGNWIGEAPYKVGVPCSSCPPS | |
| NOV4b: | 225 | YGGSCRNNLCY 235 (SEQ ID NO:74) | |
| | | YGGSC +NLC+ | |
| TRYP: | 236 | YGGSCTDNLCF 246 (SEQ ID NO:42) | |

Furthermore, a PROSITE database search of protein families and domains confirmed that NOV-4a is a member of the trypsin inhibitor family. One of the conserved regions found in trypsin inhibitors is a SCP domain, located at the C-terminal half. The pattern of this conserved domain is: [LIVMFYH]-[LIVMFY]-x-C-[NQRHS]-Y-x-[PARH]-x-[GL]-N-[LIVMFYWDN] (SEQ ID NO: 56). This pattern is found in amino acids 195-206 of SEQ ID NO: 17.

SignalPep and PSORT analyses indicate that that NOV-4b is likely located outside of the cell (certainty=0.6950), and is likely to have a cleavable N-terminal signal sequence with a cleavage site between positions 22 and 23: SQG-YL. The predicted molecular weight of NOV-4b is 55928.2 daltons.

Based on its relatedness to known members of the trypsin inhibitor family of proteins, NOV4b provides new diagnostic and therapeutic compositions useful in the treatment of disorders associated with alterations in the expression of members of the trypsin inhibitor protein family. Nucleic acids, polypeptides, antibodies, and other compositions of the present invention are useful in the treatment and diagnosis of a variety of diseases and pathologies, including, by way of nonlimiting example, those involving reproductive disorders, immunological disorders, cancer, and metabolic disorders.

NOV-4c

A NOV-4c sequence according to the invention is a nucleic acid sequence that encodes a polypeptide related to trypsin inhibitor proteins. A disclosed NOV-4c nucleic acid and its encoded polypeptide are included in Table 29. The disclosed nucleic acid (SEQ ID NO: 18) is 1669 nucleotides in length and contains an open reading frame (ORF) that begins with an ATG initiation codon at nucleotide 205, and ends with a TAG stop codon at nucleotide 1636. The representative ORF encodes a 205 amino acid polypeptide (SEQ ID NO: 19).

TABLE 29

| | |
|---|---|
| TCTGACTGCTCCTATTGAGCTGTCTGCTCGCTGTGCCCGCTGTGCCTGCTGTGCCCGCGC | (SEQ ID NO:18) |
| TGTCGCCGCTGCTACCGCGTCTACTGGACGCGGGAGACGCCAGCGAGCTGGTGATTGGAG | |
| CCCTGCGGAGAGCTCAAGCGCCCAGCTCTGCCCGAGGAGCCCAGGCTGCCCCGTGAGTCC | |
| CATAGTTGCTGCAGGAGTGGAGCCATGAGCTGCGTCCTGGGTGGTGTCATCCCCTTGGGG | |
| CTGCTGTTCCTGGTCTGCGGATCCCAAGGCTACCTCCTGCCCAACGTCACTCTCTTAGAG | |
| GAGCTGCTCAGCAAATACCAGCACAACGAGTCTCACTCCCGGGTCCGCAGAGCCATCCCC | |
| AGGGAGGACAAGGAGGAGATCCTCATGCTGCACAACAAGCTTCGGGGCCAGGTGCAGCCT | |
| CAGGCCTCCAACATGGAGTACATGACCTGGGATGACGAACTGGAGAAGTCTGCTGCAGCG | |
| TGGGCCAGTCAGTGCATCTGGGAGCACGGGCCCACCGGTCTGCTGGTGTCCATCGGGCAG | |
| AACCTGGGCGCTCACTGGGGCAGGTATCGCTCTCCGGGGTTCCATGTGCAGTCCTGGTAT | |
| GACGAGGTGAAGGACTACACCTACCCCTACCCGAGCGAGTGCAACCCCTGGTGTCCAGAG | |
| AGGTGCTCGGGGCCTATGTGCACGCACTACACACAGATAGTTTGGGCCACCACCAACAAG | |
| ATCGGTTGTGCTGTGAACACCTGCCGGAAGATGACTGTCTGGGGAGAAGTTTGGGAGAAC | |
| GCGGTCTACTTTGTCTGCAATTATTCTCCAAAGGGGAACTGGATTGGAGAAGCCCCCTAC | |
| AAGAATGGCCGGCCCTGCTCTCAGTGCCCACCCAGCTATGGAGGCAGCTGCAGGAACAAC | |
| TTGTGTTACCGAGAAGAAACCTACACTCCAAAACCTGAAACGGACGAGATGAATGAGGTG | |
| ACCAAGCCCAAGAAAACCTCTTCGGTCAACTACATGACCCAAGTCGTCTTATGTGACACC | |
| AAGATGAAGGACAGGTGCAAAGGGTCCACGTGTAACAGGTACCAGTGCCCAGCAGGCTGC | |
| CTGAACCACAAGGCGAAGATCTTTGGAACTCTGTTCTATGAAAGCTCGTCTAGCATATGC | |
| CGCGCCGCCATCCACTACGGGATCCTGGATGACAAGGGAGGCCTGGTGGATATCACCAGG | |
| AACGGGAAGGTCCCCTTCTTCGTGAAGTCTGAGAGACACGGCGTGCAGTCCCTCAGCAAA | |
| TACAAACCTTCCAGCTCATTCATGGTGTCAAAAGTGAAAGTGCAGGATTTGGACTGCTAC | |
| ACGACCGTTGCTCAGCTGTGCCCGTTTGAAAAGCCAGCAACTCACTGCCCAAGAATCCAT | |
| TGTCCGGCACACTGCAAAGACGAACCTTCCTACTGGGCTCCGGTGTTTGGAACCAACATC | |
| TATGCAGATACCTCAAGCATCTGCAAGACAGCCGTGCACGCGGGAGTCATCAGCAACGAG | |
| AGTGGGGGTGACGTGGACGTGATGCCCGTGGATAAAAAGAAGACCTACACCTGCCCGGCA | |
| GCCGCTCGAGCCCTATAGTGTAAACCGATTCGCAGCACACTGGCGCCGT | |
| MSCVLGGVIPLGLLFLVCGSQGYLLPNVTLLEELLSKYQHNESHSRVRRAIPR | (SEQ ID NO:19) |
| EDKEEILMLHNKLRGQVQPQASNMEYMTWDDELEKSAAAWASQCIWEHGPTGL | |
| LVSIGQNLGAHWGRYRSPGFHVQSWYDEVKDYTYPYPSECNPWCPERCSGPMC | |
| THYTQIVWATTNKIGCAVNTCRKMTVWGEVWENAVYFVCNYSPKGNWIGEAPY | |
| KNGRPCSQCPPSYGGSCRNNLCYREETYTPKPETDEMNEVETAPIPEENHVWL | |
| QPRVMRPTKPKKTSSVNYMTQVVLCDTKMKDRCKGSTCNRYQCPAGCLNHKAK | |
| IFGTLFYESSSSICRAAIHYGILDDKGGLVDITRNGKVPFFVKSERHGVQSLS | |
| KYKPSSSFMVSKVKVQDLDCYTTVAQLCPFEKPATHCPRIHCPAHCKDEPSYW | |
| APVFGTNIYADTSSICKTAVHAGVISNESGGDVDVMPVDKKKTYTCPAAARAL | |

The disclosed NOV-4c amino acid sequence has a high level of homology (97% identity, 97% similarity) to a human trypsin inhibitor-like protein (GenBank Accession No: CAB66795), shown in Table 30. As indicated by the “Expect” value, the probability of this alignment occurring by chance alone is 0.0, the lowest probability score.

TABLE 30
Score = 948 bits (2452), Expect = 0.0
Identities = 458/468 (97%), Positives = 460/468 (97%)

| | | | |
|---|---|---|---|
| NOV4c: 1 | MSCVLGGVIPLGLLFLVCGSQGYLLPNVTXXXXXXSKYQHNESHSRVRRAIPREDKEEIL | 60 | |
| | MSCVLGGVIPLGLLFLVCGSQGYLLPNVT SKYQHNESHSRVRRAIPREDKEEIL | | |
| TRYP: 1 | MSCVLGGVIPLGLLFLVCGSQGYLLPNVTLLEELLSKYQHNESHSRVRRAIPREDKEEIL | 60 | |
| NOV4c: 61 | MLHNKLRGQVQPQASNMEYMTWDDELEKSAAAWASQCIWEHGPTGLLVSIGQNLGAHWGR | 120 | |
| | MLHNKLRGQVQPQASNMEYMTWDDELEKSAAAWASQCIWEHGPT LLVSIGQNLGAHWGR | | |
| TRYP: 61 | MLHNKLRGQVQPQASNMEYMTWDDELEKSAAAWASQCIWEHGPTSLLVSIGQNLGAHWGR | 120 | |
| NOV4c: 121 | YRSPGFHVQSWYDEVKDYTYPYPSECNPWCPERCSGPMCTHYTQIVWATTNKIGCAVNTC | 180 | |
| | YRSPGFHVQSWYDEVKDYTYPYPSECNPWCPERCSGPMCTHYTQIVWATTNKIGCAVNTI | | |
| TRYP: 121 | YRSPGFHVQSWYDEVKDYTYPYPSECNPWCPERCSGPMCTHYTQIVWATTNKIGCAVNTC | 180 | |
| NOV4c: 181 | RKMTVWGEVWENAVYFVCNYSPKGNWIGEAPYKNGRPCSQCPPSYGGSCRNNLCYREETY | 240 | |
| | RKMTVWGEVWENAVYFVCNYSPKGNWIGEAPYKNGRPCS+CPPSYGGSCRNNLCYREETY | | |
| TRYP: 181 | RKMTVWGEVWENAVYFVCNYSPKGNWIGEAPYKNGRPCSECPPSYGGSCRNNLCYREETY | 240 | |
| NOV4c: 241 | TPKPETDEMNEVETAPIPEENHVWLQPRVMRPTKPKKTSSVNYMTQVVLCDTKMKDRCKG | 300 | |
| | TPKPETDEMNEVETAPIPEENHVWLQPRVMRPTKPKKTS+VNYMTQVV CDTKMKDRCKG | | |
| TRYP: 241 | TPKPETDEMNEVETAPIPEENHVWLQPRVMRPTKPKKTSAVNYMTQVVRCDTKMKDRCKG | 300 | |
| NOV4c: 301 | STCNRYQCPAGCLNHKAKIFGTLFYESSSSICRAAIHYGILDDKGGLVDITRNGKVPFFV | 360 | |
| | STCNRYQCPAGCLNHKAKIFGTLFYESSSSICRAAIHYGILDDKGGLVDITRNGKVPFFV | | |
| TRYP: 301 | STCNRYQCPAGCLNHKAKIFGTLFYESSSSICRAAIHYGILDDKGGLVDITRNGKVPFFV | 360 | |
| NOV4c: 361 | KSERHGVQSLSKYKPSSSFMVSKVKVQDLDCYTTVAQLCPFEKPATHCPRIHCPAHCKDE | 420 | |
| | KSERHGVQSLSKYKPSSSFMVSKVKVQDLDCYTTVAQLCPFEKPATHCPRIHCPAHCKDE | | |
| TRYP: 361 | KSERHGVQSLSKYKPSSSFMVSKVKVQDLDCYTTVAQLCPFEKPATHCPRIHCPAHCKDE | 420 | |
| NOV4c: 421 | PSYWAPVFGTNIYADTSSICKTAVHAGVISNESGGDVDVMPVDKKKTY | 468 | (SEQ ID NO:75) |
| | PSYWAPVFGTNIYADTSSICKTAVHAGVISNESGGDVDVMPVDKKKTY | | |
| TRYP: 421 | PSYWAPVFGTNIYADTSSICKTAVHAGVISNESGGDVDVMPVDKKKTY | 468 | (SEQ ID NO: 43) |

Furthermore, a PROSITE database search of protein families and domains confirmed that NOV-4c is a member of the trypsin inhibitor family. One of the conserved regions found in trypsin inhibitors is a SCP domain, located at the C-terminal half. The pattern of this conserved domain is: [LIVMFYH]-[LIVMFY]-x-C-[NQRHS]-Y-x-[PARH]-x-[GL]-N-[LIVMFYWDN] (SEQ ID NO: 56). This pattern is found in amino acids 81-92 of SEQ ID NO: 19.

In addition, SignalPep and PSORT analyses indicate that NOV-4c is likely located outside of the cell (certainty=0.8200), and is likely to have a cleavable N-terminal signal sequence with a cleavage site between positions 22 and 23: SQG-YL. The predicted molecular weight of NOV-4c is 53587.7 daltons.

Based on the relatedness between NOV-4c and the conserved trypsin inhibitor proteins, the NOV-4c protein is a novel member of the trypsin inhibitor family. NOV-4c provides new diagnostic and therapeutic compositions useful in the treatment of disorders associated with alterations in the expression of members of the trypsin inhibitor protein family. Nucleic acids, polypeptides, antibodies, and other compositions of the present invention are useful in the treatment and diagnosis of a variety of diseases and pathologies, including, by way of nonlimiting example, those involving reproductive disorders, immunological disorders, cancer, and metabolic disorders.

NOV-4d

A NOV-4d sequence according to the invention is a nucleic acid sequence that encodes a polypeptide related to trypsin inhibitor proteins. A disclosed NOV-4d nucleic acid and its encoded polypeptide are included in Table 31. The disclosed nucleic acid (SEQ ID NO: 20) is 2403 nucleotides in length and contains an open reading frame (ORF) that begins with an ATG initiation codon at nucleotide 206, and ends with a TGA stop codon at nucleotide 1700. A disclosed, representative ORF encodes a 498 amino acid polypeptide (SEQ ID NO: 21).

TABLE 31

| | |
|---|---|
| CTCTGACTGCTCCTATTGAGCTGTCTGCTCGCTGTGCCCGCTGTGCCTGCTGTGCCCGCGCTGTCGCCGCTGCTACCGCG | (SEQ ID NO:20) |
| TCTACTGGACGCGGGAGACGCCAGCGAGCTGGTGATTGGAGCCCTGCGGAGAGCTCAAGCGCCCAGCTCTGCCCGAGGAG | |
| CCCAGGCTGCCCCGTGAGTCCCATAGTTGCTGCAGGAGTGGAGCCATGAGCTGCGTCCTGGGTGGTGTCATCCCCTTGGG | |
| GCTGCTGTTCCTGGTCCGCGGATCCCAAGGCTACCTCCTGCCCAACGTCACTCTCTTAGAGGAGCTGCTCAGCAAATACC | |
| AGCACAACGAGTCTCACTCCCGGGTCCGCAGAGCCATCCCCAGGGAGGACAAGGAGGAGATCCTCATGCTGCACAACAAG | |
| CTTCGGGGCCAGGTGCAGCCTCAGGCCTCCAACATGGAGTACATGACCTGGGATGACGAACTGGAGAAGTCTGCTGCAGC | |
| GTGGGCCAGTCAGTGCATCTGGGAGCACGGGCCCACCAGTCTGCTGGTGTCCATCGGGCAGAACCTGGGCGCTCACTGGG | |
| GCAGGAGGTATCGCTCTCCGGGGTTCCATGTGCAGTCCTGGTATGACGAGGTGAAGGACTACACCTACCCCTACCCGAGC | |
| GAGTGCAACCCCTGGTGTCCAGAGAGGTGCTCGGGGCCTATGTGCACGCACTACACACAGATAGTTTGGGCCACCACCAA | |
| CAAGATCGGTTGTGCTGTGAACACCTGCCGGAAGATGACTGTCTGGGGAGAAGTTTGGGAGAACGCGGTCTACTTTGTCT | |
| GCAATTATTCTCCAAAGGGGAACTGGATTGGAGAAGCCCCCTACAAGAATGGCCGGCCCTGCTCTGAGTGCCCACCCAGC | |
| TATGGAGGCAGCTGCAGGAACAACTTGTGTTACCGAGAAGAAACCTACACTCCAAAACCTGAAACGGACGAGATGAATGA | |
| GGTGGAAACGGCTCCCATTCCTGAAGAAAACCATGTTTGGCTCCAACCGAGGGTGATGAGACCCACCAAGCCCAAGAAAA | |
| CCTCTGCGGTCAACTACATGACCCAAGTCGTCAGATGTGACACCAAGATGAAGGACAGGTGCAAAGGGTCCACGTGTAAC | |
| AGGTACCAGTGCCCAGCAGGCTGCCTGAACCACAAGGCGAAGATCTTTGGAAGTCTGTTCTATGAAAGCTCGTCTAGCAT | |
| ATGCCGCGCCGCCATCCACTACGGGATCCTGGATGACAAGGGAGGCCTGGTGGATATCACCAGGAACGGGAAGGTCCCCT | |
| TCTTCGTGAAGTCTGAGAGACACGGCGTGCAGTCCCTCAGCAAATACAAACCTTCCAGCTCATTCATGGTGTCAAAAGTG | |
| AAAGTGCAGGATTTGGACTGCTACACGACCGTTGCTCAGCTGTGCCCGTTTGAAAAGCCAGCAACTCACTGCCCAAGAAT | |
| CCATTGTCCGGCACACTGCAAAGACGAACCTTCCTACTGGGCTCCGGTGTTTGGAACCAACATCTATGCAGATACCTCAA | |
| GCATCTGCAAGACAGCCGTGCACGCGGGAGTCATCAGCAACGAGAGTGGGGGTGACGTGGACGTGATGCCCGTGGATAAA | |
| AAGAAGACCTACGTGGGCTCGCTCAGGAATGGAGTTCAGTCTGAAAGCCTGGGGACTCCTCGGGATGGAAAGGCCTTCCG | |
| GATCTTTGCTGTCAGGCAGTGAATTTCCAGCACCAGGGGAGAAGGGGCGTCTTCAGGAGGGCTTCGGGGTTTTGCTTTTA | |
| TTTTTATTTTGTCATTGCGGGGTATATGGAGAGTCAGGAAACTTCCTTTGACTGATGTTCAGTGTCCATCACTTTGTGGC | |
| CTGTGGGTGAGGTGACATCTCATCCCCTCACTGAAGCAACAGCATCCCAAGGTGCTCAGCCGGACTCCCTGGTGCCTGAT | |
| CCTGCTGGGGCCCGGGGGTCTCCATCTGGACGTCCTCTCTCCTTTAGAGATCTGAGCTGTCTCTTAAAGGGGACAGTTGC | |
| CCAAAATGTTCCTTGCTATGTGTTCTTCTGTTGGTGGAGGAAGTTGATTTCAACCTCCCTGCCAAAAGAACAAACCATTT | |
| GAAGCTCACAATTGTGAAGCATTCACGGCGTCGGAAGAGGCCTTTTGAGCAAGCGCCAATGAGTTTCAGGAATGAAGTAG | |
| AAGGTAGTTATTTAAAAATAAAAAACACAGTCCGTCCCTACCAATAGAGGAAAATGGTTTTAATGTTTGCTGGTCAGACA | |
| GACAAATGGGCTAGAGTAAGAGGGCTGCGGGTATGAGAGACCCCGGCTCCGCCCTGGCACGTGTCCTTGCTGGCGGCCCG | |
| CCACAGGCCCCCTTCAATGGCCGCATTCAGGATGGCTCTATACACAGCAGTGCTGGTTTATGTAAAGTTCAGCAGTCACT | |
| TCA | |
| MSCVLGGVIPLGLLFLVRGSQGYLLPNVTLLEELLSKYQHNESHSRVRRAIPREDKEEILMLHNKLRGQVQ | (SEQ ID NO: 21) |
| PQASNMEYMTWDDELEKSAAAWASQCIWEHGPTSLLVSIGQNLGAHWGRRYRSPGFHVQSWYDEVKDYTYP | |
| YPSECNPWCPERCSGPMCTHYTQIVWATTNKIGCAVNTCRMKTVWGEVWENAVYFVCNYSPKGNWIGEAPY | |
| KNGRPCSECPPSYGGSCRNNLCYREETYTPKPETDEMNEVETAPIPEENHVWLQPRVMRPTKPKKTSAVNY | |
| MTQVVRCDTKMKDRCKGSTCNRYQCPAGCLNHKAKIFGSLFYESSSSICRAAIHYGILDDKGGLVDITRNG | |
| KVPFFVKSERHGVQSLSKYKPSSSFMVSKVKVQDLDCYTTVAQLCPFEKPATHCPRIHCPAHCKDEPSYWA | |
| PVFGTNIYADTSSICKTAVHAGVISNESGGDVDVMPDVKKKTYVGSLRNGVQSESLGTPRDGKAFRIFAVRQ | |

The disclosed NOV-4d amino acid sequence has a high level of homology (98% identity, 98% similarity) to a human trypsin inhibitor-like protein (GenBank Accession No: CAB66795), as shown in Table 32. As indicated by the “Expect” value, the probability of this alignment occurring by chance alone is 0.0, the lowest probability score.

TABLE 32
Score = 1007 bits (2605), Expect = 0.0
Identities = 489/498 (98%), Positives = 490/498 (98%), Gaps = 1/498 (0%)

| | | | |
|---|---|---|---|
| NOV4d: 1 | MSCVLGGVIPLGLLFLVRGSQGYLLPNVTXXXXXXSKYQHNESHSRVRRAIPREDKEEIL | 60 | |
| | MSCVLGGVIPLGLLFLV GSQGYLLPNVT SKYQHNESHSRVRRAIPREDKEEIL | | |
| TRYP: 1 | MSCVLGGVIPLGLLFLVCGSQGYLLPNVTLLEELLSKYQHNESHSRVRRAIPREDKEEIL | 60 | |
| NOV4d: 61 | MLHNKLRGQVQPQASNMEYMTWDDELEKSAAAWASQCIWEHGPTSLLVSIGQNLGAHWGR | 120 | |
| | MLHNKLRGQVQPQASNMEYMTWDDELEKSAAAWASQCIWEHGPTSLLVSIGQNLGAHWG | | |
| TRYP: 61 | MLHNKLRGQVQPQASNMEYMTWDDELEKSAAAWASQCIWEHGPTSLLVSIGQNLGAHWG- | 119 | |
| NOV4d: 121 | RYRSPGFHVQSWYDEVKDYTYPYPSECNPWCPERCSGPMCTHYTQIVWATTNKIGCAVNT | 180 | |
| | RYRSPGFHVQSWYDEVKDYTYPYPSECNPWCPERCSGPMCTHYTQIVWATTNKIGCAVNT | | |
| TRYP: 120 | RYRSPGFIVQSWYDEVKDYTYPYPSECNPWCPERCSGPMCTHYTQIVWATTNKIGCAVNT | 179 | |
| NOV4d: 181 | CRKMTVWGEVWENAVYFVCNYSPKGNWIGEAPYKNGRPCSECPPSYGGSCRNNLCYREET | 240 | |
| | CRKMTVWGEVWENAVYFVCNYSPKGNWIGEAPYKNGRPCSECPPSYGGSCRNNLCYREET | | |
| TRYP: 180 | CRKMTVWGEVWENAVYFVCNYSPKGNWIGEAPYKNGRPCSECPPSYGGSCRNNLCYREET | 239 | |
| NOV4d: 241 | YTPKPETDEMNEVETAPIPEENHVWLQPRVMRPTKPKKTSAVNYMTQVVRCDTKMKDRCK | 300 | |
| | YTPKPETDEMNEVETAPIPEENHVWLQPRVMRPTKPKKTSAVNYMTQVVRCDTKMKDRCK | | |
| TRYP: 240 | YTPKPETDEMNEVETAPIPEENHVWLQPRVMRPTKPKKTSAVNYMTQVVRCDTKMKDRCK | 299 | |
| NOV4d: 301 | GSTCNRYQCPAGCLNHKAKIFGSLFYESSSSICRAAIHYGILDDKGGLVDITRNGKVPFF | 360 | |
| | GSTCNRYQCPAGCLNHKAKIFG+LFYESSSSICRAAIHYGILDDKGGLVDITRNGKVPFF | | |
| TRYP: 300 | GSTCNRYQCPAGCLNHKAKIFGTLFYESSSSICRAAIHYGILDDKGGLDVITRNGKVPFF | 359 | |
| NOV4d: 361 | VKSERHGVQSLSKYKPSSSFMVSKVKVQDLDCYTTVAQLCPFEKPATHCPRIHCPAHCKD | 420 | |
| | VKSERHGVQSLSKYKPSSSFMVSKVKVQDLDCYTTVAQLCPFEKPATHCPRIHCPAHCKD | | |
| TRYP: 360 | VKSERHGVQSLSKYKPSSSFMVSKVKVQDLDCYTTVAQLCPFEKPATHCPRIHCPAHCKD | 419 | |
| NOV4d: 421 | EPSYWAPVFGTNIYADTSSICKTAVHAGVISNESGGDVDVMPVDKKKTYVGSLRNGVQSE | 480 | |
| | EPSYWAPVFGTNIYADTSSICKTAVHAGVISNESGGDVDVMPVDKKKTYVGSLRNGVQSE | | |
| TRYP: 420 | EPSYWAPVFGTNIYADTSSICKTAVHAGVISNESGGDVDVMPVDKKKTYVGSLRNGVQSE | 479 | |
| NOV4d: 481 | SLGTPRDGKAFRIFAVRQ | 498 | (SEQ ID NO:76) |
| | SLGTPRDGKAFRIFAVRQ | | |
| TRYP: 480 | SLGTPRDGKAFRIFAVRQ | 497 | (SEQ ID NO:44) |

A PROSITE database search of protein families and domains confirmed that a NOV-4c polypeptide is a member of the trypsin inhibitor family. One of the conserved regions found in trypsin inhibitors is a SCP domain, located at the C-terminal half. The pattern of this conserved domain is: [LIVMFYH]-[LIVMFY]-x-C-[NQRHS]-Y-x-[PARH]-x-[GL]-N-[LIVMFYWDN] (SEQ ID NO: 56). This pattern is found in amino acids 196-207 of SEQ ID NO: 21.

Based on the relatedness between NOV-4d and the conserved trypsin inhibitor proteins, NOV-4d is a novel member of the trypsin inhibitor family. NOV-4d provides new diagnostic and therapeutic compositions useful in the treatment of disorders associated with alterations in the expression of members of the trypsin inhibitor protein family. Nucleic acids, polypeptides, antibodies, and other compositions of the present invention are useful in the treatment and diagnosis of a variety of diseases and pathologies, including, by way of nonlimiting example, those involving reproductive disorders, immunological disorders, cancer, and metabolic disorders.

In addition, SignalPep and PSORT analyses indicate that that NOV-4d is likely located outside of the cell (certainty=0.6950), and is likely to have a cleavable N-terminal signal sequence with a cleavage site between positions 22 and 23: SQG-YL. The predicted molecular weight of NOV-4b is 56114.4 daltons.

NOV-4e

A NOV-4e sequence according to the invention is a nucleic acid sequence that encodes a polypeptide related to trypsin inhibitor proteins. A disclosed NOV-4e nucleic acid and its encoded polypeptide are included in Table 33. The disclosed nucleic acid (SEQ ID NO: 22) is 2412 nucleotides in length and contains an open reading frame (ORF) that begins with an ATG initiation codon at nucleotide 206, and ends with a TGA stop codon at nucleotide 1709. A disclosed, representative ORF encodes a 501 amino acid polypeptide (SEQ ID NO: 23).

TABLE 33

| | |
|---|---|
| CTCTGACTGCTCCTATTGAGCTGTCTGCTCGCTGTGCCCGCTGTGCCTGCTGTGCCCGC | (SEQ ID NO:22) |
| GCTGTCGCCGCTGCTACCGCGTCTACTGGACGCGGGAGACGCCAGCGAGCTGGTGATTG | |
| GAGCCCTGCGGAGAGCTCAAGCGCCCAGCTCTGCCCGAGGAGCCCAGGCTGCCCCGTGA | |
| GTCCCATAGTTGCTGCAGGAGTGGAGCCATGAGCTGCGTCCTGGGTGGTGTCATCCCCT | |
| TGGGGCTGCTGTTCCTGGTCCGCGGATCCCAAGGCTACCTCCTGCCCAACGTCACTCTC | |
| TTAGAGGAGCTGCTCAGCAAATACCAGCACAACGAGTCTCACTCCCGGGTCCGCAGAGC | |
| CATCCCCAGGGAGGACAAGGAGGAGATCCTCATGCTGCACAACAAGCTTCGGGGCCAGG | |
| TGCAGCCTCAGGCCTCCAACATGGAGTACATGACCTGGGATGACGAACTGGAGAAGTCT | |
| GCTGCAGCGTGGGCCAGTCAGTGCATCTGGGAGCACGGGCCCACCGGTCTGCTGGTGTC | |
| CATCGGGCAGAACCTGGGCGCTCACTGGGGCAGGTATCGCTCTCCGGGGTTCCATGTGC | |
| AGTCCTGGTATGACGAGGTGAAGGACTACACCTACCCCTACCCGAGCGAGTGCAACCCC | |
| TGGTGTCCAGAGAGGTGCTCGGGGCCCATGTGCACGCACTACACACAGGTAACTCAGAT | |
| AGTTTGGGCCACCACCAACAAGATCGGTTGTGCTGTGAACACCTGCCGGAAGATGACTG | |
| TCTGGGGAGAAGTTTGGGAGAACGCGGTCTACTTTGTCTGCAATTATTCTCCAAAGAGG | |
| GGGAACTGGATTGGAGAAGCCCCCTACAAGAATGGCCGGCCCTGCTCTGAGTGCCCACC | |
| CAGCTATGGAGGCAGCTGCAGGAACAACTTGTGTTACCGAGAAGAAACCTACACTCCAA | |
| AACCTGAAACGGACGAGATGAATGAGGTGGAAACGGCTCCCATTCCTGAAGAAAACCAT | |
| GTTTGGCTCCAACCGAGGGTGATGAGACCCACCAAGCCCAAGAAAACCTCTGCGGTCAA | |
| CTACATGACCCAAGTCGTCAGATGTGACACCAAGATGAAGGACAGGTGCAAAGGGRCCA | |
| CGTGTAACAGGTACCAGTGCCCAGCAGGCTGCCTGAACCACAAGGCGAAGATCTTTGGA | |
| AGTCTGTTCTATGAAAGCTCGTCTAGCATATGCCGCGCCGCCATCCACTACGGGATCCT | |
| GGATGACAAGGGAGGCCTGGTGGATATCACCAGGAACGGGAAGGTCCCCTTCTTCGTGA | |
| AGTCTGAGAGACACGGCGTGCAGTCCCTCAGCAAATACAAACCTTCCAGCTCATTCATG | |
| GTGTCAAAAGTGAAAGTGCAGGATTTGGACTGCTACACGACCGTTGCTCAGCTGTGCCC | |
| GTTTGAAAAGCCAGCAACTCACTGCCCAAGAATCCATTGTCCGGCACACTGCAAAGACG | |
| AACCTTCCTACTGGGCTCCGGTGTTTGGAACCAACATCTATGCAGATACCTCAAGCATC | |
| TGCAAGACAGCCGTGCACGCGGGAGTCATCAGCAACGAGAGTGGGGGTGACGTGGACGT | |
| GATGCCCGTGGATAAAAAGAAGACCTACGTGGGCTCGCTCAGGAATGGAGTTCAGTCTG | |
| AAAGCCTGGGGACTCCTCGGGATGGAAAGGCCTTCCGGATCTTTGCTGTCAGGCAGTGA | |
| ATTTCCAGCACCAGGGGAGAAGGGGCGTCTTCAGGAGGGCTTCGGGGTTTTGCTTTTAT | |
| TTTTATTTTGTCATTGCGGGGTATATGGAGAGTCAGGAAACTTCCTTTGACTGATGTTC | |
| AGTGTCCATCACTTTGTGGCCTGTGGGTGAGGTGACATCTCATCCCCTCACTGAAGCAA | |
| CAGCATCCCAAGGTGCTCAGCCGGACTCCCTGGTGCCTGATCCTGCTGGGGCCCGGGGG | |
| TCTCCATCTGGACGTCCTCTCTCCTTTAGAGATCTGAGCTGTCTCTTAAAGGGGACAGT | |
| TGCCCAAAATGTTCCTTGCTATGTGTTCTTCTGTTGGTGGAGGAAGTTGATTTCAACCT | |
| CCCTGCCAAAAGAACAAACCATTTGAAGCTCACAATTGTGAAGCATTCACGGCGTCGGA | |
| AGAGGCCTTTTGAGCAAGCGCCAATGAGTTTCAGGAATGAAGTAGAAGGTAGTTATTTA | |
| AAAATAAAAAACACAGTCCGTCCCTACCAATAGAGGAAAATGGTTTTAATGTTTGCTGG | |
| TCAGACAGACAAATGGGCTAGAGTAAGAGGGCTGCGGGTATGAGAGACCCCGGCTCCGC | |
| CCTGGCACGTGTCCTTGCTGGCGGCCCGCCACAGGCCCCCTTCAATGGCCGCATTCAGG | |
| ATGGCTCTATACACAGCAGTGCTGGTTTATGTAAAGTTCAGCAGTCACTTCA | |
| MSCVLGGVIPLGLLFLVRGSQGYLLPNVTLLEELLSKYQHNESHSRVRRAIPREDKEEI | (SEQ ID NO: 23) |
| LMLHNKLRGQVQPQASNNEYMTWDDELEKSAAAWASQCIWEHGPTGLLVSIGQNLGAHW | |
| GRYRSPGFHVQSWYDEVKDYTYPYPSECNPWCPERCSGPMCTHYTQVTQIVWATTNKIG | |
| CAVNTCRKMTVWGEVWENAVYFVCNYSPKRGNWIGEAPYKNGRPCSECPPSYGGSCRNN | |
| LCYREETYTPKPETDEMNEVETAPIPEENHVWLQPRVMRPTKPKKTSAVNYMTQVVRCD | |
| TKMKDRCKGSTCNRYQCPAGCLNHKAKIFGSLFYESSSSICRAAIHYGILDDKGGLVDI | |
| TRNGKVPFFVKSERHGVQSLSKYKPSSSFMVSKVKVQDLDCYTTVAQLCPFEKPATHCP | |
| RIHCPAHCKDEPSYWAPVFGTNIYADTSSICKTAVHAGVISNESGGDVDVMPVDKKKTY | |
| VGSLRNGVQSESLGTPRDGKAFRIFAVRQ | |

The disclosed NOV-4e amino acid sequence has a high level of homology (97% identity, 97% similarity) to a human trypsin inhibitor-like protein (GenBank Accession No: CAB66795), shown in Table 34. As indicated by the “Expect” value, the probability of this alignment occurring by chance alone is 0.0, the lowest probability score.

TABLE 34
Score = 1001 bits (2588), Expect = 0.0
Identities = 488/501 (97%) , Positives = 489/501 (97%), Gaps = 4/501 (0%)

| | | | |
|---|---|---|---|
| NOV4e: 1 | MSCVLGGVIPLGLLFLVRGSQGYLLPNVTXXXXXXSKYQHNESHSRVRRAIPREDKEEIL | 60 | |
| | MSCVLGGVIPLGLLFLV GSQGYLLPNVT SKYQHNESHSRVRRAIPREDKEEIL | | |
| TRYP: 1 | MSCVLGGVIPLGLLFLVCGSQGYLLPNVTLLEELLSKYQHNESHSRVRRAIPREDKEEIL | 60 | |
| NOV4e: 61 | MLHNKLRGQVQPQASNMEYMTWDDELEKSAAAWASQCIWEHGPTGLLVSIGQNLGAHWGR | 120 | |
| | MLHNKLRGQVQPQASNMEYMTWDDELEKSAAAWASQCIWEHGPT LLVSIGQNLGAHWGR | | |
| TRYP: 61 | MLHNKLRGQVQPQASNMEYMTWDDELEKSAAAWASQCIWEHGPTSLLVSIGQNLGAHWGR | 120 | |
| NOV4e: 121 | YRSPGFHVQSWYDEVKDYTYPYPSECNPWCPERCSGPMCTHYTQVTQIVWATTNKIGCAV | 180 | |
| | YRSPGFHVQSWYDEVKDYTYPYPSECNPWCPERCSGPMCTHY TQIVWATTNKIGCAV | | |
| TRYP: 121 | YRSPGFHVQSWYDEVKDYTYPYPSECNPWCPERCSGPMCTHY---TQIVWATTNKIGCAV | 177 | |
| NOV4e: 181 | NTCRKMTVWGEVWENAVYFVCNYSPKRGNWIGEAPYKNGRPCSECPPSYGGSCRNNLCYR | 240 | |
| | NTCRKMTVWGEVWENAVYFVCNYSPK GNWIGEAPYKNGRPCSECPPSYGGSCRNNLCYR | | |
| TRYP: 178 | NTCRKMTVWGEVWENAVYFVCNYSPK-GNWIGEAPYKNGRPCSECPPSYGGSCRNNLCYR | 236 | |
| NOV4e: 241 | EETYTPKPETDEMNEVETAPIPEENHVWLQPRVMRPTKPKKTSAVNYMTQVVRCDTKMKD | 300 | |
| | EETYTPKPETDEMNEVETAPIPEENHVWLQPRVMRPTKPKKTSAVNYMTQVVRCDTKMKD | | |
| TRYP: 237 | EETYTPKPETDEMNEVETAPIPEENHVWLQPRVMRPTKPKKTSAVNYMTQVVRCDTKMKD | 296 | |
| NOV4e: 301 | RCKGSTCNRYQCPAGCLNHKAKIFGSLFYESSSSICRAAIHYGILDDKGGLVDITRNGKV | 360 | |
| | RCKGSTCNRYQCPAGCLNHKAKIFG+LFYESSSSICRAAIHYGILDDKGGLVDITRNKVG | | |
| TRYP: 297 | RCKGSTCNRYQCPAGCLNHKAKIFGTLFYESSSSICRAAIHYGILDDKGGLVDITRNGKV | 356 | |
| NOV4e: 361 | PFFVKSERHGVQSLSKYKPSSSFMVSKVKVQDLDCYTTVAQLCPFEKPATHCPRIHCPAH | 420 | |
| | PFFVKSERHGVQSLSKYKPSSSFMVSKVKVQDLDCYTTVAQLCPFEKPATHCPRIHCPAH | | |
| TRYP: 357 | PFFVKSERHGVQSLSKYKPSSSFMVSKVKVQDLDCYTTVAQLCPFEKPATHCPRIHCPAH | 416 | |
| NOV4e: 421 | CKDEPSYWAPVFGTNIYADTSSICKTAVHAGVISNESGGDVDVMPVDKKKTYVGSLRNGV | 480 | |
| | CKDEPSYWAPVFGTNIYADTSSICKTAVHAGVISNESGGDVDVMPVDKKKTYVGSLRNGV | | |
| TRYP: 417 | CKDEPSYWAPVFGTNIYADTSSICKTAVHAGVISNESGGDVDVMPVDKKKTYVGSLRNGV | 476 | |
| NOV4e: 481 | QSESLGTPRDGKAFRIFAVRQ | 501 | (SEQ ID NO:77) |
| | QSESLGTPRDGKAFRIFAVRQ | | |
| TRYP: 477 | QSESLGTPRDGKAFRIFAVRQ | 497 | (SEQ ID NO:45) |

In addition, SignalPep and PSORT analyses indicate that that NOV-4e is likely located outside of the cell (certainty=0.6950), and is likely to have a cleavable N-terminal signal sequence with a cleavage site between positions 22 and 23: SQG-YL. The predicted molecular weight of NOV-4b is 56412.8 daltons.

Based on the relatedness between NOV-4e and the conserved trypsin inhibitor proteins, the NOV-4e protein is a novel member of the trypsin inhibitor family. NOV-4e provides new diagnostic and therapeutic compositions useful in the treatment of disorders associated with alterations in the expression of members of the trypsin inhibitor protein family. Nucleic acids, polypeptides, antibodies, and other compositions of the present invention are useful in the treatment and diagnosis of a variety of diseases and pathologies, including, by way of nonlimiting example, those involving reproductive disorders, immunological disorders, cancer, and metabolic disorders.

Table 35 shows a sequence alignment between the NOV-4 polypeptides according to the invention and a human trypsin inhibitor-like protein (GenBank Accession No: CAB66795), indicating the homology between the present invention and the trypsin inhibitor family. Moreover, the PROSITE conserved SCP region found in trypsin inhibitors is found in sequences 151-162 of the trypsin inihibitor-like protein shown (shown in bold font).

TABLE 35

| | |
|---|---|
| NOV4e | MSCVLGGVIPLGLLFLVRGSQGYLLPNVTLLEELLSKYQHNESHSRVRRAIPREDKEEIL |
| NOV4a | ------------------------------------------------------------ |
| NOV4b | MSCVLGGVIPLGLLFLVRGSQGYLLPNVTLLEELLSKYQHNESHSRVPRAIPREDKEEIL |
| NOV4d | MSCVLGGVIPLGLLFLVRGSQGYLLPNVTLLEELLSKYQHNESHSRVPRAIPREDKEEIL |
| NOV4c | MSCVLGGVIPLGLLFLVCGSQGYLLPNVTLLEELLSKYQHNESHSRVPRAIPREDKEEIL |
| TRYP | --------------------------------------------ARRKRYISQNDMIAIL |
| NOV4e | MLHNKLRGQVQPQASNMEYMTWDDELEKSAAAWASQCIWEHGPTGLLVSIGQNLGAHWG- |
| NOV4a | ------------------------------------------------------MTNWG- |
| NOV4b | MLHNKLRGQVQPQASNNEYMTWDDELEKSAAAWASQCIWEHGPTGLLVSIGQNLGAHWG- |
| NOV4d | MLHNKLRGQVQPQASNMEYMTWDDELEKSAAAWASQCIWEHGPTSLLVSIGQNLGAHWGR |
| NOV4c | MLHNKLRGQVQPQASNMEYMTWDDELEKSAAAWASQCIWEHGPTGLLVSIGQNLGAHWG- |
| TRYP | DYHNQVRGKVFPPAANMEYMVWDENLAKSAEAWAATCIWDHGPSYLLRFLGQNLSVRTG- |
| .. * | |
| NOV4e | RYRSPGFHVQSWYDEVKDYTYPYPSECNPWCPERCSGPMCTHYTQVTQIVWATTNKIGCA |
| NOV4a | RYRSPGFHVQSWYDEVKDYTYPYPSECNPWCPERCSGPMCTHY---TQIVWATTNKIGCA |
| NOV4b | RYRSPGFHVQSWYDEVKDYTYPYPSECNPWCPERCSGPMCTHY---TQIVWATTNKIGCA |
| NOV4d | RYRSPGFHVQSWYDEVKDYTYPYPSECNPWCPERCSGPMCTHY---TQIVWATTNKIGCA |
| NOV4c | RYRSPGFHVQSWYDEVKDYTYPYPSECNPWCPERCSGPMCTHY---TQIVWATTNKIGCA |
| TRYP | RYRSILQLVKPWYDEVKDYAFPYPQDCNPRCPMRCFGPMCTHY---TQMVWATSNRIGCA |
| | ****////*:.********::***.:*** ** ** *******///**:****:*:**** |
| NOV4e | VNTCRKMTVWGEVWENAVYFVCNYSPKRGNWIGEAPYKNGRPCSECPPSYGGSCRNNLCY |
| NOV4a | VNTCRKMTVWGEVWENAVYFVCNYSPK-GNWIGEAPYKNGRPCSECPPSYGGSCRNNLCY |
| NOV4b | VNTCRKMTVWGEVWENAVYFVCNYSPK-GNWIGEAPYKNGRPCSECPPSYGGSCRNNLCY |
| NOV4d | VNTCRKMTVWGEVWENAVYFVCNYSPK-GNWIGEAPYKNGRPCSECPPSYGGSCRNNLCY |
| NOV4c | VNTCRKMTVWGEVWENAVYFVCNYSPK-GNWIGEAPYKNGRPCSQCPPSYGGSCRNNLCY |
| TRYP | IHTCQNMNVWGSVWRRAVYLVCNYAPK-GNWIGEAPYKVGVPCSSCPPSYGGSCTDNLCF |
| | ::**::*.***.**..***:****.** ********** * ***.********* :***: |
| NOV4e | REETYTPKPETDEMNEVETAPIPEENHVWLQPRVMRPTKPKKTSAVNYMTQVVRCDTKMK |
| NOV4a | REETYTPKPETDEMNEVETAPIPEENHVWLQPRVMRPTKPKKTSAVNYMTQVVRCDTKMK |
| NOV4b | REETYTPKPETDEMNEVETAPIPEENHVWLQPRVMRPTKPKKTSAVNYMTQVVRCDTKMK |
| NOV4d | REETYTPKPETDEMNEVETAPIPEENHVWLQPRVMRPTKPKKTSAVNYMTQVVRCDTKMK |
| NOV4c | REETYTPKPETDEMNEVETAPIPEENHVWLQPRVMRPTKPKKTSSVNYMTQVVLCDTKMK |
| TRYP | ------------------------------------------------------------ |
| NOV4e | DRCKGSTCNRYQCPAGCLNHKAKIFGSLFYESSSSICRAAIHYGILDDKGGLVDITRNGK |
| NOV4a | DRCKGSTCNRYQCPAGCLNHKAKIFGTLFYESSSSICRAAIHYGILDDKGGLVDITRNGK |
| NOV4b | DRCKGSTCNRYQCPAGCLNHKAKIFGSLFYESSSSICRAAIHYGILDDKGGLVDITRNGK |
| NOV4d | DRCKGSTCNRYQCPAGCLNHKAKIFGSLFYESSSSICRAAIHYGILDDKGGLVDITRNGK |
| NOV4c | DRCKGSTCNRYQCPAGCLNHKAKIFGTLFYESSSSICRAAIHYGILDDKGGLVDITRNGK |
| TRYP | ------------------------------------------------------------ |
| NOV4e | VPFFVKSERHGVQSLSKYKPSSSFMVSKVKVQDLDCYTTVAQLCPFEKPATHCPRIHCPA |
| NOV4a | VPFFVKSERHGVQSLSKYKPSSSFMVSKVKVQDLDCYTTVAQLCPFEKPATHCPRIHCPA |
| NOV4b | VPFFVKSERHGVQSLSKYKPSSSFMVSKVKVQDLDCYTTVAQLCPFEKPATHCPRIECPA |
| NOV4d | VPFFVKSERHGVQSLSKYKPSSSFMVSKVKVQDLDCYTTVAQLCPFEKPATHCPRIHCPA |
| NOV4c | VPFFVKSERHGVQSLSKYKPSSSFMVSKVKVQDLDCYTTVAQLCPFEKPATHCPRIHCPA |
| TRYP | ------------------------------------------------------------ |
| NOV4e | HCKDEPSYWAPVFGTNIYADTSSICKTAVHAGVISNESGGDVDVMPVDKKKTYVGSLRNG |
| NOV4a | HCKDEPSYWAPVFGTNIYADTSSICKTAVHAGVISNESGGDVDVMPVDKKKTYVGSLRNG |
| NOV4b | HCKDEPSYWAPVFGTNIYADTSSICKTAVHAGVISNESGGDVDVMPVDKKKTYVGSLRNG |
| NOV4d | HCKDEPSYWAPVFGTNIYADTSSICKTAVHAGVISNESGGDVDVMPVDKKKTYVGSLRNG |
| NOV4c | HCKDEPSYWAPVFGTNIYADTSSICKTAVHAGVISNESGGDVDVMPVDKKKTYT------ |
| TRYP | ------------------------------------------------------------ |
| NOV4e | VQSESLGTPRDGKAFRIFAVRQ (SEQ ID NO:23) |
| NOV4a | VQSESLGTPRDGKAFRIFAVRQ (SEQ ID NO:15) |
| NOV4b | VQSESLGTPRDGKAFRIFAVRQ (SEQ ID NO:17) |
| NOV4d | VQSESLGTPRDGKAFRIFAVRQ (SEQ ID NO:21) |
| NOV4c | -------CPAAARAL------- (SEQ ID NO:19) |
| TRYP | ---------------------- (SEQ ID NO:46) |

Consensus key
* - single, fully conserved residue
: - conservation of strong groups
. - conservation of weak groups
- no consensus

The expression pattern, and protein similarity information for NOV-4 suggests that the human trypsin inhibitor-like proteins described in this invention may function as a trypsin inhibitor. Therefore, the nucleic acid and protein of the invention are useful in potential therapeutic applications implicated, for example but not limited to, in allergies and infectious diseases, in cancer, in metabolic disorders like obesity, hypertension and diabetes, and other diseases and disorders.

Homology to antigenic secreted and membrane proteins suggests that antibodies directed against the novel genes may be useful in treatment and prevention of allergic reactions and infectious diseases. Expression in pituitary and adrenal gland suggests therapeutic applications in metabolic disorders like obesity, hypertension and diabetes. Similarity to a brain tumor overexpressed trypsin inhibitor suggests that the splice variants of 10093872 may be involved in the pathogenesis of these cancers. Hence it could be useful as a cancer diagnostic marker or as a target for small molecule trypsin inhibitors in cancer treatment.

Potential therapeutic uses for the invention(s) include, for example, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the human trypsin inhibitor-like protein may be useful in gene therapy, and the human trypsin inhibitor-like protein may be useful when administered to a subject in need thereof. By way of non-limiting example, the compositions of the present invention will have efficacy for treatment of patients suffering from, for example, but not limited to, in allergies and infectious diseases, in caner, in metabolic disorders like obesity, hypertension and diabetes, and other diseases and disorders. The novel nucleic acid encoding the human trypsin inhibitor-like protein, and the human trypsin inhibitor-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV-X Nucleic Acids

The nucleic acids of the invention include those that encode a NOV-X polypeptide or protein. As used herein, the terms polypeptide and protein are interchangeable.

In some embodiments, a NOV-X nucleic acid encodes a mature NOV-X polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the NOV-X nucleic acids is the nucleic acid whose sequence is provided in SEQ ID NO: 1, 3, 6, 8, 10, 12, 14, 16, 18, 20, 22, or 57, or a fragment thereof. Additionally, the invention includes mutant or variant nucleic acids of SEQ ID NO: 1, 3, 6, 8, 10, 12, 14, 16, 18, 20, 22, or 57, or a fragment thereof, any of whose bases may be changed from the corresponding bases shown in SEQ ID NO: 1, 3, 6, 8, 10, 12, 14, 16, 18, 20, 22, or 57, while still encoding a protein that maintains at least one of its NOV-X-like activities and physiological functions (i.e., modulating angiogenesis, neuronal development). The invention further includes the complement of the nucleic acid sequence of SEQ ID NO: 1, 3, 6, 8, 10, 12, 14, 16, 18, 20, 22, or 57, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode NOV-X proteins or biologically active portions thereof. Also included are nucleic acid fragments sufficient for use as hybridization probes to identify NOV-X-encoding nucleic acids (e.g., NOV-X mRNA) and fragments for use as polymerase chain reaction (PCR) primers for the amplification or mutation of NOV-X nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NOV-X nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, 3, 6, 8, 10, 12, 14, 16, 18, 20, 22, or 57, or a complement of any of this nucleotide sequence, can be isolated using standard molecular biology techniques and the sequence information provided herein. Using all or a portion of the nucleic acid sequence of SEQ ID NO: 1, 3, 6, 8, 10, 12, 14, 16, 18, 20, 22, or 57, as a hybridization probe, NOV-X nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOV-X nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO: 1, 3, 6, 8, 10, 12, 14, 16, 18, 20, 22, or 57, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO: 1, 3, 6, 8, 10, 12, 14, 16, 18, 20, 22, or 57, or a portion of this nucleotide sequence. A nucleic acid molecule that is complementary to the nucleotide sequence shown in SEQ ID NO: 1, 3, 6, 8, 10, 12, 14, 16, 18, 20, 22, or 57 is one that is sufficiently complementary to the nucleotide sequence shown in SEQ ID NO: 1, 3, 6, 8, 10, 12, 14, 16, 18, 20, 22, or 57 that it can hydrogen bond with little or no mismatches to the nucleotide sequence shown in SEQ ID NO: 1, 3, 6, 8, 10, 12, 14, 16, 18, 20, 22, or 57, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, 3, 6, 8, 10, 12, 14, 16, 18, 20, 22, or 57, e.g., a fragment that can be used as a probe or primer, or a fragment encoding a biologically active portion of NOV-X. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a NOV-X polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a NOV-X polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human NOV-X protein. Homologous nucleic acid sequences include those nucleic acid sequences that encode conservative amino acid substitutions (see below) in SEQ ID NO: 2, 4, 5, 7, 9, 11, 13, 15, 17, 19, 21, or 23, as well as a polypeptide having NOV-X activity. Biological activities of the NOV-X proteins are described below. A homologous amino acid sequence does not encode the amino acid sequence of a human NOV-X polypeptide.

The nucleotide sequence determined from the cloning of the human NOV-X gene allows for the generation of probes and primers designed for use in identifying and/or cloning NOV-X homologues in other cell types, e.g., from other tissues, as well as NOV-X homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, 3, 6, 8, 10, 12, 14, 16, 18, 20, 22, or 57; or an anti-sense strand nucleotide sequence of SEQ ID NO: 1, 3, 6, 8, 10, 12, 14, 16, 18, 20, 22, or 57; or of a naturally occurring mutant of SEQ IDNO: 1, 3, 6, 8, 10, 12, 14, 16, 18, 20, 22, or 57.

Probes based on the human NOV-X nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a NOV-X protein, such as by measuring a level of a NOV-X-encoding nucleic acid in a sample of cells from a subject e.g., detecting NOV-X mRNA levels or determining whether a genomic NOV-X gene has been mutated or deleted.

A “polypeptide having a biologically active portion of NOV-X” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of NOV-X” can be prepared by isolating aportion of SEQ ID NO: 1, 3, 6, 8, 10, 12, 14, 16, 18, 20, 22, or 57 that encodes a polypeptide having a NOV-X biological activity (biological activities of the NOV-X proteins are described below), expressing the encoded portion of NOV-X protein (e.g., by recombinant expression in vitro) and assessing the activity of the encoded portion of NOV-X. For example, a nucleic acid fragment encoding a biologically active portion of NOV-X can optionally include an ATP-binding domain. In another embodiment, a nucleic acid fragment encoding a biologically active portion of NOV-X includes one or more regions.

NOV-X Variants

The invention further encompasses nucleic acid molecules that differ from the nucleotide sequences shown in SEQ ID NO: 1, 3, 6, 8, 10, 12, 14, 16, 18, 20, 22, or 57 due to the degeneracy of the genetic code. These nucleic acids thus encode the same NOV-X protein as that encoded by the nucleotide sequence shown in SEQ ID NO: 1, 3, 6, 8, 10, 12, 14, 16, 18, 20, 22, or 57 e.g., the polypeptide of SEQ ID NO: 2, 4, 5, 7, 9, 11, 13, 15, 17, 19, 21, or 23. In another embodiment, an isolated nucleic acid molecule of the invention has a nucleotide sequence encoding a protein having an amino acid sequence shown in SEQ ID NO: 2, 4, 5, 7, 9, 11, 13,15,17, 19,21, or 23.

In addition to the human NOV-X nucleotide sequence shown in SEQ ID NO: 1, 3, 6, 8, 10, 12, 14, 16, 18, 20, 22, or 57, it will be appreciated by those skilled in the art that DNA sequence polymorphisms that lead to changes in the amino acid sequences of NOV-X may exist within a population (e.g., the human population). Such genetic polymorphism in the NOV-X gene may exist among individuals within a population due to natural allelic variation. As used herein, the terms “gene” and “recombinant gene” refer to nucleic acid molecules comprising an open reading frame encoding a NOV-X protein, preferably a mammalian NOV-X protein. Such natural allelic variations can typically result in 1-5% variance in the nucleotide sequence of the NOV-X gene. Any and all such nucleotide variations and resulting amino acid polymorphisms in NOV-X that are the result of natural allelic variation and that do not alter the functional activity of NOV-X are intended to be within the scope of the invention.

Moreover, nucleic acid molecules encoding NOV-X proteins from other species, and thus that have a nucleotide sequence that differs from the human sequence of SEQ ID NO: 1, 3, 6, 8, 10, 12, 14, 16, 18, 20, 22, or 57 are intended to be within the scope of the invention. Nucleic acid molecules corresponding to natural allelic variants and homologues of the NOV-X cDNAs of the invention can be isolated based on their homology to the human NOV-X nucleic acids disclosed herein using the human cDNAs, or a portion thereof, as a hybridization probe according to standard hybridization techniques under stringent hybridization conditions. For example, a soluble human NOV-X cDNA can be isolated based on its homology to human membrane-bound NOV-X. Likewise, a membrane-bound human NOV-X cDNA can be isolated based on its homology to soluble human NOV-X.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 3, 6, 8, 10, 12, 14, 16, 18, 20, 22, or 57. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOV-X proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO: 1, 3, 6, 8, 10, 12, 14, 16, 18, 20, 22, or 57 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 3, 6, 8, 10, 12, 14, 16, 18, 20, 22, or 57, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, N.Y., and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, 3, 6, 8, 10, 12, 14, 16, 18, 20, 22, or 57, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, N.Y., and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the NOV-X sequence that may exist in the population, the skilled artisan will further appreciate that changes can be introduced by mutation into the nucleotide sequence of SEQ ID NO: 1, 3, 6, 8, 10, 12, 14, 16, 18, 20, 22, or 57, thereby leading to changes in the amino acid sequence of the encoded NOV-X protein, without altering the functional ability of the NOV-X protein. For example, nucleotide substitutions leading to amino acid substitutions at “non-essential” amino acid residues can be made in the sequence of SEQ ID NO: 1, 3, 6, 8, 10, 12, 14, 16, 18, 20, 22, or 57. A “non-essential” amino acid residue is a residue that can be altered from the wild-type sequence of NOV-X without altering the biological activity, whereas an “essential” amino acid residue is required for biological activity. For example, amino acid residues that are conserved among the NOV-X proteins of the present invention, are predicted to be particularly unamenable to alteration.

Another aspect of the invention pertains to nucleic acid molecules encoding NOV-X proteins that contain changes in amino acid residues that are not essential for activity. Such NOV-X proteins differ in amino acid sequence from SEQ ID NO: 2, 4, 5, 7, 9, 11, 13, 15, 17, 19, 21, or 23, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO: 2, 4, 6, or 8. Preferably, the protein encoded by the nucleic acid is at least about 80% homologous to SEQ ID NO: 2, 4, 5, 7, 9, 11, 13, 15, 17, 19, 21, or 23, more preferably at least about 90%, 95%, 98%, and most preferably at least about 99% homologous to SEQ ID NO: 2, 4, 5, 7, 9, 11, 13, 15, 17, 19, 21, or 23.

An isolated nucleic acid molecule encoding a NOV-X protein homologous to the protein of can be created by introducing one or more nucleotide substitutions, additions or deletions into the nucleotide sequence of SEQ ID NO: 1, 3, 6, 8, 10, 12, 14, 16, 18, 20, 22, or 57, such that one or more amino acid substitutions, additions or deletions are introduced into the encoded protein.

Mutations can be introduced into the nucleotide sequence of SEQ ID NO: 1, 3, 6, 8, 10, 12, 14, 16, 18, 20, 22, or 57 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in NOV-X is replaced with another amino acid residue from the same side chain family. Alternatively, in another embodiment, mutations can be introduced randomly along all or part of a NOV-X coding sequence, such as by saturation mutagenesis, and the resultant mutants can be screened for NOV-X biological activity to identify mutants that retain activity. Following mutagenesis of SEQ ID NO: 1, 3, 6, 8, 10, 12, 14, 16, 18, 20, 22, or 57 the encoded protein can be expressed by any recombinant technology known in the art and the activity of the protein can be determined.

In one embodiment, a mutant NOV-X protein can be assayed for (1) the ability to form protein:protein interactions with other NOV-X proteins, other cell-surface proteins, or biologically active portions thereof, (2) complex formation between a mutant NOV-X protein and a NOV-X receptor; (3) the ability of a mutant NOV-X protein to bind to an intracellular target protein or biologically active portion thereof, (e.g., avidin proteins); (4) the ability to bind NOV-X protein; or (5) the ability to specifically bind an anti-NOV-X protein antibody.

Antisense NOV-X Nucleic Acids

Another aspect of the invention pertains to isolated antisense nucleic acid molecules that are hybridizable to or complementary to the nucleic acid molecule comprising the nucleotide sequence of SEQ ID NO: 1, 3, 6, 8, 10, 12, 14, 16, 18, 20, 22, or 57, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire NOV-X coding strand, or to only a portion thereof. Nucleic acid molecules encoding fragments, homologs, derivatives and analogs of a NOV-X protein of SEQ ID NO: 2, 4, 5, 7, 9, 11, 13, 15, 17, 19, 21, or 23 or antisense nucleic acids complementary to a NOV-X nucleic acid sequence of SEQ ID NO: 1, 3, 6, 8, 10, 12, 14, 16, 18, 20, 22, or 57 are additionally provided.

In one embodiment, an antisense nucleic acid molecule is antisense to a “coding region” of the coding strand of a nucleotide sequence encoding NOV-X. The term “coding region” refers to the region of the nucleotide sequence comprising codons which are translated into amino acid residues (e.g., the protein coding region of human NOV-X corresponds to SEQ ID NO: 2, 4, 5, 7, 9, 11, 13, 15, 17, 19, 21, or 23). In another embodiment, the antisense nucleic acid molecule is antisense to a “noncoding region” of the coding strand of a nucleotide sequence encoding NOV-X. The term “noncoding region” refers to 5′ and 3′ sequences which flank the coding region that are not translated into amino acids (i.e., also referred to as 5′ and 3′ untranslated regions).

Given the coding strand sequences encoding NOV-X disclosed herein (e.g., SEQ ID NO: 1, 3, 6, 8, 10, 12, 14, 16, 18, 20, 22, or 57), antisense nucleic acids of the invention can be designed according to the rules of Watson and Crick or Hoogsteen base pairing. The antisense nucleic acid molecule can be complementary to the entire coding region of NOV-X mRNA, but more preferably is an oligonucleotide that is antisense to only a portion of the coding or noncoding region of NOV-X mRNA. For example, the antisense oligonucleotide can be complementary to the region surrounding the translation start site of NOV-X mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOV-X protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an -anomeric nucleic acid molecule. An -anomeric nucleic acid molecule forms specific double-stranded hybrids with complementary RNA in which, contrary to the usual -units, the strands run parallel to each other (Gaultier et al. (1987) Nucleic acids Res 15: 6625-6641). The antisense nucleic acid molecule can also comprise a 2′-o-methylribonucleotide (Inoue et al. (1987) Nucleic acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

NOV-X Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave NOV-X mRNA transcripts to thereby inhibit translation of NOV-X mRNA. A ribozyme having specificity for a NOV-X-encoding nucleic acid can be designed based upon the nucleotide sequence of a NOV-X DNA disclosed herein (i.e., SEQ ID NO: 1, 3, 6, 8, 10, 12, 14, 16, 18, 20, 22, or 57). For example, a derivative of a Tetrahymena L-19 IVS RNA can be constructed in which the nucleotide sequence of the active site is complementary to the nucleotide sequence to be cleaved in a NOV-X-encoding mRNA. See, e.g., Cech et al. U.S. Pat. No. 4,987,071; and Cech et al. U.S. Pat. No. 5,116,742. Alternatively, NOV-X mRNA can be used to select a catalytic RNA having a specific ribonuclease activity from a pool of RNA molecules. See, e.g., Bartel et al., (1993) Science 261:1411-1418.

Alternatively, NOV-X gene expression can be inhibited by targeting nucleotide sequences complementary to the regulatory region of the NOV-X (e.g., the NOV-X promoter and/or enhancers) to form triple helical structures that prevent transcription of the NOV-X gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of NOV-X can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of NOV-X can be used in therapeutic and diagnostic applications. For example, PNAs can be used as antisense or antigene agents for sequence-specific modulation of gene expression by, e.g., inducing transcription or translation arrest or inhibiting replication. PNAs of NOV-X can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of NOV-X can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOV-X can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

NOV-X Polypeptides

A NOV-X polypeptide of the invention includes the NOV-X-like protein whose sequence is provided in SEQ ID NO: 2, 4, 5, 7, 9, 11, 13, 15, 17, 19, 21, or 23. The invention also includes a mutant or variant protein any of whose residues may be changed from the corresponding residue shown in SEQ ID NO: 2, 4, 5, 7, 9, 11, 13, 15, 17, 19, 21, or 23 while still encoding a protein that maintains its NOV-X-like activities and physiological functions, or a functional fragment thereof. In some embodiments, up to 20% or more of the residues may be so changed in the mutant or variant protein. In some embodiments, the NOV-X polypeptide according to the invention is a mature polypeptide.

In general, a NOV-X-like variant that preserves NOV-X-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOV-X proteins, and biologically active portions thereof, or derivatives, fragments, analogs or homologs thereof. Also provided are polypeptide fragments suitable for use as immunogens to raise anti-NOV-X antibodies. In one embodiment, native NOV-X proteins can be isolated from cells or tissue sources by an appropriate purification scheme using standard protein purification techniques. In another embodiment, NOV-X proteins are produced by recombinant DNA techniques. Alternative to recombinant expression, a NOV-X protein or polypeptide can be synthesized chemically using standard peptide synthesis techniques.

An “isolated” or “purified” protein or biologically active portion thereof is substantially free of cellular material or other contaminating proteins from the cell or tissue source from which the NOV-X protein is derived, or substantially free from chemical precursors or other chemicals when chemically synthesized. The language “substantially free of cellular material” includes preparations of NOV-X protein in which the protein is separated from cellular components of the cells from which it is isolated or recombinantly produced.

In one embodiment, the language “substantially free of cellular material” includes preparations of NOV-X protein having less than about 30% (by dry weight) of non-NOV-X protein (also referred to herein as a “contaminating protein”), more preferably less than about 20% of non-NOV-X protein, still more preferably less than about 10% of non-NOV-X protein, and most preferably less than about 5% non-NOV-X protein. When the NOV-X protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of NOV-X protein in which the protein is separated from chemical precursors or other chemicals that are involved in the synthesis of the protein. In one embodiment, the language “substantially free of chemical precursors or other chemicals” includes preparations of NOV-X protein having less than about 30% (by dry weight) of chemical precursors or non-NOV-X chemicals, more preferably less than about 20% chemical precursors or non-NOV-X chemicals, still more preferably less than about 10% chemical precursors or non-NOV-X chemicals, and most preferably less than about 5% chemical precursors or non-NOV-X chemicals.

Biologically active portions of a NOV-X protein include peptides comprising amino acid sequences sufficiently homologous to or derived from the amino acid sequence of the NOV-X protein, e.g., the amino acid sequence shown in SEQ ID NO: 2, 4, 5, 7, 9, 11, 13, 15, 17, 19, 21, or 23 that include fewer amino acids than the full length NOV-X proteins, and exhibit at least one activity of a NOV-X protein. Typically, biologically active portions comprise a domain or motif with at least one activity of the NOV-X protein. A biologically active portion of a NOV-X protein can be a polypeptide which is, for example, 10, 25, 50, 100 or more amino acids in length.

A biologically active portion of a NOV-X protein of the present invention may contain at least one of the above-identified domains conserved between the NOV-X proteins, e.g. TSR modules. Moreover, other biologically active portions, in which other regions of the protein are deleted, can be prepared by recombinant techniques and evaluated for one or more of the functional activities of a native NOV-X protein.

In an embodiment, the NOV-X protein has an amino acid sequence shown in SEQ ID NO: 2, 4, 5, 7, 9, 11, 13, 15, 17, 19, 21, or 23. In other embodiments, the NOV-X protein is substantially homologous to SEQ ID NO: 2, 4, 5, 7, 9, 11, 13, 15, 17, 19, 21, or 23 and retains the functional activity of the protein of SEQ ID NO: 2, 4, 5, 7, 9, 11, 13, 15, 17, 19, 21, or 23 yet differs in amino acid sequence due to natural allelic variation or mutagenesis, as described in detail below. Accordingly, in another embodiment, the NOV-X protein is a protein that comprises an amino acid sequence at least about 45% homologous to the amino acid sequence of SEQ ID NO: 2, 4, 5, 7, 9, 11, 13, 15, 17, 19, 2 1, or 23 and retains the functional activity of the NOV-X proteins of SEQ ID NO: 2, 4, 5, 7, 9, 11, 13, 15, 17, 19, 21, or 23.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 3, 6, 8, 10, 12, 14, 16, 18, 20, 22, or 57.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides NOV-X chimeric or fusion proteins. As used herein, a NOV-X “chimeric protein” or “fusion protein” comprises a NOV-X polypeptide operatively linked to a non-NOV-X polypeptide. An “NOV-X polypeptide” refers to a polypeptide having an amino acid sequence corresponding to NOV-X, whereas a “non-NOV-X polypeptide” refers to a polypeptide having an amino acid sequence corresponding to a protein that is not substantially homologous to the NOV-X protein, e.g., a protein that is different from the NOV-X protein and that is derived from the same or a different organism. Within a NOV-X fusion protein the NOV-X polypeptide can correspond to all or a portion of a NOV-X protein. In one embodiment, a NOV-X fusion protein comprises at least one biologically active portion of a NOV-X protein. In another embodiment, a NOV-X fusion protein comprises at least two biologically active portions of a NOV-X protein. Within the fusion protein, the term “operatively linked” is intended to indicate that the NOV-X polypeptide and the non-NOV-X polypeptide are fused in-frame to each other. The non-NOV-X polypeptide can be fused to the N-terminus or C-terminus of the NOV-X polypeptide.

For example, in one embodiment a NOV-X fusion protein comprises a NOV-X polypeptide operably linked to the extracellular domain of a second protein. Such fusion proteins can be further utilized in screening assays for compounds that modulate NOV-X activity (such assays are described in detail below).

In another embodiment, the fusion protein is a GST-NOV-X fusion protein in which the NOV-X sequences are fused to the C-terminus of the GST (i.e., glutathione S-transferase) sequences. Such fusion proteins can facilitate the purification of recombinant NOV-X.

In another embodiment, the fusion protein is a NOV-X-immunoglobulin fusion protein in which the NOV-X sequences comprising one or more domains are fused to sequences derived from a member of the immunoglobulin protein family. The NOV-X-immunoglobulin fusion proteins of the invention can be incorporated into pharmaceutical compositions and administered to a subject to inhibit an interaction between a NOV-X ligand and a NOV-X protein on the surface of a cell, to thereby suppress NOV-X-mediated signal transduction in vivo. In one nonlirniting example, a contemplated NOV-X ligand of the invention is the NOV-X receptor. The NOV-X-immunoglobulin fusion proteins can be used to affect the bioavailability of a NOV-X cognate ligand. Inhibition of the NOV-X ligand/NOV-X interaction may be useful therapeutically for both the treatment of proliferative and differentiative disorders, e,g., cancer as well as modulating (e.g., promoting or inhibiting) cell survival, as well as acute and chronic inflammatory disorders and hyperplastic wound healing, e.g. hypertrophic scars and keloids. Moreover, the NOV-X-immunoglobulin fusion proteins of the invention can be used as immunogens to produce anti-NOV-X antibodies in a subject, to purify NOV-X ligands, and in screening assays to identify molecules that inhibit the interaction of NOV-X with a NOV-X ligand.

A NOV-X chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOV-X-encoding nucleic acid can be cloned into such an expression vector such that the fusion moiety is linked in-frame to the NOV-X protein.

NOV-X Agonists and Antagonists

The present invention also pertains to variants of the NOV-X proteins that function as either NOV-X agonists (mimetics) or as NOV-X antagonists. Variants of the NOV-X protein can be generated by mutagenesis, e.g., discrete point mutation or truncation of the NOV-X protein. An agonist of the NOV-X protein can retain substantially the same, or a subset of, the biological activities of the naturally occurring form of the NOV-X protein. An antagonist of the NOV-X protein can inhibit one or more of the activities of the naturally occurring form of the NOV-X protein by, for example, competitively binding to a downstream or upstream member of a cellular signaling cascade which includes the NOV-X protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOV-X proteins.

Variants of the NOV-X protein that function as either NOV-X agonists (mimetics) or as NOV-X antagonists can be identified by screening combinatorial libraries of mutants, e.g., truncation mutants, of the NOV-X protein for NOV-X protein agonist or antagonist activity. In one embodiment, a variegated library of NOV-X variants is generated by combinatorial mutagenesis at the nucleic acid level and is encoded by a variegated gene library. A variegated library of NOV-X variants can be produced by, for example, enzymatically ligating a mixture of synthetic oligonucleotides into gene sequences such that a degenerate set of potential NOV-X sequences is expressible as individual polypeptides, or alternatively, as a set of larger fusion proteins (e.g., for phage display) containing the set of NOV-X sequences therein. There are a variety of methods which can be used to produce libraries of potential NOV-X variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOV-X sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the NOV-X protein coding sequence can be used to generate a variegated population of NOV-X fragments for screening and subsequent selection of variants of a NOV-X protein. In one embodiment, a library of coding sequence fragments can be generated by treating a double stranded PCR fragment of a NOV-X coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the NOV-X protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOV-X proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify NOV-X variants (Arkin and Yourvan (1992) PNAS 89:7811-7815; Delgrave et al. (1993) Protein Engineering 6:327-331).

NOV-X Antibodies

Also included in the invention are antibodies to NOV-X proteins, or fragments of NOV-X proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOV-X-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 2, 4, 6, 8 ,10, 12, 14, 16, 18, or 20, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOV-X-related protein that is located on the surface of the protein, e.g., a hydrophilic region. A hydrophobicity analysis of the human NOV-X-related protein sequence will indicate which regions of a NOV-X-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin.

Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach 5 is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. Nature 368 856-859 (1994)); Morrison (Nature 368, 812-13 (1994)); Fishwild et al, (Nature Biotechnology 1, 845-51 (1996)); Neuberger (Nature Biotechnology 14, 826 (1996)); and

Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the fill complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers. Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (Fc R), such as Fe RI (CD64), Fe RII (CD32) and Fc RIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tunor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

NOV-X Recombinant Expression Vectors and Host Cells

Another aspect of the invention pertains to vectors, preferably expression vectors, containing a nucleic acid encoding a NOV-X protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOV-X proteins, mutant forms of NOV-X proteins, fusion proteins, etc.).

The recombinant expression vectors of the invention can be designed for expression of NOV-X proteins in prokaryotic or eukaryotic cells. For example, NOV-X proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 1 id (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOV-X expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOV-X can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the -fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOV-X mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOV-X protein can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOV-X or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOV-X protein. Accordingly, the invention further provides methods for producing NOV-X protein using the host cells of the invention. In one embodiment, the method comprises culturing the host cell of invention (into which a recombinant expression vector encoding NOV-X protein has been introduced) in a suitable medium such that NOV-X protein is produced. In another embodiment, the method further comprises isolating NOV-X protein from the medium or the host cell.

Transgenic NOV-X Animals

The host cells of the invention can also be used to produce non-human transgenic animals. For example, in one embodiment, a host cell of the invention is a fertilized oocyte or an embryonic stem cell into which NOV-X protein-coding sequences have been introduced. Such host cells can then be used to create non-human transgenic animals in which exogenous NOV-X sequences have been introduced into their genome or homologous recombinant animals in which endogenous NOV-X sequences have been altered. Such animals are useful for studying the function and/or activity of NOV-X protein and for identifying and/or evaluating modulators of NOV-X protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOV-X gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOV-X-encoding nucleic acid into the male pronuclei of a fertilized oocyte (e.g., by microinjection, retroviral infection) and allowing the oocyte to develop in a pseudopregnant female foster animal. Sequences including SEQ ID NO: 1, 3, 6, 8, 10, 12, 14, 16, 18, 20, 22, or 57 can be introduced as a transgene into the genome of a non-human animal. Alternatively, a non-human homologue of the human NOV-X gene, such as a mouse NOV-X gene, can be isolated based on hybridization to the human NOV-X cDNA (described further supra) and used as a transgene. Intronic sequences and polyadenylation signals can also be included in the transgene to increase the efficiency of expression of the transgene. A tissue-specific regulatory sequence(s) can be operably-linked to the NOV-X transgene to direct expression of NOV-X protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOV-X transgene in its genome and/or expression of NOV-X mRNA in tissues or cells of the animals. A transgenic founder animal can then be used to breed additional animals carrying the transgene. Moreover, transgenic animals carrying a transgene-encoding NOV-X protein can further be bred to other transgenic animals carrying other transgenes.

To create a homologous recombinant animal, a vector is prepared which contains at least a portion of a NOV-X gene into which a deletion, addition or substitution has been introduced to thereby alter, e.g., functionally disrupt, the NOV-X gene. The NOV-X gene can be a human gene (e.g., the DNA of SEQ ID NO: 1, 3, 6, 8, 10, 12, 14, 16, 18, 20, 22, or 57), but more preferably, is a non-human homologue of a human NOV-X gene. For example, a mouse homologue of human NOV-X gene of SEQ ID NO: 1, 3, 6, 8, 10, 12, 14, 16, 18, 20, 22, or 57 can be used to construct a homologous recombination vector suitable for altering an endogenous NOV-X gene in the mouse genome. In one embodiment, the vector is designed such that, upon homologous recombination, the endogenous NOV-X gene is functionally disrupted (i.e., no longer encodes a functional protein; also referred to as a “knock out” vector).

Alternatively, the vector can be designed such that, upon homologous recombination, the endogenous NOV-X gene is mutated or otherwise altered but still encodes functional protein (e.g., the upstream regulatory region can be altered to thereby alter the expression of the endogenous NOV-X protein). In the homologous recombination vector, the altered portion of the NOV-X gene is flanked at its 5′- and 3′-termini by additional nucleic acid of the NOV-X gene to allow for homologous recombination to occur between the exogenous NOV-X gene carried by the vector and an endogenous NOV-X gene in an embryonic stem cell. The additional flanking NOV-X nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOV-X gene has homologously-recombined with the endogenous NOV-X gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOV-X nucleic acid molecules, NOV-X proteins, and anti-NOV-X antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL^{TM} (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOV-X protein or anti-NOV-X antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., 1993 Proc. Natl. Acad. Sci. USA, 90: 7889-7893. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Screening and Detection Methods

The isolated nucleic acid molecules of the invention can be used to express NOV-X protein (e.g., via a recombinant expression vector in a host cell in gene therapy applications), to detect NOV-X mRNA (e.g., in a biological sample) or a genetic lesion in a NOV-X gene, and to modulate NOV-X activity, as described further, below. In addition, the NOV-X proteins can be used to screen drugs or compounds that modulate the NOV-X protein activity or expression as well as to treat disorders characterized by insufficient or excessive production of NOV-X protein or production of NOV-X protein forms that have decreased or aberrant activity compared to NOV-X wild-type protein. In addition, the anti-NOV-X antibodies of the invention can be used to detect and isolate NOV-X proteins and modulate NOV-X activity. For example, NOV-X activity includes growth and differentiation, antibody production, and tumor growth.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOV-X proteins or have a stimulatory or inhibitory effect on, e.g., NOV-X protein expression or NOV-X protein activity. The invention also includes compounds identified in the screening assays described herein.

In one embodiment, the invention provides assays for screening candidate or test compounds which bind to or modulate the activity of the membrane-bound form of a NOV-X protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOV-X protein, or a biologically-active portion thereof, on the cell surface is contacted with a test compound and the ability of the test compound to bind to a NOV-X protein determined. The cell, for example, can be of mammalian origin or a yeast cell. Determining the ability of the test compound to bind to the NOV-X protein can be accomplished, for example, by coupling the test compound with a radioisotope or enzymatic label such that binding of the test compound to the NOV-X protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOV-X protein, or a biologically-active portion thereof, on the cell surface with a known compound which binds NOV-X to form an assay mixture, contacting the assay mixture with a test compound, and determining the ability of the test compound to interact with a NOV-X protein, wherein determining the ability of the test compound to interact with a NOV-X protein comprises determining the ability of the test compound to preferentially bind to NOV-X protein or a biologically-active portion thereof as compared to the known compound.

In another embodiment, an assay is a cell-based assay comprising contacting a cell expressing a membrane-bound form of NOV-X protein, or a biologically-active portion thereof, on the cell surface with a test compound and determining the ability of the test compound to modulate (e.g., stimulate or inhibit) the activity of the NOV-X protein or biologically-active portion thereof. Determining the ability of the test compound to modulate the activity of NOV-X or a biologically-active portion thereof can be accomplished, for example, by determining the ability of the NOV-X protein to bind to or interact with a NOV-X target molecule. As used herein, a “target molecule” is a molecule with which a NOV-X protein binds or interacts in nature, for example, a molecule on the surface of a cell which expresses a NOV-X interacting protein, a molecule on the surface of a second cell, a molecule in the extracellular milieu, a molecule associated with the internal surface of a cell membrane or a cytoplasmic molecule. A NOV-X target molecule can be a non-NOV-X molecule or a NOV-X protein or polypeptide of the invention In one embodiment, a NOV-X target molecule is a component of a signal transduction pathway that facilitates transduction of an extracellular signal (e.g. a signal generated by binding of a compound to a membrane-bound NOV-X molecule) through the cell membrane and into the cell. The target, for example, can be a second intercellular protein that has catalytic activity or a protein that facilitates the association of downstream signaling molecules with NOV-X.

Determining the ability of the NOV-X protein to bind to or interact with a NOV-X target molecule can be accomplished by one of the methods described above for determining direct binding.

In one embodiment, determining the ability of the NOV-X protein to bind to or interact with a NOV-X target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOV-X-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOV-X protein or biologically-active portion thereof with a test compound and determining the ability of the test compound to bind to the NOV-X protein or biologically-active portion thereof. Binding of the test compound to the NOV-X protein can be determined either directly or indirectly as described above.

In one such embodiment, the assay comprises contacting the NOV-X protein or biologically-active portion thereof with a known compound which binds NOV-X to form an assay mixture, contacting the assay mixture with a test compound, and determining the ability of the test compound to interact with a NOV-X protein, wherein determining the ability of the test compound to interact with a NOV-X protein comprises determining the ability of the test compound to preferentially bind to NOV-X or biologically-active portion thereof as compared to the known compound.

In still another embodiment, an assay is a cell-free assay comprising contacting NOV-X Protein or biologically-active portion thereof with a test compound and determining the ability of he test compound to modulate (e.g. stimulate or inhibit) the activity of the NOV-X protein or Bologically-active portion thereof. Determining the ability of the test compound to modulate the activity of NOV-X can be accomplished, for example, by determining the ability of the NOV-X protein to bind to a NOV-X target molecule by one of the methods described above for determining direct binding. In an alternative embodiment, determining the ability of the test compound to modulate the activity of NOV-X protein can be accomplished by determining the ability of the NOV-X protein further modulate a NOV-X target molecule. For example, the catalytic/enzymatic activity of the target molecule on an appropriate substrate can be determined as described above.

In yet another embodiment, the cell-free assay comprises contacting the NOV-X protein or biologically-active portion thereof with a known compound which binds NOV-X protein to form an assay mixture, contacting the assay mixture with a test compound, and determining the ability of the test compound to interact with a NOV-X protein , wherein determining the ability of the test compound to interact with a NOV-X protein comprises determining the ability of the NOV-X protein to preferentially bind to or modulate the activity of a NOV-X target molecule.

The cell-free assays of the invention are amenable to use of both the soluble form or the membrane-bound form of NOV-X protein. In the case of cell-free assays comprising the membrane-bound form of NOV-X protein, it may be desirable to utilize a solubilizing agent such that the membrane-bound form of NOV-X protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylarminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOV-X protein or its target molecule to facilitate separation of complexed from uncomplexed forms of one or both of the proteins, as well as to accommodate automation of the assay. Binding of a test compound to NOV-X protein, or interaction of NOV-X protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOV-X fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOV-X protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOV-X protein binding or activity determined using standard techniques.

Other techniques for immobilizing proteins on matrices can also be used in the screening assays of the invention. For example, either the NOV-X protein or its target molecule can be immobilized utilizing conjugation of biotin and streptavidin. Biotinylated NOV-X protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOV-X protein or target molecules, but which do not interfere with binding of the NOV-X protein to its target molecule, can be derivatized to the wells of the plate, and unbound target or NOV-X protein trapped in the wells by antibody conjugation. Methods for detecting such complexes, in addition to those described above for the GST-immobilized complexes, include immunodetection of complexes using antibodies reactive with the NOV-X protein or target molecule, as well as enzyme-linked assays that rely on detecting an enzymatic activity associated with the NOV-X protein or target molecule.

In another embodiment, modulators of NOV-X protein expression are identified in a method wherein a cell is contacted with a candidate compound and the expression of NOV-X mRNA or protein in the cell is determined. The level of expression of NOV-X niRNA or protein in the presence of the candidate compound is compared to the level of expression of NOV-X mRNA or protein in the absence of the candidate compound. The candidate compound can then be identified as a modulator of NOV-X mRNA or protein expression based upon this comparison. For example, when expression of NOV-X mRNA or protein is greater (i.e., statistically significantly greater) in the presence of the candidate compound than in its absence, the candidate compound is identified as a stimulator of NOV-X mRNA or protein expression. Alternatively, when expression of NOV-X mRNA or protein is less (statistically significantly less) in the presence of the candidate compound than in its absence, the candidate compound is identified as an inhibitor of NOV-X mRNA or protein expression. The level of NOV-X mRNA or protein expression in the cells can be determined by methods described herein for detecting NOV-X mRNA or protein.

In yet another aspect of the invention, the NOV-X proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOV-X (“NOV-X-binding proteins” or “NOV-X-bp”) and modulate NOV-X activity. Such NOV-X-binding proteins are also likely to be involved in the propagation of signals by the NOV-X proteins as, for example, upstream or downstream elements of the NOV-X pathway.

The two-hybrid system is based on the modular nature of most transcription factors, which consist of separable DNA-binding and activation domains. Briefly, the assay utilizes two different DNA constructs. In one construct, the gene that codes for NOV-X is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOV-X-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOV-X.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) identify an individual from a minute biological sample (tissue typing); and (ii) aid in forensic identification of a biological sample. Some of these applications are described in the subsections, below.

Tissue Typing

The NOV-X sequences of the invention can be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOV-X sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOV-X sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO: 1, 3, 6, 8, 10, 12, 14, 16, 18, 20, 22, or 57 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOV-X protein and/or nucleic acid expression as well as NOV-X activity, in the context of a biological sample (e.g., blood, serum, cells, tissue) to thereby determine whether an individual is afflicted with a disease or disorder, or is at risk of developing a disorder, associated with aberrant NOV-X expression or activity. Disorders associated with aberrant NOV-X expression of activity include, for example, disorders of olfactory loss, e.g. trauma, HIV illness, neoplastic growth, and neurological disorders, e.g. Parkinson's disease and Alzheimer's disease.

The invention also provides for prognostic (or predictive) assays for determining whether an individual is at risk of developing a disorder associated with NOV-X protein, nucleic acid expression or activity. For example, mutations in a NOV-X gene can be assayed in a biological sample. Such assays can be used for prognostic or predictive purpose to thereby prophylactically treat an individual prior to the onset of a disorder characterized by or associated with NOV-X protein, nucleic acid expression, or biological activity.

Another aspect of the invention provides methods for determining NOV-X protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOV-X in clinical trials. These and other agents are described in further detail in the following sections.

Diagnostic Assays

An exemplary method for detecting the presence or absence of NOV-X in a biological sample involves obtaining a biological sample from a test subject and contacting the biological sample with a compound or an agent capable of detecting NOV-X protein or nucleic acid (e.g., mRNA, genomic DNA) that encodes NOV-X protein such that the presence of NOV-X is detected in the biological sample. An agent for detecting NOV-X mRNA or genomic DNA is a labeled nucleic acid probe capable of hybridizing to NOV-X mRNA or genomic DNA. The nucleic acid probe can be, for example, a full-length NOV-X nucleic acid, such as the nucleic acid of SEQ ID NO: 1, 3, 6, 8, 10, 12, 14, 16, 18, 20, 22, or 57, or a portion thereof, such as an oligonucleotide of at least 15, 30, 50, 100, 250 or 500 nucleotides in length and sufficient to specifically hybridize under stringent conditions to NOV-X mRNA or genomic DNA. Other suitable probes for use in the diagnostic assays of the invention are described herein.

One agent for detecting NOV-X protein is an antibody capable of binding to NOV-X protein, preferably an antibody with a detectable label. Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds.

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOV-X mRNA, protein, or genomic DNA in a biological sample in vitro as well as in vivo. For example, in vitro techniques for detection of NOV-X mRNA include Northern hybridizations and in situ hybridizations. In vitro techniques for detection of NOV-X protein include enzyme linked immunosorbent assays (ELISAs), Western blots, immunoprecipitations, and immunofluorescence. In vitro techniques for detection of NOV-X genomic DNA include Southern hybridizations. Furthermore, in vivo techniques for detection of NOV-X protein include introducing into a subject a labeled anti-NOV-X antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject. In one embodiment, the methods further involve obtaining a control biological sample from a control subject, contacting the control sample with a compound or agent capable of detecting NOV-X protein, mRNA, or genomic DNA, such that the presence of NOV-X protein, mRNA or genomic DNA is detected in the biological sample, and comparing the presence of NOV-X protein, mRNA or genomic DNA in the control sample with the presence of NOV-X protein, mRNA or genomic DNA in the test sample.

The invention also encompasses kits for detecting the presence of NOV-X in a biological sample. For example, the kit can comprise: a labeled compound or agent capable of detecting NOV-X protein or mRNA in a biological sample; means for determining the amount of NOV-X in the sample; and means for comparing the amount of NOV-X in the sample with a standard. The compound or agent can be packaged in a suitable container. The kit can further comprise instructions for using the kit to detect NOV-X protein or nucleic acid.

Prognostic Assays

The diagnostic methods described herein can furthermore be utilized to identify subjects having or at risk of developing a disease or disorder associated with aberrant NOV-X expression or activity. For example, the assays described herein, such as the preceding diagnostic assays or the following assays, can be utilized to identify a subject having or at risk of developing a disorder associated with NOV-X protein, nucleic acid expression or activity. Such disorders include for example, disorders of olfactory loss, e.g. trauma, HIV illness, neoplastic growth, and neurological disorders, e.g. Parkinson's disease and Alzheimer's disease.

Alternatively, the prognostic assays can be utilized to identify a subject having or at risk for developing a disease or disorder. Thus, the invention provides a method for identifying a disease or disorder associated with aberrant NOV-X expression or activity in which a test sample is obtained from a subject and NOV-X protein or nucleic acid (e.g., mRNA, genomic DNA) is detected, wherein the presence of NOV-X protein or nucleic acid is diagnostic for a subject having or at risk of developing a disease or disorder associated with aberrant NOV-X expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOV-X expression or activity. For example, such methods can be used to determine whether a subject can be effectively treated with an agent for a disorder. Thus, the invention provides methods for determining whether a subject can be effectively treated with an agent for a disorder associated with aberrant NOV-X expression or activity in which a test sample is obtained and NOV-X protein or nucleic acid is detected (e.g., wherein the presence of NOV-X protein or nucleic acid is diagnostic for a subject that can be administered the agent to treat a disorder associated with aberrant NOV-X expression or activity).

The methods of the invention can also be used to detect genetic lesions in a NOV-X gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOV-X-protein, or the misexpression of the NOV-X gene. For example, such genetic lesions can be detected by ascertaining the existence of at least one of: (i) a deletion of one or more nucleotides from a NOV-X gene; (ii) an addition of one or more nucleotides to a NOV-X gene; (iii) a substitution of one or more nucleotides of a NOV-X gene, (iv) a chromosomal rearrangement of a NOV-X gene; (v) an alteration in the level of a messenger RNA transcript of a NOV-X gene, (vi) aberrant modification of a NOV-X gene, such as of the methylation pattern of the genomic DNA, (vii) the presence of a non-wild-type splicing pattern of a messenger RNA transcript of a NOV-X gene, (viii) a non-wild-type level of a NOV-X protein, (ix) allelic loss of a NOV-X gene, and (x) inappropriate post-translational modification of a NOV-X protein. As described herein, there are a large number of assay techniques known in the art which can be used for detecting lesions in a NOV-X gene. A preferred biological sample is a peripheral blood leukocyte sample isolated by conventional means from a subject. However, any biological sample containing nucleated cells may be used, including, for example, buccal mucosal cells. In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOV-X-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOV-X gene under conditions such that hybridization and amplification of the NOV-X gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOV-X gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOV-X can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOV-X can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. In yet another embodiment, any of a variety of sequencing reactions known in the art can be used to directly sequence the NOV-X gene and detect mutations by comparing the sequence of the sample NOV-X with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOV-X gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOV-X sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOV-X cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOV-X sequence, e.g., a wild-type NOV-X sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOV-X genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOV-X nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOV-X gene.

Furthermore, any cell type or tissue, preferably peripheral blood leukocytes, in which NOV-X is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOV-X activity (e.g., NOV-X gene expression), as identified by a screening assay described herein can be administered to individuals to treat (prophylactically or therapeutically) disorders (e.g. disorders of olfactory loss, e.g. trauma, HIV illness, neoplastic growth, and neurological disorders, e.g. Parkinson's disease and Alzheimer's disease). In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOV-X protein, expression of NOV-X nucleic acid, or mutation content of NOV-X genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOV-X protein, expression of NOV-X nucleic acid, or mutation content of NOV-X genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOV-X modulator, such as a modulator identified by one of the exemplary screening assays described herein.

Monitoring of Effects During Clinical Trials

Monitoring the influence of agents (e.g., drugs, compounds) on the expression or activity of NOV-X (e.g., the ability to modulate aberrant cell proliferation) can be applied not only in basic drug screening, but also in clinical trials. For example, the effectiveness of an agent determined by a screening assay as described herein to increase NOV-X gene expression, protein levels, or upregulate NOV-X activity, can be monitored in clinical trails of subjects exhibiting decreased NOV-X gene expression, protein levels, or downregulated NOV-X activity. Alternatively, the effectiveness of an agent determined by a screening assay to decrease NOV-X gene expression, protein levels, or downregulate NOV-X activity, can be monitored in clinical trails of subjects exhibiting increased NOV-X gene expression, protein levels, or upregulated NOV-X activity. In such clinical trials, the expression or activity of NOV-X and, preferably, other genes that have been implicated in, for example, a cellular proliferation or immune disorder can be used as a “read out” or markers of the immune responsiveness of a particular cell.

By way of example, and not of limitation, genes, including NOV-X, that are modulated in cells by treatment with an agent (e.g., compound, drug or small molecule) that modulates NOV-X activity (e.g., identified in a screening assay as described herein) can be identified. Thus, to study the effect of agents on cellular proliferation disorders, for example, in a clinical trial, cells can be isolated and RNA prepared and analyzed for the levels of expression of NOV-X and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOV-X or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOV-X protein, mRNA, or genomic DNA in the preadministration sample; (iii) obtaining one or more post-administration samples from the subject; (iv) detecting the level of expression or activity of the NOV-X protein, mRNA, or genomic DNA in the post-administration samples; (v) comparing the level of expression or activity of the NOV-X protein, mRNA, or genomic DNA in the pre-administration sample with the NOV-X protein, mRNA, or genomic DNA in the post administration sample or samples; and (vi) altering the administration of the agent to the subject accordingly. For example, increased administration of the agent may be desirable to increase the expression or activity of NOV-X to higher levels than detected, i.e., to increase the effectiveness of the agent. Alternatively, decreased administration of the agent may be desirable to decrease expression or activity of NOV-X to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOV-X expression or activity. Disorders associated with aberrant NOV-X expression include, for example, disorders of olfactory loss, e.g. trauma, HIV illness, neoplastic growth, and neurological disorders, e.g. Parkinson's disease and Alzheimer's disease.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOV-X expression or activity, by administering to the subject an agent that modulates NOV-X expression or at least one NOV-X activity. Subjects at risk for a disease that is caused or contributed to by aberrant NOV-X expression or activity can be identified by, for example, any or a combination of diagnostic or prognostic assays as described herein. Administration of a prophylactic agent can occur prior to the manifestation of symptoms characteristic of the NOV-X aberrancy, such that a disease or disorder is prevented or, alternatively, delayed in its progression. Depending upon the type of NOV-X aberrancy, for example, a NOV-X agonist or NOV-X antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOV-X expression or activity for therapeutic purposes. The modulatory method of the invention involves contacting a cell with an agent that modulates one or more of the activities of NOV-X protein activity associated with the cell. An agent that modulates NOV-X protein activity can be an agent as described herein, such as a nucleic acid or a protein, a naturally-occurring cognate ligand of a NOV-X protein, a peptide, a NOV-X peptidomimetic, or other small molecule. In one embodiment, the agent stimulates one or more NOV-X protein activity. Examples of such stimulatory agents include active NOV-X protein and a nucleic acid molecule encoding NOV-X that has been introduced into the cell. In another embodiment, the agent inhibits one or more NOV-X protein activity. Examples of such inhibitory agents include antisense NOV-X nucleic acid molecules and anti-NOV-X antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOV-X protein or nucleic acid molecule. In one embodiment, the method involves administering an agent (e.g., an agent identified by a screening assay described herein), or combination of agents that modulates (e.g., up-regulates or down-regulates) NOV-X expression or activity. In another embodiment, the method involves administering a NOV-X protein or nucleic acid molecule as therapy to compensate for reduced or aberrant NOV-X expression or activity.

Stimulation of NOV-X activity is desirable in situations in which NOV-X is abnormally downregulated and/or in which increased NOV-X activity is likely to have a beneficial effect. One example of such a situation is where a subject has a disorder characterized by aberrant cell proliferation and/or differentiation (e.g., cancer or immune associated ). Another example of such a situation is where the subject has an immunodeficiency disease (e.g., AIDS).

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

The invention will be further described in the following examples, which do not limit the scope of the invention described in the claims.

### EXAMPLES

### Example 1

Quantitative Expression Analysis of NOV-1. NOV-2, NOV-3, and NOV-4 in Various Cells and Tissues.

RTQ-PCR Panel Descriptions

Panel 1

As shown in the expression data in Tables 39, 40, and 41, Panel 1 of each table is composed of RNA or cDNA isolated from various human cells or cell lines from normal and cancerous tissue. These cells and cell lines have been extensively characterized by investigators in both academia and the commercial sectorregarding their tumorgenicity, metastatic potential, drug resistance, invasive potential, and other cancer-related properties. They serve as suitable tools for pre-clinical evaluation of anti-cancer agents and promising therapeutic strategies.

Panel 2

In Tables 39, 40, and 41, Panel 2 of each table includes 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated, many malignant tissues have “matched margins”, which is non-cancerous tissue adjacent to the tumor. These are termed normal adjacent tissues and are denoted “NAT” in Tables 39, 40, and 41. The tumor tissue and the matched margins are evaluated by two independent pathologists at NDRI or CHTN. This analysis provides a gross histopathological assessment of tumor differentiation grade. Moreover, most samples include the original surgical pathology report that provides information regarding the clinical stage of the patient. In addition, these RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, CA), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3

Panel 3 in Tables 39, 40, and 41, include samples on a 96 well plate (2 control wells, 94 test samples) composed of RNA or cDNA isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M ) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 106 cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD9SL (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Methods:

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 3 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 μl and incubated for 30 min. at 48° C. cDNA (5 μl) was then transferred to a separate plate for the TAQMAN& reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 μl using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 μl) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nrM, primer melting temperature (Tₘ) range=58°-60° C., primer optimal Tm=59° C., maximum primer difference=20° C., probe does not have 5′ G, probe Tₘ must be 100° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

The Taqman oligonucleotide set Ag756 for NOV-1, NOV-2, and NOV-2b (i.e., 10132038) include the forward probe and reverse oligomers shown below:

TABLE 36

| | | | | Start |
|---|---|---|---|---|
| Primers | Sequences | TM | Length | osition |
| Forward | 5-GGAGCAGTTCCTCACTTATCG-3′(SEQ ID NO:47) | 59 | 21 | 248 |
| Probe | TET-5′-TET-5′- (SEQ ID NO:48) | 68.6 | 27 | 272 |
| | TGATGACCAGACCTCAAGAAACACTCG-3′-TAMRA | | | |
| Reverse | 5′-CAGTTGCCATCTTTGTCTTCAT-3′(SEQ ID NO:49) | 59.2 | 22 | 304 |

The Taqman oligonucleotide set Ag756 for NOV-3a through NOV-3d (i.e., 18552586) include the forward probe and reverse oligomers shown below:

TABLE 37

| | | | | | Start |
|---|---|---|---|---|---|
| Primers | Sequences | | TM | Length | Position |
| Forward | 5′-AATGCTGAGGTCAAGCTAGGT-3′ | (SEQ ID NO: 50) | 58.1 | 21 | 121 |
| Probe | TET-5′-CTCCTTCTGAGGCTGACGAGGACCT-3′-TAMRA | (SEQ ID NO: 51) | 69.3 | 25 | 149 |
| Reverse | 5′-CATTCTCTGTTCTGGAGGTGAA-3′ | (SEQ ID NO: 52) | 59.3 | 22 | 174 |

The Taqman oligonucleotide set Ag756 for NOV-4a, NOV-4b, NOV-4c, NOV-4d, and NOV-4e (ie., 10093872) include the forward probe and reverse oligomers shown below:

TABLE 38

| Primer | Sequences | | Length |
|---|---|---|---|
| Forward | 5′-GGACTCCTCGGGATGGAAAG-3′ | (SEQ ID NO: 53) | 20 |
| Probe | FAM-5′-CGGCCTTGGTCTCGGAGATCCC-3′-TAMRA | (SEQ ID NO: 54) | 23 |
| Reverse | 5′-CTCCCCTGGTGCTGGAAATT-3′ | (SEQ ID NO: 55) | 20 |

PCR Conditions

Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

TABLE 39
NOV-1, NOV-2, NOV-2b Taqman Results

In panel 1 of the results, the following abbreviations are used:

| | |
|---|---|
| ca. | =carcinoma, |
| * | =established from metastasis, |
| met | =metastasis, |
| s cell var | =small cell variant, |
| non-s | =non-sm =non-small, |
| squam | =squamous, |
| pl. eff | =pl effusion =pleural effusion, |
| glio | =glioma, |
| astro | =astrocytoma, and |
| neuro | =neuroblastoma. |

In panel 2 of the results, the following abbreviations are used:

| | |
|---|---|
| Cca: | Colon Cancer |
| PCa: | Prostate Cancer |
| Lca: | Lung Cancer |
| RCC: | Renal Cell Carcinoma |
| UtCa: | Uterine Cancer |
| ThyCa: | Thyroid Cancer |
| BrCa: | Breast Cancer |
| HCC: | Hepatic Cell Carcinoma |
| TCC: | Transitional Cell Carcinoma of the bladder |
| OvCa: | Ovarian Cancer |
| GaCa: | Gastric Cancer |

| | Panel 1 | | | | | |
|---|---|---|---|---|---|---|
| | Run 1 | | | ag 75 | | |
| | Run 2 | | | 6% | | ag 75 |
| | ag 756 | g 756 | | Rel. | | 6% |
| | % Rel. | % Rel. | Panel 2 | Exp | Panel 3 | Rel. |
| Tissue_Name | Expn. | Expn. | Tissue_Name | n. | Tissue_Name | Expn |
| Endothelial | 0.0 | 0.0 | Normal Colon | 78.5 | 93768_Secondary | 0 |
| cells | | | | | Th1_anti-CD28/anti-CD3 | |
| Endothelial | 12.2 | 54.7 | CCa 1 | 1.0 | 93769_Secondary | 0 |
| cells (treated) | | | | | Th2_anti-CD28/anti-CD3 | |
| Pancreas | 27.6 | 5.4 | CCa 1 | 7.9 | 93770_Secondary | 0 |
| | | | Margin | | Tr1_anti-CD28/anti-CD3 | |
| Pancreatic | 0.0 | 0.0 | CCa 2 | 3.7 | 93573_Secondary | 0 |
| ca.CAPAN 2 | | | | | Th1_resting day | |
| | | | | | 4-6 in IL-2 | |
| Adrenal Gland | 9.3 | 29.3 | CCa 2 | 15.2 | 93572_Secondary | 0 |
| (new lot*) | | | Margin | | Th2_resting day | |
| | | | | | 4-6 in IL-2 | |
| Thyroid | 8.0 | 6.5 | CCa 3 | 0.4 | 93571_Secondary | 0 |
| | | | | | Tr1_resting day | |
| | | | | | 4-6 in IL-2 | |
| Salavary gland | 6.8 | 19.9 | CCa 3 | 35.6 | 93568_primary | 0 |
| | | | Margin | | Th1_anti-CD28/anti-CD3 | |
| Pituitary gland | 3.2 | 7.8 | CCa 4 | 10.1 | 93569_primary | 0 |
| | | | | | Th2_anti-CD28/anti-CD3 | |
| Brain (fetal) | 3.4 | 18.4 | CCa 4 | 11.6 | 93570_primary | 0 |
| | | | Margin | | Tr1_anti-CD28/anti-CD3 | |
| Brain (whole) | 6.9 | 27.4 | CCa 5 | 7.2 | 93565_primary | 0 |
| | | | Metastasis | | Th1_resting dy | |
| | | | | | 4-6 in IL-2 | |
| Brain. | 2.5 | 13.8 | CCa 5 | 52.9 | 93566_primary | 0 |
| (amygdala) | | | Margin | | Th2_resting | |
| | | | (Liver) | | dy 4-6 in IL-2 | |
| Brain | 2.0 | 28.7 | CCa 6 | 2.5 | 93567_primary | 0 |
| (cerebellum) | | | | | Tr1_resting | |
| | | | | | dy 4-6 in IL-2 | |
| Brain | 3.8 | 20.9 | CCa 6 | 14.1 | 93351_CD45RA CD4 | 0 |
| (hippocampus) | | | Margin | | lymphocyte_anti- | |
| | | | (Lung) | | CD28/anti-CD3 | |
| Brain | 3.0 | 11.0 | Normal | 10.0 | 93352_CD45RO CD4 | 0 |
| (thalamus) | | | Prostate | | lymphocyte_anti- | |
| | | | | | CD28/anti-CD3 | |
| Cerebral | 7.0 | 61.1 | PCa 1 | 10.7 | 93251_CD8 | 0 |
| Cortex | | | | | Lymphocytes_anti- | |
| | | | | | CD28/anti-CD3 | |
| Spinal cord | 8.6 | 27.0 | PCa 1 | 37.6 | 93353_chronic CD8 | 0 |
| | | | Margin | | Lymphocytes 2ry_resting | |
| | | | | | dy 4-6 in IL-2 | |
| CNS | 0.0 | 0.0 | PCa 2 | 100.0 | 93574_chronic CD8 | 0 |
| ca.(glio/astro) | | | | | Lymphocytes 2ry_activated | |
| U87-MG | | | | | CD3/CD28 | |
| CNS | 0.2 | 0.0 | PCa 2 | 89.5 | 93354_CD4_none | 0 |
| ca.(glio/astro) | | | Margin | | | |
| U-118-MG | | | | | | |
| CNS | 0.0 | 0.0 | Normal Lung | 51.1 | 93252_Secondary | 0 |
| ca.(astro) | | | | | Th1/Th2/Tr1_anti-CD95 | |
| SW1783 | | | | | CH11 | |
| CNS ca.* | 0.0 | 0.0 | LCa 1 | 1.0 | 93103_LAK cells_resting | 0 |
| (neuro; met) | | | Metastasis | | | |
| SK-N-AS | | | | | | |
| CNS ca. | 0.1 | 0.0 | LCa 1 | 11.3 | 93788_LAK cells_IL-2 | 0 |
| (astro)SF-539 | | | Margin | | | |
| | | | (Muscle) | | | |
| CNS ca. | 0.3 | 0.3 | LCa 2 | 8.5 | 93787_LAK cells_IL-2 + | 0 |
| (astro)SNB-75 | | | | | IL-12 | |
| CNS ca. | 0.1 | 0.0 | LCa 2 | 31.6 | 93789_LAK cells_IL-2 + | 0 |
| (glio)SNB-19 | | | Margin | | IFN gamma | |
| CNS ca. | 0.0 | 0.0 | LCa 3 | 5.8 | 93790_LAK cells_IL-2 + | 0 |
| (glio)U251 | | | | | IL-18 | |
| CNS ca. | 0.0 | 0.0 | LCa 3 | 28.3 | 93104_LAK | 0 |
| (glio)SF-295 | | | Margin | | cells_PMA/ionomycin and | |
| | | | | | IL-18 | |
| Heart | 28.5 | 77.9 | LCa 4 | 1.6 | 93578_NK Cells | 0 |
| | | | | | IL-2_resting | |
| Skeletal | 16.3 | 15.7 | LCa 5 | 4.2 | 93109_Mixed Lymphocyte | 0 |
| Muscle | | | | | Reaction_Two Way MLR | |
| (new lot*) | | | | | | |
| Bone marrow | 0.7 | 0.9 | LCa 5 | 29.5 | 93110_Mixed Lymphocyte | 0 |
| Margin | | | | | Reaction_Two Way MLR | |
| Thymus | 1.1 | 2.7 | Ocular | 15.9 | 93111_Mixed Lymphocyte | 0 |
| | | | Melanoma | | Reaction_Two Way MLR | |
| | | | Metastasis | | | |
| Spleen | 0.9 | 2.1 | Ocular | 38.7 | 93112_Mononuclear Cells | 0 |
| | | | Melanoma | | (PBMCs)_resting | |
| | | | Margin | | | |
| | | | (Liver) | | | |
| Lymph node | 3.6 | 10.2 | Melanoma | 0.0 | 93113_Mononuclear Cells | 0 |
| | | | Metastasis | | (PBMCs)_PWM | |
| Colorectal | 2.2 | 11.4 | Melanoma | 32.3 | 93114_Mononuclear Cells | 0 |
| | | | Margin | | (PBMCs)_PHA-L | |
| | | | (Lung) | | | |
| Stomach | 11.8 | 34.4 | Normal | 56.3 | 93249_Ramos | 0 |
| | | | Kidney | | (B cell)_none | |
| Small | 11.7 | 18.7 | RCC 1 | 71.2 | 93250_Ramos (B | 0 |
| intestine | | | | | cell)_ionomycin | |
| Colon | 0.0 | 0.0 | RCC 1 | 26.1 | 93349_B | 0 |
| ca.SW480 | | | Margin | | lymphocytes_PWM | |
| Colon ca.* | 0.0 | 0.0 | RCC 2 | 63.7 | 93350_B | 0 |
| (SW480 | | | | | lymphoytes_CD40L and | |
| met)SW620 | | | | | IL-4 | |
| Colon ca. | 0.0 | 0.0 | RCC 2 | 28.3 | 92665_EOL-1 | 0 |
| HT29 | | | Margin | | (Eosinophil_dbcAMP | |
| | | | | | differentiated | |
| Colon ca. | 0.0 | 0.0 | RCC 3 | 37.1 | 93248_EOL-1 | 0 |
| HCT-116 | | | | | (Eosinophil_dbcAMP/PMA | |
| | | | | | ionomycin | |
| Colon | 0.0 | 0.0 | RCC 3 | 33.7 | 93356_Dendritic | 0 |
| ca.CaCo-2 | | | Margin | | Cells_none | |
| 83219 CC Well | 0.3 | 1.7 | RCC 4 | 5.7 | 93355_Dendritic Cells_LPS | 0 |
| to Mod Diff | | | | | 100 ng/ml | |
| (ODO3866) | | | | | | |
| Colon ca. | 0.0 | 0.0 | RCC 4 | 18.2 | 93775_Dendritic | 0 |
| HCC-2998 | | | Margin | | Cells_anti-CD40 | |
| Gastric ca.* | 0.0 | 0.0 | RCC 5 | 8.5 | 93774_Monocytes_resting | 0 |
| (liver met) NCl- | | | | | | |
| N87 | | | | | | |
| Bladder | 16.4 | 29.3 | RCC 5 | 5.5 | 93776_Monocytes_LPS | 0 |
| | | | Margin | | 50 ng/ml | |
| Trachea | 4.2 | 12.7 | RCC 6 | 1.0 | 93581_Macrophages_resting | 0 |
| Kidney | 5.3 | 14.3 | RCC 6 | 18.7 | 93582_Macrophages_LPS | 0 |
| | | | Margin | | 100 ng/ml | |
| Kidney (fetal) | 7.8 | 24.3 | RCC 7 | 6.0 | 93098_HUVEC | 0 |
| | | | | | (Endothelial)_none | |
| Renal ca. | 0.5 | 2.4 | RCC 7 | 8.5 | 93099_HUVEC | 0 |
| 786-0 | | | Margin | | (Endothelial)_starved | |
| Renal ca.A498 | 0.2 | 0.0 | RCC 8 | 0.3 | 93100_HUVEC | 0 |
| | | | | | (Endothelial)_IL-1b | |
| Renal ca. | 6.0 | 18.6 | RCC 8 | 14.1 | 93779_HUVEC | 0.1 |
| RXF 393 | | | Margin | | (Endothelial)_IFN gamma | |
| Renal | 15.0 | 28.7 | RCC 9 | 6.3 | 93102_HUVEC | 0.0 |
| ca.ACHN | | | | | (Endothelial)_TNF alpha + | |
| | | | | | IFN gamma | |
| Renal ca.UO- | 1.2 | 4.2 | RCC 9 | 19.6 | 93101_HUVEC | 0.0 |
| 31 | | | Margin | | (Endothelial)_TNF alpha + | |
| | | | | | IL4 | |
| Renal ca. | 10.3 | 21.2 | Normal | 7.0 | 93781_HUVEC | 0.0 |
| TK-10 | | | Uterus | | (Endothelial)_IL-11 | |
| Liver | 12.9 | 48.0 | UtCa 1 | 46.0 | 93583_Lung Microvascular | 37.1 |
| | | | | | Endothelial Cells_none | |
| Liver (fetal) | 4.7 | 17.7 | Normal | 6.1 | 93584_Lung Microvascular | 12.9 |
| | | | Thyroid | | Endothelial Cells_TNFa | |
| | | | | | (4 ng/ml) and IL 1b | |
| | | | | | (1 ng/ml)** | |
| Liver ca. | 0.0 | 0.0 | ThyCa 1 | 6.1 | 92662_Microvascular | 81.2 |
| (hepatoblast) | | | | | Dermal endothelium_none | |
| HepG2 | | | | | | |
| Lung | 7.4 | 25.4 | ThyCa 2 | 0.8 | 92663_Microsvasular | 38.7 |
| | | | | | Dermal endothelium_TNFa | |
| | | | | | (4 ng/ml) and IL 1b | |
| | | | | | (1 ng/ml) | |
| Lung (fetal) | 9.6 | 19.0 | ThyCa 2 | 22.5 | 93773_Bronchial | 0.0 |
| | | | Margin | | epithelium_TNFa | |
| | | | | | (4 ng/ml) and IL 1b (1 ng/ml) | |
| Lung ca. (small | 0.0 | 0.0 | Normal | 12.1 | 93347_Small Airway | 0.0 |
| cell) LX-1 | | | Breast | | Epithelium_none | |
| Lung ca. (small | 0.0 | 0.0 | BrCa 1 | 7.5 | 93348_Small Airway | 0.5 |
| cell) NCl-H69 | | | | | Epithelium_TNFa (4 ng/ml) | |
| | | | | | and IL 1b (1 ng/m1) | |
| Lung ca. (s.cell | 0.0 | 0.0 | BrCa 2 | 4.0 | 92668_Coronery Artery | 0.6 |
| var.) SHP-77 | | | | | SMC_resting | |
| Lung Ca. (large | 0.1 | 0.0 | BrCa 3 | 15.1 | 92669_Coronery Artery | 0.1 |
| cell)NCl-H460 | | | Metastasis | | SMC_TNFa (4 ng/ml) and | |
| | | | | | IL 1b (1 ng/ml) | |
| Lung Ca. (non- | 0.2 | 0.0 | BrCa 4 | 18.4 | 93107_astrocytes_resting | 20.0 |
| sm. cell) A549 | | | Metastasis | | | |
| Lung Ca. (non- | 0.4 | 2.4 | BrCa 5 | 11.7 | 93108_astrocytes_TNFa | 17.0 |
| s.cell) NCl-H23 | | | | | (4 ng/ml) and IL 1b (1 ng/ml) | |
| Lung ca (non- | 1.3 | 0.8 | BrCa 6 | 3.1 | 92666_KU-812 | 0.0 |
| s.cell) HOP-62 | | | | | (Basophil)_resting | |
| Lung Ca. (non- | 100.0 | 100.0 | BrCa 6 | 5.3 | 92667_KU-812 | 0.0 |
| s.cl) NCl-H522 | | | Margin | | (Basophil)_PMA/ionoycin | |
| Lung ca. | 0.7 | 0.8 | BrCa 7 | 6.8 | 93579_CCD1106 | 0.0 |
| (squam.) | | | | | (Keratinocytes)_none | |
| SW 900 | | | | | | |
| Lung ca. | 0.0 | 0.0 | BrCa 7 | 11.7 | 93580_CCD1106 | 4.1 |
| (squam.) | | | Margin | | (Keratinocytes)_TNFa and | |
| NCl-H596 | | | | | IFNg** | |
| Mammary | 5.0 | 7.5 | Normal Liver | 37.1 | 93791_Liver Cirrhosis | 8.1 |
| gland | | | | | | |
| Breast ca.* (pl. | 2.7 | 12.0 | HCC 1 | 47.0 | 93792_Lupus Kidney | 18.1 |
| effusion) | | | | | | |
| MCF-7 | | | | | | |
| Breast ca.* | 0.0 | 0.0 | HCC 2 | 34.2 | 93577_NCl-H292 | 1.9 |
| (pl.ef) MDA- | | | | | | |
| MB-231 | | | | | | |
| Breast ca.* (pl. | 0.2 | 0.0 | HCC 3 | 5.2 | 93358_NCl-H292_IL-4 | 4.6 |
| effusion) T47D | | | | | | |
| Breast ca. | 0.0 | 0.0 | HCC 4 | 27.6 | 93360_NCl-H292_IL-9 | 0.9 |
| BT-549 | | | | | | |
| Breast ca. | 0.0 | 0.0 | HCC 4 | 3.6 | 93359_NCl-H292_IL-13 | 1.7 |
| MDA-N | | | Margin | | | |
| Ovary | 5.5 | 18.4 | HCC 5 | 5.3 | 93357_NCl-H292_IFN | 4.6 |
| | | | | | gamma | |
| Ovarian | 11.8 | 21.2 | HCC 5 | 15.9 | 93777_HPAEC_- | 0.0 |
| ca.OVCAR-3 | | | Margin | | | |
| Ovarian | 4.6 | 12.5 | Normal | 27.0 | 93778_HPAEC_IL-1 | 0.0 |
| ca.OVCAR-4 | | | Bladder | | beta/TNA alpha | |
| Ovarian | 0.2 | 0.0 | TCC 1 | 2.1 | 93254_Normal Human | 0.3 |
| ca.OVCAR-5 | | | | | Lung Fibroblast_none | |
| Ovarian | 4.5 | 21.5 | TCC 2 | 1.1 | 93253_Normal Human | 0.8 |
| ca.OVCAR-8 | | | | | Lung Fibroblast_TNFa | |
| | | | | | (4 ng/ml) and IL-1b (1 ng/ml) | |
| Ovarian | 4.3 | 5.4 | TCC 3 | 2.1 | 93257_Normal Human | 0.5 |
| ca.IGROV-1 | | | | | Lung Fibroblast_IL-4 | |
| Ovarian ca.* | 50.0 | 92.7 | TCC 3 | 52.1 | 93256_Normal Human | 0.3 |
| (ascites) | | | Margin | | Lung Fibroblast_IL-9 | |
| SK-OV-3 | | | | | | |
| Uterus | 7.6 | 24.2 | Normal | 7.7 | 93255_Normal Human | 1.7 |
| | | | Ovary | | Lung Fibroblast_IL-13 | |
| Plancenta | 17.0 | 31.4 | OvCa 1 | 89.5 | 93258_Normal Human | 10.2 |
| | | | | | Lung Fibroblast_IFN | |
| | | | | | gamma | |
| Prostate | 5.3 | 15.5 | OvCa 2 | 45.1 | 93106_Dermal Fibroblasts | 0.0 |
| | | | | | CCD1070_resting | |
| Prostate ca.* | 14.7 | 42.6 | OvCa 2 | 8.7 | 93361_Dermal Fibroblasts | 0.3 |
| (bone met) | | | Margin | | CCD1070_TNF alpha | |
| PC-3 | | | | | 4 ng/ml | |
| Testis | 10.2 | 13.1 | Normal | 25.7 | 93105_Dermal Fibroblasts | 0.0 |
| | | | Stomach | | CCD1070_IL-1 beta | |
| | | | | | 1 ng/ml | |
| Melanoma | 0.0 | 0.0 | Normal | 15.6 | 93772_dermal | 0.8 |
| Hs688(A).T | | | Stomach | | fibroblast_IFN gamma | |
| Melanoma* | 0.1 | 0.0 | GaCa 1 | 26.6 | 93771_dermal | 0.5 |
| (met) | | | | | fibroblast_IL-4 | |
| Hs688(B).T | | | | | | |
| MelanomaUAC | 0.2 | 0.0 | GaCa 1 | 31.0 | 93259_IBD Colitis 1** | 19.3 |
| C-62 | | | Margin | | | |
| Melanoma | 0.0 | 0.0 | GaCa 2 | 15.4 | 93260_IBD Colitis 2 | 6.1 |
| M14 | | | | | | |
| Melanoma | 0.0 | 0.0 | GaCa 2 | 5.2 | 93261_IBD Crohns | 3.7 |
| LOX IMVI | | | Margin | | | |
| Melanoma* | 0.1 | 0.0 | GaCa 3 | 13.7 | 735010_Colon_normal | 26.1 |
| (met) | | | | | | |
| SK-MEL-5 | | | | | | |
| Adipose | 2.5 | 29.9 | | | 735019_Lung_none | 90.1 |
| | | | | | 64028-1_Thymus_none | 100.0 |
| | | | | | 64030-1_Kidney_none | 16.0 |

TABLE 40
NOV-3a, NOV-3b, NOV-3c Taqman Results

| | | | Panel 2 | | |
|---|---|---|---|---|---|
| | Panel 1 | | ag 66 | | Panel 3 |
| | ag 664 | | 4 | | ag 664 |
| | % Rel. | | % Rel. | | % Rel. |
| Tissue_Name | Expn. | Tissue_Name | Expn. | Tissue_Name | Expn. |
| Liver | 13.6 | Normal Colon | 70.2 | 93768_Secondary Th1_anti- | 16.4 |
| adenocarcinoma | | | | CD28/anti-CD3 | |
| Heart (fetal) | 6.5 | CCa 1 | 22.7 | 93769_Secondary Th2_anti- | 12.9 |
| | | | | CD28/anti-CD3 | |
| Pancreas | 6.4 | CCa 1 | 9.0 | 93770_Secondary Tr1_anti- | 18.3 |
| | | Margin | | CD28/anti-CD3 | |
| Pancreatic ca. | 1.6 | CCa 2 | 14.0 | 93573_Secondary Th1_resting | 22.1 |
| CAPAN 2 | | | | day 4-6 in IL-2 | |
| Adrenal gland | 10.5 | CCa 2 | 6.5 | 93572_Secondary Th2_resting | 13.1 |
| | | Margin | | day 4-6 in IL-2 | |
| Thyroid | 5.6 | CCa 3 | 42.6 | 93571_Secondary Tr1_resting | 23.0 |
| | | | | day 4-6 in IL-2 | |
| Salivary gland | 4.8 | CCa 3 | 20.2 | 93568_primary Th1_anti- | 11.5 |
| | | Margin | | CD28/anti-CD3 | |
| Pituitary gland | 14.3 | CCa 4 | 27.6 | 93569_primary Th2_anti- | 15.9 |
| | | | | CD28/anti-CD3 | |
| Brain (fetal) | 27.6 | CCa 4 | 10.2 | 93570_primary Tr1_anti- | 16.5 |
| | | Margin | | CD28/anti-CD3 | |
| Brain (whole) | 22.5 | CCa 5 | 38.4 | 93565_primary Th1_resting dy | 73.7 |
| | | Metastasis | | 4-6 in IL-2 | |
| Brain (amygdala) | 22.7 | CCa 5 | 7.3 | 93566_primary Th2_resting dy | 47.0 |
| | | Margin (Liver) | | 4-6 in IL-2 | |
| Brain (cerebellum) | 13.0 | CCa 6 | 34.4 | 93567_primary Tr1_resting dy | 26.4 |
| | | | | 4-6 in IL-2 | |
| Brain (hippocampus) | 100.0 | CCa 6 | 5.9 | 93351_CD45RA CD4 | 8.5 |
| | | Margin (Lung) | | lymphocyte_anti-CD28/anti-CD3 | |
| Brain (thalamus) | 22.4 | Normal | 20.7 | 93352_CD45RO CD4 | 19.3 |
| | | Prostate | | lymphocyte_anti-CD28/anti-CD3 | |
| Cerebral Cortex | 24.3 | PCa 1 | 26.6 | 93251_CD8 Lymphocytes_anti- | 8.0 |
| | | | | CD28/anti-CD3 | |
| Spinal cord | 22.7 | PCa 1 | 32.8 | 93353_chronic CD8 Lymphocytes | 9.9 |
| | | Margin | | 2ry_resting dy 4-6 in IL-2 | |
| glio/astro U87-MG | 2.8 | PCa 2 | 47.3 | 93574_chronic CD8 Lymphocytes | 6.7 |
| | | | | 2ry_activated CD3/CD28 | |
| glio/astro U-118-MG | 22.7 | PCa 2 | 36.9 | 93354_CD4_none | 17.4 |
| | | Margin | | | |
| astro SW1783 | 5.4 | Normal Lung | 100.0 | 93252_Secondary | 20.7 |
| | | | | Th1/Th2/Tr1_anti-CD95 CH11 | |
| neuro; met SK-N-AS | 26.8 | LCa 1 | 12.5 | 93103_LAK cells_resting | 20.5 |
| | | Metastasis | | | |
| astro SF-539 | 12.8 | LCa 1 Margin | 3.8 | 93788_LAK cells_IL-2 | 19.3 |
| | | (Muscle) | | | |
| astro SNB-75 | 5.4 | LCa 2 | 24.2 | 93787_LAK cells_IL-2 + IL-12 | 6.8 |
| glio SNB-19 | 7.4 | LCa 2 | 40.9 | 93789_LAK cells_IL-2 + IFN | 16.0 |
| | | Margin | | gamma | |
| glio U251 | 4.0 | LCa 3 | 13.6 | 93790_LAK cells_IL-2 + IL-18 | 24.2 |
| glio SF-295 | 4.5 | LCa 3 | 7.8 | 93104_LAK cells_PMA/ionomycin | 1.5 |
| | | Margin | | and IL-18 | |
| Heart | 2.4 | LCa 4 | 10.4 | 93578_NK Cells IL-2_resting | 18.7 |
| Skeletal muscle | 0.9 | LCa 5 | 32.3 | 93109_Mixed Lymphocyte | 23.7 |
| | | | | Reaction_Two Way MLR | |
| Bone marrow | 17.0 | LCa 5 | 12.1 | 93110_Mixed Lymphocyte | 5.8 |
| | | Margin | | Reaction_Two Way MLR | |
| Thymus | 20.3 | Ocular | 6.8 | 93111_Mixed Lymphocyte | 10.2 |
| | | Melanoma | | Reaction_Two Way MLR | |
| | | Metastasis | | | |
| Spleen | 25.4 | Ocular | 8.0 | 93112_Mononuclear Cells | 8.6 |
| | | Melanoma | | (PBMCs)_resting | |
| | | Margin (Liver) | | | |
| Lymph node | 29.9 | Melanoma | 18.2 | 93113_Mononuclear Cells | 24.5 |
| | | Metastasis | | (PBMCs)_PWM | |
| Colorectal | 15.9 | Melanoma | 16.4 | 93114_Mononuclear Cells | 18.6 |
| | | Margin (Lung) | | (PBMCs)_PHA-L | |
| Stomach | 24.8 | Normal Kidney | 40.9 | 93249_Ramos (B cell)_none | 5.2 |
| Small intestine | 14.4 | RCC 1 | 32.8 | 93250_Ramos (B cell)_ionomycin | 17.8 |
| Colon 5W480 | 4.9 | RCC 1 Margin | 30.6 | 93349_B lymphocytes_PWM | 26.2 |
| Colon | 9.3 | RCC 2 | 63.3 | 93350_B lymphoytes_CD40L and | 30.6 |
| SW620 (SW480 met) | | | | IL-4 | |
| Colon HT29 | 6.6 | RCC 2 Margin | 9.7 | 92665_EOL-1 | 9.7 |
| | | | | (Eosinophil)_dbcAMP | |
| | | | | differentiated | |
| Colon HCT-116 | 3.2 | RCC 3 | 31.2 | 93248_EOL-1 | 9.7 |
| | | | | (Eosinophil)_dbcAMP/PMA | |
| | | | | ionomycin | |
| Colon CaCo-2 | 3.7 | RCC 3 Margin | 18.6 | 93356_Dendritic Cells_none | 12.1 |
| Colon Ca | 18.7 | RCC 4 | 4.5 | 93355_Dendritic Cells_LPS 100 | 20.0 |
| tissue(ODO3866) | | | | ng/ml | |
| Colon HCC-2998 | 32.5 | RCC 4 Margin | 12.2 | 93775_Dendritic Cells_anti-CD40 | 17.7 |
| Gastric(liver met) | 11.0 | RCC 5 | 11.6 | 93774_Monocytes_resting | 22.4 |
| NCl-N87 | | | | | |
| Bladder | 6.9 | RCC 5 Margin | 3.9 | 93776_Monocytes_LPS 50 ng/ml | 2.0 |
| Trachea | 35.6 | RCC 6 | 15.8 | 93581_Macrophages_resting | 11.8 |
| Kidney | 6.4 | RCC 6 Margin | 14.6 | 93582_Macrophages_LPS 100 | 3.9 |
| | | | | ng/ml | |
| Kidney (fetal) | 13.7 | RCC 7 | 9.2 | 93098_HUVEC | 6.0 |
| | | | | (Endothelial)_none | |
| Renal 786-0 | 0.0 | RCC 7 Margin | 5.8 | 93099_HUVEC | 15.0 |
| | | | | (Endothelial)_starved | |
| Renal A498 | 20.7 | RCC 8 | 20.9 | 93100_HUVEC (Endothelial)_IL-1b | 5.3 |
| Renal RXF 393 | 1.5 | RCC 8 Margin | 10.7 | 93779_HUVEC (Endothelial)_IFN | 13.2 |
| | | | | gamma | |
| Renal ACHN | 1.8 | RCC 9 | 23.0 | 93102_HUVEC | 9.0 |
| | | | | (Endothelial)_TNF | |
| | | | | alpha + IFN gamma | |
| Renal UO-31 | 1.6 | RCC 9 Margin | 21.0 | 93101_HUVEC | 5.3 |
| | | | | (Endothelial)_TNF | |
| | | | | alpha + IL4 | |
| Renal TK-10 | 2.3 | Normal Uterus | 5.5 | 93781_HUVEC | 5.5 |
| | | | | (Endothelial)_IL-11 | |
| Liver | 10.5 | UtCa 1 | 31.9 | 93583_Lung Microvascular | 7.3 |
| | | | | Endothelial Cells_none | |
| Liver (fetal) | 21.8 | Normal Thyroid | 13.8 | 93584_Lung Microvascular | 6.5 |
| | | | | Endothelial Cells_TNFa (4 ng/ml) | |
| | | | | and IL 1b (1 ng/ml) | |
| Liver (hepatoblast) | 14.1 | ThyCa 1 | 6.3 | 92662_Microvascular Dermal | 8.0 |
| HepG2 | | | | endothelium_none | |
| Lung | 48.6 | ThyCa 2 | 7.9 | 92663_Microsvasular Dermal | 10.2 |
| | | | | endothelium_TNFa (4 ng/ml) and | |
| | | | | IL 1b (1 ng/ml) | |
| Lung (fetal) | 24.3 | ThyCa 2 | 7.0 | 93773_Bronchial | 12.2 |
| | | Margin | | epithelium_TNFa (4 ng/ml) and | |
| | | | | IL 1b (1 ng/ml)** | |
| Lung (small cell) | 4.7 | Normal Breast | 36.9 | 93347_Small Airway | 4.9 |
| LX-1 | | | | Epithelium_none | |
| Lung (small cell) | 1.1 | BrCa 1 | 10.7 | 93348_Small Airway | 32.5 |
| NCl-H69 | | | | Epithelium_TNFa (4 ng/ml) and | |
| | | | | IL 1b (1 ng/ml) | |
| Lung (s.cell var.) | 24.5 | BrCa 2 | 11.2 | 92668_Coronery Artery | 2.4 |
| SHP-77 | | | | SMC_resting | |
| Lung (large cell)NCl- | 1.6 | BrCa 3 | 32.8 | 92669_Coronery Artery | 0.0 |
| H460 | | Metastasis | | SMC_TNFa (4 ng/ml) and IL 1b | |
| | | | | (1 ng/ml) | |
| Lung (non-sm. cell) | 1.4 | BrCa 4 | 13.7 | 93107_astrocytes_resting | 4.4 |
| A549 | | Metastasis | | | |
| Lung (non-s.cell) | 10.7 | BrCa 5 | 19.8 | 93108_astrocytes_TNFa | 4.2 |
| NCl-H23 | | | | (4 ng/ml) and IL 1b (1 ng/ml) | |
| Lung (non-s.cell) | 32.3 | BrCa 6 | 29.1 | 92666_KU-812 (Basophil)_resting | 2.5 |
| HOP-62 | | | | | |
| Lung (non-s.cl) NCl- | 1.7 | BrCa 6 Margin | 17.2 | 92667_KU-812 | 7.2 |
| H522 | | | | (Basophil)_PMA/ionoycin | 7.2 |
| Lung (squam.) SW | 3.6 | BrCa 7 | 13.9 | 93579_CCD1106 | 6.6 |
| 900 | | | | (Keratinocytes)_none | |
| Lung (squam.) NCl- | 0.9 | BrCa 7 Margin | 25.5 | 93580_CCD1106 | 3.0 |
| H596 | | | | (Keratinocytes)_TNFa and IFNg** | |
| Mammary gland | 27.2 | Normal Liver | 8.3 | 93791_Liver Cirrhosis | 5.4 |
| Breast (pl.ef) MCF-7 | 12.4 | HCC 1 | 14.1 | 93792_Lupus Kidney | 2.2 |
| Breast (pl.ef) MDA- | 18.7 | HCC 2 | 14.5 | 93577_NCl-H292 | 41.5 |
| MB-231 | | | | | |
| Breast (pl.ef) T47D | 0.4 | HCC 3 | 7.9 | 93358_NCl-H292_IL-4 | 62.4 |
| Breast BT-549 | 28.9 | HCC 4 | 16.0 | 93360_NCl-H292_IL-9 | 53.2 |
| Breast MDA-N | 20.6 | HCC 4 Margin | 19.3 | 93359_NCl-H292_IL-13 | 21.3 |
| Ovary | 12.2 | HCC 5 | 4.0 | 93357_NCl-H292_IFN gamma | 19.9 |
| Ovarian OVCAR-3 | 2.1 | HCC 5 Margin | 2.0 | 93777_HPAEC_- | 6.5 |
| Ovarian OVCAR-4 | 0.5 | Normal Bladder | 44.4 | 93778_HPAEC_IL-1 beta/TNA | 9.7 |
| | | | | alpha | |
| Ovarian OVCAR-5 | 0.6 | TCC 1 | 24.5 | 93254_Normal Human Lung | 2.2 |
| | | | | Fibroblast_none | |
| Ovarian OVCAR-8 | 6.5 | TCC 2 | 16.4 | 93253_Normal Human Lung | 3.0 |
| | | | | Fibroblast_TNFa (4 ng/ml) and IL- | |
| | | | | 1b (1 ng/ml) | |
| Ovarian IGROV-1 | 3.5 | TCC 3 | 22.7 | 93257_Normal Human Lung | 4.0 |
| | | | | Fibroblast_IL-4 | |
| Ovarian (ascites) | 2.3 | TCC 3 Margin | 13.4 | 93256_Normal Human Lung | 3.2 |
| SK-OV-3 | | | | Fibroblast_IL-9 | |
| Uterus | 17.2 | Normal Ovary | 12.7 | 93255_Normal Human Lung | 4.8 |
| | | | | Fibroblast_IL-13 | |
| Plancenta | 12.9 | OvCa 1 | 23.3 | 93258_Normal Human Lung | 4.0 |
| | | | | Fibroblast_IFN gamma | |
| Prostate | 8.5 | OvCa 2 | 72.2 | 93106_Dermal Fibroblasts | 1.5 |
| | | | | CCD1070_resting | |
| Prostate (bone | 3.3 | OvCa 2 Margin | 4.1 | 93361_Dermal Fibroblasts | 42.6 |
| met)PC-3 | | | | CCD1070_TNF alpha 4 ng/ml | |
| Testis | 4.1 | Normal | 20.2 | 93105_Dermal Fibroblasts | 7.3 |
| | | Stomach | | CCD1070_IL-1 beta 1 ng/ml | |
| Melanoma | 0.6 | Normal | 5.2 | 93772_dermal fibroblast_IFN | 4.3 |
| Hs688(A).T | | Stomach | | gamma | |
| Melanoma (met) | 0.5 | GaCa 1 | 8.4 | 93771_dermal fibroblast_IL-4 | 10.4 |
| Hs688(B).T | | | | | |
| Melanoma UACC-62 | 1.6 | GaCa 1 Margin | 15.9 | 93259_IBD Colitis 1** | 3.2 |
| Melanoma M14 | 0.6 | GaCa 2 | 38.4 | 93260_IBD Colitis 2 | 0.0 |
| Melanoma LOX | 1.7 | GaCa 2 Margin | 4.5 | 93261_IBD Crohns | 0.0 |
| IMVI | | | | | |
| Melanoma (met) SK- | 6.2 | GaCa 3 | 55.5 | 735010_Colon_normal | 23.0 |
| MEL-5 | | | | | |
| Adipose | 6.0 | | | 735019_Lung_none | 6.4 |
| | | | | 64028-1_Thymus_none | 21.2 |
| | | | | 64030-1_Kidney_none | 100.0 |

TABLE 41
NOV-4a, NOV-4b, NOV-4c, NOV-4d, and NOV-4e Taqman results

| | Panel 1 | | Panel 2 |
|---|---|---|---|
| | ag 538 | | ag 538 |
| | % Rel. | | % Rel. |
| Tissue_Name | expn. | Tissue_Name | expn. |
| Adipose | 12.6 | Normal Colon GENPAK | 9.7 |
| | | 061003 | |
| Adrenal gland | 19.9 | 83219 CC Well to Mod Diff | 4.3 |
| | | (ODO3866) | |
| Bladder | 100.0 | 83220 CC NAT (ODO3866) | 3.3 |
| Bone marrow | 4.8 | 83221 CC Gr.2 rectosigmoid | 2.9 |
| | | (ODO3868) | |
| Endothelial cells | 0.0 | 83222 CC NAT (ODO3868) | 2.1 |
| Endothelial cells | 4.5 | 83235 CC Mod Diff | 8.0 |
| (treated) | | (ODO3920) | |
| Liver | 9.3 | 83236 CC NAT (ODO3920) | 4.6 |
| Liver (fetal) | 4.1 | 83237 CC Gr.2 ascend colon | 3.4 |
| | | (ODO3921) | |
| Spleen | 4.4 | 83238 CC NAT (ODO3921) | 2.4 |
| Thymus | 2.3 | 83241 CC from Partial | 2.8 |
| | | Hepatectomy (ODO4309) | |
| Thyroid | 14.0 | 83242 Liver NAT (ODO4309) | 4.5 |
| Trachea | 7.6 | 87472 Colon mets to lung | 7.0 |
| | | (ODO4451-01) | |
| Testis | 10.4 | 87473 Lung NAT | 17.2 |
| | | ODO4451-02) | |
| Spinal cord | 8.7 | Normal Prostate Clontech A+ | 6.2 |
| | | 6546-1 | |
| Salavary gland | 13.7 | 84140 Prostate Cancer | 13.0 |
| | | (ODO4410) | |
| Brain (amygdala) | 0.2 | 84141 Prostate NAT | 100.0 |
| | | (ODO4410) | |
| Brain (cerebellum) | 0.8 | 87073 Prostate Cancer | 20.2 |
| | | (ODO4720-01) | |
| Brain (hippocampus) | 1.2 | 87074 Prostate NAT | 6.0 |
| | | (ODO4720-02) | |
| Brain (substantia | 7.9 | Normal Lung GENPAK | 2.7 |
| nigra) | | 061010 | |
| Brain (thalamus) | 1.2 | 83239 Lung Met to Muscle | 0.5 |
| | | (ODO4286) | |
| Cerebral Cortex | 1.0 | 83240 Muscle NAT | 9.8 |
| | | (ODO4286) | |
| Brain (whole) | 0.4 | 84136 Lung Malignant | 2.0 |
| | | Cancer (ODO3126) | |
| Brain (fetal) | 0.1 | 84137 Lung NAT (ODO3126) | 3.1 |
| CNS ca. (glio/astro) | 1.3 | 84871 Lung Cancer | 2.0 |
| U-118-MG | | (ODO4404) | |
| CNS ca. (astro)SF-539 | 0.4 | 84872 Lung NAT (ODO4404) | 13.2 |
| CNS ca. (astro) | 1.0 | 84875 Lung Cancer | 9.8 |
| SNB-75 | | (ODO4565) | |
| CNS ca. (astro) | 4.7 | 85950 Lung Cancer | 4.2 |
| SW1783 | | (ODO4237-01) | |
| CNS ca. (glio) U251 | 0.0 | 85970 Lung NAT | 13.3 |
| | | (ODO4237-02) | |
| CNS ca. (glio) SF-295 | 2.1 | 83255 Ocular Mel Met to | 0.7 |
| | | Liver (ODO4310) | |
| CNS ca. (glio)SNB-19 | 0.0 | 83256 Liver NAT (ODO4310) | 8.7 |
| CNS Ca. | 0.0 | 84139 Melanoma Mets to | 1.2 |
| (glio/astro)U87-MG | | Lung (ODO4321) | |
| CNS ca.* (neuro; met) | 0.1 | 84138 Lung NAT (ODO4321) | 6.0 |
| SK-N-AS | | | |
| Small intestine | 31.4 | Normal Kidney GENPAK | 7.5 |
| | | 061008 | |
| Colorectal | 29.7 | 83786 Kidney Ca, Nuclear | 8.8 |
| | | grade 2 (ODO4338) | |
| Colon ca. HT29 | 0.2 | 83787 Kidney NAT | 16.5 |
| | | (ODO4338) | |
| Colon ca.CaCo-2 | 0.0 | 83788 Kidney Ca Nuclear | 3.9 |
| | | grade ½ (ODO4339) | |
| Colon ca.HCT-15 | 0.4 | 83789 Kidney NAT | 6.9 |
| | | (ODO4339) | |
| Colon ca.HCT-116 | 0.0 | 83790 Kidney Ca, Clear cell | 8.0 |
| | | type (ODO4340) | |
| Colon ca. HCC-2998 | 0.8 | 83791 Kidney NAT | 8.8 |
| | | (ODO4340) | |
| Colon ca. SW480 | 0.3 | 83792 Kidney Ca, Nuclear | 3.9 |
| | | grade 3 (ODO4348) | |
| Colon ca.* (SW480 | 0.0 | 83793 Kidney NAT | 13.3 |
| met)SW620 | | (ODO4348) | |
| Fetal Skeletal | 16.5 | 87474 Kidney Cancer | 5.2 |
| | | (ODO4622-01) | |
| Skeletal muscle | 20.9 | 87475 Kidney NAT | 9.1 |
| | | (ODO4622-03) | |
| Heart | 33.9 | 85973 Kidney Cancer | 4.4 |
| | | (ODO4450-01) | |
| Stomach | 19.8 | 85974 Kidney NAT | 11.3 |
| | | (ODO4450-03) | |
| Gastric ca.* (liver met) | 2.2 | Kidney Cancer Clontech | 2.1 |
| NCl-N87 | | 8120607 | |
| Kidney | 15.8 | Kidney NAT Clontech | 5.0 |
| | | 8120608 | |
| Kidney (fetal) | 8.1 | Kidney Cancer Clontech | 0.1 |
| | | 8120613 | |
| Renal Ca. 786-0 | 3.0 | Kidney NAT Clontech | 3.6 |
| | | 8120614 | |
| Renal ca. A498 | 3.9 | Kidney Cancer Clontech | 6.5 |
| | | 9010320 | |
| Renal ca.ACHN | 97.3 | Kidney NAT Clontech | 5.6 |
| | | 9010321 | |
| Renal ca. TK-10 | 0.4 | Normal Uterus GENPAK | 8.9 |
| | | 061018 | |
| Renal ca. UO-31 | 10.4 | Uterus Cancer GENPAK | 6.1 |
| | | 064011 | |
| Renal ca. RXF 393 | 6.4 | Normal Thyroid Clontech A+ | 2.3 |
| | | 6570-1** | |
| Pancreas | 13.1 | Thyroid Cancer GENPAK | 1.0 |
| | | 064010 | |
| Pancreatic ca. | 0.1 | Thyroid Cancer | 10.2 |
| CAPAN 2 | | INVITROGEN A302152 | |
| Ovary | 23.8 | Thyroid NAT INVITROGEN | 6.5 |
| | | A302153 | |
| Ovarian ca.IGROV-1 | 0.0 | Normal Breast GENPAK | 8.1 |
| | | 061019 | |
| Ovarian ca.OVCAR-3 | 26.6 | 84877 Breast Cancer | 6.0 |
| | | (ODO4566) | |
| Ovarian ca.OVCAR-4 | 1.4 | 85975 Breast Cancer | 8.0 |
| | | (ODO4590-01) | |
| Ovarian ca.OVCAR-5 | 3.4 | 85976 Breast Cancer Mets | 7.2 |
| | | (ODO4590-03) | |
| Ovarian ca.OVCAR-8 | 0.0 | 87070 Breast Cancer | 2.2 |
| | | Metastasis (ODO4655-05) | 2.2 |
| Ovarian ca.* (ascites) | 0.0 | GENPAK Breast Cancer | 19.2 |
| SK-OV-3 | | 064006 | |
| Prostate | 56.3 | Breast Cancer Clontech | 4.0 |
| | | 9100266 | |
| Prostate ca.* (bone | 0.0 | Breast NAT Clontech | 6.6 |
| met)PC-3 | | 9100265 | |
| Plancenta | 66.0 | Breast Cancer INVITROGEN | 4.7 |
| | | A209073 | |
| Pituitary gland | 4.5 | Breast NAT INVITROGEN | 9.0 |
| | | A2090734 | |
| Uterus | 22.4 | Normal Liver GENPAK | 4.6 |
| | | 061009 | |
| | | Liver Cancer GENPAK | 1.1 |
| | | 064003 | |
| | | Liver Cancer Research | 4.5 |
| | | Genetics RNA 1025 | |
| | | Liver Cancer Research | 4.6 |
| | | Genetics RNA 1026 | |
| | | Paired Liver Cancer Tissue | 3.9 |
| | | Research Genetics RNA | |
| | | 6004-T | |
| | | Paired Liver Tissue Research | 3.6 |
| | | Genetics RNA 6004-N | |
| | | Paired Liver Cancer Tissue | 5.4 |
| | | Research Genetics RNA | |
| | | 6005-T | |
| | | Paired Liver Tissue Research | 5.1 |
| | | Genetics RNA 6005-N | |
| | | Normal Bladder GENPAK | 10.4 |
| | | 061001 | |
| | | Bladder Cancer Research | 5.7 |
| | | Genetics RNA 1023 | |
| | | Bladder Cancer | 2.5 |
| | | INVITROGEN A302173 | |
| | | 87071 Bladder Cancer | 4.9 |
| | | (ODO4718-01) | |
| | | 87072 Bladder Normal | 11.4 |
| | | Adjacent (ODO4718-03) | |
| | | Normal Ovary Res. Gen. | 3.8 |
| | | Ovarian Cancer GENPAK | 19.1 |
| | | 064008 | |
| | | 87492 Ovary Cancer | 2.1 |
| | | (ODO4768-07) | |
| | | 87493 Ovary NAT | 23.8 |
| | | (ODO4768-08) | |
| | | Normal Stomach GENPAK | 12.3 |
| | | 061017 | |
| | | NAT Stomach Clontech | 12.2 |
| | | 9060359 | |
| | | Gastric Cancer Clontech | 8.1 |
| | | 9060395 | |
| | | NAT Stomach Clontech | 18.3 |
| | | 9060394 | |
| | | Gastric Cancer Clontech | 7.7 |
| | | 9060397 | |
| | | NAT Stomach Clontech | 8.5 |
| | | 9060396 | |
| | | Gastric Cancer GENPAK | 15.4 |
| | | 064005 | |

The Taqman Results Are Summarized in Table 42.

TABLE 42

| | Internal | |
|---|---|---|
| | Accession | |
| NOYX | Number | Results |
| NOV-1 | 10132038.0.67 | Normal adjacent tissue to colon cancer |
| NOV-2a | 10132038.0.139 | tissue showed a higher expression of the |
| NOV-2b | 10132038.0.136 | gene as compared to colon cancer tissue |
| | | itself. The results also demonstrate a |
| | | similar profile for lung and ocular |
| | | melanoma. |
| NOV-3a | 18552586_EXT1 | High level of expression in brain and |
| NOV-3b | 18552586_EXT2 | moderate expression in lung and trachea, |
| NOV-3c | 18552586_EXT3 | suggesting its potential role in diseases |
| NOV-3d | 18552586_EXT4 | involving these tissues. Increased expres- |
| | | sion in normal colon as compared to colon |
| | | cancer tissue. Cancerous uterus and ovary |
| | | tissues exhibited significantly higher |
| | | expression than their normal counterparts. |
| NOV-4a | 10093872.0.107 | Increased expression in normal bladder |
| NOV-4b | 10093872.1 | and moderate expression in prostate, heart, |
| NOV-4c | 10093872.0.38 | placenta, small intestine, and colorectal |
| NOV-4d | 10093872.2 | cells. Normal adjacent tissue (NAT) of |
| NOV-4e | 10093872.3 | prostate showed maximum expression. |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
a) a mature form of the amino acid sequence selected from the group consisting of SEQ ID NO: 2, 4, 5, 7, 9, 11, 13, 15, 17, 19, 21, or 23;
b) a variant of a mature form of the amino acid sequence selected from the group consisting of SEQ ID NO: 2, 4, 5, 7, 9, 11, 13, 15, 17, 19, 21, or 23, wherein any amino acid in the mature form is changed to a different amino acid, provided that no more than 15% of the amino acid residues in the sequence of the mature form are so changed;
c) the amino acid sequence selected from the group consisting of SEQ ID NO: 2, 4, 5, 7, 9, 11, 13, 15, 17, 19, 21, or 23;
d) a variant of the amino acid sequence selected from the group consisting of SEQ ID NO: 2, 4, 5, 7, 9, 11, 13, 15, 17, 19, 21, or 23 wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO: 2, 4, 5, 7, 9, 11, 13, 15, 17, 19, 21, or 23.

3. The polypeptide of claim 2, wherein the variant is the translation of a single nucleotide polymorphism.

4. The polypeptide of claim 1 that is a variant polypeptide described therein, wherein any amino acid specified in the chosen sequence is changed to provide a conservative substitution.

5. An isolated nucleic acid molecule comprising a nucleic acid sequence encoding a polypeptide comprising an amino acid sequence selected from the group consisting of:
a) a mature form of the amino acid sequence given SEQ ID NO: 2, 4, 5, 7, 9, 11, 13, 15, 17, 19, 21, or23;
b) a variant of a mature form of the amino acid sequence selected from the group consisting of SEQ ID NO: 2, 4, 5, 7, 9, 11, 13, 15, 17, 19, 21, or 23 wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO: 2, 4, 5, 7, 9, 11, 13, 15, 17, 19, 21, or23;
d) a variant of the amino acid sequence selected from the group consisting of SEQ ID NO: 2, 4, 5, 7, 9, 11, 13, 15, 17, 19, 21, or 23, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO: 2, 4, 5, 7, 9, 11, 13, 15, 17, 19, 21, or 23 or any variant of said polypeptide wherein any amino acid of the chosen sequence is changed to a different amino acid, provided that no more than 10% of the amino acid residues in the sequence are so changed; and
f) the complement of any of said nucleic acid molecules.

6. The nucleic acid molecule of claim 5, wherein the nucleic acid molecule comprises the nucleotide sequence of a naturally occurring allelic nucleic acid variant.

7. The nucleic acid molecule of claim 5 that encodes a variant polypeptide, wherein the variant polypeptide has the polypeptide sequence of a naturally occurring polypeptide variant.

8. The nucleic acid molecule of claim 5, wherein the nucleic acid molecule comprises a single nucleotide polymorphism encoding said variant polypeptide.

9. The nucleic acid molecule of claim 5, wherein said nucleic acid molecule comprises a nucleotide sequence selected from the group consisting of
a) the nucleotide sequence selected from the group consisting of SEQ ID NO: 1, 3, 6, 8, 10, 12, 14, 16, 18, 20, 22, or 57;
b) a nucleotide sequence wherein one or more nucleotides in the nucleotide sequence selected from the group consisting of SEQ ID NO: 1, 3, 6, 8, 10, 12, 14, 16, 18, 20, 22, or 57 is changed from that selected from the group consisting of the chosen sequence to a different nucleotide provided that no more than 15% of the nucleotides are so changed;
c) a nucleic acid fragment of the sequence selected from the group consisting of SEQ ID NO: 1, 3, 6, 8, 10, 12, 14, 16, 18, 20, 22, or 57; and
d) a nucleic acid fragment wherein one or more nucleotides in the nucleotide sequence selected from the group consisting of SEQ ID NO: 1, 3, 6, 8, 10, 12, 14, 16, 18, 20, 22, or 57 is changed from that selected from the group consisting of the chosen sequence to a different nucleotide provided that no more than 15% of the nucleotides are so changed.

10. The nucleic acid molecule of claim 5, wherein said nucleic acid molecule hybridizes under stringent conditions to the nucleotide sequence selected from the group consisting of SEQ ID NO: 1, 3, 6, 8, 10, 12, 14, 16, 18, 20, 22, or 57, or a complement of said nucleotide sequence.

11. The nucleic acid molecule of claim 5, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

12. A vector comprising the nucleic acid molecule of claim 11.

13. The vector of claim 12, further comprising a promoter operably linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing said sample;
(b) introducing said sample to a probe that binds to said nucleic acid molecule; and
(c) determining the presence or amount of said probe bound to said nucleic acid molecule, thereby determining the presence or amount of the nucleic acid molecule in said sample.

20. A method of identifying an agent that binds to the polypeptide of claim 1, the method comprising:
(a) introducing said polypeptide to said agent; and
(b) determining whether said agent binds to said polypeptide.

21. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

22. A method for modulating the activity of the polypeptide of claim 1, the method comprising introducing a cell sample expressing the polypeptide of said claim with a compound that binds to said polypeptide in an amount sufficient to modulate the activity of the polypeptide.

23. A method of treating or preventing a pathology associated with the polypeptide of claim 1, said method comprising administering the polypeptide of claim 1 to a subject in which such treatment or prevention is desired in an amount sufficient to treat or prevent said pathology in said subject.

24. The method of claim 23, wherein said subject is a human.

25. A method of treating or preventing a pathology associated with the polypeptide of claim 1, said method comprising administering to a subject in which such treatment or prevention is desired a NOVX nucleic acid in an amount sufficient to treat or prevent said pathology in said subject.

26. The method of claim 25, wherein said subject is a human.

27. A method of treating or preventing a pathology associated with the polypeptide of claim 1, said method comprising administering to a subject in which such treatment or prevention is desired a NOVX antibody in an amount sufficient to treat or prevent said pathology in said subject.

28. The method of claim 27, wherein the subject is a human.

29. A pharmaceutical composition comprising the polypeptide of claim 1 and a pharmaceutically acceptable carrier.

30. A pharmaceutical composition comprising the nucleic acid molecule of claim 5 and a pharmaceutically acceptable carrier.

31. A pharmaceutical composition comprising the antibody of claim 15 and a pharmaceutically acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a pathology associated with the polypeptide of claim 1, wherein said therapeutic is the polypeptide of claim 1.

36. The use of a therapeutic in the manufacture of a medicament for treating a syndrome associated with a human disease, the disease selected from a pathology associated with the polypeptide of claim 1, wherein said therapeutic is a NOVX nucleic acid.

37. The use of a therapeutic in the manufacture of a medicament for treating a syndrome associated with a human disease, the disease selected from a pathology associated with the polypeptide of claim 1, wherein said therapeutic is a NOVX antibody.

38. A method for screening for a modulator of activity or of latency or predisposition to a pathology associated with the polypeptide of claim 1, said method comprising:
a) administering a test compound to a test animal at increased risk for a pathology associated with the polypeptide of claim 1, wherein said test animal recombinantly expresses the polypeptide of claim 1;
b) measuring the activity of said polypeptide in said test animal after administering the compound of step (a); and
c) comparing the activity of said protein in said test animal with the activity of said polypeptide in a control animal not administered said polypeptide, wherein a change in the activity of said polypeptide in said test animal relative to said control animal indicates the test compound is a modulator of latency of, or predisposition to, a pathology associated with the polypeptide of claim 1.

39. The method of claim 38, wherein said test animal is a recombinant test animal that expresses a test protein transgene or expresses said transgene under the control of a promoter at an increased level relative to a wild-type test animal, and wherein said promoter is not the native gene promoter of said transgene.

40. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease, wherein an alteration in the expression level of the polypeptide in the first subject as compared to the control sample indicates the presence of or predisposition to said disease.

41. A method for determining the presence of or predisposition to a disease associated with altered levels of the nucleic acid molecule of claim 5 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

42. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2, 4, 5, 7, 9, 11, 13, 15, 17, 19, 21, or 23 or a biologically active fragment thereof.

43. A method of treating a pathological state in a mammal, the method comprising administering to the mammal the antibody of claim 15 in an amount sufficient to alleviate the pathological state.
